(19) United States
(12) Patent Application Publication
(10) Pub. No.: US 2023/0348895 A1
(43) Pub. Date: Nov. 2, 2023

(54) COMPOSITIONS AND METHODS FOR DETERMINING PROVENANCE

(71) Applicant: PRESIDENT AND FELLOWS OF HARVARD COLLEGE, Cambridge, MA (US)

(72) Inventors: Michael SPRINGER, Cambridge, MA (US); David Z. RUDNER, Cambridge, MA (US)

(73) Assignee: PRESIDENT AND FELLOWS OF HARVARD COLLEGE, Cambridge, MA (US)

(21) Appl. No.: 17/791,319

(22) PCT Filed: Jan. 8, 2021
(86) PCT No.: PCT/US2021/012805

§ 371 Date: Jul. 7, 2022

Related U.S. Application Data
(60) Provisional application No. 62/958,512, filed on Jan. 8, 2020.

(51) Int. Cl. C12N 15/10; C12N 1/20; C12N 1/18; C12N 3/00

(52) U.S. Cl. CPC C12N 15/1065; C12N 1/205; C12N 1/185; C12N 3/00; C12R 2001/075

(57) ABSTRACT

The technology described herein is directed to compositions and methods for determining provenance of an item, a non-limiting example being a food item. In one aspect described herein is an engineered microorganism comprising at least one genetic barcode element, essential gene mutations, and/or germination gene mutations. In another aspect described herein is a method of determining the provenance of an item comprising contacting the item with an engineered microorganism and later detecting the genetic barcode element to determine the provenance of the item. In another aspect, described herein is a method of determining the path of an item or individual across a surface.

### CROSS-REFERENCE TO RELATED APPLICATIONS

This application is a 35 U.S.C. § 371 National Phase Entry Application of International Patent Application No. PCT/US2021/012805 filed on Jan. 8, 2021, which designated the U.S., and which claims benefit under 35 U.S.C. § 119(e) of U.S. Provisional Application No. 62/958,512 filed Jan. 8, 2020, the contents of which are incorporated herein by reference in their entireties.

### GOVERNMENT SUPPORT

This invention was made with Government support under HR0011-18-2-0014 awarded by the Department of Defense/DARPA. The Government has certain rights in the invention.

### SEQUENCE LISTING

The instant application contains a Sequence Listing which has been submitted electronically in ASCII format and is hereby incorporated by reference in its entirety. Said ASCII copy, created on Jan. 27, 2021, is named 002806-095470WOPT_SL.txt and is 142,641 bytes in size.

### TECHNICAL FIELD

The technology described herein relates to compositions and methods for determining the provenance of an item.

### BACKGROUND

Globalization of supply chains has dramatically complicated the process of determining the origin of agricultural products and manufactured goods. Determining the origin of these objects is critical, as in cases of foodborne illness, but current labelling technologies can be prohibitively labor-intensive and are easy to remove or replace (see e.g., Wognum et al., Systems for sustainability and transparency of food supply chains—Current status and challenges, Advanced Engineering Informatics (2011), 25(1), 65-76). Similarly, law enforcement benefits from tools that label unknown persons or objects passing through a location of interest as a complement to fingerprinting and video surveillance (Gooch et al., Taggant materials in forensic science: A review, TrAC Trends in Analytical Chemistry (2016) 83(Part B), 49-54).

Microbial communities offer an alternative to standard approaches of labeling. Any object placed in and interacting with a particular environment gradually adopts the naturally occurring microbes present in that environment (see e.g., Lax et al., Longitudinal analysis of microbial interaction between humans and the indoor environment. Science 345, 1048-1052 (2014)) Jiang et al., Dynamic human environmental exposome revealed by longitudinal personal monitoring. Cell 175, 277-291.e31 (2018)); thus, it has been suggested that the microbial composition of an object could be used to determine object provenance (see e.g., Lax et al., Forensic analysis of the microbiome of phones and shoes. Microbiome 3, 21 (2015)). However, the compositions of microbial communities in different areas are not reliably large or stable enough to uniquely identify specific locations; moreover, using natural microbes requires extensive, expensive, and time-consuming mapping of natural environments.

### SUMMARY

Determining where an object has been or is from is a fundamental challenge for human health, commerce, and food safety. Location-specific microbes offer a cheap and highly sensitive way to determine object provenance. Described herein is a synthetic, scalable microbial spore system that identifies object provenance in under one hour at meter-scale resolution and near single spore sensitivity, which can be safely introduced into and recovered from the environment. This system solves key challenges in object provenance: persistence in the environment, scalability, rapid and facile decoding, and bio-containment. The system can comprise both field-deployable sensors and sequencing-based readouts (e.g., SHERLOCK, a Cas13a RNA-guided nucleic acid detection assay, among others), facilitating its implementation in a wide range of applications (e.g., tracing object trajectories, or identifying the point of origin of the object). The engineered microorganisms exhibit at least the following benefits: 1) they are compatible with growth at industrial scale; 2) they persist in the environment and reliably label objects that pass through it; 3) they are bio-contained and not viable in the wild to prevent adverse ecological effects or cross-contamination; and 4) the encoding and decoding of information regarding object provenance is rapid, sensitive and specific.

The technology described herein is directed to compositions and methods for determining provenance of an item, as a non-limiting example, a food item. In one aspect, described herein is an engineered microorganism comprising one or more barcodes, auxotrophy mutation, and/or germination mutation. In another aspect described herein is a method of determining the provenance of an item comprising contacting the item with an engineered microorganism and later detecting the one or more barcodes to determine the provenance of the item. In another aspect described herein is a method of determining the path of an item or individual across a surface.

In one aspect, described herein is a microorganism engineered to comprise at least one genetic barcode element and at least one of: (a) an inactivating modification of at least one essential gene; or (b) an inactivating modification of at least one germination gene.

In some embodiments of any of the aspects, the microorganism is engineered to comprise a genetic barcode element, an inactivating modification of at least one essential gene, and an inactivating modification of at least one germination gene.

In some embodiments of any of the aspects, the microorganism is engineered to comprise a genetic barcode element and an inactivating modification of at least one essential gene.

In some embodiments of any of the aspects, the microorganism is a yeast or a bacterium.

In some embodiments of any of the aspects, the microorganism is a Saccharomyces yeast or a Bacillus bacterium.

In some embodiments of any of the aspects, the microorganism is Saccharomyces cerevisiae, Bacillus subtilis, or Bacillus thuringiensis.

In some embodiments of any of the aspects, the microorganism is engineered from Saccharomyces cerevisiae strain BY4743, Bacillus subtilis strain 168, or Bacillus thuringiensis strain HD-73.

In some embodiments of any of the aspects, the genetic barcode element comprises: (a) a first primer binding sequence; (b) at least one barcode region; (c) a Cas enzyme scaffold; (d) a transcription initiation site; and (e) a second primer binding sequence.

In some embodiments of any of the aspects, the genetic barcode element comprises: (a) a first primer binding sequence; (b) at least one barcode region; (c) a transcription initiation site; and (d) a second primer binding sequence.

In some embodiments of any of the aspects, the genetic barcode element comprises: (a) a first primer binding sequence; (b) at least one barcode region; and (c) a second primer binding sequence.

In some embodiments of any of the aspects, the microorganism is engineered to comprise first and second barcode regions.

In some embodiments of any of the aspects, the first barcode region indicates that an item on which the microorganism is detected is from one of a group of known sources, and the second barcode region indicates that an item on which the microorganism is detected is from a particular source of said group of sources.

In some embodiments of any of the aspects, the first primer binding sequence and second primer binding sequence comprise sites for binding of PCR or RPA primers.

In some embodiments of any of the aspects, the barcode region comprises 20-40 base pairs.

In some embodiments of any of the aspects, the barcode region comprises a Hamming distance of at least 5 base pairs relative to barcode regions comprised by other items marked with an engineered microorganism as described herein.

In some embodiments of any of the aspects, the barcode region is unique or distinguishable from at least one other barcode region comprised by other items marked with an engineered microorganism as described herein.

In some embodiments of any of the aspects, the Cas enzyme scaffold comprises a scaffold for Cas13.

In some embodiments of any of the aspects, the transcription initiation site comprises a T7 transcription initiation site.

In some embodiments of any of the aspects, the at least one essential gene comprises a conditional essential gene.

In some embodiments of any of the aspects, the at least one conditional essential gene comprises an essential compound synthesis gene.

In some embodiments of any of the aspects, the at least one essential compound synthesis gene comprises an amino acid synthesis gene.

In some embodiments of any of the aspects, the at least one essential compound synthesis gene comprises a nucleotide synthesis gene.

In some embodiments of any of the aspects, the at least one essential compound synthesis gene comprises a synthesis gene for threonine, methionine, tryptophan, phenylalanine, histidine, leucine, lysine, or uracil.

In some embodiments of any of the aspects, the at least one essential compound synthesis gene is selected from the group consisting of thrC, metA, trpC, pheA, HIS3, LEU2, LYS2, MET15, and URA3.

In some embodiments of any of the aspects, an engineered microorganism as described herein comprises an inactivating modification of at least two or more essential compound synthesis genes.

In some embodiments of any of the aspects, the at least one germination gene is selected from the group consisting of cwlJ, sleB, gerAB, gerBB, and gerKB.

In some embodiments of any of the aspects, an engineered microorganism as described herein comprises an inactivating modification of two or more germination genes.

In some embodiments of any of the aspects, the engineered microorganism is inactivated through boiling prior to use.

In another aspect described herein is a method of determining the provenance of an item, the method comprising: (a) contacting an item with at least one engineered microorganism as described herein; (b) isolating nucleic acid from the item; (c) detecting the genetic barcode element of the at least one isolated engineered microorganism; and (d) determining the provenance of the item based on the detected genetic barcode element of the at least one isolated engineered microorganism.

In some embodiments of any of the aspects, the method further comprises inactivating the at least one engineered microorganism prior to step (a).

In some embodiments of any of the aspects, the method further comprises distributing the item in between step (a) and step (b).

In another aspect described herein is a method of determining the provenance of an item, the method comprising: (a) isolating nucleic acid from the item; and (b) detecting the presence of a genetic barcode element, wherein the presence of the genetic barcode element indicates the presence of at least one engineered microorganism comprising a genetic barcode element and an inactivating modification of at least one essential compound synthesis gene or an inactivating modification of at least one germination gene, wherein the presence of the at least one engineered microorganism determines the provenance of the item.

In another aspect described herein is a method of marking the provenance of an item, the method comprising contacting the item with at least one engineered microorganism as described herein.

In some embodiments of any of the aspects, the microorganism comprises first and second barcode regions, wherein the first barcode region indicates that an item on which the microorganism is detected is from one of a group of known sources, and the second barcode region indicates that an item on which the microorganism is detected is from a particular source of said group of sources.

In some embodiments of any of the aspects, the method comprises detecting the presence of the first barcode region in a nucleic acid sample from an item, thereby determining that the item is from a group of known sources.

In some embodiments of any of the aspects, the method further comprises detecting the presence of the second barcode region in the same or different nucleic acid sample from the item, thereby determining that the item is from a particular member of said group of known sources.

In some embodiments of any of the aspects, the item is a food item.

In some embodiments of any of the aspects, the step of detecting the genetic barcode element comprises a method selected from the group consisting of sequencing, hybridization with fluorescent or colorimetric DNA, and SHERLOCK.

In some embodiments of any of the aspects, the sequence of the barcode region of the engineered microorganism is specific for the item or group of items.

In some embodiments of any of the aspects, the sequence of the barcode region of the engineered microorganism is specific for a point of origin of an item or group of items.

In some embodiments of any of the aspects, the step of detecting the genetic barcode element of the isolated nucleic acid comprises: (a) detecting the first barcode region; and (b) if the first barcode region is detected, then detecting the second barcode region; or if the first barcode region is not detected, then determining that no engineered microorganism is present on the item.

In another aspect described herein is a method of determining the path of an item or an individual across a surface, comprising: (a) contacting a surface with at least two engineered microorganisms as described herein; (b) allowing the item or individual to contact the surface in a continuous or discontinuous path; (c) isolating nucleic acid from the item or individual; (d) detecting the genetic barcode elements of the at least two isolated engineered microorganisms; and (e) determining the path of the item or individual across the surface based on the detected genetic barcode element of the at least two isolated engineered microorganisms.

In some embodiments of any of the aspects, the surface comprises sand, soil, carpet, or wood.

In some embodiments of any of the aspects, the surface is divided into a grid comprising grid sections, wherein each grid section comprises at least one engineered microorganism that is distinguishable from all other engineered microorganism on the surface.

In some embodiments of any of the aspects, each grid section comprises at least two distinguishable engineered microorganisms.

In some embodiments of any of the aspects, each grid section comprises at least three distinguishable engineered microorganisms.

In some embodiments of any of the aspects, each grid section comprises at least four distinguishable engineered microorganisms.

In some embodiments of any of the aspects, the item or individual is determined to have contacted a specific grid section if at least one engineered microorganism originating from the specific grid section is detected on the item or individual.

In some embodiments of any of the aspects, the path of the item or individual across the surface comprises the specific grid sections that the item or individual is determined to have contacted.

In some embodiments of any of the aspects, the item or individual is determined to not have contacted a specific grid section if none of the engineered microorganisms originating from the specific grid section are detected on the item or individual.

In some embodiments of any of the aspects, the path of the item or individual across the surface does not comprise the specific grid sections that the item or individual is determined to not have contacted.

### BRIEF DESCRIPTION OF THE DRAWINGS

FIG. 1 is schematic showing an example of an engineered microorganism as described herein.

FIG. 2A-2B are a series of schematics and graphs showing the group barcode+unique barcode design. FIG. 2A is a schematic showing the group barcode design. FIG. 2B is a test showing the group barcode detection followed by universal detection. Dark grey indicates detection. Light grey is background.

FIG. 3A-3D are a series of schematics and graphs showing that Barcoded Microbial Spores can be specifically and sensitively detected. FIG. 3A is a schematic of the Barcoded Microbial Spores (BMS) application and detection pipeline. FIG. 3B is a heatmap showing endpoint fluorescence values from in vitro SHERLOCK reactions of all combinations of 22 barcodes and 22 crRNAs assessing specificity of each barcode-crRNA pair. FIG. 3C is a series of bar graphs showing the detection limit of B. subtilis and S. cerevisiae BMS by SHERLOCK (each of the 8 biological replicates for each spore concentration are shown). Spore numbers are calculated on a per reaction basis. FIG. 3D is a heatmap showing endpoint fluorescence values from in vitro SHERLOCK reactions testing specificity of four barcodes for group 1 crRNA and four barcodes for group 2 crRNA as detected by either unique or group crRNA.

FIG. 4A-4G is a series of graphs and schematics showing the persistence, transferability, and maintenance of BMS. FIG. 4A is a series of line graphs showing that BMS persisted on sand, soil, carpet, and wood over three months in ˜1 m² surfaces performed in incubators. y-axis: B. subtilis BMS number relative to week 1 levels. Error bars represent standard deviations. Perturbation: simulated wind, rain, vacuuming, or sweeping. The top line graph of FIG. 4A shows testing with sand and soil, with the black solid line showing perturbed sand, the black dotted line showing unperturbed sand, the grey solid line showing perturbed soil, and the grey dotted line showing unperturbed soil. The bottom line graph of FIG. 4A shows testing with carpet and wood, with the dark grey solid line showing perturbed wood, the dark grey dotted line showing unperturbed wood, the light grey solid line showing perturbed carpet, and the light grey dotted line showing unperturbed carpet. The top panel of FIG. 4B shows a photograph of a large-scale (˜100 m²) sandpit. The bottom panel of FIG. 4B shows a schematic of the sandpit. B. subtilis BC-24 and BC-25 BMS, and S. cerevisiae BC-49 and BC-50 BMS were inoculated in the shaded dark grey region. The light grey shadow represents the area over which the top two inches of sand from a 1.5 m² area of the inoculation region was redistributed after a large fan fell over (see e.g., FIG. 12). FIG. 4C is a bar graph showing the positive SHERLOCK signal from the 4 BMS from the inoculated region “a” in the large-scale experiment. Dashed line is the threshold for positive calls. BC-19 is negative control. FIG. 4D is a schematic showing that BMS persist at collection point “a” (within the inoculated region) and do not spread to collection points “d” or “e.” The heatmap depicts the number of BMS (out of 4) detected by SHERLOCK at each collection point over 13 weeks. (see e.g., FIG. 13A-13B). FIG. 4E is a series of bar graphs showing that BMS persist on grass in an outdoor environment for at least 5 months. Grass region was inoculated with B. subtilis BC-14 and 15 BMS. Samples from actual BMS-inoculated region, 12 ft, 24 ft and 100 ft away from the inoculated region were tested by SHERLOCK using crRNA 14 and 15 (each of the 5 biological replicates for each grass region are shown). FIG. 4F is a schematic showing BMS were transferred onto shoes by walking in the inoculated region “a” in the in the sandpit, and were detected by SHERLOCK. FIG. 4G is a dot plot showing the abundance of BC-25 BMS on shoes after up to 240 minutes of walking on non-inoculated outdoor areas; y-axis is BMS count based on qPCR standard curve (see e.g., FIG. 15A-15D).

FIG. 5A-5D is series of schematics and graphs showing the determination of object provenance using BMS and field-deployable detection. FIG. 5A is a schematic of experiment design and field-deployable method to determine previous locations of an object. Each region was inoculated with 1, 2, or 4 unique BMS. Arrows indicate the object path through a subset of the regions. SHERLOCK reactions were imaged using a mobile phone camera to photograph the reaction plate through a filter, under portable blue light illumination. The top panels of FIG. 5B and FIG. 5C are each a schematic showing the path of an object overlaid on a reaction plate. The bottom panels of FIG. 5B and FIG. 5C are each a photograph of SHERLOCK reaction plate, overlaid with correct or incorrect calls. The call for each region is denoted by color (check: true positive, white cross: false negative, dark grey cross: false positive). FIG. 5D is a table showing the statistics for SHERLOCK provenance predictions of objects traversing regions inoculated with 1, 2, or 4 unique BMS per region.

FIG. 6A-6D is series of graphs and schematics showing the determination of the provenance of produce using BMS. FIG. 6A is a schematic showing that 18 plants were inoculated with distinct B. subtilis BMS, inoculated once a week (4 times total). FIG. 6B is a series of bar graphs showing detection of BMS on plants and soil after harvesting by SHERLOCK with a group crRNA; y-axis: endpoint fluorescence values. Plants A to S were sprayed with BMS; plant T was not sprayed. (−): negative control without DNA template; (+) positive control from DNA. Dashed line is the threshold for positive calls. FIG. 6C is a schematic showing that leaves from plants inoculated with different BMS were mixed together, SHERLOCK was used to confirm the presence of the BMS, and then Sanger sequencing was used to identify the origin of each leaf. The left panel of FIG. 6D is a bar graph showing that leaves were screened for presence of BMS by SHERLOCK using a group crRNA, y-axis: endpoint fluorescence values. (+): group 2 positive DNA; (−): group 1 DNA; (H₂O): water control. The left panel of FIG. 6D is an image showing that Sanger sequencing identified the plant of origin of the mixed leaves. FIG. 6D discloses SEQ ID NOs: 341, 341, 342, and 342-344, respectively, in order of appearance.

FIG. 7A-7D is a series of schematics and graphs showing screening for cross-reactive crRNA-barcode pairs. FIG. 7A is a schematic of the BMS detection pipeline using SHERLOCK. The schematic also shows that DNA barcode region design (160 bp) with RPA primers. Specific barcodes (28 bp) are “Barcode 1” and “Barcode 2.” The group barcode (22 bp) is “Group 1.” FIG. 7B is a series of bar graphs showing an in vitro DNA barcode and crRNA cross-reactivity assay. Bars depict SHERLOCK signal from reactions using different DNA megamer at equimolar template concentrations, with the n−1 barcode reactions in black, barcode-specific reactions in grey, and H₂O RPA reactions in white. * denotes crRNA with high background or cross-reactivity. (n=3 technical replicates, error bars represent mean+s.e.m.). FIG. 7C is a bar graph showing an in vivo BMS and crRNA cross-reactivity assay. Bars are the SHERLOCK signal from reactions with the n−1 BMS reactions in black and H₂O RPA reactions in white. * denotes crRNA with high background or cross-reactivity (n=3 technical replicates, bars represent mean+s.e.m.). FIG. 7D is a bar graph showing an in vivo BMS and crRNA cross-reactivity assay performed with equimolar spore concentrations. n−1 barcodes reactions in dark grey, barcode specific reactions in light grey, and H₂O RPA reactions in yellow.

FIG. 8A-8F is a series of images showing that germination defective BMS are bio-contained. FIG. 8A is an image showing that B. subtilis Δ9 BMS remain stable and dormant over 4 months when stored in PBS at room temperature. At the indicated time points, spores were analyzed by phase-contrast microscopy. Scale bar indicates 2 μm. FIG. 8B is an image showing that S. cerevisiae BMS remain stable after 10 weeks. Images of BMS at day 0 and week 10 are shown. Asci remained intact and no cell lysis was observed. Scale bar indicates 10 μm. FIG. 8C is an image and table showing that B. subtilis Δ9 BMS are unable to germinate, outgrow, and form colonies on nutrient rich medium. ˜2×10⁹ WT and Δ9 BMS were 10-fold serially diluted in PBS and 10 μL spotted on LB agar. Plates were incubated at 37° C. for 16 hours. At the indicated time points, ˜2×10⁹ Δ9 BMS with the indicated barcodes (BC-1 and BC-2) were plated on LB agar. After 16 h at 37° C. the number of colony-forming units were counted. FIG. 8D is an image showing that S. cerevisiae BMS are unable to germinate, outgrow, and form colonies on nutrient rich medium after boiling. 2.5×10⁶ cells before and after 1 hour of boiling were either serially diluted at 2-fold and spotted on YPD. Note that the dim circles in the boiled sample on the YPD plate are cell debris and do not indicate growth. FIG. 8E is an image showing S. cerevisiae cultured in liquid YPD and incubated at 30° C. overnight. Untreated cells showed substantial growth while boiled BMS showed no growth. FIG. 8F shows representative images of the effect of boiling on S. cerevisiae vegetative cells and BMS. Even after 1 min, the majority of the intact cells were BMS. At 30 min, all the intact cells observed were BMS. Scale bar indicates 10 μm.

FIG. 9A-9B is a series of images and graphs showing changes in microbial composition of non-inoculated and inoculated sand and soil. FIG. 9A is a series of stacked bar graphs showing relative abundance of soil bacterial taxa in samples taken from sand or soil surfaces during a 2-month long experiment (see e.g., FIG. 11A-11G). Relative abundance was measured using 16S metagenomics and classified to the class level. The specific classes are stacked in the same order as the legend. The sampling locations differed in surface material, wet/dry status, and inoculation status. Sand samples had extremely low biomass, and most reads from inoculated samples aligned to Bacilli. In all other samples, Bacilli comprised <0.010% of the library. FIG. 9B is a bar graphs showing weighted UniFrac distance calculations for soil samples paired in different ways in order to independently compare the effects of time, wet/dry status, and inoculation status. Each bar represents the average distance between pairs differing only in the indicated variable.

FIG. 10A-10C is a series of images showing the optimization of B. subtilis BMS lysis. To rapidly assess the efficacy of lysis, Δ9 BMS harboring a cytoplasmic red fluorescent protein (mScarlet) were analyzed by fluorescence and phase-contrast microscopy after treatment. FIG. 10A is an image showing ˜2×10⁶ Δ9 BMS that were resuspended in 50 μL of NaOH at the indicated concentrations and heated for 10 min at 95° C. FIG. 10B is an image showing ˜2×10⁶ Δ9 BMS that were resuspended in 50 μL of 200 mM NaOH and heated at the indicated temperatures for 10 min. FIG. 10C is an image showing ˜2×10⁶ Δ9 BMS that were resuspended in 50 μL of 200 mM NaOH and heated at 95° C. for the indicated amount of time. After treatment, BMS were pelleted, washed and resuspended in PBS. An aliquot was then analyzed by fluorescence and phase-contrast microscopy. Loss of fluorescence correlated with the transition from phase-bright to phase-dark BMS. Scale bar indicates 2 μm.

FIG. 11A-11G is a series of images, schematics, and graphs showing the persistence, transferability and maintenance of BMS. FIG. 11A shows photos and schematic of lab incubator scale experiments and simulated wind, rain, and vacuuming. FIG. 11B is a series of dot plots of real-time qPCR Ct values (left y-axis) and BMS numbers (right y-axis) based on a qPCR standard curve (see e.g., FIG. 15A-15D). Each dot represents a different sampling location each week, grouped by trays in the same treatment group for all 12 weeks. FIG. 11C is a series of line graphs and dot plots showing that BMS persisted on sand, soil, carpet and wood surfaces for at least three months. BMS count number (relative to week 1 values) and qPCR Ct values. FIG. 11D-11G is a series of graphs showing that BMS were transferable for at least three months after inoculation from all four test surfaces. Rubber and wood objects placed on inoculated surfaces were used for testing BMS transferability. Line graphs show relative BMS count numbers, and dot plots show qPCR Ct values. The top line graph of FIG. 11C, both line graphs of FIG. 11D, and both line graphs of FIG. 11F show testing with sand and soil, with the black solid line showing perturbed sand, the black dotted line showing unperturbed sand, the grey solid line showing perturbed soil, and the grey dotted line showing unperturbed soil. The bottom line graph of FIG. 11C, both line graphs of FIG. 11E, and both line graphs of FIG. 11G show testing with carpet and wood, with the dark grey solid line showing perturbed wood, the dark grey dotted line showing unperturbed wood, the light grey solid line showing perturbed carpet, and the light grey dotted line showing unperturbed carpet. Throughout FIG. 11A-11G, “P” indicates “perturbation,” and “NP” indicates “no perturbation.”

FIG. 12 is a schematic showing a catastrophic disturbance. Photographs of the large scale sandpit, with the disturbance area from the fan falling over indicated in the shaded light grey area.

FIG. 13A-13B is a series of graphs showing the persistence of BMS over time. FIG. 13A is a series of dot plots showing that SHERLOCK detected BC-24, 25, 49, 50 BMS from samples taken immediately after BMS inoculation (i.e., time 0 from BMS inoculation region); the graphs show fluorescence time courses. BC-19, H₂O, and (−) RPA were negative controls. FIG. 13B is a series of plots showing that SHERLOCK detection indicated BMS persistence over three months with or without perturbation. SHERLOCK time course data (y-axis: fluorescence, x-axis: time in minutes) is shown for each barcode at each of 20 collection spots (a-e, 1-4 in FIG. 4B) for 13 weeks. Signal above the threshold were counted and scored in FIG. 4D. The Cas13a batch was changed at week 6, which changed baseline activity, but did not change conclusions of the experiment.

FIG. 14A-14B is a series of graphs showing the transfer of BMS overtime. SHERLOCK can detect BMS on shoes (see e.g., FIG. 14A) and wood (see e.g., FIG. 14B) that come in contact with an inoculated surface. SHERLOCK time course data (y-axis: fluorescence, x-axis: time in minutes) for transfer onto shoes or wood is shown for each barcode (colors), for two replicates from two different inoculated locations (a1 and a3 in FIG. 4B) over 13 weeks. BC-19 is a negative control. The Cas13a batch was changed at week 6, which changed baseline activity, but did not change conclusions of the experiment.

FIG. 15A-15D is a series of graphs showing the persistence of BMS on object after transfer. FIG. 15A is a series of bar graphs showing SHERLOCK fluorescence and FIG. 15B is a series of bar graphs showing qPCR. FIG. 15A-15B show that BC-24 and BC-25 B. subtilis BMS and BC-49 and BC-50 S. cerevisiae BMS were retained on the shoe after up to 4 hours of walking on non-inoculated areas. FIG. 15C are dot plots showing standard curves constructed from known BMS quantities using either PowerSoil™ or NaOH lysis methods. FIG. 15D is a series of dot plots estimating the number of BMS on each shoe from Ct values using the qPCR standard curve.

FIG. 16A-16C is a series of schematics and graphs showing the retransfer of BMS to non-inoculated surfaces. FIG. 16A is a schematic showing that Sandbox A was inoculated with a 4 BMS mixture. Shoe A stepped in inoculated sandbox A and subsequently stepped into 3 clean sandboxes B, C, and D. Sand from sandboxes B, C, and D after shoe A stepping was sampled and qPCR was performed using BMS-specific primers to quantify BMS. New shoes B, C, and D were stepped in sandboxes B, C, and D, respectively after shoe A had stepped in each sandbox. Shoes B, C, and D were then sampled for BMS. FIG. 16B is a dot plot showing the qPCR results of BMS in sand samples from sandboxes A, B, C, and D. (−): non-inoculated sand; H₂O: qPCR water negative control; the horizontal dashed line is the threshold of detection. FIG. 16C is a dot plot showing the qPCR results of BMS from swabbed shoes A, B, C, and D. (−): non-inoculated sand; H₂O: qPCR water negative control; the horizontal dashed line is the threshold of detection.

FIG. 17A-17E is a series of schematics, images, and graphs showing object provenance using 4 unique BMS per region. FIG. 17A is an image showing the layout used to test the field-deployable detection system: a portable light source and an orange acrylic filter were used for imaging SHERLOCK signals. A mobile phone (not shown) was used for photographing the SHERLOCK reaction plates. FIG. 17B is a schematic showing that four trays were filled with sand, and inoculated by spraying either 4 unique BMS or H₂O. FIG. 17C shows SHERLOCK reaction plate images for six shoe samples that had stepped in one of the 4 trays. Reaction signal matches expectation shown on left. FIG. 17D-17E is a series of schematics and images showing a subsequent experiment, in which 12 sand regions (squares: a-l) were inoculated with 4 unique BMS each. 15 shoe samples took different paths through the regions and were tested for all possible BMS using SHERLOCK. Endpoint fluorescence values are plotted, with expected positives to the left of the vertical dashed line and expected negatives to the right of the vertical dashed line. The call for each lettered region is denoted as indicated (shown to the left of the vertical dashed line, dark grey: true positive, and light grey: false negative; shown to the right of the vertical dashed line, medium grey: false positive, and white: true negative); the horizontal dashed line is the threshold for positive calls. FIG. 17D shows results for Objects 1 and 2. FIG. 17E shows results for Objects 3-15.

FIG. 18A-18D is a series of schematics, images, and graphs showing object provenance using 2 unique BMS per region. FIG. 18A is a schematic showing that a grid of 24 regions was laid out on a clean area of sand, and each region was inoculated with 2 unique BMS. FIG. 18B is a series of top schematics, showing the path of object overlaid on reaction plate, and bottom images, showing a photograph of SHERLOCK reaction plate, overlaid with correct or incorrect calls. The call for each region is indicated in the corresponding area of the plate (check: true positive; white cross: false negative; dark grey cross: false positive). FIG. 18C-18D is a series of schematics and images showing a subsequent experiment, in which 18 trays of sand (squares: a-r) were inoculated with 2 unique barcodes each. 16 shoe samples that took different paths through the regions were tested using SHERLOCK. Endpoint fluorescence values are plotted, with expected positives to the left of the vertical dashed line and expected negatives to the right of the vertical dashed line. The call for each lettered region is denoted as indicated (shown to the left of the vertical dashed line, dark grey: true positive, and light grey: false negative; shown to the right of the vertical dashed line, medium grey: false positive, and white: true negative); the horizontal dashed line is the threshold for positive calls. FIG. 18C shows results for Objects 1 and 2. FIG. 18D shows results for Objects 3-16.

FIG. 19 is a series of images, schematics, and graphs showing object provenance using 1 unique BMS per region. A grid of 20 regions (squares: a-t) were laid out on clean sand, soil, carpet, and wood surfaces, and each region was inoculated with a single unique BMS. 8 remote control car samples, and 24 shoe samples that took different paths through the regions on different surfaces were tested using SHERLOCK. Endpoint fluorescence values are plotted, with expected positives to the left of the vertical dashed line and expected negatives to the right of the vertical dashed line. The call for each lettered region is denoted by as indicated (shown to the left of the vertical dashed line, dark grey: true positive, and light grey: false negative; shown to the right of the vertical dashed line, medium grey: false positive, and white: true negative); the horizontal dashed line is the threshold for positive calls.

FIG. 20 is a table showing statistics for determining provenance with different numbers of BMS. The experiments in FIGS. 17-19 and FIG. 5A-5D were collated together, and the false positive and false negative rates were calculated using the listed threshold criteria for a positive call. Grey highlighting indicates a potential usage.

FIG. 21A-21D is a series of images and schematics showing detection of BMS from plants grown in laboratory farm. FIG. 21A shows photographs of produce at the time of first inoculation (1 month after seed planting) and before harvest (2 months). FIG. 21B shows a photograph of SHERLOCK reaction plate from FIG. 6B detecting BMS on leaf and soil samples from laboratory farm. Plant T was not inoculated. (−): negative control without DNA template; (+) positive control from DNA. FIG. 21C is a schematic showing that plant samples E & G have variant group barcode sequences. FIG. 21C discloses SEQ ID NOs: 345-347, respectively, in order of appearance. FIG. 21D is a schematic showing an alignment of Sanger sequenced leaf and soil samples to the BMS inoculated on plants reference sequence. FIG. 21D discloses SEQ ID NOs: 20-21, 20-21, 20-21, 19, 14, 19, 14, 19, 14, 18, 13, 18, 13, 18, 13, 17, 12, 17, 12, 17, 12, 15, 11, 15, 11, 15, 11, 10, 22, 10, 22, 10, 22, 9, 24, 9, 24, 9, 24, 7, 25, 7, 25, 7, 25, 5-6, 5-6, and 5-6, respectively, in order of appearance.

FIG. 22A-22D is a series of graphs showing that BMS remain on plant leaves and can determine leaf provenance. FIG. 22A is a dot plot showing qPCR measurement of different BMS-inoculated leaves that had been mixed with each other, as shown in FIG. 6C. Black (left-most data set of each time point): different BMS-inoculated leaves that were mixed; qPCR signal of the specific BMS inoculated on the sampled leaf (leaf numbers, n=7 for week 1, n=3 for weeks 4 and 6). Dark grey (middle data set of each time point): different BMS-inoculated leaves that were mixed. qPCR signal from a different BMS than that of the sampled leaf (leaf numbers, n=10 for week 1, n=12 for weeks 4 and 6). Light grey (right-most data set of each time point): qPCR signal of non-mixed and non-inoculated (leaf numbers, n=3 for week 1, 4 and 6). FIG. 22B is a dot plot showing qPCR measurement of BMS from swabbing directly the inoculated plant, swabbing the gloves after the glove touched the inoculated plant, or DNA extractions of different BMS sprayed cabbage and spinach. (−): non-BMS sprayed plant is the negative control. FIG. 22C is a bar graph showing SHERLOCK endpoint fluorescence values for BMS-inoculated leaves after mixing. Detection reactions were performed for multiple BMS for each leaf, though each leaf had been inoculated with a single BMS. FIG. 22D is a schematic showing Sanger sequencing alignment identifies the barcode that had been inoculated (as indicated by dotted lines in FIG. 22C). Non-inoculated leaves that had been mixed with inoculated leaves had positive SHERLOCK signal, but did not specifically align to any of the barcode references. FIG. 22D discloses SEQ ID NOs: 341, 341-342, 342-344, 348, 348-349, 349-350, and 350-351, respectively, in order of appearance.

FIG. 23A-23C is a series of images and tables showing detection of Bacillus thuringiensis (Bt) from produce of known history. FIG. 23A shows PCR gel images from Bt genomic DNA, a pool of genomic DNA from other microbes, and genomic DNA extracted from soil. (−): no template negative control. FIG. 23B is a table showing a summary of PCR detection results for produce known to be inoculated or non-inoculated with Bt, and produce purchased from a store with a priori unknown Bt status (see e.g., FIG. 25A-25C). FIG. 23C shows PCR gel images from produce of known Bt status, watercress, and romaine lettuce found to have Bt. Positive control: Bt genomic DNA; negative control: H20.

FIG. 24A-24C is a series of images, schematics, and tables showing detection of Bacillus thuringiensis from produce bought at a market. FIG. 24A shows PCR gel images of store-bought produce samples. Grey box: Bt Cry1A band; positive control: Bt spores; negative control: H₂O. FIG. 24B is a schematic showing Sanger sequencing of PCR-amplified products from 6 produce aligned to Bt cry1A sequences. Note that the variable region (in grey box) reveals the existence of 3 Bt variants confirming that Bt detection could not solely be attributed to potential cross contamination in the lab. FIG. 24B discloses SEQ ID NOs: 352-358, respectively, in order of appearance. FIG. 24C is a table showing a summary of produce type, origin, and Bt status based on PCR results in B. (−): undetected Bt; (+): detected Bt.

FIG. 25A-25C is a series of graphs showing B. subtilis BMS and Bacillus thuringiensis retention after treatments. FIG. 25A is a dot plot showing BMS retention measured by qPCR, normalized to no-treatment. BMS inoculated by spraying on plant samples were retained after a brief rinsing, 1-hour washing, sonicating or boiling (see e.g., Methods). (−): H₂O negative control. FIG. 25B shows a standard curve of qPCR with Bt gDNA. The estimated equivalent number of spores was calculated by dividing the amount of gDNA in the qPCR reactions by 6.5 fg, the approximate amount of gDNA in a single Bt spore. FIG. 25C is a dot plot showing results (e.g., qPCR results) from Romaine lettuce and watercress, both positive for Bt, that were washed, boiled, fried, or microwaved. Bt remained detectable after these treatments. (n=2 technical replicates, n=4 biological replicates for each treatment, n=2 biological replicates for non-treated control samples).

FIG. 26A-26C is a series of schematics showing template mutagenesis, for example of the primer binding region of a genetic barcode element. FIG. 26A is a schematic showing an exemplary input library and, then after RPA amplification, an output library. The input library comprises genetic barcode elements, each genetic barcode element comprising: a first primer binding region (e.g., comprising variations in the 4 nucleotides at the 3′ end, resulting in a one bp mismatch with the corresponding primer); a unique 7-mer barcode; and a constant second primer binding region. After RPA amplification, the proportion of genetic barcode elements that are successfully amplified is detected in the output library. FIG. 26B is a table, graph, and schematic showing the proportion of different variations in the first primer binding region in the input and output libraries. Note that the two nucleotides at the 3′-most end of the primer binding region, when mismatched with the primer, displayed a significant reduction in abundance between the input library and post RPA (i.e., output) library. FIG. 26C is a schematic showing a Miseq™ library check of the input library (˜74K reads) and output library (˜−139K reads).

FIG. 27 shows an exemplary schematic of a system as described herein.

FIG. 28A-28C is a series of images showing detection of Bacillus thuringiensis from controls. FIG. 28A is a table showing that positive controls of Bt (i.e. produce sprayed with Bt during growth) all tested positive and negative controls (i.e. plants from personal gardens or other plants known not to be sprayed with Bt) all tested negative with PCR. FIG. 28B shows PCR gel images of Bt positive and negative control samples from FIG. 28A. Grey box indicates the band that corresponds to Bt Cry1A. Bt gDNA was used as a positive control template (+); water was used as negative control template (−). FIG. 28C is a gel image showing specificity of PCR-based Bt detection. Different template samples including Bt gDNA, a DNA pool of gDNA from non-Bt microbes (Streptomyces hygroscopicus, S. cerevisiae, B. subtilis, E. coli and Pseudomonas), a soil sample (soil) or water (−) were subjected to PCR-based Bt detection.

FIG. 29 is a series of PCR gel images showing detection of Bacillus thuringiensis from produce bought at a market. Grey box indicates the band that corresponds to Bt Cry1A. Samples 10 and 13 to 17 were negative control samples. The summary of Bt detection from produce bought from stores is shown in Table 1. Note that non-specific bands did not affect Sanger sequencing (data not shown); produce known not to contain Bt yielded no priming when the PCR products were sent to Sanger sequencing. Water was used as negative control (−) for PCR. An alignment of PCR-amplified products from 6 produce samples is in FIG. 24B, showing that the PCR products were genuine Bt cry1A sequences. BLAST results found exclusively Bt in the top 100 hits.

FIG. 30A-30D is a series of images and graphs showing detection of Bacillus subtilis from plants grown in laboratory farm. FIG. 30A is a series of bar graphs showing quantification of the SHERLOCK signal from reaction performed on leaf and soil samples using a group crRNA. Note: Plant T was not sprayed. FIG. 30B shows PCR gel images of BMS from leaf samples from plants grown in the lab farm. Grey box indicates the band that corresponds to Bt Cry1A. FIG. 30C shows PCR gel images of BMS from the soil samples from the pots in the lab farm. FIG. 30D is a bar graph showing BMS retention measured by qPCR, normalized to no-treatment. BMS sprayed on leaf samples were retained after rinsing, washing, sonication or boiling (see e.g., Methods). Water was used as negative control (−). n.d.=not detected.

FIG. 31 is a schematic showing the HD73_5011 locus in B. thuringiensis HD-73.

FIG. 32 is a schematic showing the genome of B. thuringiensis HD-73 and the location of the HD73_5011 locus.

FIG. 33A-33B is a series of schematics showing design of plasmid to transform B. thuringiensis HD-73. FIG. 33A is a schematic showing generation of flanking regions of HD73_5011 by PCR. FIG. 33B is a schematic showing Gibson assembly of the vector.

FIG. 34 is a schematic showing a modified pMiniMAD plasmid.

FIG. 35 is an image showing isolated Bt colonies after transformation.

FIG. 36 is an image showing molecular corroboration of the barcoded Bt strain.

FIG. 36 is a pair of images showing the Bt barcoded spores before and after spore purification.

### DETAILED DESCRIPTION

Embodiments of the technology described herein are include engineered strains of Bacillus (e.g., Bacillus subtilis, Bacillus thuringiensis) and Saccharomyces cerevisiae that are safe for environmental release and contain a sequence that allows for rapid tracking and identification. Also described herein are methods of using such engineered strains to determine the provenance of an item (e.g., a food item).

### Engineered Microorganism

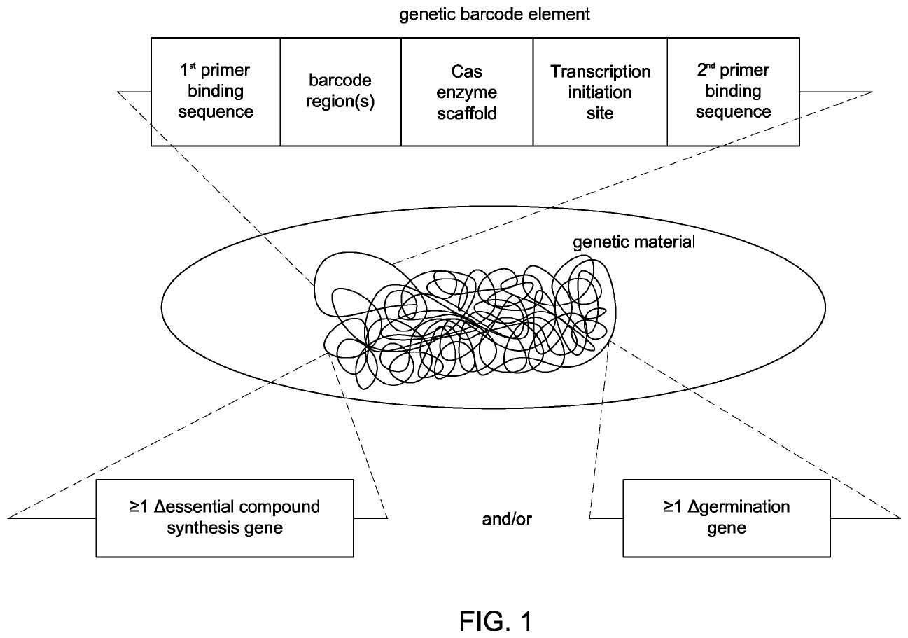

In one aspect of any of the embodiments, described herein is an engineered microorganism. In one aspect of any of the embodiments, the engineered microorganism comprises at least one genetic barcode element and at least one inactivating modification of at least one essential compound synthesis gene and/or at least one inactivating modification of at least one germination gene (see e.g., FIG. 1). As used herein “inactivating modification” refers to a mutation, including an insertion, deletion, or substitution that decreases or eliminates the expression and/or activity of the gene product. In some embodiments of any of the aspects, the inactivating modification is achieved using a Cre-Lox deletion system as known in the art.

In one aspect of any of the embodiments, the engineered microorganism comprises a genetic barcode element and at least one inactivating modification of at least one essential compound synthesis gene. In one aspect of any of the embodiments, the engineered microorganism comprises a genetic barcode element and at least one inactivating modification of at least one germination gene. In one aspect of any of the embodiments, the engineered microorganism comprises a genetic barcode element, at least one inactivating modification of at least one essential compound synthesis gene, and at least one inactivating modification of at least one germination gene.

In some embodiments of any of the aspects, the engineered microorganism is a yeast or a bacterium. In some embodiments of any of the aspects, the microorganism is a Saccharomyces yeast or a Bacillus bacterium. In some embodiments of any of the aspects, the microorganism is Saccharomyces cerevisiae, Bacillus subtilis, or Bacillus thuringiensis. In some embodiments of any of the aspects, the microorganism is naturally non-pathogenic (i.e., non-disease-causing) or, in the case of pathogenic species, engineered to be non-pathogenic through inactivating modifications of pathogen-associated genes.

In some embodiments of any of the aspects, the microorganism is selected from the group consisting of Saccharomyces arboricolus, Saccharomyces bayanus, Saccharomyces boulardii, Saccharomyces bulderi, Saccharomyces cariocanus, Saccharomyces cariocus, Saccharomyces cerevisiae, Saccharomyces chevalieri, Saccharomyces dairenensis, Saccharomyces ellipsoideus, Saccharomyces eubayanus, Saccharomyces exiguous, Saccharomyces florentinus, Saccharomyces fragilis, Saccharomyces kluyveri, Saccharomyces kudriavzevii, Saccharomyces martiniae, Saccharomyces mikatae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, Saccharomyces zonatus, Bacillus acidiceler, Bacillus acidicola, Bacillus acidiproducens, Bacillus acidocaldarius, Bacillus acidoterrestris, Bacillus aeolius, Bacillus aerius, Bacillus aerophilus, Bacillus agaradhaerens, Bacillus agri, Bacillus aidingensis, Bacillus akibai, Bacillus alcalophilus, Bacillus algicola, Bacillus alginolyticus, Bacillus alkalidiazotrophicus, Bacillus alkalinitrilicus, Bacillus alkalisediminis, Bacillus alkalitelluris, Bacillus altitudinis, Bacillus alveayuensis, non-pathogenic Bacillus alvei, Bacillus amyloliquefaciens, Bacillus a. subsp. amyloliquefaciens, Bacillus a. subsp. plantarum, Bacillus aminovorans, Bacillus amylolyticus, Bacillus andreesenii, Bacillus aneurinilyticus, non-pathogenic Bacillus anthracis, Bacillus aquimaris, Bacillus arenosi, Bacillus arseniciselenatis, Bacillus arsenicus, Bacillus aurantiacus, Bacillus arvi, Bacillus aryabhattai, Bacillus asahii, Bacillus atrophaeus, Bacillus axarquiensis, Bacillus azotofixans, Bacillus azotoformans, Bacillus badius, Bacillus barbaricus, Bacillus bataviensis, Bacillus beijingensis, Bacillus benzoevorans, Bacillus beringensis, Bacillus berkeleyi, Bacillus beveridgei, Bacillus bogoriensis, Bacillus boroniphilus, Bacillus borstelensis, Bacillus brevis Migula, Bacillus butanolivorans, Bacillus canaveralius, Bacillus carboniphilus, Bacillus cecembensis, Bacillus cellulosilyticus, Bacillus centrosporus, non-pathogenic Bacillus cereus, Bacillus chagannorensis, Bacillus chitinolyticus, Bacillus chondroitinus, Bacillus choshinensis, Bacillus chungangensis, Bacillus cibi, Bacillus circulans, Bacillus clarkii, Bacillus clausii, Bacillus coagulans, Bacillus coahuilensis, Bacillus cohnii, Bacillus composti, Bacillus curdlanolyticus, Bacillus cycloheptanicus, Bacillus cytotoxicus, Bacillus daliensis, Bacillus decisifrondis, Bacillus decolorationis, Bacillus deserti, Bacillus dipsosauri, Bacillus drentensis, Bacillus edaphicus, Bacillus ehimensis, Bacillus eiseniae, Bacillus enclensis, Bacillus endophyticus, Bacillus endoradicis, Bacillus farraginis, Bacillus fastidiosus, Bacillus fengqiuensis, Bacillus firmus, Bacillus flexus, Bacillus foraminis, Bacillus fordii, Bacillus formosus, Bacillus fortis, Bacillus fumarioli, Bacillus funiculus, Bacillus fusiformis, Bacillus galactophilus, Bacillus galactosidilyticus, Bacillus galliciensis, Bacillus gelatini, Bacillus gibsonii, Bacillus ginsengi, Bacillus ginsengihumi, Bacillus ginsengisoli, Bacillus glucanolyticus, Bacillus gordonae, Bacillus gottheilii, Bacillus graminis, Bacillus halmapalus, Bacillus haloalkaliphilus, Bacillus halochares, Bacillus halodenitrificans, Bacillus halodurans, Bacillus halophilus, Bacillus halosaccharovorans, Bacillus hemicellulosilyticus, Bacillus hemicentroti, Bacillus herbersteinensis, Bacillus horikoshii, Bacillus horneckiae, Bacillus horti, Bacillus huizhouensis, Bacillus humi, Bacillus hwajinpoensis, Bacillus idriensis, Bacillus indicus, Bacillus infantis, Bacillus infernus, Bacillus insolitus, Bacillus invictae, Bacillus iranensis, Bacillus isabeliae, Bacillus isronensis, Bacillus jeotgali, Bacillus kaustophilus, Bacillus kobensis, Bacillus kochii, Bacillus kokeshiiformis, Bacillus koreensis, Bacillus korlensis, Bacillus kribbensis, Bacillus krulwichiae, Bacillus laevolacticus, Bacillus larvae, non-pathogenic Bacillus laterosporus, Bacillus lautus, Bacillus lehensis, Bacillus lentimorbus, Bacillus lentus, non-pathogenic Bacillus licheniformis, Bacillus ligniniphilus, Bacillus litoralis, Bacillus locisalis, Bacillus luciferensis, Bacillus luteolus, Bacillus luteus, Bacillus macauensis, Bacillus macerans, Bacillus macquariensis, Bacillus macyae, Bacillus malacitensis, Bacillus mannanilyticus, Bacillus marisflavi, Bacillus marismortui, Bacillus marmarensis, Bacillus massiliensis, non-pathogenic Bacillus megaterium, Bacillus mesonae, Bacillus methanolicus, Bacillus methylotrophicus, Bacillus migulanus, Bacillus mojavensis, Bacillus mucilaginosus, Bacillus muralis, Bacillus murimartini, Bacillus mycoides, Bacillus naganoensis, Bacillus nanhaiensis, Bacillus nanhaiisediminis, Bacillus nealsonii, Bacillus neidei, Bacillus neizhouensis, Bacillus niabensis, Bacillus niacini, Bacillus novalis, Bacillus oceanisediminis, Bacillus odysseyi, Bacillus okhensis, Bacillus okuhidensis, Bacillus oleronius, Bacillus oryzaecorticis, Bacillus oshimensis, Bacillus pabuli, Bacillus pakistanensis, Bacillus pallidus, Bacillus pallidus, Bacillus panacisoli, Bacillus panaciterrae, Bacillus pantothenticus, Bacillus parabrevis, Bacillus paraflexus, Bacillus pasteurii, Bacillus patagoniensis, Bacillus peoriae, Bacillus persepolensis, Bacillus persicus, Bacillus pervagus, Bacillus plakortidis, Bacillus pocheonensis, Bacillus polygoni, Bacillus polymyxa, Bacillus popilliae, Bacillus pseudalcalophilus, Bacillus pseudofirmus, Bacillus pseudomycoides, Bacillus psychrodurans, Bacillus psychrophilus, Bacillus psychrosaccharolyticus, Bacillus psychrotolerans, Bacillus pulvifaciens, non-pathogenic Bacillus pumilus, Bacillus purgationiresistens, Bacillus pycnus, Bacillus qingdaonensis, Bacillus qingshengii, Bacillus reuszeri, Bacillus rhizosphaerae, Bacillus rigui, Bacillus ruris, Bacillus safensis, Bacillus salarius, Bacillus salexigens, Bacillus saliphilus, Bacillus schlegelii, Bacillus sediminis, Bacillus selenatarsenatis, Bacillus selenitireducens, Bacillus seohaeanensis, Bacillus shacheensis, Bacillus shackletonii, Bacillus siamensis, Bacillus silvestris, Bacillus simplex, Bacillus siralis, Bacillus smithii, Bacillus soli, Bacillus solimangrovi, Bacillus solisalsi, Bacillus songklensis, Bacillus sonorensis, non-pathogenic Bacillus sphaericus, Bacillus sporothermodurans, Bacillus stearothermophilus, Bacillus stratosphericus, Bacillus subterraneus, non-pathogenic Bacillus subtilis, Bacillus s. subsp. inaquosorum, Bacillus s. subsp. spizizenii, Bacillus s. subsp. subtilis, Bacillus taeanensis, Bacillus tequilensis, Bacillus thermantarcticus, Bacillus thermoaerophilus, Bacillus thermoamylovorans, Bacillus thermocatenulatus, Bacillus thermocloacae, Bacillus thermocopriae, Bacillus thermodenitrificans, Bacillus thermoglucosidasius, Bacillus thermolactis, Bacillus thermoleovorans, Bacillus thermophilus, Bacillus thermoruber, Bacillus thermosphaericus, Bacillus thiaminolyticus, Bacillus thioparans, Bacillus thuringiensis, Bacillus tianshenii, Bacillus trypoxylicola, Bacillus tusciae, Bacillus validus, Bacillus vallismortis, Bacillus vedderi, Bacillus velezensis, Bacillus vietnamensis, Bacillus vireti, Bacillus vulcani, Bacillus wakoensis, Bacillus xiamenensis, Bacillus xiaoxiensis, and Bacillus zhanjiangensis.

In some embodiments of any of the aspects, the engineered microorganism is engineered from a sporulating (e.g., spore-forming, endospore-forming) microorganism. Non-limiting examples of sporulating microorganisms include a species from a genus selected from the group consisting of Acetonema, Actinomyces, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridiisalibacter, Cohnella, Coxiella (i.e. Coxiella burnetii), Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halobacillus, Halonatronum, Heliobacterium, Heliophilum, Laceyella, Lentibacillus, Lysinibacillus, Mahella, Metabacterium, Moorella, Natroniella, Oceanobacillus, Orenia, Ornithinibacillus, Oxalophagus, Oxobacter, Paenibacillus, Paraliobacillus, Pelospora, Pelotomaculum, Piscibacillus, Planiflum, Pontibacillus, Propionispora, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribacillus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, and Vulcanobacillus.

In some embodiments of any of the aspects, the microorganism is engineered from Saccharomyces cerevisiae strain BY4743. In some embodiments of any of the aspects, the microorganism is engineered from Saccharomyces cerevisiae strain BY4741 or BY4742. Saccharomyces cerevisiae strain BY4743 is diploid and has the genotype MATa/α his3 Δ1/his3 Δ1 leu2 Δ0/leu2 Δ0 LYS2/lys2 Δ0 met15 Δ0/MET15 ura3 Δ0/ura3 Δ0. BY4741 (genotype: MATa his3 Δ1 leu2 Δ0 met15 Δ0 ura3 Δ0) and BY4742 (genotype: MATα his3 Δ1 leu2 Δ0 lys2 Δ0 ura3 Δ0) are haploid. BY4741-BY4743 are part of a set of deletion strains derived from S288C in which selectable marker genes were deleted by design in order to minimize or eliminate homology to the corresponding marker genes in commonly used vectors without significantly affecting adjacent gene expression. The yeast strains were all directly descended from FY2, which is itself a direct descendant of S288C. Nucleotide variation between BY4741-BY4743 and S288C is minimal (see e.g., NCBI:txid1266529; see e.g., Saccharomyces cerevisiae strains BY4741-4742, whole genome shotgun sequencing project, GenBank: JRIS00000000.1; see e.g., the reference genome for Saccharomyces cerevisiae S288C chromosomes I-XVI and MT: NCBI Reference Sequences: NC_001133.9, NC_001134.8, NC_001135.5, NC 001136.10, NC_001137.3, NC 001138.5, NC_001139.9, NC_001140.6, NC_001141.2, NC 001142.9, NC_001143.9, NC 001144.5, NC 001145.3, NC_001146.8, NC_001147.6, NC_001148.4, NC_001224.1). See e.g., Harsh et al., FEMS Yeast Res. 2010 February; 10(1):72-82, the content of which is incorporated herein by reference in its entirety.

In some embodiments of any of the aspects, the microorganism is engineered from Bacillus subtilis strain 168 (see e.g., Bacillus subtilis subsp. subtilis str. 168 complete genome, NCBI Reference Sequence: NC_000964.3; see e.g., NCBI taxid:224308). In some embodiments of any of the aspects, the microorganism is engineered from Bacillus thuringiensis strain HD-73 or Bacillus thuringiensis subsp. kurstaki strain HD73 (see e.g., NCBI taxid:29339; see e.g., GenBank accession numbers: CP004069 (chromosome) or NC_020238.1 (chromosome), CP004070 (pHT73), CP004071 (pHT77), CP004073 (pHT11), CP004074 (pHT8_1), CP004075 (pHT8_2), and CP004076 (pHT7); see e.g., Liu et al., Genome Announc. 2013 March-April; 1(2): e00080-13, the content of which is incorporated herein by reference in its entirety).

In some embodiments of any of the aspects, the engineered microorganism does not comprise a gene conferring antibiotic resistance. Non-limiting examples of common antibiotics for which resistance genes are used in genetic manipulation include ampicillin, kanamycin, geneticin, erythromycin, triclosan, and/or chloramphenicol. For uses involving release into the wild or applications where human or agricultural or companion animal contact is inherent or likely (e.g., for food applications), it is preferred that an engineered microorganisms carry no known antibiotic resistance genes. In some embodiments of any of the aspects, the engineered microorganism does not comprise a beta-lactamase gene, a kanamycin resistance (KanR) gene, an erythromycin resistance (ErmR) gene, a G418 (geneticin) resistance gene, a Neo gene (e.g., neomycin and/or kanamycin resistance cassette, e.g., from Tn5), or a mutant FabI gene.

In some embodiments of any of the aspects, prior to contacting an item or surface with an engineered microorganism as described herein, the engineered microorganism (e.g., an engineered S. cerevisiae) is inactivated (e.g., killed) through heating, e.g., in an aqueous solution. As a non-limiting example, the engineered microorganism is heated in an aqueous solution (e.g., boiled) for at least one hour. As another non-limiting example, the engineered microorganism is exposed to a temperature of at least 100° C., at least 101° C., at least 102° C., at least 103° C., at least 104° C., at least 105° C., at least 110° C., at least 125° C., or at least 150° C. for at least 1 hour. As another non-limiting example, the engineered microorganism is exposed to an aqueous solution of at least 100° C. for at least 1 minute, at least 5 minutes, at least 10 minutes, at least 20 minutes, at least 30 minutes, at least 40 minutes, at least 50 minutes, at least 1 hour, at least 1.5 hours, or at least 2 hours.

### Genetic Barcode Element

In some embodiments of any of the aspects, an engineered microorganism comprises a genetic barcode element, also referred to herein as a “Unique Tracking Sequence” or UTS. As used herein, the term “genetic barcode element” refers to an artificial sequence engineered into the genetic material of the microorganism, for the purpose of tracking the microorganism. In some embodiments of any of the aspects, the genetic barcode element comprises at least a first primer binding sequence, at least one barcode region, and a second primer binding sequence. In some embodiments of any of the aspects, a genetic barcode element further comprises one or more of a transcription start site, a Cas enzyme scaffold, and one or more additional barcode regions, or any combination thereof.

In some embodiments of any of the aspects, the genetic barcode element comprises the following: (i) a first primer binding sequence; (ii) a first barcode region; (iii) a Cas enzyme scaffold; (iv) a transcription initiation site (v) a second barcode region; and (vi) a second primer binding sequence. The first and second primer binding sites (also referred to herein as forward and reverse primer binding sequences) will generally flank that barcode region(s). Additional components can be located between primer binding sequences in varying orders, non-limiting examples of which are discussed herein below.

In some embodiments of any of the aspects, the genetic barcode element comprises the following: (i) a first primer binding sequence; (ii) a transcription initiation site; (iii) at least one barcode region; and (vi) a second primer binding sequence. In some embodiments of any of the aspects, a second nucleic acid (e.g., a crRNA) comprises a Cas enzyme scaffold and a region that is complementary to and/or hybridizes to a barcode region of the genetic barcode element.

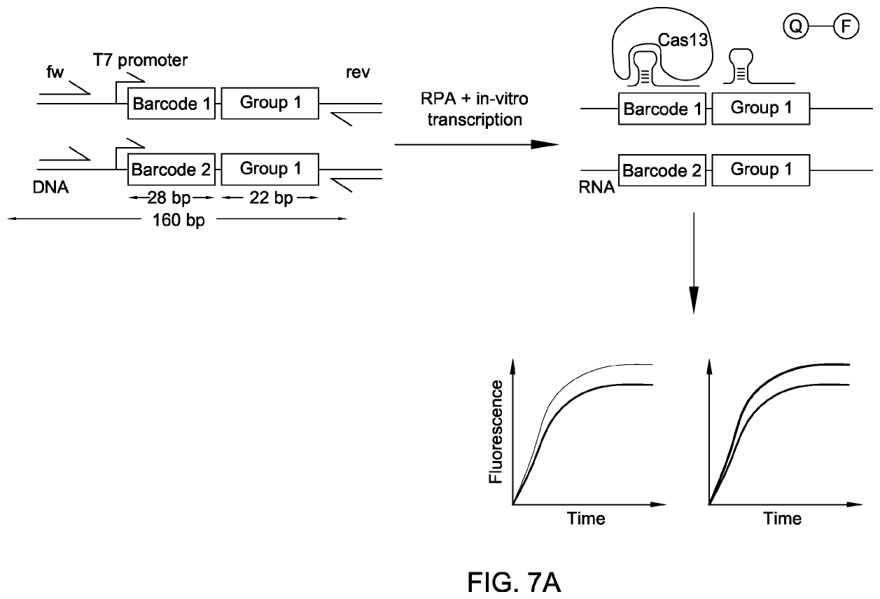

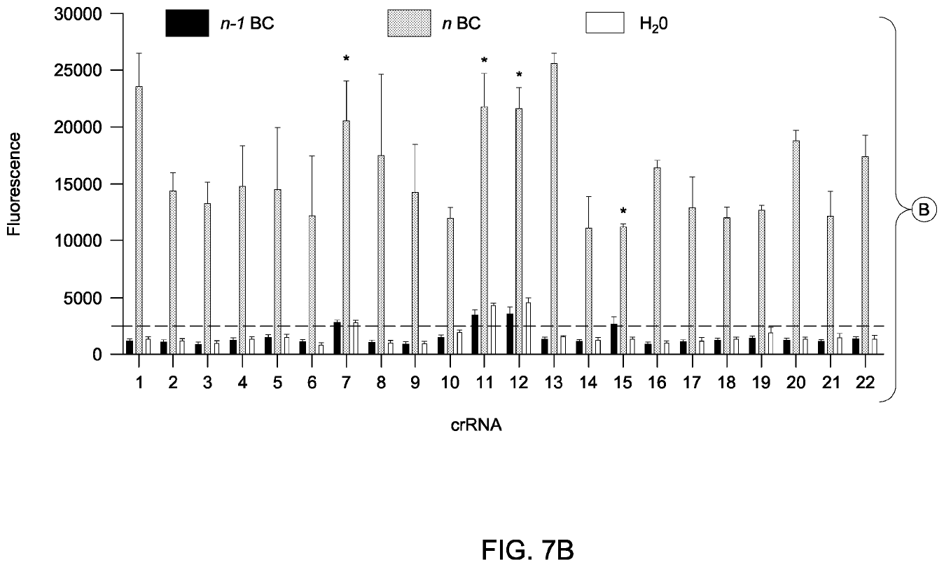

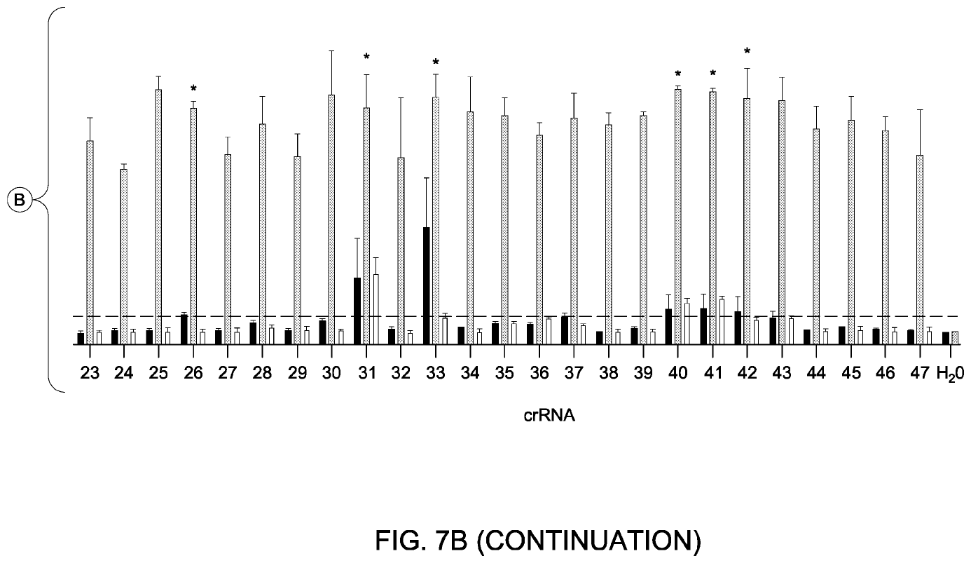

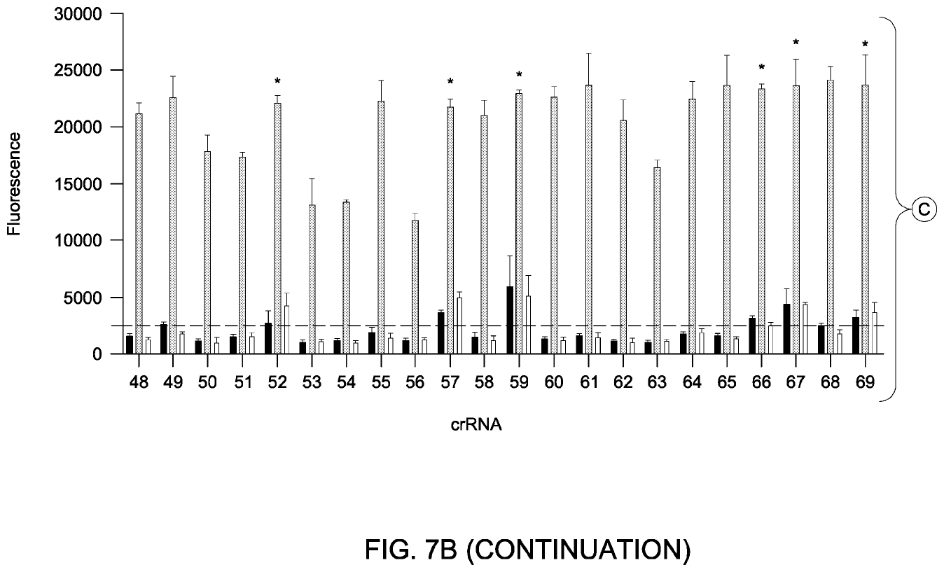

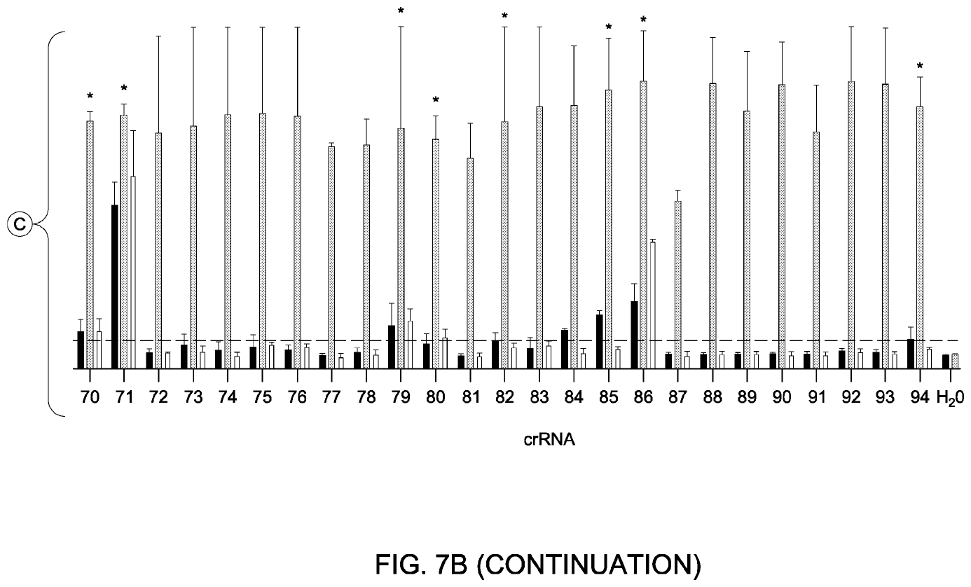

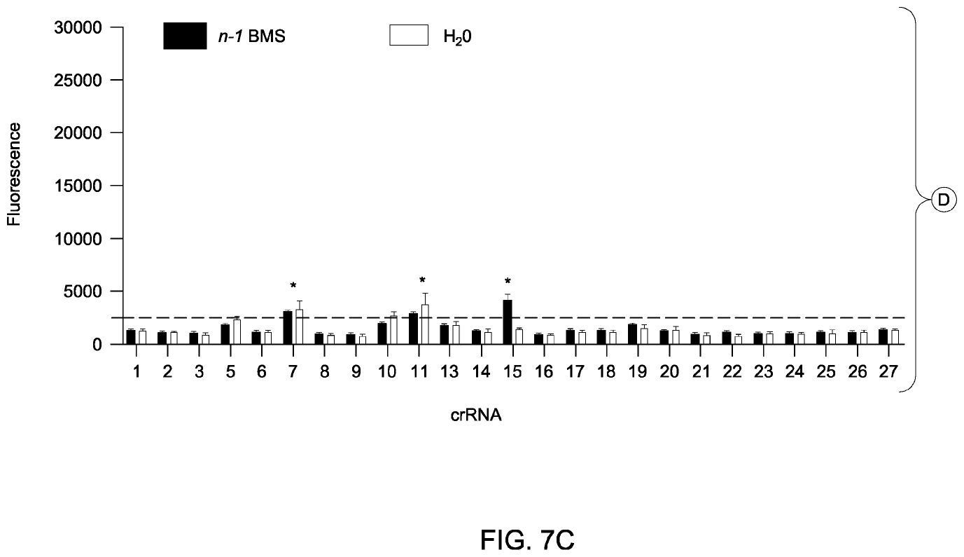

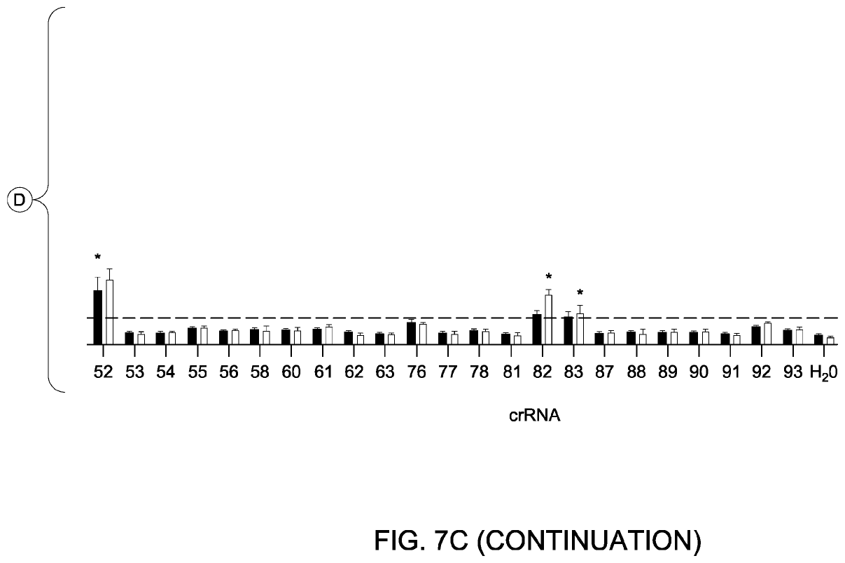

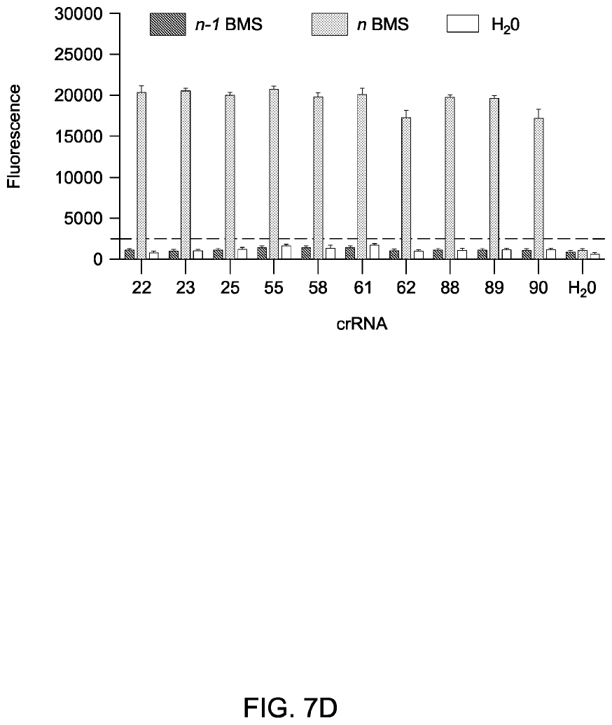

In some embodiments of any of the aspects, the genetic barcode element comprises the following in order from 5′ to 3′: (i) a first primer binding sequence; (ii) a transcription initiation site; (iii) a first barcode region; and (iv) a second primer binding sequence. In some embodiments of any of the aspects, the genetic barcode element comprises the following in order from 5′ to 3′: (i) a first primer binding sequence; (ii) a transcription initiation site; (iii) first and second barcode regions; and (iv) a second primer binding sequence (see e.g., FIG. 7A). In some embodiments of any of the aspects, the genetic barcode element comprises the following in order from 5′ to 3′: (i) a first primer binding sequence; (ii) a transcription initiation site; (iii) a Cas enzyme scaffold; (iv) at least one barcode region; and (v) a second primer binding sequence. In some embodiments of any of the aspects, the genetic barcode element comprises the following in order from 5′ to 3′: (i) a first primer binding sequence; (ii) at least one barcode region; (iii) a Cas enzyme scaffold; (iv) a transcription initiation site; and (v) a second primer binding sequence (see e.g., FIG. 1 and Example 1).

In some embodiments of any of the aspects, the genetic barcode element is selected from the sequences in Table 6. In some embodiments of any of the aspects, the genetic barcode element comprises one of SEQ ID NOs: 222-315 or a nucleic acid sequence that is at least 97.5%, at least 98%, at least 98.5%, at least 99%, at least 99.5%, or more identical to one of SEQ ID NOs: 222-315 that maintains the same function (e.g., priming, barcode identification, Cas enzyme scaffold, and/or transcription initiation site).

In some embodiments of any of the aspects, the genetic barcode element does not comprise one of barcodes 7, 11, 12, 15, 26, 31, 33, 40, 41, 42, 52, 57, 59, 66, 67, 69, 70, 71, 79, 80, 82, 83, 85, 86, 94 (see e.g., FIG. 7B-7C). In some embodiments of any of the aspects, the genetic barcode element does not comprise one of SEQ ID NOs: 228, 232, 233, 236, 247, 252, 254, 261, 262, 263, 273, 278, 280, 287, 288, 290, 291, 292, 300, 301, 303, 304, 306, 307, 315 or a nucleic acid sequence that is at least 97.5%, at least 98%, at least 98.5%, at least 99%, at least 99.5%, or more identical to one of SEQ ID NOs: 228, 232, 233, 236, 247, 252, 254, 261, 262, 263, 273, 278, 280, 287, 288, 290, 291, 292, 300, 301, 303, 304, 306, 307, or 315.

In some embodiments of any of the aspects, the genetic barcode element comprises one of barcodes 1-6, 8-10, 13-14, 16-25, 27-30, 32, 34-39, 43-51, 53-56, 58, 60-65, 68, 72-78, 81, 84, 87-93, or Universal 2. In some embodiments of any of the aspects, the genetic barcode element comprises one of SEQ ID NOs: 222-227, 229-231, 234-235, 237-246, 248-251, 253, 255-260, 264-272, 274-277, 279, 281-286, 289, 293-299, 302, 305, 308-314 or a nucleic acid sequence that is at least 97.5%, at least 98%, at least 98.5%, at least 99%, at least 99.5%, or more identical to one of SEQ ID NOs: 222-227, 229-231, 234-235, 237-246, 248-251, 253, 255-260, 264-272, 274-277, 279, 281-286, 289, 293-299, 302, 305, or 308-314.

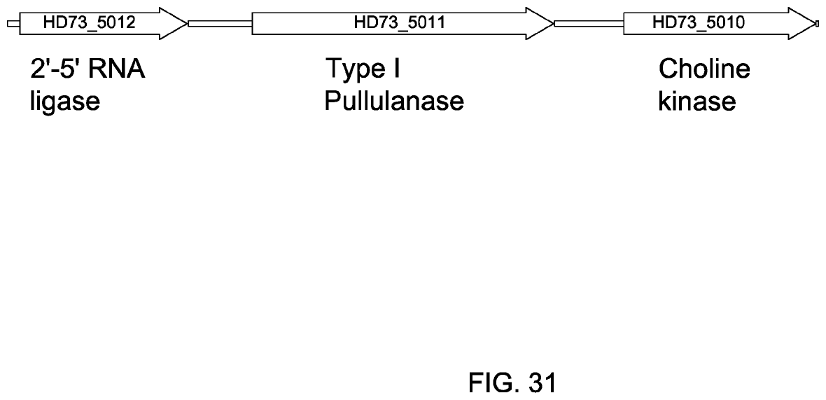

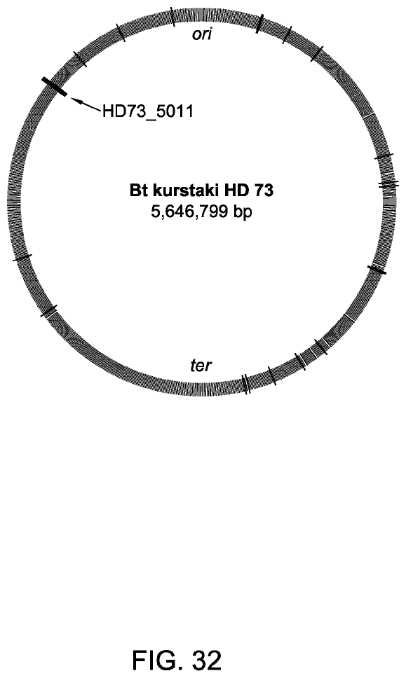

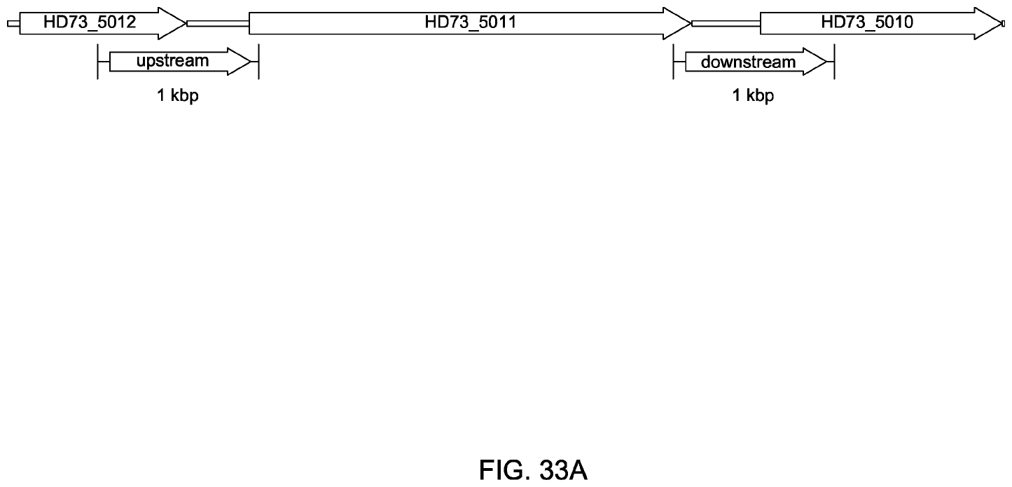

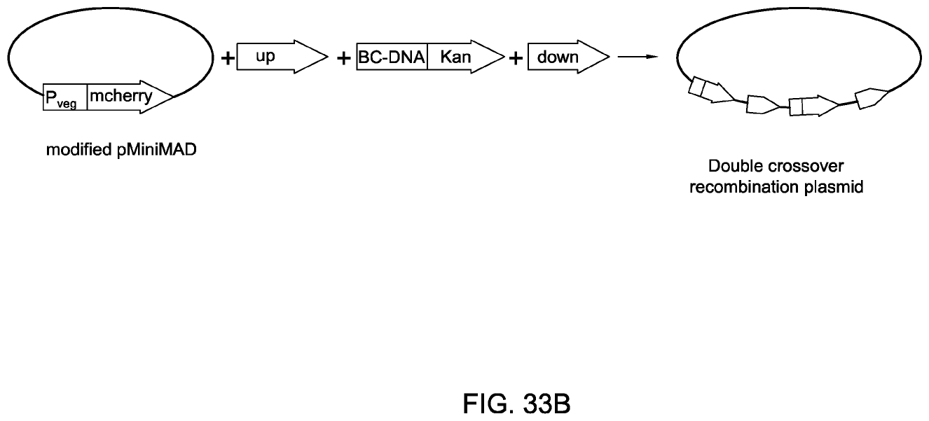

In some embodiments of any of the aspects, the genetic barcode element is integrated into the genome of the engineered microorganism, such that it is stably expressed, maintained, and/or replicated in the microorganism. Integration of the genetic barcode element into the genome can partially or fully interrupt or delete a natural gene or locus of the microorganism. The locus for integration of the genetic barcode element can be chosen based on at least one of the following criteria: (1) a non-essential gene or locus (i.e., integrating the genetic barcode element into the locus will not result in death or significantly decreased fitness of the engineered microorganism); (2) a gene or locus not involved in sporulation (e.g., if a sporulating microorganism is used such as a Bacillus species); and/or (3) a gene or locus that is near the origin of replication of the microorganism's genome (e.g., within 1 million base pairs of the ori, see e.g., FIG. 32). Non-limiting examples of such loci include: ho (e.g., for S. cerevisiae; HOmothallic switching endonuclease, see e.g., Systematic Name YDL227C, SGD ID SGD:S000002386, nt 46271-48031 (complement) ofNC_001136.10 Reference assembly, see e.g., SEQ ID NO: 316); ycgO (e.g., for B. subtilis; a Sodium/proline symporter, also known as putP, see e.g., nt 347165 to 348577 of GenBank: CP053102.1; see e.g., SEQ ID NO: 317); or HD73_5011 (e.g., for B. thuringiensis; HD73_5011 can also be referred to as HD73_RS24940, a Type I pullulanase; see e.g., NC_020238.1 (4806125-4808266, complement); see e.g., SEQ ID NO: 318); (see e.g., Table 2, FIG. 31, FIG. 33A).

In some embodiments of any of the aspects, the integration locus for the genetic barcode element comprises one of SEQ ID NOs: 316-318 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to one of SEQ ID NOs: 316-318, that maintains the same criteria (e.g., non-essential, not involved with sporulation, and/or near the ori).

SEQ ID NO: 316, Saccharomyces cerevisiae S288C
chromosome IV, complete sequence NCBI Reference
Sequence: NC_001136.10, complement(46271-48031),
gene HO, locus tag YDL227C, GeneID: 851371, 1761
base pair (bp)
ATGCTTTCTGAAAACACGACTATTCTGATGGCTAACGGTGAAATTAAAGACATCGCAAAC
GTCACGGCTAACTCTTACGTTATGTGCGCAGATGGCTCCGCTGCCCGCGTCATAAATGTC
ACACAGGGCTATCAGAAAATCTATAATATACAGCAAAAAACCAAACACAGAGCTTTTGA
AGGTGAACCTGGTAGGTTAGATCCCAGGCGTAGAACAGTTTATCAGCGTCTTGCATTACA
ATGTACTGCAGGTCATAAATTGTCAGTCAGGGTCCCTACCAAACCACTGTTGGAAAAAA
GTGGTAGAAATGCCACCAAATATAAAGTGAGATGGAGAAATCTGCAGCAATGTCAGACG
CTTGATGGTAGGATAATAATAATTCCAAAAAACCATCATAAGACATTCCCAATGACAGTT
GAAGGTGAGTTTGCCGCAAAACGCTTCATAGAAGAAATGGAGCGCTCTAAAGGAGAATA
TTTCAACTTTGACATTGAAGTTAGAGATTTGGATTATCTTGATGCTCAATTGAGAATTTCT
AGCTGCATAAGATTTGGTCCAGTACTCGCAGGAAATGGTGTTTTATCTAAATTTCTCACT
GGACGTAGTGACCTTGTAACTCCTGCTGTAAAAAGTATGGCTTGGATGCTTGGTCTGTGG
TTAGGTGACAGTACAACAAAAGAGCCAGAAATCTCAGTAGATAGCTTGGATCCTAAGCT
AATGGAGAGTTTAAGAGAAAATGCGAAAATCTGGGGTCTCTACCTTACGGTTTGTGACG
ATCACGTTCCGCTACGTGCCAAACATGTAAGGCTTCATTATGGAGATGGTCCAGATGAAA
ACAGGAAGACAAGGAATTTGAGGAAAAATAATCCATTCTGGAAAGCTGTCACAATTTTA
AAGTTTAAAAGGGATCTTGATGGAGAGAAGCAAATCCCTGAATTTATGTACGGCGAGCA
TATAGAAGTTCGTGAAGCATTCTTAGCCGGCTTGATCGACTCAGATGGGTACGTTGTGAA
AAAGGGCGAAGGCCCTGAATCTTATAAAATAGCAATTCAAACTGTTTATTCATCCATTAT
GGACGGAATTGTCCATATTTCAAGATCTCTTGGTATGTCAGCTACTGTGACGACCAGGTC
AGCTAGGGAGGAAATCATTGAAGGAAGAAAAGTCCAATGTCAATTTACATACGACTGTA
ATGTTGCTGGGGGAACAACTTCACAGAATGTTTTGTCATATTGTCGAAGTGGTCACAAAA
CAAGAGAAGTTCCGCCAATTATAAAAAGGGAACCCGTATATTTCAGCTTCACGGATGATT
TCCAGGGTGAGAGTACTGTATATGGGCTTACGATAGAAGGCCATAAAAATTTCTTGCTTG
GCAACAAAATAGAAGTGAAATCATGTCGAGGCTGCTGTGTGGGAGAACAGCTTAAAATA
TCACAAAAAAAGAATCTAAAACACTGTGTTGCTTGTCCCAGAAAGGGAATCAAGTATTTT
TATAAAGATTGGAGTGGTAAAAATCGAGTATGTGCTAGATGCTATGGAAGATACAAATT
CAGCGGTCATCACTGTATAAATTGCAAGTATGTACCAGAAGCACGTGAAGTGAAAAAGG
CAAAAGACAAAGGCGAAAAATTGGGCATTACGCCCGAAGGTTTGCCAGTTAAAGGACCA
GAGTGTATAAAATGTGGCGGAATCTTACAGTTTGATGCTGTCCGCGGGCCTCATAAGAGT
TGTGGTAACAACGCAGGTGCGCGCATCTGCTAA
SEQ ID NO: 317, Bacillus subtilis subsp. subtilis str. 168
chromosome, complete genome GenBank: CP053102.1, region:
347126-348580, gene putP, locus tag HIR77_01870, 1455 bp
CTGATTATTTCGATTTGTATTTATATGGCGGGAATGCTGCTAATCGGCTACTTTGCTTACA
AGCGTACGTCGAATCTGACGGATTATATGCTGGGAGGACGCTCTTTGGGTCCGGCAGTAA
CCGCTCTCAGTGCCGGCGCTGCCGATATGAGCGGCTGGCTGCTAATGGGGCTTCCGGGCG
CCATGTTTTCTACTGGTCTGAGCGGCGCTTGGATCGTCATCGGACTTTGCCTTGGAGCGTG
GGCGAACTGGCTTTACGTCGCACCACGGCTGAGAACCTATACTGAAAAAGCGGGGAATT
CTATTACAATTCCCGGATTCCTGGAAAATCGCTTCGGAGATCAAACAAAGCTTCTCAGAC
TGTTTTCGGGAATTGTCATTTTAGTCTTTTTCACGTTTTATGTATCGTCCGGAATGGTGTCC
GGCGGCGTCTTATTCAACAGCATCTTGGGGATGGATTATCACACCGGCCTGTGGATTGTG
ACGGGTGTTGTTGTGGCATATACCCTGTTTGGCGGCTTTTTGGCAGTCAGCTGGACAGAT
TTTGTTCAGGGGATTATTATGTTTGCCGCACTCATTCTTGTGCCGATCGTTACGTTTTTCC
ACACGGGAGGAGCAGGTGATACAGTTGCTGAAATCCGCTCTGTAGATCCTGATATGTTTA
ATATTTTCAAAGGAACGAGCGTCCTTGGCATTATTTCTTTGTTTGCCTGGGGGCTGGGAT
ACTTTGGCCAGCCGCATATTATTGTGCGTTTTATGGCGATAACGTCTGTCAAAGAGATCA
AAAGAGCGCGCAGAATTGGGATGGGCTGGATGATTTTGTCTGCAGTAGGCGCAGTGCTG
ACCGGTTTGGGCGGAATCGCTTATTACCATCAGCGCGGCATGACACTGAAGGACCCAGA
GACGATTTTTATTCAATTAGGGAATATCTTGTTCCATCCGATTATCACGGGTTTTCTGATT
TCAGCGATTTTGGCCGCGATTATGAGTACAATTTCTTCCCAGCTGCTTGTGACATCAAGCT
CTCTGGTAGAAGATTTGTATAAGTCGATGTTCAGACGCTCAGCTTCAGATAAAGAACTGG
TGTTTTTAGGCCGACTGGCTGTTCTGGCTGTTTCAATTGTCGCGTTGGTTCTGGCTTGGGA
GAAAAATAACACCATCCTTGGATTGGTCAGCTACGCGTGGGCGGGCTTCGGTGCATCATT
CGGGCCAGTTGTACTGCTCAGTCTGTTTTGGAAACGAATGACCAAATGGGGGGCACTCGC
CGGCATGATTGTGGGAGCAGCGACTGTAATCATTTGGGCGAATGCCGGTCTTTCGGACTT
TCTGTATGAAATGATACCTGGTTTTGCTGCGAGTCTATTATCTGTCTTTTTTGTCAGCATA
TTGACGCAGGCTCCGTCACAAGCTGTCACAGACCAGTTCAACGACTACCAAGATACAAT
GTCGCAATAA
SEQ ID NO: 318, Bacillus thuringiensis serovar kurstaki
str. HD73, complete sequence, NCBI Reference Sequence:
NC_020238.1 region: 4806125-4808266 (complement), gene pulA,
locus tag HD73_RS24940, old locus tag HD73_5011, 2142 bp
(ATGTTGAAAGTAAAACGTCCATTTGATGCCTATTTAGATGAAATGAATAAAATTACAAT
TTTGCTCCCACATGCGTATGGAACAAGTCGGACATTTCGTTTGCAAGAAGGAAGTAATGT
GAAAGATTTACCGATTGCTCATACTATTGCGCTTCCAGATGCAACGAAGTATGAGTGCTT
TATAGAAGAGCCGCTAGATGTGGGGAAGTATTATACAGTACGAGATGAACGGAATGAAG
AAACTGATTTACAAATAGGTGCTGTTATTCGAACAAAAATTTTCGATGAAAAGTATTATT
ACGAAGGTACAGATTTAGGTGCTGTGTATCAAAAACAAGAAACGACATTTAAAGTATGG
GCCCCAACTGCAAGGCTTGCGAAAGTAAGAATTTATAAGAGTGATAAAGAATATACCGA
TCATGAAATGTACAGGGGAGAAAACGGAGTTTGGATTCAAACATTACAAGGTGATTTAG
ACGGAGCACGCTATACATTTCTTGTTTGTATTAATTTAATATGGAACGAAGCGGTGGATC
CTTACGCAAAGTCAGTGACTGTAAATGGAAAGTATGGCATTGTTATCGATTTAGAAAAG
ACGACTGTAACGAAACGAGAGCAATTACCACCATTACAAGCAATGACAGATGCCATATT
GTATGAATTGCATATTCGTGATGCTACTATTCATCCGAACAGTGGAGTGAGTAAAAAAGG
GACGTATAAAGGGCTAATGGAAGAAGGAACAACAGGACGAAATGGGACATTGACAGGT
TTGTCTCATATAAAAGATTTAGGCGTTACGCATGTTGAATTATTACCTTTATATTGTTTCG
GTGGTGTAGATGAGGCGAATCCTTCGTCCGCCTATAATTGGGGATATAATCCATTATATT
ACAATGCACCAACAGGATTTTATGCTACAAACCCCTCTGATCCGTATAACAGGATAGTTG
AGTGCAAACAACTAATTGAAACATTTCATGAACAAGGTATTAGGGTGATTATAGATGTTG
TATATAATCATGTTTATGAAAGAGAGCTATCATCATTTGAGAAGCTTGTTCCAGGATATT
ATTTTCGTCATGGTGAAGATGGTATGCCTTCTAATGGGACGGGAGTCGGAAATGATGTAG
CGTCTGAACGGAAAATGATGAGGAAATTCATTGTAGAGTCTATTTTATATTGGCTTACTG
AATACAATGTCGATGGATTCCGATTCGACTTAATGGGAATTTTAGATGTTGATACAATGA
ATATGATAGAGAAAGAAGTGCGAAACATAAAACGAGATGCTTTGTTATTAGGAGAAGGA
TGGGATTTACAAACACCGCTTCCTCTTGAGGAAAAAGCAACGTTAAATAATGCGAAGAA
AATGCCACATATCGCGCAGTTTAATGATCAATTTCGTGATGGGATAAAAGGGAGTACATT
TCATATAAATAAGCGTGGTTTTGCATTTGGTGGGCATGTAGACTGTAATCATTTGCAGTA
TATTGCAGCAGGAAGTCTTTTAAGTATGAAAGAAACAGGGCTGTTTTTAGAGCCGGCGC
AAAGCATTAACTATGTAGAATGTCATGATAATATGACGATGTGGGATAAACTAGTGCGC
AGTAATGAAGAATCAGAAACGATTTTGAAAAAACGTCACCTATTAGCGAGCGCAATGGT
TATTCTTTCACAAGGCATTCCTTTCTTACACGCAGGGCAAGAGTTTTATCGTACGAAGAA
AGGAAATGAAAATAGTTATAATGCAAATGATGAGATCAATCAATTAGATTGGGATCAAA
AAGAGAAAGAGATAGAGACCGTTAACTATATAAAAGGTTTAATTGCGATTCGTAAAGAG
CATGGGGCATTCCGATTACAAAATGCAGACCTTATAAAAAAACATATGACTTTTTTACAA
ACATCTACAGAAGTACTTGCATATCACCTAGAGCATGCAGAATCGTTTGGGCCGTGGAA
AGAGATTATCGTCTTATTTAATAGCGGTTTAGAAGCTAAAACTGTGCAACTTCCGAAAGA
AGCAACGTGGCATGTGTTAGTAAACGAAAACGAAGCGAAAATAGAGCCAATTTCTTCAT
TTAGAGGAAAGGAACTTAGGCTCGCTCCAATTAGCACGTATATATTGACGATAATGTGA

In some embodiments of any of the aspects, the genetic barcode element is integrated into the genome of the microorganism using transformation with a vector, e.g., a vector that allows for double crossover recombination. Non-limiting examples of such vectors include pCB018 (see e.g., Example 2 Methods; e.g., for B. subtilis), or a modified pMiniMAD plasmid (e.g., pFR51; see e.g., Example 3; e.g., for B. thuringiensis). In some embodiments of any of the aspects, the genetic barcode element is integrated into the genome of the microorganism using genetic editing tools (e.g., CRISPR, TALENs, zinc-finger nucleases (ZFNs), homing endonucleases or meganucleases, or other gene editing tools as known in the art). In some embodiments of any of the aspects, the genetic barcode element is integrated into the genome of the microorganism using CRISPR-Cas (see e.g., Example 2 Methods; e.g., for S. cerevisiae). In some embodiments of any of the aspects, the genetic barcode element and a selection marker (e.g., a gene conferring antibiotic resistance such as to kanamycin, erythromycin, or geneticin, or a detectable marker such as a fluorophore) are integrated into the genome of the microorganism. In some embodiments of any of the aspects, the selection marker is removed after the genetic barcode element is integrated into the genome of the engineered microorganism.

### Primer Binding Sequences

In some embodiments of any of the aspects, the first and second primer binding sequences comprise sites for binding of PCR or RPA primers. In some embodiments of any of the aspects, the first primer binding sequence comprises RPA primer 1 (e.g., GATAAACACAGGAAACAGCTATGACCATGATTACG, SEQ ID NO: 1), and/or the second primer binding sequence comprises RPA primer 2 (e.g., GGGATCCTCTAGAAATATGGATTACTTGGTAGAACAG, SEQ ID NO: 4). In some embodiments of any of the aspects, the primer binding sequence comprises one of SEQ ID NOs: 1, 4, or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to one of SEQ ID NOs: 1 or 4, that maintains the same function (e.g., primer binding). In some embodiments of any of the aspects, the first primer binding sequence and/or the second primer binding sequence comprises any primer or primer pair known in the art to be used for recombinase polymerase amplification (RPA) or any other isothermal amplification method.

As used herein, the term “primer” denotes a single-stranded nucleic acid that hybridizes to a nucleic acid region of interest and provides a starting point for nucleic acid synthesis, i.e. for enzymatic synthesis of a nucleic acid strand complementary to a template, e.g., the genetic barcode element. In some embodiments of any of the aspects, the primer can be DNA, RNA, modified DNA, modified RNA, synthetic DNA, synthetic RNA, or another synthetic nucleic acid. In some embodiments, the primer is about 17-35 nucleotides long. As a non-limiting example, the primer is 17 nucleotides (nt) long, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, or 35 nt long. In some embodiments of any of the aspects, the primer is complementary or has complete identity to the priming binding sequence. In some embodiments of any of the aspects, at least one primer is selected from the sequences in Table 3.

In some embodiments of any of the aspects, methods described herein comprise isothermal amplification. Non-limiting examples of isothermal amplification include: Recombinase Polymerase Amplification (RPA), Loop Mediated Isothermal Amplification (LAMP), Helicase-dependent isothermal DNA amplification (HDA), Rolling Circle Amplification (RCA), Nucleic acid sequence-based amplification (NASBA), strand displacement amplification (SDA), nicking enzyme amplification reaction (NEAR), and polymerase Spiral Reaction (PSR). See e.g., Yan et al., Isothermal amplified detection of DNA and RNA, March 2014, Molecular BioSystems 10(5), DOI: 10.1039/c3mb70304e, the content of which is incorporated herein by reference in its entirety. In some embodiments of any of the aspects, the genetic barcode element of an engineered microorganism as described herein is amplified using isothermal amplification (e.g., RPA). In some embodiments of any of the aspects, methods described herein comprise polymerase chain reaction (PCR) amplification. In some embodiments of any of the aspects, the genetic barcode element of an engineered microorganism as described herein is amplified using PCR.

As well known to those of skill in the art, standard assay (e.g., RPA, PCR) conditions or parameters can comprise preferred values for product size, primer size, primer Tₘ, Tₘ difference, product Tₘ, and/or primer GC % (i.e., the percentage of G or C bases compared to total bases). In regard to isothermal amplification, as a non-limiting example, primer Tₘ and/or product Tₘ can be about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., or about 45° C., with a preferred primer Tₘ between about 30° C. and 37° C. In regard to PCR amplification, as a non-limiting example, primer Tₘ and/or product Tₘ can be about 57° C., about 58° C., about 59° C., about 60° C., about 61° C., about 62° C., or about 63° C., with a preferred primer Tₘ of about 60° C.

With regard to isothermal amplification (e.g., RPA) or PCR, as a non-limiting example, the maximum difference between the Tₘ's of the forward primer and the reverse primer can be about 0.5° C., about 1° C., about 2° C., about 3° C., about 4° C., about 5° C., about 6° C., about 7° C., about 8° C., about 9° C., or about 10° C. As a non-limiting example, GC % (e.g., of the primers) can be about 20%, about 25%, about 30%, about 35%, about 40%, about 45%, about 50%, about 55%, about 60%, about 65%, about 70%, about 75%, or about 80%. Methods for calculating Tₘ are well known to those of skill in the art (see e.g., Panjkovich and Melo, Bioinformatics, Volume 21, Issue 6, 15 Mar. 2005, Pages 711-722, which is incorporated by reference herein in its entirety).

In some embodiments of any of the aspects, primers are compared for specificity versus sequences in the genome of the microorganism, genome of the item (if it comprises genetic material; e.g., a food item), or any known nucleic acid (e.g., the BLAST Nucleotide Collection (nr/nt) available on the world wide web at blast.ncbi.nlm.nih.gov/Blast.cgi) using alignment software (e.g., primer blast (NCBI™); isPCR (UCSC)). Only those primers predicted to be specific for their respective targets (e.g., hybridizing only to the primer binding sequence) and not specific to non-target (e.g., the genome of the microorganism, genome of the item, or any known nucleic acid) are kept. While hybridization is influenced by GC content as well as overall complementarity, in general a primer that is specific for a single target should have no more than about 80% sequence identity with sequences that are not target sequences. As a non-limiting example, the primer can have about 0%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% or less sequence identity with a non-target sequence (e.g., in the genome of the microorganism, the genome of the item, or any known sequence).

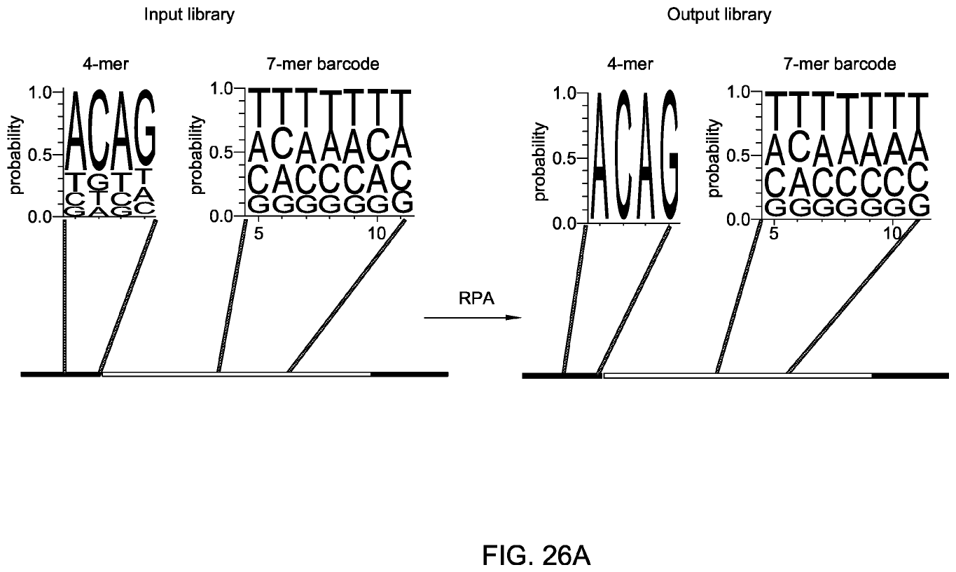

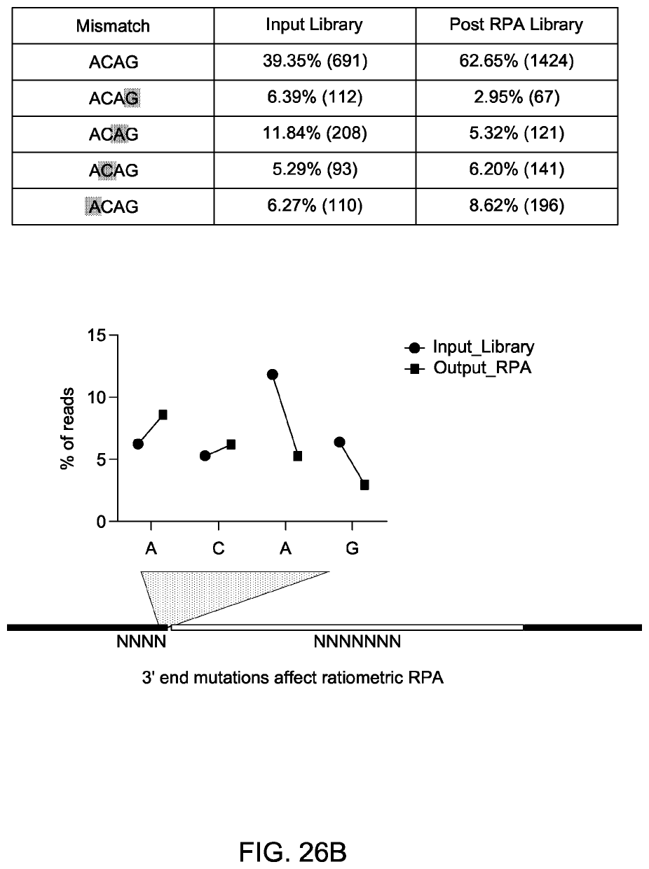

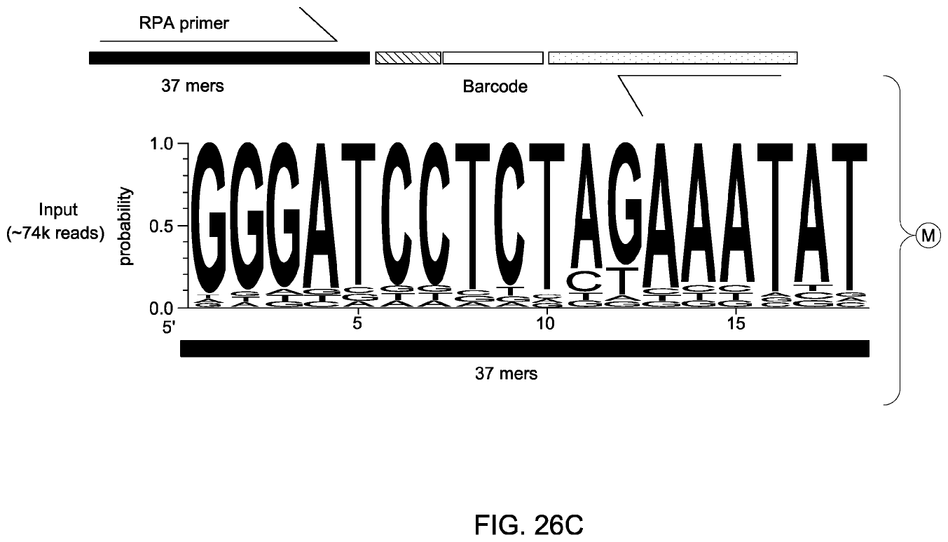

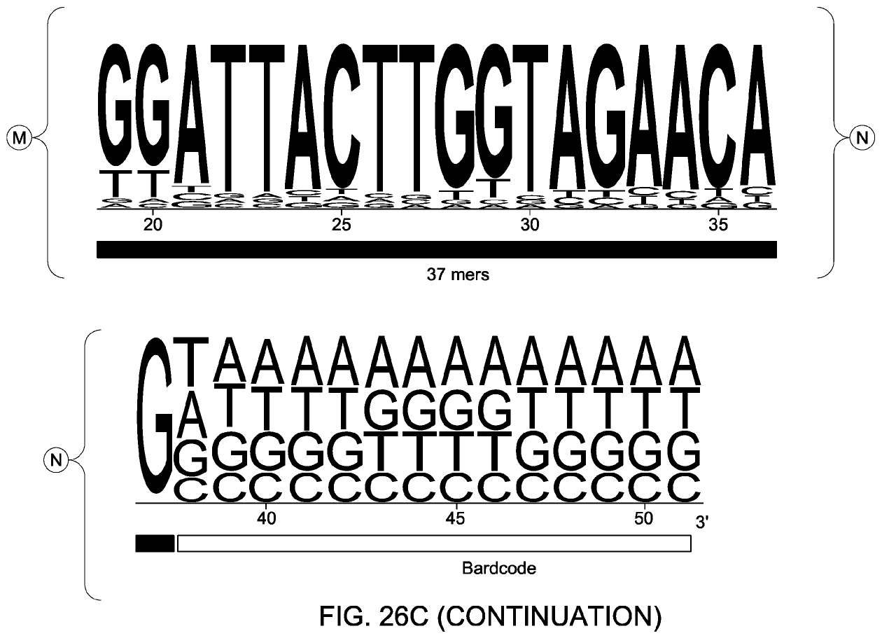

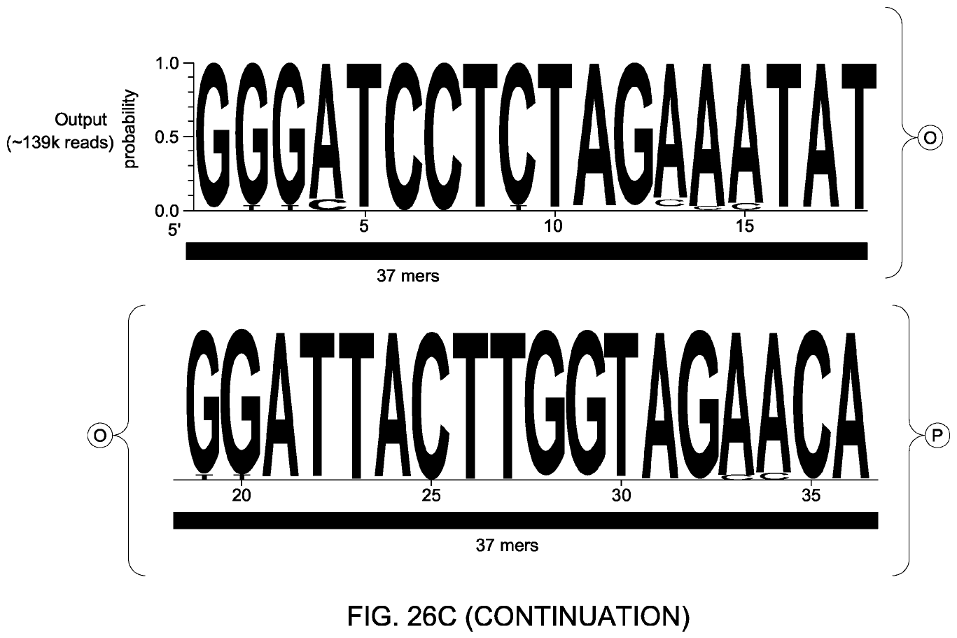

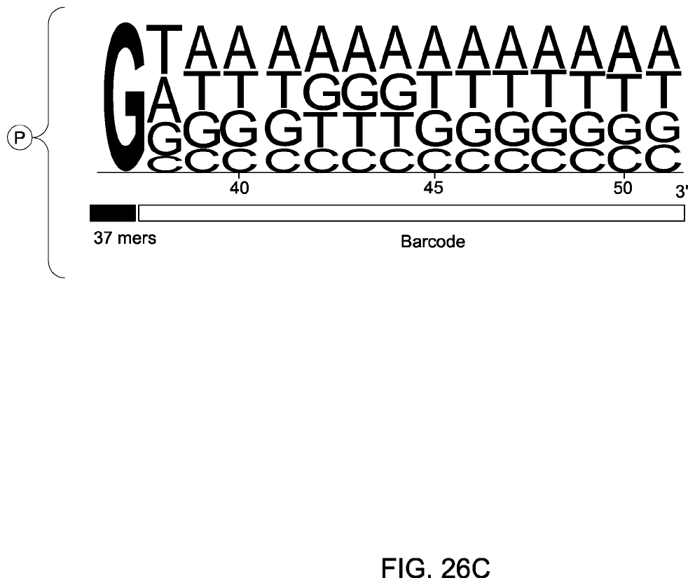

In some embodiments of any of the aspects, the primer comprises a Hamming distance of at least 5 base pairs from a non-target sequence (e.g., the genome of the microorganism, genome of the item, or any known nucleic acid). As used herein, the term “Hamming distance” refers to the number of positions (e.g., base pairs) at which the corresponding sequences are different. As a non-limiting example, the barcode region comprises a Hamming distance of at least 5 base pairs, at least 6 base pairs, at least 7 base pairs, at least 8 base pairs, at least 9 base pairs, or at least 10 base pairs from a non-target sequence.

Where amplification depends upon extension of a primer, it is important that the last nucleotide (e.g., at the 3′ end) of a primer hybridizes to the template (e.g., the genetic barcode element as described herein, specifically a primer binding sequence within a genetic barcode element). A mismatch of the last nucleotide (e.g., at the 3′ end) of the primer will generally preclude extension. Mismatches between the primer and primer binding region can occur, for example, through mutation or genetic drift of the engineered microorganism, e.g., in the environment or on an item. In practice, it can be helpful if at least the last two nucleotides at the 3′ end of a primer are complementary to the template (see e.g., FIG. 26A-26C). Thus, a primer can tolerate some degree of mismatch as noted, but it is necessary for the last nucleotide at the 3′ end of a primer as described herein to be complementary to the template and beneficial if at least the last two nucleotides are complementary.

Additional assay conditions that can be considered when necessary or desired during primer selection include but are not limited to off-product reactions (e.g., primer dimers, i.e., primer molecules that have hybridized to each other due to region of complementarity in the primers), primer self-complementarity, primer 3′ self-complementarity, primer #N's (e.g., consecutive repeated nucleotides), primer mispriming similarity, primer sequence quality, primer 3′ sequence quality, and/or primer 3′ stability. Preferred values for each of the aforementioned conditions can be set or determined by one of skill in the art or by the specific primer selection algorithm (e.g., Primer 3™ Oligo Analyzer™, NetPrimer™, or Oligo Calculator™)

### Barcode Region

In some embodiments of any of the aspects, the genetic barcode element comprises at least one barcode region. As a non-limiting example, the genetic barcode element comprises at least 1 barcode region, at least 2 barcode regions, at least 3 barcode regions, at least 4 barcode regions, or at least 5 barcode regions.

In some embodiments of any of the aspects, the barcode region comprises 20-40 base pairs (bp). As a non-limiting example, the barcode region can be 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, or 40 bp long.

In some embodiments of any of the aspects, the barcode region comprises a Hamming distance of at least 5 base pairs from another barcode. A Hamming distance of 5 base pairs permits the creation of a set of approximately 2.9*10\hat{ }9 barcodes that can be co-used for a 28 base pair barcode. This Hamming distance permits accurate detection and differentiation of the barcode region by any of a number of detection methods. As a non-limiting example, the barcode region comprises a Hamming distance of at least 5 base pairs, at least 6 base pairs, at least 7 base pairs, at least 8 base pairs, at least 9 base pairs, or at least 10 base pairs from another barcode region relative to barcode regions comprised by other items marked with an engineered microorganism as described herein.

In some embodiments of any of the aspects, barcode regions are compared for specificity versus the genome of the microorganism, the genome of the marked item (if it comprises genetic material; e.g., a food item), or any known nucleic acid (e.g., the BLAST Nucleotide Collection (nr/nt) available on the world wide web at blast.ncbi.nlm.nih.gov/Blast.cgi) using alignment software (e.g., primer blast (NCBI™); isPCR (UCSC)). Only those barcode regions that are unique (e.g., less than 80% sequence identity) are kept and used. In some embodiments of any of the aspects, the barcode region comprises no more than 80% sequence identity with the genome of the microorganism, the genome of the item, or any known sequence. As a non-limiting example, the barcode region can have about 0%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% or less sequence identity with a sequence in the genome of the microorganism, the genome of the item, or any known sequence.

In some embodiments of any of the aspects, the barcode region comprises a Hamming distance of at least 5 base pairs from a non-target sequence (e.g., the genome of the microorganism, genome of the item, or any known nucleic acid). As a non-limiting example, the barcode region comprises a Hamming distance of at least 5 base pairs, at least 6 base pairs, at least 7 base pairs, at least 8 base pairs, at least 9 base pairs, or at least 10 base pairs from a non-target sequence.

In some embodiments of any of the aspects, the genetic barcode element comprises two different barcode regions. In such an embodiment, a first barcode region indicates a group of, for example, marked items, and the second barcode region marks a sub-group or class of the group. For example, then, in some embodiments of any of the aspects, all engineered microorganisms that share the same purpose (e.g., all assigned to a specific food item, but each assigned to a specific distribution location of the food item) can share a first barcode region (e.g., specific for the food item) but each microorganism (e.g., indicating a different distribution location) comprises a second barcode region that is unique (e.g., to that location). Accordingly, in some embodiments of any of the aspects, the microorganism is engineered to comprise first and second barcode regions. In some embodiments of any of the aspects, the first barcode region indicates that an item on which the microorganism is detected is from one of a group of known sources, and the second barcode region indicates that an item on which the microorganism is detected is from a particular source of said group of sources. In some embodiments of any of the aspects, the genetic barcode element comprises greater than two barcode regions, with each barcode region corresponding to a specific locational indicator (e.g., country, region, state, county, city, block, factory, field, farm, or any other locational indicator as needed).

In some embodiments of any of the aspects, the first barcode region is 5′ (e.g., with regard to the coding strand) to the second barcode region. In some embodiments of any of the aspects, the first barcode region is 3′ (e.g., with regard to the coding strand) to the second barcode region. In some embodiments of any of the aspects, the first and second barcode regions are tandem, i.e., arranged immediately next to each other. In some embodiments of any of the aspects, the first and second barcode regions are not tandem, e.g., intervening sequences (e.g., Cas enzyme scaffold, transcription initiation site) can be located in between the first and second barcode regions.

In some embodiments of any of the aspects, the first and second barcode regions are in the same genetic barcode element. Note that the first and second barcode regions, as described herein, are not required to be in the same genetic barcode element, but to avoid their possible separation by loss of one but not the other (e.g., through mutation of the engineered microorganism), it is envisioned that the two barcodes are closely linked (i.e., in close proximity; e.g., within the same locus or gene; e.g., within 1,000 base pairs of each other). Accordingly, in some embodiments of any of the aspects, the first and second barcode regions are in different, but closely linked genetic barcode elements. In one embodiment, the first and second barcode regions are in the same genetic barcode element, i.e., flanked by a single pair of engineered primer binding sequences.

In some embodiments of any of the aspects, the barcode region is selected from the sequences in Table 5. In some embodiments of any of the aspects, the barcode region comprises one of SEQ ID NOs: 5-31 or SEQ ID NOs: 154-221 or a nucleic acid sequence that is at least 85%, at least 90%, at least 91%, at least 92%, at least 93%, at least 94%, at least 95%, at least 96%, at least 97%, at least 98%, at least 99%, or more identical to one of SEQ ID NOs: 5-31 or SEQ ID NOs: 154-221, that maintains the same function (e.g., unique identification sequence).

In some embodiments of any of the aspects, the barcode region does not comprise one of barcodes 7, 11, 12, 15, 26, 31, 33, 40, 41, 42, 52, 57, 59, 66, 67, 69, 70, 71, 79, 80, 82, 83, 85, 86, 94 (see e.g., FIG. 7B-7C). In some embodiments of any of the aspects, the barcode region does not comprise one of SEQ ID NOs: 11, 15, 16, 19, 30, 157, 159, 166, 167, 168, 178, 183, 185, 192, 193, 195, 196, 197, 205, 206, 208, 209, 211, 212, 220 or a nucleic acid sequence that is at least 85%, at least 90%, at least 91%, at least 92%, at least 93%, at least 94%, at least 95%, at least 96%, at least 97%, at least 98%, at least 99%, or more identical to one of SEQ ID NOs: 11, 15, 16, 19, 30, 157, 159, 166, 167, 168, 178, 183, 185, 192, 193, 195, 196, 197, 205, 206, 208, 209, 211, 212, or 220.

In some embodiments of any of the aspects, the barcode region comprises one of barcodes 1-6, 8-10, 13-14, 16-25, 27-30, 32, 34-39, 43-51, 53-56, 58, 60-65, 68, 72-78, 81, 84, 87-93, or Universal 2. In some embodiments of any of the aspects, the barcode region comprises one of SEQ ID NOs: 5-10, 12-14, 17-18, 20-29, 31, 154-156, 160-165, 169-177, 179-182, 184, 186-191, 194, 198-204, 207, 210, 213-219, 221 or a nucleic acid sequence that is at least 85%, at least 90%, at least 91%, at least 92%, at least 93%, at least 94%, at least 95%, at least 96%, at least 97%, at least 98%, at least 99%, or more identical to one of SEQ ID NOs: 5-10, 12-14, 17-18, 20-29, 31, 154-156, 160-165, 169-177, 179-182, 184, 186-191, 194, 198-204, 207, 210, 213-219, or 221.

As used herein, the term “first barcode region” (also called a “group barcode region”) refers to a barcode region that can be shared by at least two different types of engineered microorganisms as described herein. In some embodiments of any of the aspects, at least one engineered microorganism comprises a first barcode region. In some embodiments of any of the aspects, the first barcode region can comprise SEQ ID NO: 5 or SEQ ID NO: 221. In some embodiments of any of the aspects, the first barcode region can comprise a sequence selected from the group consisting of SEQ ID NOs: 5-31 and SEQ ID NOs: 154-221.

As used herein, the term “second barcode region” (also called a “unique barcode region”) refers to a barcode region that is unique and/or distinguishable under conditions used in the assay from at least one other barcode region, e.g., comprised by other items marked with an engineered microorganism as described herein. While there are many other barcode region sequences that can be used, in some embodiments of any of the aspects, the second barcode region is selected from the group consisting of SEQ ID NOs: 5-31 and SEQ ID NOs: 154-221.

### Cas Enzyme Scaffold

In some embodiments of any of the aspects, the genetic barcode element comprises a Cas enzyme scaffold. A Cas enzyme scaffold is an RNA molecule comprising a sequence that permits the formation of secondary structure permitting specific binding by a Cas enzyme polypeptide. The Cas enzyme polypeptide/RNA scaffold complex is the configuration of the Cas enzyme that permits binding to and cleavage of the target nucleic acid sequences.

In some embodiments of any of the aspects, the genetic barcode element does not comprise a Cas enzyme scaffold, and a second nucleic acid provides a Cas enzyme scaffold. In some embodiments of any of the aspects, a crRNA (also referred to as a CRISPR RNA, a guide RNA, or a gRNA) comprises a Cas enzyme scaffold and a region that is complementary and/or hybridizes to a barcode region as described herein. In some embodiments of any of the aspects, at least one crRNA is selected from the sequences in Table 4. Accordingly, described herein are systems comprising a genetic barcode element (see e.g., Table 6) and at least one crRNA (see e.g., Table 4).

Cas enzyme scaffold sequences specific for numerous different Cas enzymes are known in the art, In some embodiments of any of the aspects, the Cas enzyme scaffold comprises a scaffold for Cas13. In some embodiments of any of the aspects, the Cas enzyme scaffold comprises a scaffold for Cas13a (previously known as C2c2), Cas13b, Cas13c, Cas12a, and/or Csm6. In some embodiments of any of the aspects, the Cas enzyme scaffold comprises GTTTTAGTCCCCTTCGTTTTTGGGGTAGTCTAAATC (SEQ ID NO: 2) or any Cas enzyme scaffold known in the art. In some embodiments of any of the aspects, the Cas enzyme scaffold comprises SEQ ID NO: 2 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to SEQ ID NO: 2, that maintains the same function (e.g., Cas enzyme binding).

### Transcription Initiation Site

In some embodiments of any of the aspects, the genetic barcode element comprises a transcription initiation site. Such sites permit recognition and transcript initiation by an RNA polymerase, e.g., a bacterial or bacteriophage polymerase. Eukaryotic polymerases can also be used. Sequences for transcription initiation sites are known for a variety of polymerases. In some embodiments of any of the aspects, the transcription initiation site comprises a T7 initiation site. In some embodiments of any of the aspects, the transcription initiation site comprises a SP6 initiation site, a T3 initiation site, or any other transcription initiation site known in the art. In some embodiments of any of the aspects, the transcription initiation site comprises CCCTATAGTGAGTCGTATTAGAATT (SEQ ID NO: 3). In some embodiments of any of the aspects, the transcription initiation site comprises SEQ ID NO: 3 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to SEQ ID NO: 3, that maintains the same function (e.g., transcription initiation).

### Essential Genes

In one aspect of any of the embodiments, an engineered microorganism comprises at least one inactivating modification of at least one essential gene. As used herein, the term “essential gene” refers to a gene of an organism that is critical for an organism's survival; in order for an organism to survive, an organism, comprising at least one inactivating modification of an essential gene, must be supplied the essential gene or its product(s) in some form (e.g., DNA, RNA, protein, product of the encoded enzyme).

In some embodiments of any of the aspects, the essential gene comprises a conditional essential gene. As used here, the term “conditional essential gene” refers to a gene that is essential under specific conditions or circumstances, e.g., the presence or absence of a gene product of the conditional essential gene. As a non-limiting example, a conditional essential gene is one that is essential when a product of the gene is not present in the environment, and a conditional essential gene is non-essential when a product of the gene is present in the environment. As another non-limiting example, a conditional essential gene (e.g., a lysine synthesis gene) is essential when a product of the gene (e.g., lysine) is not present in the environment, and the conditional essential gene (e.g., the lysine synthesis gene) is non-essential when a product of the gene (e.g., lysine) is present in the environment.

In some embodiments of any of the aspects, the conditional essential gene comprises an essential compound synthesis gene. As used herein, the term “essential compound” refers to any substance that the microorganism is dependent upon for growth, metabolism, or other cellular processes, and that must be obtained from the environment or synthesized by the microorganism in order for a microorganism to grow or survive. Non-limiting examples of essential compounds include amino acids, nucleotides, certain sugars, and vitamins.

In some embodiments of any of the aspects, the at least one essential compound synthesis gene comprises an amino acid synthesis gene. In some embodiments of any of the aspects, the at least one essential compound synthesis gene comprises a synthesis gene for a nucleotide (e.g., deoxyribonucleotide, ribonucleotide). In some embodiments of any of the aspects, the engineered microorganism comprises at least one inactivating modification of at least one amino acid synthesis gene and/or at least one inactivating modification of at least one nucleotide synthesis gene. In some embodiments of any of the aspects, the at least one essential compound synthesis gene comprises a vitamin synthesis gene that comprises at least one inactivating modification.

In some embodiments of any of the aspects, the engineered microorganism is an auxotroph for at least one essential compound, i.e. cannot grow in an environment lacking the compound(s) for which they are auxotrophic, i.e. can only grow in an environment containing the compound(s) for which they are auxotrophic.

In some embodiments of any of the aspects, the at least one essential compound synthesis gene comprises a synthesis gene for alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, adenine, guanine, cytosine, thymine, and/or uracil.

In some embodiments of any of the aspects, the at least one essential compound synthesis gene is selected from the group consisting of thrC, metA, trpC, pheA, HIS3, LEU2, LYS2, MET15, and URA3.

In some embodiments of any of the aspects, the essential compound synthesis gene comprises one of SEQ ID NOs: 319-331 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to one of SEQ ID NOs: 319-331, that maintains the same function (e.g., essential compound synthesis).

SEQ ID NO: 319, B. subtilis 168 thrC, threonine synthase,
Gene ID: 936660, NCBI Reference Sequence: NC_000964.3
complement (3313770-3314828), 1059 bp
ATGTGGAAAGGACTTATCCATCAATATAAAGAATTTTTACCTGTAACAGATCAAACACCG
GCGCTAACTTTACATGAAGGAAACACACCTCTTATTCACCTGCCGAAGCTGTCTGAGCAG
CTCGGAATTGAGCTTCATGTCAAAACGGAAGGCGTCAATCCTACGGGATCATTTAAAGAT
CGCGGAATGGTTATGGCTGTGGCAAAGGCAAAAGAAGAAGGCAATGACACGATTATGTG
CGCGTCAACAGGTAACACTTCCGCTGCTGCGGCAGCATATGCAGCCCGTGCTAACATGA
AATGCATTGTCATCATCCCGAACGGAAAAATTGCATTTGGAAAACTCGCTCAAGCTGTCA
TGTACGGAGCCGAGATTATCGCAATTGACGGAAACTTTGACGATGCGCTTAAAATTGTCC
GTTCCATCTGTGAGAAATCACCGATTGCCCTTGTCAACTCAGTCAACCCTTACCGCATTG
AAGGCCAAAAAACTGCTGCCTTCGAAGTGTGCGAACAGCTGGGGGAAGCGCCTGATGTT
TTAGCGATCCCGGTCGGAAATGCTGGAAACATCACTGCGTACTGGAAGGGCTTCAAGGA
ATATCATGAGAAAAACGGCACAGGCCTTCCGAAAATGCGCGGCTTTGAAGCTGAGGGTG
CGGCGGCAATCGTGCGCAATGAAGTGATTGAAAATCCGGAAACAATAGCGACAGCCATT
CGTATCGGAAACCCGGCAAGCTGGGACAAAGCTGTAAAGGCAGCCGAGGAATCCAATG
GGAAAATTGACGAAGTCACTGATGATGAAATCCTTCACGCATATCAGCTGATCGCCCGTG
TAGAAGGCGTGTTTGCAGAACCAGGTTCTTGCGCGTCTATCGCAGGAGTGCTGAAACAG
GTGAAATCCGGAGAAATTCCGAAAGGCAGCAAGGTCGTAGCTGTGTTAACAGGAAACGG
ACTGAAAGATCCGAACACAGCGGTCGACATTTCAGAAATCAAGCCTGTCACATTGCCGA
CTGATGAAGACAGCATCCTTGAATATGTAAAAGGAGCGGCCCGTGTATGA
SEQ ID NO: 320, B. subtilis 168 metA, metAA homoserine
O-acetyltransferase, Gene ID: 939083, NCBI Reference
Sequence: NC_000964.3 region: 2305378-2306283, 906 bp
TTGCCTATTAATATACCAACACACCTGCCGGCAAAACAGGTGCTTGAAAGTGAACATATA
TTTGTAATGGATGAAAGCAGAGCATTTCATCAGGATATCCGGCCGCAAAAAATTATTATA
TTAAATTTGATGCCGAAAAAAATCCAAACAGAGACTCAGCTTCTCAGATTGCTTGGAAAT
TCGCCTTTACAGGTTCATTTCACATTTTTAATCCCAAGCACACACACGCCGAAAAATACT
GCGAGAGAACACCTTGACGAATTTTACACGACTTTTTCCAATATCCGTCACAAGAGGTTT
GACGGCATGATTATCACCGGTGCGCCGATCGAGCATTTGGCGTTTGAAGAGGTTTCATAT
TGGGAAGAGCTCAAGGAAATCATGGAGTGGAGCAAAACAAATGTCACTTCAACCTTGCA
TATTTGCTGGGGTGCTCAAGCAGGTTTGTATTACCATTACGGTGTGGAAAAAATTCAAAT
GCCGAAAAAAATCTTCGGTGTGTTCGAACATACCGTTCTTTCAAAACACGAAAGATTGGT
AAGAGGTTTTGACGAGCTATACTACGTGCCGCATTCCCGGCATACAGATATAAATATGGA
ACAGCTTCAAGCAGTGCCCGAGTTAAACATTCTTACCGCATCAAAGGAAGCCGGGGTTT
GCTTAATTGTGTCTAAAGACGAAAAACAAGTCTTTTTGACAGGGCACCCTGAATATGATA
CAAATACGCTTCTTCAAGAGTATGAAAGAGATTTGGAACGAAACCTTTCTACTGTCGAGG
CCCCTAAACATTATTTTGCAAAAGGCAGCAACGAGCCAGTGAATCGCTGGAAAGCGCAT
GCCACATTATTGTTTATGAATTGGCTGAATTATTACGTTTACCAAGAAACTCCTTATGAAT
GGGACTAA
SEQ ID NO: 321, B. subtilis 168 trpC, indole-3-glycerol-
phosphate synthase, GenBank: EF191535.1 region: 4920-5669,
750 bp
ATGCTTGAAAAAATCATCAAACAAAAGAAAGAAGAAGTGAAAACACTGGTTCTGCCGGT
AGAGCAGCCTTTCGAGAAACGTTCATTTAAGGAGGCGCTGGCAAGCCCGAATCGGTTTA
TCGGGTTGATTGCCGAAGTGAAGAAAGCATCGCCGTCAAAAGGGCTTATTAAAGAGGAT
TTTGTACCTGTGCAGATTGCAAAAGACTATGAGGCTGCGAAGGCAGATGCGATTTCCGTT
TTAACAGACACCCCGTTTTTTCAAGGGGAAAACAGCTATTTATCAGACGTAAAGCGTGCT
GTTTCGATTCCTGTACTTAGAAAAGATTTTATTGATTCTCTTCAAGTAGAGGAATCAAGA
AGAATCGGAGCGGATGCCATATTGTTAATCGGCGAGGTGCTTGATCCCTTACACCTTCAT
GAATTATATCTTGAAGCAGGTGAAAAGGGGATGGACGTGTTAGTGGAGGTTCATGATGC
ATCAACGCTAGAACAAATATTGAAAGTGTTCACACCCGACATTCTCGGCGTAAATAATCG
AAACCTAAAAACGTTTGAAACATCTGTAAAGCAGACAGAACAAATCGCATCTCTCGTTC
CGAAAGAATCCTTGCTTGTCAGCGAAAGCGGAATCGGTTCTTTAGAACATTTAACATTTG
TCAATGAACATGGGGCGCGAGCTGTACTTATCGGTGAATCATTGATGAGACAAACTTCTC
AGCGTAAAGCAATCCATGCTTTGTTTAGGGAGTGA
SEQ ID NO: 322, B. subtilis 168 pheA, prephenate
dehydratase, Gene ID: 937513, NCBI Reference Sequence:
NC_000964.3 region: complement(2851283-2852140), 858 bp
ATGAAAGTCGGTTATTTAGGTCCAGCAGCTACATTTACACATCTAGCAGTCAGTTCTTGT
TTTCAAAACGGCGCCGAACATGTTGCTTACCGCACCATTCCGGAGTGTATAGATGCAGCC
GTTGCAGGCGAAGTTGATTTTGCTTTTGTTCCTTTGGAAAATGCTTTAGAAGGATCTGTTA
ATCTAACAATAGACTATTTAATACATGAACAGCCTTTGCCAATCGTGGGTGAAATGACGT
TGCCGATTCACCAGCACTTGCTCGTCCATCCCTCAAGAGAGAATGCATGGAAAGAGCTCG
ACAAAATTTACTCACATTCACACGCGATTGCGCAATGCCATAAATTTCTTCATCGACATT
TTCCTTCCGTTCCATATGAATACGCCAATTCTACCGGGGCGGCAGCAAAGTTTGTCAGTG
ACCATCCCGAGCTGAATATCGGGGTCATTGCCAATGATATGGCAGCTTCTACATACGAAT
TAAAAATCGTGAAACGGGATATACAGGATTATAGGGACAATCATACAAGATTTGTTATC
CTGTCTCCCGATGAAAACATATCTTTTGAAGTGAATTCAAAATTGAGCTCTAGGCCCAAA
ACGACCTTAATGGTCATGCTGCCGCAGGATGATCAGTCCGGGGCGCTGCATAGAGTGCT
GTCTGCATTTTCTTGGAGAAATTTAAACCTGTCAAAAATTGAGTCACGTCCGACTAAAAC
CGGATTAGGCCATTATTTCTTTATTATTGATATTGAGAAAGCGTTTGATGATGTATTGATT
CCAGGGGCCATGCAGGAGCTTGAAGCACTCGGCTGCAAAGTGAGGCTTCTGGGTGCATA
CCAGTCTTACCAATTATAA
SEQ ID NO: 323, B. thuringiensis serovar kurstaki str. HD73
thrC, HD73_2131, GenBank: CP004069.1, region: 2037098-
2038153, 1056 bp
ATGTATAAAGGACTATTAAAACAATATGCGTCTTATTTACCGGTGAATGAAAATACACCT
GATGTCAGCTTAATGGAAGGAAACACACCACTTATTCCATTATTAAATATATCAAAACAA
TTAGGGATTCAGCTATATGGTAAGTACGAAGGAGCGAATCCGACAGGTTCTTTTAAAGAT
CGTGGTATGGTCATGGCAGTTGCTAAGGCGAAAGAAGAAGGTTCAGAAGCAATCATTTG
TGCATCAACAGGTAATACATCAGCGTCAGCTGCAGCATATGCGGCACGTCTTGGGATGA
AATGCATTATCGTAATACCAGAAGGAAAGATTGCACATGGAAAATTAGCGCAAGCGGTC
GCTTATGGAGCTGAAATTATTICAATAGAAGGGAATTTCGATGATGCGCTTAAGGCTGTA
AGAAATATTGCTGCAGAAGAGCCGATTACGTTAGTAAACTCAGTAAATCCTTATCGAATT
GAAGGGCAAAAAACAGCAGCGTTTGAAATTTGTGACCAATTGCAAAATGCACCAGATGT
TCTAGCTATCCCTGTTGGGAATGCAGGGAATATTACAGCGTATTGGAAAGGCTTCTGTGA
ATATGAGAAAGAAAAAGGTTATAAAAAGCCAAGAATTCATGGCTTTGAAGCGGAAGGA
GCAGCTGCAATTGTAAAAGGGCATGTAATTGAAGAGCCTGAAACAATTGCAACAGCGAT
TCGCATCGGTAACCCAGCAAGTTGGTCGTATGCAGTAGAGGCTGCCGAGCAGTCTCGTG
GTGAAATAGATATGGTATCAGATGAAGAAATTTTACATGCGTATAGGTTATTAGCAAAA
ACTGAAGGAGTTTTCGCTGAACCAGGATCAAATGCTTCATTAGCCGGCGTAATTAAACAT
GTTCAATCTGGGAAAATCAAAAAGGGAGAAACAGTTGTTGCGGTATTAACTGGAAATGG
TTTGAAGGATCCTGATATCGCTATTTCTTCAAATCAACTAGACATCGCAAGTGTGTCAAA
TGATATAGAACAAATTAAAGATCATATTAAAGGGGTGATTATGTCG
SEQ ID NO: 324, B. thuringiensis serovar kurstaki str. HD73
metA, HD73_5818, homoserine O-succinyltransferase, GenBank:
CP004069.1 region: 5552114-5553016, 903 bp
ATGCCGATCATAATTGATAAAGATTTACCGGCTCGCAAAGTGTTACAAAAAGAAAATAT
TTTTGTAATGACGAAGGAGCGAGCGGAAACACAAGATATACGCGCTTTGAAAATTGCTA
TATTAAATTTAATGCCAACGAAGCAAGACACAGAGGCACAATTACTTCGCTTAATTGGA
AACACACCGTTACAATTAGATGTTCATTTGCTTCATATGGAATCACATCTATCTCGTAATG
TAACGCAAGAACATTTAACAAGCTTTTATAAAACGTTTCGTGATATTGAAAATGAAAAGT
TTGATGGGCTCATTATTACAGGAGCGCCAGTGGAGACCCTTGCTTTTGAAGAGGTAGATT
ATTGGGAAGAGCTTAAACATATTATGGAGTATTCAAAAACGAATGTAACATCTACGCTCC
ATATTTGCTGGGGAGCACAGGCTGGCTTGTACTATCATTATGGTGTTCCAAAGTATCCCC
TTAAGGAAAAAATGTTCGGTGTATTTGAACATGAAGTTTGCGAGCAACATGTGAAATTGT
TACAAGGATTTGATGAATTGTTTTTTGCTCCGCATTCTCGTCATACAGAAGTACGAGAGA
ATGATATTAGAGGAGTGAAAGACTTAACATTGTTAGCAAACTCTGAAGAAGCAGGTGTT
CACCTTGTTATTGGGCCAGAAGGAAAGCAAGTGTTTGCCCTTGGACATAGCGAATATAGT
TGTGAGACGTTAAAACAAGAATACGAACGAGATCGTGACAAGGGATTAAATATTGATGT
GCCAAAAAACTATTTTAAACATAATAATCCAGATGAAAAGCCGCTTGTTAGGTGGAGGA
GTCATGGTAATTTATTGTTTTCTAATTGGTTGAATTATTACGTGTATCAAGAAACCCCTTA
TATATTG
SEQ ID NO: 325, B. thuringiensis serovar kurstaki str. HD73
trpC, HD73_1467, Indole-3-glycerol phosphate synthase,
GenBank: CP004069.1 region: 1414090-1414848, 759 bp
GTGGGGACGATTTTAGATAAAATTGTAGAACAGAAGAAAAAGGAAGTIGCGGAGTTATA
TGAAATATATACACCAGTAAAAACAAAAAGAAAGACACATTCACTTGTAGAAGCGTTAC
AGCAGTTTACTGTCATTGCAGAAGTAAAGCGAGCATCACCATCAAAAGGAGATATCAAT
TTACACGTTGACGTACGAAAACAAGTGGGAACATATGAGAAATGTGGTGCAGGAGCAGT
ATCTGTTTTAACAGATGGTCAATTTTTTAAAGGATCGTTTCACGATTTACAAACAGCAAG
AGAAGAAAGTAACATTCCGCTTTTATGTAAAGATTTCATAATCGATAAGATTCAAATCGA
TAGAGCGTATGAAGCGGGAGCAGATATTATTTTACTAATCGTAGCAGCGTTAACGAAAG
AGAAGTTAAAAGAGTTGTATAGCTACGTGTTAGAAAAAGGATTGGAAGCGATTGTTGAA
GTTCATAATGAACAGGAATTAGAAACTGCGATTGTATTAAATCCGCACGTTATTGGTATT
AATAACCGTAATTTAAAAACGTTCGAAGTAGATTTAAGCCAAACTGAAAAGCTTGGAAA
AAGATTGAATGAGGAGAAATTACTTTGGATTAGTGAAAGCGGGATTCATTCAAAAGAGG
ATATTATTCGTGTTAAAAAAGCAGGAGCAAAAGGTGTATTAATTGGAGAGGCACTTATG
ACATCATCTTCTATTAGTAACTTTTTTGAAGACTGTAAGGTGAATATA
SEQ ID NO: 326, B. thuringiensis serovar kurstaki str. HD73
pheA, HD73_4742, prephenate dehydratase, GenBank: CP004069.1
region: 4549095-4549940, 846 bp
ATGATTCGAGTAGGATATTTAGGACCAGAAGCAACATTTACAAATATGGCGGTGAGTCG
TTTTTTTCCGGAAGCAGAGCATGTACCATATCGAACGATTCCAGATTGTATAGATGCAGC
TGCAAATGAAAATGTAGACTTCGCAGTIGTACCGTTAGAAAATGCAATAGAAGGTTCAG
TGAATATAACGGTTGATTACCTTGTACATGAGCAGCCGCTTTCCATTGTAGGAGAAATTA
CAGTACCAATTCAGCAGCATTTACTTGTACATCCGCAGTATGCAGAAGTGTGGGATGAAG
TATATGCAGTGCATTCTCATCCGCATGCCATTGCACAGTGCCATAAATTTTTAAATGAAG
AATTAAAAGGAGTAACTGTTCGAGATATGACATCTACAAGTGCTGCTGCGCAATATGTG
AAAGAACATCCTGAAGAGAAAATTGCCGCTATTGCAAATGAAGCGGCTGCAGAAAAATA
CGGATTAACAATTGTGAGACGTAGTATTCATACGCATAAAAATAATCACACACGTTTCTT
GGTACTCCATAAGAAAAAGAAAGCAGTACTCCCGAATAACGGAGAGAATCGCGGAGAG
AAAACGACGCTTATGATAACGTTACCTGCAGATTATGCAGGTGCCCTATATCAAGTGTTA
TCAGCTTTCGCATGGAGAAAATTAAACTTATCAAAAATCGAATCGCGTCCGATGAAAAC
AGGTCTTGGAAATTACTTTTTCTTAATTGATGTTGATAAAGCATACGATGACGTATTATTG
CCAGGCGTAACGATGGAACTTGAAGCACTCGGTTTTTCTGTTACTGTGCTGGGGAGTTAT
TCTTCTTATTGGTTG
SEQ ID NO: 327, S. cerevisiae (S288C) HIS3,
imidazoleglycerol-phosphate dehydratase HIS3, Gene ID:
854377, NCBI Reference Sequence: NC_001147.6 region:
721946-722608, 663 bp
ATGACAGAGCAGAAAGCCCTAGTAAAGCGTATTACAAATGAAACCAAGATTCAGATTGC
GATCTCTTTAAAGGGTGGTCCCCTAGCGATAGAGCACTCGATCTTCCCAGAAAAAGAGG
CAGAAGCAGTAGCAGAACAGGCCACACAATCGCAAGTGATTAACGTCCACACAGGTATA
GGGTTTCTGGACCATATGATACATGCTCTGGCCAAGCATTCCGGCTGGTCGCTAATCGTT
GAGTGCATTGGTGACTTACACATAGACGACCATCACACCACTGAAGACTGCGGGATTGC
TCTCGGTCAAGCTTTTAAAGAGGCCCTAGGGGCCGTGCGTGGAGTAAAAAGGTTTGGAT
CAGGATTTGCGCCTTTGGATGAGGCACTTTCCAGAGCGGTGGTAGATCTTTCGAACAGGC
CGTACGCAGTTGTCGAACTTGGTTTGCAAAGGGAGAAAGTAGGAGATCTCTCTTGCGAG
ATGATCCCGCATTTTCTTGAAAGCTTTGCAGAGGCTAGCAGAATTACCCTCCACGTTGAT
TGTCTGCGAGGCAAGAATGATCATCACCGTAGTGAGAGTGCGTTCAAGGCTCTTGCGGTT
GCCATAAGAGAAGCCACCTCGCCCAATGGTACCAACGATGTTCCCTCCACCAAAGGTGTT
CTTATGTAG
SEQ ID NO: 328, S. cerevisiae (S288C) LEU2, 3-
isopropylmalate dehydrogenase, Gene ID: 850342,
NCBI Reference Sequence: NC_001135.5 region: 91324-92418,
1095 bp
ATGTCTGCCCCTAAGAAGATCGTCGTTTTGCCAGGTGACCACGTTGGTCAAGAAATCACA
GCCGAAGCCATTAAGGTTCTTAAAGCTATTTCTGATGTTCGTTCCAATGTCAAGTTCGATT
TCGAAAATCATTTAATTGGTGGTGCTGCTATCGATGCTACAGGTGTTCCACTTCCAGATG
AGGCGCTGGAAGCCTCCAAGAAGGCTGATGCCGTTTTGTTAGGTGCTGTGGGTGGTCCTA
AATGGGGTACCGGTAGTGTTAGACCTGAACAAGGTTTACTAAAAATCCGTAAAGAACTT
CAATTGTACGCCAACTTAAGACCATGTAACTTTGCATCCGACTCTCTTTTAGACTTATCTC
CAATCAAGCCACAATTTGCTAAAGGTACTGACTTCGTTGTTGTCAGAGAATTAGTGGGAG
GTATTTACTTTGGTAAGAGAAAGGAAGACGATGGTGATGGTGTCGCTTGGGATAGTGAA
CAATACACCGTTCCAGAAGTGCAAAGAATCACAAGAATGGCCGCTTTCATGGCCCTACA
ACATGAGCCACCATTGCCTATTTGGTCCTTGGATAAAGCTAATGTTTTGGCCTCTTCAAG
ATTATGGAGAAAAACTGTGGAGGAAACCATCAAGAACGAATTCCCTACATTGAAGGTTC
AACATCAATTGATTGATTCTGCCGCCATGATCCTAGTTAAGAACCCAACCCACCTAAATG
GTATTATAATCACCAGCAACATGTTTGGTGATATCATCTCCGATGAAGCCTCCGTTATCC
CAGGTTCCTTGGGTTTGTTGCCATCTGCGTCCTTGGCCTCTTTGCCAGACAAGAACACCGC
ATTTGGTTTGTACGAACCATGCCACGGTTCTGCTCCAGATTTGCCAAAGAATAAGGTCAA
CCCTATCGCCACTATCTTGTCTGCTGCAATGATGTTGAAATTGTCATTGAACTTGCCTGAA
GAAGGTAAGGCCATTGAAGATGCAGTTAAAAAGGTTTTGGATGCAGGTATCAGAACTGG
TGATTTAGGTGGTTCCAACAGTACCACCGAAGTCGGTGATGCTGTCGCCGAAGAAGTTAA
GAAAATCCTTGCTTAA
SEQ ID NO: 329, S. cerevisiae (S288C) LYS2, L-aminoadipate-
semialdehyde dehydrogenase, Gene ID: 852412, NCBI
Reference Sequence: NC_001134.8 region: complement
(469748-473926), 4179 bp
ATGACTAACGAAAAGGTCTGGATAGAGAAGTTGGATAATCCAACTCTTTCAGTGTTACCA
CATGACTTTTTACGCCCACAACAAGAACCTTATACGAAACAAGCTACATATTCGTTACAG
CTACCTCAGCTCGATGTGCCTCATGATAGTTTTTCTAACAAATACGCTGTCGCTTTGAGTG
TATGGGCTGCATTGATATATAGAGTAACCGGTGACGATGATATTGTTCTTTATATTGCGA
ATAACAAAATCTTAAGATTCAATATTCAACCAACGTGGTCATTTAATGAGCTGTATTCTA
CAATTAACAATGAGTTGAACAAGCTCAATTCTATTGAGGCCAATTTTTCCTTTGACGAGC
TAGCTGAAAAAATTCAAAGTTGCCAAGATCTGGAAAGGACCCCTCAGTTGTTCCGTTTGG
CCTTTTTGGAAAACCAAGATTTCAAATTAGACGAGTTCAAGCATCATTTAGTGGACTTTG
CTTTGAATTTGGATACCAGTAATAATGCGCATGTTTTGAACTTAATTTATAACAGCTTACT
GTATTCGAATGAAAGAGTAACCATTGTTGCGGACCAATTTACTCAATATTTGACTGCTGC
GCTAAGCGATCCATCCAATTGCATAACTAAAATCTCTCTGATCACCGCATCATCCAAGGA
TAGTTTACCTGATCCAACTAAGAACTTGGGCTGGTGCGATTTCGTGGGGTGTATTCACGA
CATTTTCCAGGACAATGCTGAAGCCTTCCCAGAGAGAACCTGTGTTGTGGAGACTCCAAC
ACTAAATTCCGACAAGTCCCGTTCTTTCACTTATCGCGACATCAACCGCACTTCTAACAT
AGTTGCCCATTATTTGATTAAAACAGGTATCAAAAGAGGTGATGTAGTGATGATCTATTC
TTCTAGGGGTGTGGATTTGATGGTATGTGTGATGGGTGTCTTGAAAGCCGGCGCAACCTT
TTCAGTTATCGACCCTGCATATCCCCCAGCCAGACAAACCATTTACTTAGGTGTTGCTAA
ACCACGTGGGTTGATTGTTATTAGAGCTGCTGGACAATTGGATCAACTAGTAGAAGATTA
CATCAATGATGAATTGGAGATTGTTTCAAGAATCAATTCCATCGCTATTCAAGAAAATGG
TACCATTGAAGGTGGCAAATTGGACAATGGCGAGGATGTTTTGGCTCCATATGATCACTA
CAAAGACACCAGAACAGGTGTTGTAGTTGGACCAGATTCCAACCCAACCCTATCTTTCAC
ATCTGGTTCCGAAGGTATTCCTAAGGGTGTTCTTGGTAGACATTTTTCCTTGGCTTATTAT
TTCAATTGGATGTCCAAAAGGTTCAACTTAACAGAAAATGATAAATTCACAATGCTGAGC
GGTATTGCACATGATCCAATTCAAAGAGATATGTTTACACCATTATTTTTAGGTGCCCAA
TTGTATGTCCCTACTCAAGATGATATTGGTACACCGGGCCGTTTAGCGGAATGGATGAGT
AAGTATGGTTGCACAGTTACCCATTTAACACCTGCCATGGGTCAATTACTTACTGCCCAA
GCTACTACACCATTCCCTAAGTTACATCATGCGTTCTTTGTGGGTGACATTTTAACAAAAC
GTGATTGTCTGAGGTTACAAACCTTGGCAGAAAATTGCCGTATTGTTAATATGTACGGTA
CCACTGAAACACAGCGTGCAGTTTCTTATTTCGAAGTTAAATCAAAAAATGACGATCCAA
ACTTTTTGAAAAAATTGAAAGATGTCATGCCTGCTGGTAAAGGTATGTTGAACGTTCAGC
TACTAGTTGTTAACAGGAACGATCGTACTCAAATATGTGGTATTGGCGAAATAGGTGAG
ATTTATGTTCGTGCAGGTGGTTTGGCCGAAGGTTATAGAGGATTACCAGAATTGAATAAA
GAAAAATTTGTGAACAACTGGTTTGTTGAAAAAGATCACTGGAATTATTTGGATAAGGAT
AATGGTGAACCTTGGAGACAATTCTGGTTAGGTCCAAGAGATAGATTGTACAGAACGGG
TGATTTAGGTCGTTATCTACCAAACGGTGACTGTGAATGTTGCGGTAGGGCTGATGATCA
AGTTAAAATTCGTGGGTTCAGAATCGAATTAGGAGAAATAGATACGCACATTTCCCAAC
ATCCATTGGTAAGAGAAAACATTACTTTAGTTCGCAAAAATGCCGACAATGAGCCAACA
TTGATCACATTTATGGTCCCAAGATTTGACAAGCCAGATGACTTGTCTAAGTTCCAAAGT
GATGTTCCAAAGGAGGTTGAAACTGACCCTATAGTTAAGGGCTTAATCGGTTACCATCTT
TTATCCAAGGACATCAGGACTTTCTTAAAGAAAAGATTGGCTAGCTATGCTATGCCTTCC
TTGATTGTGGTTATGGATAAACTACCATTGAATCCAAATGGTAAAGTTGATAAGCCTAAA
CTTCAATTCCCAACTCCCAAGCAATTAAATTTGGTAGCTGAAAATACAGTTTCTGAAACT
GACGACTCTCAGTTTACCAATGTTGAGCGCGAGGTTAGAGACTTATGGTTAAGTATATTA
CCTACCAAGCCAGCATCTGTATCACCAGATGATTCGTTTTTCGATTTAGGTGGTCATTCTA
TCTTGGCTACCAAAATGATTTTTACCTTAAAGAAAAAGCTGCAAGTTGATTTACCATTGG
GCACAATTTTCAAGTATCCAACGATAAAGGCCTTTGCCGCGGAAATTGACAGAATTAAAT
CATCGGGTGGATCATCTCAAGGTGAGGTCGTCGAAAATGTCACTGCAAATTATGCGGAA
GACGCCAAGAAATTGGTTGAGACGCTACCAAGTTCGTACCCCTCTCGAGAATATTTTGTT
GAACCTAATAGTGCCGAAGGAAAAACAACAATTAATGTGTTTGTTACCGGTGTCACAGG
ATTTCTGGGCTCCTACATCCTTGCAGATTTGTTAGGACGTTCTCCAAAGAACTACAGTTTC
AAAGTGTTTGCCCACGTCAGGGCCAAGGATGAAGAAGCTGCATTTGCAAGATTACAAAA
GGCAGGTATCACCTATGGTACTTGGAACGAAAAATTTGCCTCAAATATTAAAGTTGTATT
AGGCGATTTATCTAAAAGCCAATTTGGTCTTTCAGATGAGAAGTGGATGGATTTGGCAAA
CACAGTTGATATAATTATCCATAATGGTGCGTTAGTTCACTGGGTTTATCCATATGCCAA
ATTGAGGGATCCAAATGTTATTTCAACTATCAATGTTATGAGCTTAGCCGCCGTCGGCAA
GCCAAAGTTCTTTGACTTTGTTTCCTCCACTTCTACTCTTGACACTGAATACTACTTTAATT
TGTCAGATAAACTTGTTAGCGAAGGGAAGCCAGGCATTTTAGAATCAGACGATTTAATG
AACTCTGCAAGCGGGCTCACTGGTGGATATGGTCAGTCCAAATGGGCTGCTGAGTACATC
ATTAGACGTGCAGGTGAAAGGGGCCTACGTGGGTGTATTGTCAGACCAGGTTACGTAAC
AGGTGCCTCTGCCAATGGTTCTTCAAACACAGATGATTTCTTATTGAGATTTTTGAAAGG
TTCAGTCCAATTAGGTAAGATTCCAGATATCGAAAATTCCGTGAATATGGTTCCAGTAGA
TCATGTTGCTCGTGTTGTTGTTGCTACGTCTTTGAATCCTCCCAAAGAAAATGAATTGGCC
GTTGCTCAAGTAACGGGTCACCCAAGAATATTATTCAAAGACTACTTGTATACTTTACAC
GATTATGGTTACGATGTCGAAATCGAAAGCTATTCTAAATGGAAGAAATCATTGGAGGC
GTCTGTTATTGACAGGAATGAAGAAAATGCGTTGTATCCTTTGCTACACATGGTCTTAGA
CAACTTACCTGAAAGTACCAAAGCTCCGGAACTAGACGATAGGAACGCCGTGGCATCTT
TAAAGAAAGACACCGCATGGACAGGTGTTGATTGGTCTAATGGAATAGGTGTTACTCCA
GAAGAGGTTGGTATATATATTGCATTTTTAAACAAGGTTGGATTTTTACCTCCACCAACT
CATAATGACAAACTTCCACTGCCAAGTATAGAACTAACTCAAGCGCAAATAAGTCTAGTT
GCTTCAGGTGCTGGTGCTCGTGGAAGCTCCGCAGCAGCTTAA
SEQ ID NO: 330, S. cerevisiae (S288C) MET15, bifunctional
cysteine synthase/O-acetylhomoserine aminocarboxypropyl-
transferase (also referred to as MET17), Gene ID: 851010,
NCBI Reference Sequence: NC_001144.5 region: 732542-733876,
1335 bp
ATGCCATCTCATTTCGATACTGTTCAACTACACGCCGGCCAAGAGAACCCTGGTGACAAT
GCTCACAGATCCAGAGCTGTACCAATTTACGCCACCACTTCTTATGTTTTCGAAAACTCT
AAGCATGGTTCGCAATTGTTTGGTCTAGAAGTTCCAGGTTACGTCTATTCCCGTTTCCAAA
ACCCAACCAGTAATGTTTTGGAAGAAAGAATTGCTGCTTTAGAAGGTGGTGCTGCTGCTT
TGGCTGTTTCCTCCGGTCAAGCCGCTCAAACCCTTGCCATCCAAGGTTTGGCACACACTG
GTGACAACATCGTTTCCACTTCTTACTTATACGGTGGTACTTATAACCAGTTCAAAATCTC
GTTCAAAAGATTTGGTATCGAGGCTAGATTTGTTGAAGGTGACAATCCAGAAGAATTCG
AAAAGGTCTTTGATGAAAGAACCAAGGCTGTTTATTTGGAAACCATTGGTAATCCAAAGT
ACAATGTTCCGGATTTTGAAAAAATTGTTGCAATTGCTCACAAACACGGTATTCCAGTTG
TCGTTGACAACACATTTGGTGCCGGTGGTTACTTCTGTCAGCCAATTAAATACGGTGCTG
ATATTGTAACACATTCTGCTACCAAATGGATTGGTGGTCATGGTACTACTATCGGTGGTA
TTATTGTTGACTCTGGTAAGTTCCCATGGAAGGACTACCCAGAAAAGTTCCCTCAATTCT
CTCAACCTGCCGAAGGATATCACGGTACTATCTACAATGAAGCCTACGGTAACTTGGCAT
ACATCGTTCATGTTAGAACTGAACTATTAAGAGATTTGGGTCCATTGATGAACCCATTTG
CCTCTTTCTTGCTACTACAAGGTGTTGAAACATTATCTTTGAGAGCTGAAAGACACGGTG
AAAATGCATTGAAGTTAGCCAAATGGTTAGAACAATCCCCATACGTATCTTGGGTTTCAT
ACCCTGGTTTAGCATCTCATTCTCATCATGAAAATGCTAAGAAGTATCTATCTAACGGTTT
CGGTGGTGTCTTATCTTTCGGTGTAAAAGACTTACCAAATGCCGACAAGGAAACTGACCC
ATTCAAACTTTCTGGTGCTCAAGTTGTTGACAATTTAAAGCTTGCCTCTAACTTGGCCAAT
GTTGGTGATGCCAAGACCTTAGTCATTGCTCCATACTTCACTACCCACAAACAATTAAAT
GACAAAGAAAAGTTGGCATCTGGTGTTACCAAGGACTTAATTCGTGTCTCTGTTGGTATC
GAATTTATTGATGACATTATTGCAGACTTCCAGCAATCTTTTGAAACTGTTTTCGCTGGCC
AAAAACCATGA
SEQ ID NO: 331, S. cerevisiae (S288C) URA3, orotidine-5′-
phosphate decarboxylase, Gene ID: 856692, NCBI Reference
Sequence: NC_001137.3 region: 116167-116970, 804 bp
ATGTCGAAAGCTACATATAAGGAACGTGCTGCTACTCATCCTAGTCCTGTTGCTGCCAAG
CTATTTAATATCATGCACGAAAAGCAAACAAACTTGTGTGCTTCATTGGATGTTCGTACC
ACCAAGGAATTACTGGAGTTAGTTGAAGCATTAGGTCCCAAAATTTGTTTACTAAAAACA
CATGTGGATATCTTGACTGATTTTTCCATGGAGGGCACAGTTAAGCCGCTAAAGGCATTA
TCCGCCAAGTACAATTTTTTACTCTTCGAAGACAGAAAATTTGCTGACATTGGTAATACA
GTCAAATTGCAGTACTCTGCGGGTGTATACAGAATAGCAGAATGGGCAGACATTACGAA
TGCACACGGTGTGGTGGGCCCAGGTATTGTTAGCGGTTTGAAGCAGGCGGCGGAAGAAG
TAACAAAGGAACCTAGAGGCCTTTTGATGTTAGCAGAATTGTCATGCAAGGGCTCCCTAG
CTACTGGAGAATATACTAAGGGTACTGTTGACATTGCGAAGAGCGACAAAGATTTTGTTA
TCGGCTTTATTGCTCAAAGAGACATGGGTGGAAGAGATGAAGGTTACGATTGGTTGATTA
TGACACCCGGTGTGGGTTTAGATGACAAGGGAGACGCATTGGGTCAACAGTATAGAACC
GTGGATGATGTGGTCTCTACAGGATCTGACATTATTATTGTTGGAAGAGGACTATTTGCA
AAGGGAAGGGATGCTAAGGTAGAGGGTGAACGTTACAGAAAAGCAGGCTGGGAAGCAT
ATTTGAGAAGATGCGGCCAGCAAAACTAA

In some embodiments of any of the aspects, the inactivating modification of at least one essential compound synthesis gene only disrupts the synthesis pathway of one essential compound. As a non-limiting example, in some species isoleucine, valine, and leucine share the beginning of a common synthesis pathway, with each having a distinct end of a synthesis pathway for each amino acid. As such, in some embodiments of any of the aspects, the inactivating modification does not occur in a synthesis gene in a common synthesis pathway (e.g., for isoleucine, valine, and leucine) but rather in a synthesis pathway specific for one essential compound.

In some embodiments of any of the aspects, the engineered microorganism comprises an inactivating modification of at least two or more essential compound synthesis genes. In some embodiments of any of the aspects, the engineered microorganism comprises at least one inactivating modification of at least 1, at least 2, at least 3, at least 4, at least 5, at least 6, at least 7, at least 8, at least 9, or at least 10 essential compound synthesis genes. In some embodiments of any of the aspects, the engineered microorganism comprises an inactivating modification of at least two, but not more than 10, not more than 9, not more than 8, not more than 7, not more than 6, not more than 5, not more than 4, or not more than 3 essential compound synthesis genes. In some embodiments of any of the aspects, the engineered microorganism comprises at least 1, at least 2, at least 3, at least 4, at least 5, at least 6, at least 7, at least 8, at least 9, or at least 10 inactivating modifications of at least 1 essential compound synthesis gene.

In some embodiments of any of the aspects, the at least one essential gene of the engineered microorganism (e.g., a Bacillus species) is selected from the essential genes identified for B. subtilis in Koo et al., Construction and Analysis of Two Genome-scale Deletion Libraries for Bacillus subtilis, Cell Syst. 2017 Mar. 22, 4(3): 291-305.e7 (see e.g., Supplementary Table 5); the content of which is incorporated herein by reference in its entirety. In some embodiments of any of the aspects, the at least one essential compound synthesis gene of the engineered microorganism (e.g., a Bacillus species) is selected from Table 8.

TABLE 8
Auxotrophic genes in B. subtilis (and or/B. thuringiensis)

| gene | product |
|---|---|
| guaB | inosine-monophosphate dehydrogenase |
| cysK | cysteine synthase |
| cysE | serine acetyltransferase |
| guaA | GMP synthetase |
| purE | phosphoribosylaminoimidazole carboxylase I |
| purK | phosphoribosylaminoimidazole carboxylase ATPase subunit |
| purB | adenylosuccinate lyase |
| purC | phosphoribosylaminoimidazole succinocarboxamide synthetase |
| purS | factor required for phosphoribosylformylglycinamidine synthetase |
| | activity |
| purQ | phosphoribosylformylglycinamidine synthetase I |
| purL | phosphoribosylformylglycinamidine synthetase II |
| purF | glutamine phosphoribosylpyrophosphate amidotransferase |
| purM | phosphoribosylaminoimidazole synthetase |
| purH | fused phosphoribosylaminoimidazole carboxy formyl |
| | formyltransferase inosine-monophosphate cyclohydrolase |
| purD | phosphoribosylglycinamide synthetase |
| serC | phosphoserine aminotransferase |
| yitJ | homocysteine S-methyltransferase using (R,S)AdoMet and |
| | methylenetetrahydrofolate reductase [NAD(P)H] |
| argC | N-acetylglutamate gamma-semialdehyde dehydrogenase |
| argJ | ornithine acetyltransferase amino-acid acetyltransferase |
| argB | N-acetylglutamate 5-phosphotransferase (acetylglutamate kinase) |
| argD | N-acetylornithine aminotransferase |
| argF | ornithine carbamoyltransferase |
| metI | cystathionine gamma-synthase and O-acetylhomoserine thiolyase |
| proB | glutamate 5-kinase |
| proA | gamma-glutamyl phosphate reductase |
| metE | cobalamin-independent methionine synthase |
| pdhA | pyruvate dehydrogenase (E1 alpha subunit) |
| pdhB | pyruvate dehydrogenase (E1 beta subunit) |
| pycA | pyruvate carboxylase |
| pyrB | aspartate carbamoyltransferase |
| pyrC | dihydroorotase |
| pyrK | dihydroorotate dehydrogenase (electron transfer subunit) |
| pyrD | dihydroorotate dehydrogenase (catalytic subunit) |
| pyrF | orotidine 5-phosphate decarboxylase |
| pyrE | orotate phosphoribosyltransferase |
| sirB | sirohydrochlorin ferrochelatase |
| glnA | glutamine synthetase |
| tkt | transketolase |
| citB | aconitate hydratase (aconitase) |
| gltB | glutamate synthase (small subunit) |
| gltA | glutamate synthase (large subunit) |
| gltC | transcriptional regulator (LysR family) |
| ilvA | threonine dehydratase |
| ilvD | dihydroxy-acid dehydratase |
| metA | putative homoserine O-acetyltransferase |
| aspB | putative aspartate aminotransferase |
| panC | pantothenate synthetase |
| tyrA | prephenate dehydrogenase |
| hisC | histidinol-phosphate aminotransferase tyrosine/phenylalanine |
| | aminotransferase |
| trpA | tryptophan synthase (alpha subunit) |
| trpB | tryptophan synthase (beta subunit) |
| trpF | phosphoribosyl anthranilate isomerase |
| trpC | indol-3-glycerol phosphate synthase |
| trpD | anthranilate phosphoribosyltransferase |
| trpE | anthranilate synthase |
| aroB | 3-dehydroquinate synthase |
| gpsA | NAD(P)H-dependent glycerol-3-phosphate dehydrogenase |
| serA | 3-phosphoglycerate dehydrogenase |
| aroC | 3-dehydroquinate dehydratase |
| lysA | diaminopimelate decarboxylase |
| folD | methylenetetrahydrofolate dehydrogenase methenyltetrahydrofolate |
| | cyclohydrolase |
| aroD | shikimate 5-dehydrogenase |
| nadA | quinolinate synthetase |
| pheA | prephenate dehydratase |
| leuD | 3-isopropylmalate dehydratase (small subunit) |
| leuC | 3-isopropylmalate dehydratase (large subunit) |
| leuB | 3-isopropylmalate dehydrogenase |
| leuA | 2-isopropylmalate synthase |
| ilvC | acetohydroxy-acid isomeroreductase |
| ilvH | acetolactate synthase (acetohydroxy-acid synthase) (small subunit) |
| ilvB | acetolactate synthase (acetohydroxy-acid synthase) (large subunit) |
| ysaA | putative phosphatase |
| icd | isocitrate dehydrogenase |
| argH | argininosuccinate lyase |
| argG | argininosuccinate synthase |
| hisJ | histidinol phosphate phosphatase |
| aroA | 3-deoxy-D-arabino-heptulosonate 7-phosphate synthase chorismate |
| | mutase-isozyme 3 |
| yugH | putative aspartate aminotransferase |
| thrB | homoserine kinase |
| thrC | threonine synthase |
| hom | homoserine dehydrogenase |
| cysI | sulfite reductase (hemoprotein beta-subunit) |
| cysJ | sulfite reductase (flavoprotein alpha-subunit) |
| tpiA | triose phosphate isomerase |
| pgk | phosphoglycerate kinase |
| hisI | phosphoribosyl-AMP cyclohydrolase phosphoribosyl-ATP |
| | pyrophosphohydrolase |
| hisF | imidazole glycerol phosphate synthase subunit |
| hisA | phosphoribosylformimino-5-aminoimidazole carboxamide ribotide |
| | isomerase |
| hisH | amidotransferase (glutaminase) |
| hisB | imidazoleglycerol-phosphate dehydratase |
| hisD | histidinol dehydrogenase |
| hisG | ATP phosphoribosyltransferase |
| hisZ | histidyl-tRNA synthetase component of ATP |
| | phophoribosyltransferase |
| glyA | serine hydroxymethyltransferase |
| ywlF | ribose 5-phosphate epimerase |
| fbaA | fructose-1,6-bisphosphate aldolase |
| cysL | regulator of sulfur assimilation CysL, activates cysJI expression |
| purA | adenylosuccinate synthetase |

In some embodiments of any of the aspects, the at least one essential compound synthesis gene of the engineered microorganism (e.g., a Saccharomyces species) is selected from the group consisting of ade1, ade2, can1, his3, leu2, lys2, met15, trp1, trp5, ura3, ura4. In some embodiments of any of the aspects, the at least one inactivating modification of at least one essential compound synthesis gene of the engineered microorganism (e.g., a Bacillus species) is selected from Table 9; see e.g., Brachmann et al. (1998) “Designer deletion strains derived from Saccharomyces cerevisiae S288C: a useful set of strains and plasmids for PCR-mediated gene disruption and other applications.” Yeast 14:115-132; the content of which is incorporated herein by reference in its entirety.

TABLE 9
List of auxotrophic mutations in S. cerevisiae

| | Deleted | | | | |
|---|---|---|---|---|---|
| Allele | ORF? | Reverts? | Notes | Molecular Description | Reference |
| ade1-14 | no | yes | red colonies | TGG-to-TGA nonsense | Nakayashiki et |
| | | | | change at codon 244; | al. 2001 |
| | | | | GGA-to-GAA missense | |
| | | | | change at codon 185 | |
| ade1-101 | no | | simple point mutation | | Botstein et al. |
| | | | causing a requirement | | 1979 |
| | | | for adenine | | |
| ade2-1 | no | yes | ochre mutation | TTA-to-TTG silent | Rodney |
| | | | | change at codon 9, GAA- | Rothstein, |
| | | | | to-TAA ochre nonsense | Personal |
| | | | | change at codon 64, | communication |
| | | | | AGA-to-GGA missense | to SGD |
| | | | | change at codon 101, | |
| | | | | GTT-to-GTC silent | |
| | | | | change at codon 124, | |
| | | | | ACG-to-ACA silent | |
| | | | | change at codon 539 | |
| ade2-101 | no | yes | ochre mutation, red | G to T transversion at | Gai and |
| | | | colonies | nucleotide 190, changing | Voytas, 2005 |
| | | | | codon 64 from a Glu to a | |
| | | | | Stop | |
| ade 2- | no | no | red colonies | frameshift (BglII site | Engebrecht |
| BglII | | | | filled in at position 592) | and Roeder |
| | | | | | 1990 |
| can1-100 | no | yes | ochre mutation | AAA-to-TAA ochre | Rodney |
| | | | | nonsense change at codon | Rothstein, |
| | | | | 47 | Personal |
| | | | | | communication |
| | | | | | to SGD |
| his3delta200 | yes | no | Cold sensitive; high | 1 kb deletion (−205 to | Struhl |
| | | | frequency of petite | 835) | 1985; Fasullo |
| | | | formation, especially | | and Davis |
| | | | during | | 1988 |
| | | | transformation. Note | | |
| | | | that this deletion | | |
| | | | damages the PET56 | | |
| | | | promoter. See Zhang | | |
| | | | et al. (2003) for a | | |
| | | | discussion of this | | |
| | | | issue. | | |
| his3delta1 | partial | no | chemically | 187 bp HindIII-HindIII | Scherer and |
| | | | constructed deletion; | internal deletion (305 to | Davis |
| | | | reversion is | 492) | 1979 Botstein |
| | | | undetectable (<10(E−9) | | et al. 1979 |
| | | | revertants in a | | |
| | | | culture). Causes | | |
| | | | histidine to be | | |
| | | | required for growth; | | |
| | | | allows selection of | | |
| | | | vectors carrying the | | |
| | | | HIS3 gene. | | |
| his3- | no | no | double mutant | G deletion at nucleotide | Rodney |
| 11,15 | | | | 208, G deletion at | Rothstein, |
| | | | | nucleotide 319 | Personal |
| | | | | | communication |
| | | | | | to SGD |
| his3delta | yes | no | In the S288C | complete deletion | Ding et al. |
| | | | background, the his3 | | 2013 |
| | | | null mutation confers | | |
| | | | sensitivity to acetic | | |
| | | | acid. | | |
| leu2delta1 | partial | no | | EcoRI-ClaI internal | Sikorski and |
| | | | | deletion (163 to 649, 0.6 | Hieter 1989 |
| | | | | kb) | |
| leu2- | no | no | double mutant; leu2-3 | GTC-to-GTT silent | Hinnen et al. |
| 3,112 | | | and leu2-112 are each | change at codon 56, GTT- | 1978; Gaber |
| | | | frameshift mutations; | to-GCT missense change | and Culbertson |
| | | | the double mutants | at codon 69, G insertion at | 1982; Meira |
| | | | revert very | nucleotide 249, G | L B et al., |
| | | | infrequently (less | insertion at nucleotide | 1995; Rodney |
| | | | than 10(E−8) | 792, GTT-to-GTC silent | Rothstein, |
| | | | revertants in a | change at codon 299, | Personal |
| | | | culture) and cause | GAC-to-AAC missense | communication |
| | | | leucine to be required | change at codon 300 | to SGD Botstein |
| | | | for growth. This | | et al. 1979 |
| | | | allows the selection | | |
| | | | of vectors carrying | | |
| | | | the LEU2 gene. | | |
| leu2delta | yes | no | In the S288C | complete deletion | Ding et al. |
| | | | background, the leu2 | | 2013 |
| | | | null mutation confers | | |
| | | | sensitivity to acetic | | |
| | | | acid. | | |
| lys2-801 | no | yes | amber mutation | | |
| lys2delta202 | partial | no | | XhoI-HpaI internal | Winston et al. |
| | | | | deletion (1813 to 2864, | 1995 |
| | | | | 1.0 kb) | |
| lys2delta | yes | no | In the S288C | complete deletion | Ding et al. |
| | | | background, the lys2 | | 2013 |
| | | | null mutation confers | | |
| | | | sensitivity to acetic | | |
| | | | acid. | | |
| trp1delta1 | yes | no | cold sensitive, weak | 1.45 kb deletion, EcoRI- | Sikorski and |
| | | | galactose inducer | EcoRI (−102 to 1352) | Hieter 1989 |
| | | | (deletes GAL3 UAS), | | |
| | | | removes ARS1, also | | |
| | | | called trp1-901 | | |
| trp1delta63 | partial | no | cold sensitive | 0.6 kb deletion, EcoRI- | Sikorski and |
| | | | | HindIII (−102 to 513) | Hieter 1989 |
| trp1-1 | no | yes | amber mutation | GAG-to-TAG amber | McDonald et |
| | | | | nonsense change at codon | al. 1997 |
| | | | | 83 | |
| trp1-1 | no | yes | cold sensitive | C to T at residue 403 of | Brian Green |
| | | | | the coding sequence, | and Joachim |
| | | | | changing residue 135 | Li, unpublished |
| | | | | from glutamine to an | results |
| | | | | amber stop codon | |
| trp1delta | yes | no | In the S288C | complete deletion | Ding et al. |
| | | | background, the trp1 | | 2013 |
| | | | null mutation confers | | |
| | | | sensitivity to acetic | | |
| | | | acid. | | |
| trp1-289 | no | yes | Reverts at low | Allows the selection of | Botstein et al. |
| | | | frequency (about | vectors carrying the TRP1 | 1979 |
| | | | 10(E−7) revertants in | gene. | |
| | | | a culture) and causes | | |
| | | | tryptophan to be | | |
| | | | required for growth. | | |
| trp5delta | yes | no | In the S288C | complete deletion | Ding et al. |
| | | | background, the trp5 | | 2013 |
| | | | null mutation confers | | |
| | | | sensitivity to acetic | | |
| | | | acid. | | |
| ura3-52 | no | no | | Ty1 insertion (transcribing | Rose and |
| | | | | left to right) at pos. 121 | Winston 1984 |
| ura3-1 | no | yes | | G to A transition at | Yan Li, Glenn |
| | | | | residue 701 of the coding | Manthey, and |
| | | | | sequence, changing | Adam Bailis, |
| | | | | residue 234 from glycine | unpublished |
| | | | | to glutamate | results |
| ura3delta | yes | no | In the S288C | complete deletion | Ding et al. |
| | | | background, the ura3 | | 2013 |
| | | | null mutation confers | | |
| | | | sensitivity to acetic | | |
| | | | acid. | | |
| ura4delta | yes | no | In the S288C | complete deletion | Ding et al. |
| | | | background, the ura4 | | 2013 |
| | | | null mutation confers | | |
| | | | sensitivity to acetic | | |
| | | | acid. | | |
| ade2delta::hisG | no | no | | | Brachmann et |
| | | | | | al. 1998, |
| | | | | | (Aparicio et al. |
| | | | | | 1991) |
| leu2delta0 | yes | no | designer deletion | | Brachmann et |
| | | | | | al. 1998 |
| lys2delta0 | yes | no | designer deletion | | Brachmann et |
| | | | | | al. 1998 |
| met15delta0 | yes | no | designer deletion | | Brachmann et |
| | | | | | al. 1998 |
| ura3delta0 | yes | no | designer deletion | | Brachmann et |
| | | | | | al. 1998 |

### Germination Genes

In one aspect of any of the embodiments, an engineered microorganism comprises at least one inactivating modification of at least one germination gene. As used herein, the term germination refers to the process by which an endospore loses spore-specific properties, e.g., loss of dormancy, loss of spore wall, regained growth capabilities. Germination genes express products that are essential, alone or in combination, for germination to occur. In some embodiments of any of the aspects, the at least one germination gene is selected from the group consisting of cwlJ, sleB, gerAB, gerBB, and gerKB (e.g., from a Bacillus species).

CwlJ and SleB are enzymes that are needed to degrade the spore cell wall during germination. ΔcwlJ ΔsleB mutants are deficient in their ability or unable to degrade the spore cell wall during germination. GerA, GerB, and GerK are germinant receptors that sense and respond to nutrients. ΔgerAB ΔgerBB ΔgerKB mutants thus has a decreased ability or inability to sense and respond to nutrients for germination. In some embodiments of any of the aspects, the at least one germination gene is GerD, SpoVA, and/or a gene encoding a cortex-lytic enzyme (CLE); see e.g., Setlow et al., J Bacteriol. 2014 April, 196(7):1297-305; Paidhungat et al., J Bacteriol. 2001 August, 183(16):4886-93; each of which is incorporated herein by reference in its entirety.

In some embodiments of any of the aspects, the germination gene comprises one of SEQ ID NOs: 332-340 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to one of SEQ ID NOs: 332-340, that maintains the same function (e.g., germination of spores).

B. subtilis 168 cwlJ, spore cortex
cell wall hydrolase, Gene ID:
938399, NCBI Reference Sequence:
NC_000964.3 region: 282469-282897,
429 bp
SEQ ID NO: 332
ATGGCGGTCGTGAGAGCAACGAGTGCGGATGTCGATTTGA
TGGCAAGGCTGCTCAGAGCGGAAGCGGAAGGCGAAGGCAA
GCAGGGGATGCTGCTTGTCGGCAACGTTGGAATTAATCGG
CTGCGGGCGAATTGCTCAGATTTTAAAGGCCTCCGCACCA
TCAGGCAGATGATTTATCAGCCACACGCGTTTGAGGCTGT
GACTCATGGATATTTTTATCAAAGGGCGCGAGATAGCGAG
CGTGCCCTTGCACGCCGGTCGATTAATGGTGAAAGGCGCT
GGCCTGCAAAATTTAGTTTATGGTACTTCAGGCCGCAGGG
GGACTGTCCAGCCCAGTGGTATAACCAGCCGTTTGTGGCC
AGATTTAAGTCACACTGCTTTTATCAGCCGACGGCGGAGA
CGTGTGAAAATGTATATAACACATTTTAG,
B. subtilis 168 sleB, spore germination
cortex-lytic enzyme, Gene ID:
938979, NCBI Reference Sequence: NC_000964.3
region: complement (2399152-2400069), 918 bp
SEQ ID NO: 333
ATGAAGTCCAAAGGATCGATTATGGCATGTCTCATCCTTT
TTTCCTTTACAATAACGACGTTTATTAATACTGAAACGAT
CTCTGCCTTTTCGAATCAGGTCATTCAAAGAGGGGCAACA
GGGGATGATGTGGTCGAGCTTCAGGCGCGTCTTCAATACA
ACGGATATTATAACGGAAAAATTGACGGGGTTTATGGATG
GGGGACGTACTGGGCAGTTCGAAATTTTCAGGATCAATTC
GGGTTAAAAGAGGTTGACGGCCTTGTAGGAGCTAAAACAA
AGCAAACCTTAATATGTAAATCAAAATACTATCGTGAATA
TGTCATGGAACAGCTCAATAAAGGGAATACATTCACGCAT
TACGGAAAAATTCCGCTAAAGTATCAGACGAAACCATCAA
AAGCAGCAACACAAAAGGCAAGACAACAAGCAGAAGCACG
GCAGAAACAGCCTGCGGAAAAAACAACGCAGAAGCCTAAA
GCGAATGCGAATAAACAGCAAAACAATACACCAGCAAAAG
CAAGAAAACAGGATGCGGTAGCAGCGAACATGCCTGGTGG
ATTTTCCAACAACGATATCAGGCTGCTTGCTCAAGCGGTT
TATGGCGAAGCCCGGGGCGAGCCGTACGAGGGGCAGGTTG
CTATTGCAGCAGTCATTTTAAACCGTTTGAACAGCCCGTT
ATTTCCAAATTCAGTAGCGGGGGTTATTTTTGAGCCGCTT
GCCTTCACAGCAGTAGCCGACGGACAAATTTACATGCAGC
CGAATGAAACGGCACGAGAAGCAGTGCTGGATGCCATCAA
TGGCTGGGACCCATCAGAGGAAGCACTTTACTACTTTAAT
CCGGATACGGCTACAAGTCCGTGGATTTGGGGGCGTCCGC
AGATTAAAAGAATCGGTAAACACATTTTCTGTGAGTAG,
B. subtilis 168 gerAB, component of the
germination receptor GerA;
putative transporter, Gene ID: 935948,
NCBI Reference Sequence: NC_000964.3
region: 3392199-3393296, 1098 bp
SEQ ID NO: 334
ATGAGCCAAAAACAGACTCCACTGAAACTTAATACCTTTC
AAGGTATTTCTATTGTTGCAAATACAATGCTTGGGGCCGG
ACTTTTAACATTGCCGCGAGCGCTGACCACTAAGGCTAAT
ACGCCTGACGGCTGGATCACGCTGATATTAGAAGGTTTCA
TTTTTATTTTCTTCATTTATTTAAACACGCTCATTCAGAA
AAAACATCAATACCCTTCACTCTTTGAATATTTGAAGGAA
GGGCTTGGGAAATGGATCGGCAGCATCATCGGCCTTTTGA
TCTGCGGCTATTTCCTCGGCGTAGCCAGCTTCGAGACACG
GGCAATGGCTGAAATGGTGAAGTTTTTCCTGCTGGAGAGA
ACCCCAATTCAAGTCATTATTTTAACGTTTATTTGCTGCG
GCATTTATTTAATGGTTGGGGGCTTAAGCGATGTGTCGCG
GCTGTTTCCCTTTTATTTAACGGTAACCATTATTATTTTG
CTGATTGTGTTCGGGATCAGTTTTAAAATTTTTGATATCA
ATAATTTGCGTCCTGTTTTAGGCGAAGGCCTTGGACCCAT
TGCAAACTCCCTTACCGTCGTTTCCATCTCTTTTTTAGGA
ATGGAAGTGATGCTGTTTCTTCCTGAACATATGAAGAAAA
AGAAATACACGTTCAGATATGCGTCTCTAGGATTTCTGAT
TCCGATTATCTTATATATCCTTACGTATATTATCGTTGTC
GGAGCTTTGACCGCTCCCGAGGTGAAAACGCTGATTTGGC
CGACTATTTCTCTCTTTCAGTCCTTTGAGCTTAAAGGCAT
ATTTATTGAACGGTTTGAATCCTTTTTACTGGTGGTCTGG
ATTATCCAGTTTTTCACCACATTTGTCATTTACGGATACT
TTGCCGCTAACGGGCTAAAGAAGACATTTGGATTATCGAC
TAAAACAAGCATGGTGATTATCGGCATAACGGTCTTTTAT
TTTTCTCTCTGGCCGGATGACGCCAATCAAGTCATGATGT
ATAGCGATTATCTTGGCTATATTTTTGTCTCCCTGTTTCT
GCTTCCGTTCATTCTCTTTTTCATTGTAGCTCTCAAGAGG
AGGATTACAACAAAATGA,
B. subtilis 168 gerBB, component of
germinant receptor B, Gene ID:
936827, NCBI Reference Sequence:
NC_000964.3 region: 3690269-3691375,
1107 bp
SEQ ID NO: 335
ATGAGGAAATCAGAGCATAAACTGACATTTATGCAGACGC
TCATTATGATCAGCAGCACATTGATTGGTGCCGGGGTGCT
GACCCTTCCCCGCTCAGCCGCCGAAACCGGCAGTCCGAGC
GGATGGCTAATGATACTGCTCCAGGGCGTTATTTTTATTA
TCATCGTTCTGCTTTTTTTGCCTTTTCTTCAAAAAAACAG
CGGAAAAACTCTTTTTAAGCTCAACAGCATTGTAGCTGGA
AAATTCATCGGCTTTCTATTGAATTTATATATCTGTCTAT
ATTTCATTGGGATTGTTTGCTTTCAAGCTCGGATTTTGGG
AGAGGTTGTCGGATTCTTTTTGTTGAAAAATACGCCAATG
GCAGTIGTGGTGTTTATATTTCTTGCAGTTGCCATCTATC
ATGTAGGCGGAGGCGTTTATTCAATTGCAAAAGTATACGC
TTATATTTTTCCTATAACCCTTATTATTTTTATGATGCTT
CTGATGTTCAGCTTTCGCTTGTTCCAGCTTGATTTTATCC
GGCCGGTATTTGAAGGAGGCTATCAAAGCTTTTTCTCTTT
ATTCCCAAAAACATTATTATATTTCTCCGGATTTGAAATC
ATTTTTTACCTGGTCCCCTTTATGAGAGATCCAAAGCAAG
TGAAAAAGGCTGTTGCTCTGGGCATCGCGACTTCCACATT
GTTCTACAGCATTACTTTGCTCATTGTGATTGGCTGTATG
ACTGTGGCTGAGGCAAAAACGGTGACATGGCCGACCATTT
CCCTTATTCACGCATTAGAGGTTCCGGGTATTTTTATTGA
ACGATTTGATTTGTTCTTACAGCTGACCTGGACAGCCCAG
CAATTTGCCTGTATGCTCGGATCCTTTAAAGGCGCCCATA
TCGGGTTAACGGAAATCTTTCATCTAAAAAATAAAAACAA
TGCATGGCTGCTGACTGCAATGCTGGCTGCGACGTTTTTC
ATAACGATGTATCCCAAGGACTTGAATGACGTGTTTTATT
ACGGGACGCTTCTTGGATATGCTTTCTTAATTGTGATTAC
AATTCCATTCTTCGTCTGGTTTTTAAGCTGGATACAGAAA
AAAATAGGGAGGGGGCAGCTGCAATGA,
B. subtilis 168 gerKB, spore germination
receptor subunit, Gene ID:
938282, NCBI Reference Sequence:
NC_000964.3 region: 422982-424103, 1122 bp
SEQ ID NO: 336
GGCAGCTCCTTATTGATTACACCGGGATCAATGGCGGGCA
GGGATGCTTGGATAGCAGTTTTATTAGGCTGTGCGATCGG
ACTGTTTCTTTTCTATTTGTATCAAGGCATTTATCAATGT
TATCCGAATTCTTCTCCGAAAGAATATATGGATGATATGC
TGGGAACCAAGCTGAGCTGGCTGTTTTCATTTCTCTATAT
CTTGTACTTTGCCTATATTGCCGCAAGGGTGCTGCGTGAC
TTTGGAGAAATGCTGCTGACGTTTGCTTATCATGATACAC
CTATCATTATTGTAAATGCTTTATTAATGGTAGTAAGCAT
ATATGCCGTAAGAAAAGGAATTGAAGTACTCGCACGCGCG
GCCGAACTTCTTTTCGGGGCCATGTATTTACTGGGCGCGA
TCGGCCTTGTGCTGATTATTGTCTCAGGGACTATAGACCC
GCACAATTTAAAGCCGGTTTTGGCAAACGGCATTTCTCCT
GTCCTTCACTCTGTTTTCACACAGACCATGTACGTTCCAT
TTGGTGAAGTTGTTTTATTTGTGATGATTTTCCCTAACCT
GAATGACAGAAAAGATGTGAAAAAAATGGGGATGATCGCC
ATGGCTATTAGCGGATTGATTGTGGCGCTCACCGTGGCGA
TTAATATTAGCGTGCTTGATGTTGATCTTACACTCAGATC
CCAGTTTCCGCTTTTAAGCACGATCCAGACAATCAAGGTC
GAAGAATTTTTAGACCGGCTCGATGTCTTTTTTATGCTGG
CGCTGATCATCGGCGGCTTCTTTAAGGTCAGCCTCTACTT
ATATGCCACTGTGGTTGGGACATCTACGCTCTTTAAGGAA
AAGAACCCCTCTCAATTGGCTTATCCGATGGGATTGGGCA
TCTTAATCCTTTCTATAACGATCGCGACTAACTTTTCGGA
GCACTTGAATGAAGGCCTGAACGTGGTGCCGCTGTATATT
CATTTGCCTTTTCAATTATTGTTTCCGCTCTTTCTGTTTA
TTGTGGCCGTTTGGAAAAAGAAACGAAGAGAGAAGTCAAA
AGGGGAGGAAGCAAAAAAATGA,
B. thuringiensis serovar kurstaki str.
HD73 cw/J, HD73_4049, Cell
wall hydrolase, GenBank: CP004069.1
region: 3897606-3898400, 795 bp
SEQ ID NO: 337
ATGAAATTACTAAAACTAAAACATATAATCCCTTTATCTG
CAGCTGCAATTACATTCGTATGTAGTCAAAGCACAGCTGA
AGCTTCTACCATTCATACAGTAAAAAAGAACGATACACTT
TGGGGCATTAGTAAACAATACGGAGTTTCAATACAATCCA
TTAAACAGGCTAATAATAAAGGAAACGATCAAACCTTTAT
TGGCGAACAGTTACATATTCCAGGGTCCGTGAAATCAAAT
GAAATCACTGTTCCTCAAAACGCCAAGCCTGCGAACATTT
CTGGACAAATTATTTATCAAGTACAACCGGGAGATTCATT
AGAAACGATAGCAAGGCGTTACAATGTCACTGTCCAATCT
ATAAAACAAATTAACAATACAATCGGAAACAAGCTTTATA
CAGGACAACATTTAAGAAGCAGGTGGCGAATCCTATAAAG
GAAAAGTAGCAGTAGCGAAAGTTATACTAAATCGTGTAAA
TGCAAAAGGTTTTCCAAATACAATAACAGGCGTTATTTAT
GAACCTATTAAATACGGATATGCATTTACTCCTGTTACAG
ATGGAAGAATTAATCAGCCTGCAAGCGCAGAAGCAAAAAT
GGCTGTAGAAGAGGCTATCTCCACAAATGGAGTACATTCT
GACTGGCTTTATTTCTACAATCCAAAAACATCAACAGACA
AGTGGATTACGACACGTCAGACAGTAGCAGTAATTGGTAA
TCATGTGTTTGCTAAA,
B. thuringiensis serovar
kurstaki str. HD73 sleB, HD73_3242, Spore
cortex-lytic enzyme, GenBank: CP004069.1
region: 3106881-3107657, 777 bp
SEQ ID NO: 338
ATGCGCCAAAAAGCTATTTTTAAAATAGCAGTTTTACTTG
CGTTCATAGGACTGTCTTTAATGGTCAGTAGTATACAATT
AAAGAATGTAGAGGCTTTTTCTAATCAAGTTATTCAAAGA
GGAGCATCTGGCGAAGATGTCATTGAACTGCAATCTCGTT
TGAAATATAACGGGTTTTATACGGGAAAAGTGGATGGTGT
TTTCGGATGGGGTACATATTGGGCACTTCGGAATTTTCAA
GAGAAATTCGGATTACCCGTTGATGGTTTAGCTGGAGCTA
AAACGAAGCAAATGCTCGTGAAGGCAACGAAGTATGACAA
GTCAACTGCTAATAAAGGGACGACAACGAATAAAGGAAAT
AGCGGTGGCACTGCACAAGAGAATAAACCACTTCAAAATA
AAGGGACAAATGTTCCGAACGGTTATTCTCAAAATGATAT
TCAGCTTATGGCAAACGCAGTATACGGAGAATCGCGTGGT
GAACCGTATTTAGGACAAGTIGCAGTAGCTGCCGTTATTT
TGAATCGTGTTACAAGTGCGTCATTCCCAAATACTGTTTC
AGGTGTAATCTTTGAACCTAGAGCATTTACAGCGGTAGCC
GATGGACAAATATATTTAACGCCAAATGAAACAGCAAAAA
AAGCAGTTTTAGATGCGATTAACGGATGGGATCCAACAGG
AAATGCTTTATATTATTTCAATCCAGATACTGCGACTAGT
AAATGGATTTGGACTCGTCCACAAATTAAAAAAATCGGTA
AACATATTTTCTGTAAA,
B. thuringiensis serovar
kurstaki str. HD73 GerAB/ArcD/ProY family
transporter, HD73_5042, Spore germination
protein IB, GenBank: CP004069.1 region:
4836255-4837352 1098 bp
SEQ ID NO: 339
ATGAGTAAAGTTCAAGAAAAATATCAAATATCTCCTGTCT
TTGTTTTCTTCTTAATTCACGGTGCACAGTTTGGTGCTGG
CGTTCTCGGCTTCGCTCGAATCATCGCAAAGGCCGCTGGA
TACGACGGATGGATGGGCGTTCTAATCACGGGACTCTGTA
TCCATCTCCTTATATGGATGATGTATTTTTTATTAAAAGA
AACAAATGGAAATTTAATTGATTTACAAAGGCAAACGTTC
GGAAAGTGGATAGGGAATATCTTTAATATCATCTTTGCAG
CTTATTTTTTAAACGTCAGCATTTCGGTTATTCGAACATA
TGTTGAAATTATTCAAGTATGGATGTTTCCTACCGCTTCC
ACCTTTATGTTAACACTCTTTTTATGCTTAGTTAGCTATT
ACATTATCTCTTCTGGATTTCGTGTCATTACTGGCATTTG
TGTCATCTCTGTTGGTGGTACATTAGGATATTTATTTCTA
AGCTTATTCGTTTTGAAATATTCACATTGGGATAATTTAC
TACCTATATTCACACATTCTTTTTCAGACATTTTAAAATC
CGCTCAACTGTCAATCTACAGTATGACCGGTTTTGAGATT
TACCTCATGGTATATCCATTTGTGAAAAAGCCTATGCAAT
CTCATAAATACGCTCAGCTTGGAGCACTATTTTCAAATCT
TCTATATTTATTTAGCACCCTTTTAGCCTTTGCATTTTTT
AGTGAAAAACAATTATTAAAAACGATATGGGCACAACTTT
CTATGACCCAAGTCATTCAATTACCATTTATAGAAAGACT
CGAATATATTGCAATTTCAGGCTACGCACTCGTTATTATT
TCAAGTTTTATCCTCCCTTTATGGGCGTCAATGAGAGCAA
CTCATGAAATATTCCGTGTAAAACAAAGAGGGGTTTTAAT
CGCTTTTATCCTTATCACCCTCATTGTTTCACAACTTTTA
ACAAACAGACACGATATTAATAACTTTATTAGTAATGTAT
CAAAGGTTAGTTTTTGGCTCATTTATGTATACATCCCAAT
CCTATTTATTATTGTGTGGGTGAAAAGAAAATGGAAAAAA
TCAAAAGCACAAGCAAAT,
B. thuringiensis serovar
kurstaki str. HD73 gerKB, HD73_0710, spore
germination protein, GenBank: CP004069.1
region: 727566-728669, 1104 bp
SEQ ID NO: 340
GTGACAAACACGCAAAGTAAAATTTCACTTGTTCAATTTA
CCTTTTTCGTTATTCAATGCCAAATTGGGGTCGGTATACT
ATCTCTACCTAACCGGCTTCATCCGATAGCAAAAGGTGGT
GGTTGGATTTCTGCGCTCATCGCGGGGCTTGCCATTCAAC
TTATTATTTTACTTATGTGGCTTTTCTTAAAACGATTTCC
TGATGCAGATATGTATAAAAGTGTTTGTATGCTATTTGGT
AATAAGTTAGGAAAACTATTGGGCTTTTCTTATGTTTTTT
ATTTTACCCTTATCGGTATGACGGTTATGTTGAATGCTTG
TAACGTCATTAAAGTATGGGTTTTACACGCAACTCCGTGG
TCTGCAATTCTCATATTATGTACAGTAGCTTGTTGTTATG
TAGCTTATAACACATTTAAAGTCATCGTACGATTTTATGT
CATGGCCTCTATTCTCATTATTCCAATGGCCCTTTTAATT
GCCCTAGGGCTTTCACGAGCTGATTTCTCTTATATTTTTC
CTATTACAGAAGCCGGATGGTGGAATATAATTCAAGCATC
CAAGGAAACAATTACAGCCATGTATGGTTTTGAAATTATT
CTTATCGCTTTCCCAAAAGTAAATGGGAGTTCTATTGCAA
AACTGAAGGCCATTTCTATCGCCAATGGATTTGTAACTCT
TTTTTATACTTTTACCGTTTGGATTTGCTTTATCGTATTT
AGTCCAAAGCAAATTGAGCTTATTCCAGAGCCAGTTGCTT
ACTTATTACGCTCCTTGCATATTGGAATTATAGATCGAAC
GGACCTAATCTTCATCCCAATTTGGATGATAACAGTTGTC
GCTTCTATTGCTAGTTATTACTGCGCTGCTTCAATCGGAA
TTGGACATATATTCAACCTTGCTAATCATAAAAAGGCTGT
TCCAATCGTTGGTATTATTGCTTTTAGTGTAGCCTTATTT
ATAGATACACCTGAAGAATTAAAAGTAATTTCAACTTTCA
CTGATAAGTTCACCTATATTTTCATCGTTGTCCTCCCTCT
TTTATTTCTACTTTATTCAATAATAAGAAAGAAGAAAGGA
GAACAGTATGTTCCCAAAAAGAGT,

In some embodiments of any of the aspects, the engineered microorganism comprises an inactivating modification of at least two or more germination genes. In some embodiments of any of the aspects, the engineered microorganism comprises at least one inactivating modification of at least 1, at least 2, at least 3, at least 4, at least 5, at least 6, at least 7, at least 8, at least 9, or at least 10 germination genes. In some embodiments of any of the aspects, the engineered microorganism comprises an inactivating modification of at least two, but not more than 10, not more than 9, not more than 8, not more than 7, not more than 6, not more than 5, not more than 4, or not more than 3 germination genes. In some embodiments of any of the aspects, the engineered microorganism comprises at least 1, at least 2, at least 3, at least 4, at least 5, at least 6, at least 7, at least 8, at least 9, or at least 10 inactivating modifications of at least 1 germination gene.

### Methods of Determining Provenance

In one aspect of any of the embodiments, described herein is a method of determining the provenance of an item. As used herein, “provenance” refers to the place of origin of an item, e.g., a factory, a farm, a distributor, a laboratory, etc. “Source” can used as another equivalent term for provenance. As such, the phrase “determining the provenance of an item” refers to determining the place of origin of an item, especially when there are no other indicators on the item as can be the case with food items.

In one aspect of any of the embodiments, the method comprises: (a) contacting an item with at least one engineered microorganism as described herein; (b) isolating nucleic acid from the item; (c) detecting the genetic barcode element of the isolated nucleic acid; and (d) determining the provenance of the item based on the detected genetic barcode element of the isolated nucleic acid.

In one aspect of any of the embodiments, the method comprises: (a) contacting an item with at least one engineered microorganism as described herein; (b) isolating the at least one engineered microorganism from the item; (c) detecting the genetic barcode element of the at least one isolated engineered microorganism; and (d) determining the provenance of the item based on the detected genetic barcode element of the at least one isolated engineered microorganism.

In some embodiments of any of the aspects, the method further comprises distributing the item in between step a (i.e., contacting the item with at least one engineered microorganism as described herein) and step b (i.e., isolating the nucleic acid and/or the engineered microorganism from the item), e.g., moving an item from its place of origin to a distributor location, a store, a company, a residence, etc.

In one aspect of any of the embodiments, the method comprises: (a) isolating nucleic acid from the item; and (b) detecting the presence of a genetic barcode element, wherein the presence of the genetic barcode element indicates the presence of an engineered microorganism comprising a genetic barcode element and an inactivating modification of at least one essential compound synthesis gene or an inactivating modification of at least one germination gene, wherein the presence of the engineered microorganism determines the provenance of the item.

In one aspect of any of the embodiments, described herein is a method of marking the provenance of an item, the method comprising contacting the item with at least one engineered microorganism as described herein.

In some embodiments of any of the aspects, the microorganism comprises first and second barcode regions. In some embodiments of any of the aspects, the first barcode region indicates that an item on which the microorganism is detected is from one of a group of known sources. In some embodiments of any of the aspects, the second barcode region indicates that an item on which the microorganism is detected is from a particular source of said group of sources.

In some embodiments of any of the aspects, the method further comprises detecting the presence of the first barcode region in a nucleic acid sample from an item, thereby determining that the item is from a group of known sources. In some embodiments of any of the aspects, the method further comprises detecting the presence of the second barcode region in the same or different nucleic acid sample from the item, thereby determining that the item is from a particular member of said group of known sources.

In some embodiments of any of the aspects, the method further comprises inactivating the engineered microorganism prior to contacting an item with an engineered microorganism as described herein. In some embodiments of any of the aspects, the engineered microorganism (e.g., an engineered S. cerevisiae) is inactivated (e.g., killed) through heating (e.g., in an aqueous solution) prior to use. As a non-limiting example, the engineered microorganism is heated in an aqueous solution (e.g., boiled) for at least one hour. As another non-limiting example, the engineered microorganism is exposed to a temperature of at least 100° C., at least 101° C., at least 102° C., at least 103° C., at least 104° C., at least 105° C., at least 110° C., at least 125° C., or at least 150° C. for at least 1 hour. As another non-limiting example, the engineered microorganism is exposed to an aqueous solution of at least 100° C. for at least 1 minute, at least 5 minutes, at least 10 minutes, at least 20 minutes, at least 30 minutes, at least 40 minutes, at least 50 minutes, at least 1 hour, at least 1.5 hours, or at least 2 hours.

In some embodiments of any of the aspects, the step of contacting the item with the engineered microorganism comprises spraying the item with a solution comprising at least one engineered microorganism as described herein. In some embodiments of any of the aspects, the step of contacting the item with at least one engineered microorganism comprises dusting, submerging, spraying (see e.g., U.S. Pat. No. 10,472,676 “Compositions for use in security marking”, the content of which is incorporated herein by reference in its entirety), or otherwise exposing the item to the at least one microorganism. In some embodiments of any of the aspects, the microorganism is only exposed to the external surface of the item.

In some embodiments of any of the aspects, an item is contacted with at least one engineered microorganism as described herein that is distinguishable from other microorganisms. In some embodiments of any of the aspects, an item is contacted with at least 2, at least 3, at least 4, or at least 5 distinguishable engineered microorganisms as described herein.

In some embodiments of any of the aspects, the item is a food item. In some embodiments of any of the aspects, the food item is a produce item. Non-limiting examples of produce items include farm-produced crops, fruits, vegetables, grains, oats, and the like. In some embodiments of any of the aspects, after being contacted with an engineered microorganism as described herein and prior to the detection of the engineered microorganism, the food item is rinsed, washed, boiled, fried, sonicated, cooked, microwaved, or otherwise prepared for consumption, and the engineered microorganism can still be detected.

In some embodiments of any of the aspects, the step of isolating the engineered microorganism comprises contacting the item with a device or implement to collect a sample of the engineered microorganism from the surface of the item (e.g., swabbing). In some embodiments of any of the aspects, the step of isolating the engineered microorganism further comprises isolating nucleic acids from the isolated microorganism. Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials (Roiff, A et al. PCR: Clinical Diagnostics and Research, Springer (1994)).

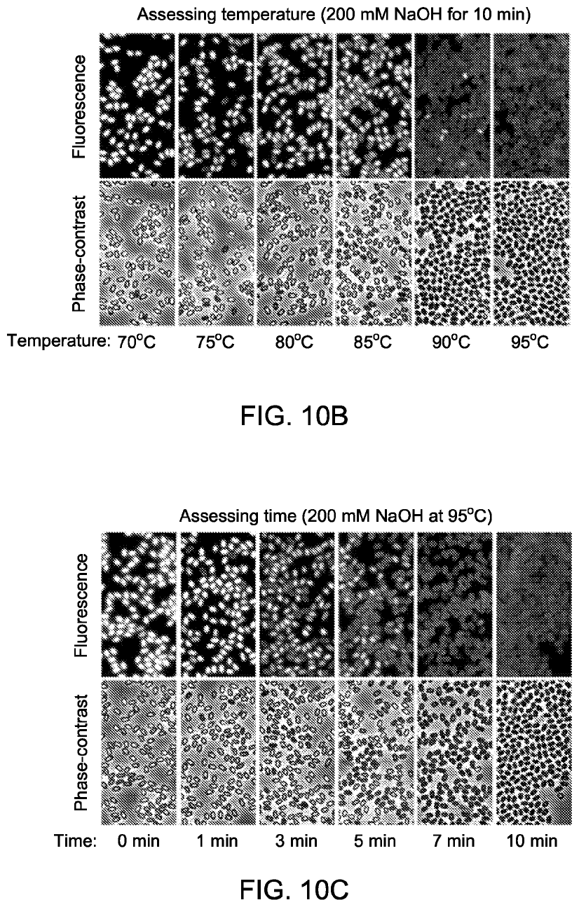

In some embodiments of any of the aspects, the step of isolating the engineered microorganism and/or nucleic acid of the engineered microorganism comprises a lysis procedure as described further herein. As a non-limiting example, the lysis protocol can comprise: (a) resuspending the engineered microorganism in an alkaline solution (e.g., NaOH); and (b) heating the alkaline solution to at least 90° C. for at least 7 minutes. In some embodiments of any of the aspects, the alkaline solution (e.g., NaOH) is at a concentration of at least 20 mM, at least 50 mM, at least 100 mM, at least 200 mM, or at least 300 mM (see e.g., FIG. 10A) and is heated to a temperature of at least 90° C. for at least 7 minutes. In some embodiments of any of the aspects, the alkaline solution (e.g., at least 200 mM NaOH) is heated to a temperature of at least 70° C., at least 75° C., at least 80° C., at least 85° C., at least 90° C., or at least 95° C. for at least 7 minutes (see e.g., FIG. 10B). In some embodiments of any of the aspects, the alkaline solution (e.g., at least 200 mM NaOH) is heated to a temperature of at least 90° C. for at least 1 minute, at least 2 minutes, at least 3 minutes, at least 4 minutes, at least 5 minutes, at least 6 minutes, at least 7 minutes, at least 8 minutes, at least 9 minutes, or at least 10 minutes (see e.g., FIG. 10C).

In some embodiments of any of the aspects, the step of detecting the genetic barcode element comprises a method selected from the group consisting of sequencing, hybridization with fluorescent or colorimetric DNA, and SHERLOCK. Specific High-sensitivity Enzymatic Reporter unLOCKing (SHERLOCK) is a method that can be used to detect specific RNA/DNA at low attomolar concentrations (see e.g., U.S. Pat. Nos. 10,266,886; 10,266,887; Gootenberg et al., Science. 2018 Apr. 27; 360(6387):439-444; Gootenberg et al., Science. 2017 Apr. 28; 356(6336):438-44; each of which is incorporated herein by reference in its entirety). Briefly, a method comprising detection of an engineered microorganism as described herein (e.g., wherein the genetic barcode element of the engineered microorganism does not comprise a Cas enzyme scaffold) using SHERLOCK comprises the following steps: (a) isolating DNA from an item; (b) amplifying the DNA with an isothermal amplification method (e.g., RPA); (c) contacting the amplified DNA with an RNA polymerase to promote the production of complementary RNA; (d) contacting the RNA with: (i) a crRNA comprising a Cas enzyme scaffold and a region that hybridizes to a barcode region of the engineered microorganism; (ii) a Cas enzyme (e.g., Cas13a (previously known as C2c2), Cas13b, Cas13c, Cas12a, and/or Csm6); and (iii) a detection molecule cleavable by the Cas enzyme; (e) detecting cleavage of the detection molecule, wherein said cleavage indicates presence of the barcode region of the engineered microorganism.

In some embodiments of any of the aspects, the RNA is contacted with at least one crRNA selected from the sequences in Table 4. In some embodiments of any of the aspects, the crRNA specifically hybridizes to one of SEQ ID NOs: 5-31, 154-221, or 222-315. In some embodiments of any of the aspects, the crRNA comprises one of SEQ ID NOs: 59-153 or a nucleic acid sequence that is at least 95%, at least 96%, at least 97%, at least 98%, at least 99%, or more identical to one of SEQ ID NOs: 59-153, that maintains the same function (e.g., specific hybridization with a barcode region or genetic barcode element as described herein).

In some embodiments of any of the aspects, the crRNA does not specifically hybridize to one of barcodes 7, 11, 12, 15, 26, 31, 33, 40, 41, 42, 52, 57, 59, 66, 67, 69, 70, 71, 79, 80, 82, 83, 85, 86, 94 (see e.g., FIG. 7B-7C). In some embodiments of any of the aspects, the crRNA does not comprise one of SEQ ID NOs: 65, 69, 70, 73, 84, 89, 91, 98, 99, 100, 110, 115, 117, 124, 125, 127, 128, 129, 137, 138, 140, 141, 143, 144, 152 or a nucleic acid sequence that is at least 95%, at least 96%, at least 97%, at least 98%, at least 99%, or more identical to one of SEQ ID NOs: 65, 69, 70, 73, 84, 89, 91, 98, 99, 100, 110, 115, 117, 124, 125, 127, 128, 129, 137, 138, 140, 141, 143, 144, or 152.

In some embodiments of any of the aspects, the crRNA hybridizes to one of barcodes 1-6, 8-10, 13-14, 16-25, 27-30, 32, 34-39, 43-51, 53-56, 58, 60-65, 68, 72-78, 81, 84, 87-93, or Universal 2. In some embodiments of any of the aspects, the crRNA hybridizes to one of SEQ ID NOs: 222-227, 229-231, 234-235, 237-246, 248-251, 253, 255-260, 264-272, 274-277, 279, 281-286, 289, 293-299, 302, 305, or 308-314. In some embodiments of any of the aspects, the crRNA hybridizes to one of SEQ ID NOs: 5-10, 12-14, 17-18, 20-29, 31, 154-156, 160-165, 169-177, 179-182, 184, 186-191, 194, 198-204, 207, 210, 213-219, or 221. In some embodiments of any of the aspects, the crRNA comprises one of SEQ ID NOs: 59-64, 66-68, 71-72, 74-83, 85-88, 90, 92-97, 101-109, 111-114, 116, 118-123, 126, 130-136, 139, 142, 145-151, 153, or a nucleic acid sequence that is at least 95%, at least 96%, at least 97%, at least 98%, at least 99%, or more identical to one of SEQ ID NOs: 59-64, 66-68, 71-72, 74-83, 85-88, 90, 92-97, 101-109, 111-114, 116, 118-123, 126, 130-136, 139, 142, 145-151, or 153.

As another example, a method comprising detection of an engineered microorganism as described (e.g., wherein the genetic barcode element of the engineered microorganism comprises a Cas enzyme scaffold) herein using SHERLOCK comprises the following steps: (a) isolating DNA from an item; (b) amplifying the DNA with an isothermal amplification method (e.g., RPA); (c) contacting the amplified DNA with an RNA polymerase to promote the production of complementary RNA; (d) contacting the RNA with: (i) a Cas enzyme (e.g., Cas13a (previously known as C2c2), Cas13b, Cas13c, Cas12a, and/or Csm6), wherein the Cas enzyme specifically binds to the Cas enzyme scaffold of the genetic barcode element; and (ii) a detection molecule cleavable by the Cas enzyme; (e) detecting cleavage of the detection molecule, wherein said cleavage indicates presence of the engineered microorganism.

Certain genetic barcode elements as described herein (e.g., those comprising a Cas enzyme scaffold and/or a transcription start site) are compatible with detection through SHERLOCK methods.

In some embodiments of any of the aspects, the genetic barcode elements as described herein are compatible with detection through hybridization-based detection systems (e.g., microarrays and microarray-like assays). While a hybridization-based detection system can be used to detect any genetic barcode element, such an approach to detection can be preferred, for example, when the genetic barcode element does not comprise a Cas enzyme scaffold or a transcription start site. As a non-limiting example, a hybridization-based detection system can comprise a solid support linked to localizations of nucleic acids that are each complementary and/or hybridize to a specific barcode region of a genetic barcode element.

In some embodiments of any of the aspects, methods of detecting an engineered microorganism comprise first detecting the presence of a first barcode region, and then if the first barcode is detected, detecting the presence or identity of a second barcode region. In some embodiments of any of the aspects, the sequence of the barcode region of the engineered microorganism is specific for the item or group of items. In some embodiments of any of the aspects, the sequence of the barcode region of the engineered microorganism is specific for a point of origin of item or group of items.

In some embodiments of any of the aspects, the step of detecting the genetic barcode element of the isolated nucleic acid comprises: detecting a first barcode region (i.e., assaying to detect the first barcode region). In some embodiments of any of the aspects, if the first barcode region is detected, then the second barcode region is detected (i.e., assayed to detect). In some embodiments of any aspects, if the first barcode region is not detected, then it is determined that no engineered microorganism is present on the item. In some embodiments of any aspects, if the second barcode region is not detected, then it is determined that no engineered microorganism is present on the item. In some embodiments of any aspects, if no barcode region as described herein (e.g., first and second barcode regions) is detected, then it is determined that no engineered microorganism is present on the item.

In general, the compositions and methods described herein permit the determination of the provenance of an item to determine the location at which the barcode-modified organism as described herein was applied to the item. However, it has been determined that the application of barcode-modified microorganisms as described herein can permit the determination of provenance or path down to meter-scale or smaller resolution. Accordingly, in one aspect described herein is a method of determining the path of an item or an individual across a surface, comprising: (a) contacting a surface with at least two engineered microorganisms as described herein; (b) allowing the item or individual to contact the surface in a continuous or discontinuous path; (c) isolating nucleic acid from the item or individual; (d) detecting the genetic barcode elements of the at least two isolated engineered microorganisms; and (e) determining the path of the item or individual across the surface based on the detected genetic barcode element of the at least two isolated engineered microorganisms.

In some embodiments of any of the aspects, the surface comprises sand, soil, carpet, or wood. In some embodiments of any of the aspects, the surface is divided into a grid comprising grid sections, wherein each grid section comprises at least one engineered microorganism that is distinguishable from all other engineered microorganism on the surface. In some embodiments of any of the aspects, each grid section comprises at least two distinguishable engineered microorganisms. In some embodiments of any of the aspects, each grid section comprises at least three distinguishable engineered microorganisms. In some embodiments of any of the aspects, each grid section comprises at least four distinguishable engineered microorganisms. In some embodiments of any of the aspects, each grid section comprises at least 4, at least 5, at least 6, at least 7, at least 8, at least 9, or at least 10 distinguishable engineered microorganisms.

In some embodiments of any of the aspects, the item or individual is determined to have contacted a specific grid section if at least one engineered microorganism originating from the specific grid section is detected on the item or individual. In some embodiments of any of the aspects, the path of the item or individual across the surface comprises the specific grid sections that the item or individual is determined to have contacted. In some embodiments of any of the aspects, the item or individual is determined to not have contacted a specific grid section if none of the engineered microorganisms originating from the specific grid section are detected on the item or individual. In some embodiments of any of the aspects, the path of the item or individual across the surface does not comprise the specific grid sections that the item or individual is determined to not have contacted.

In some embodiments of any of the aspects, a method of detecting an engineered microorganism as described (e.g., methods of tracking an item or individual) herein exhibits meter-scale resolution. As a non-limiting example, a method of detection as described herein can be used to detect the provenance or path of an engineered microorganism within 1 meter (m) from its original location. As a non-limiting example, a method of detection as described herein can be used to detect an engineered microorganism to within 1 centimeter (cm), within 10 cm, within 1 m, within 2 m, within 3 m, within 4 m, within 5 m, within 6 m, within 7 m, within 8 m, within 9 m, or within 10 m from its original location.

In some embodiments of any of the aspects, a method of detecting an engineered microorganism as described herein exhibits single-spore sensitivity. As a non-limiting example, in some embodiments of any of the aspects, a method of detection as described herein can be used to detect at least one spore of an engineered microorganism as described herein. As a non-limiting example, a method of detection as described herein can be used to detect at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, or at least 100 spores of an engineered microorganism as described herein.

### Detection Assays

Any of a number of different assays can be used to detect the barcode-engineered microorganisms as described herein. It should be understood that “detecting” as the term is used herein necessarily encompasses the performance of steps such as nucleic acid collection or isolation and/or amplification, hybridization, transcription, cleavage, etc. that generate a signal indicative of the presence (or absence) of a given genetic barcode element or barcode region in a sample collected from, e.g., a marked or tracked item.

In some embodiments of any of the aspects, measurement of the level of a target (e.g., engineered microorganism or genetic barcode element as described herein) and/or detection of the level or presence of a target, e.g. of an expression product (nucleic acid or polypeptide of one of the genes described herein) or a mutation can comprise a transformation. As used herein, the term “transforming” or “transformation” refers to changing an object or a substance, e.g., biological sample, nucleic acid or protein, into another substance. The transformation can be physical, biological or chemical. Exemplary physical transformation includes, but is not limited to, pre-treatment of a biological sample, e.g., from whole blood to blood serum by differential centrifugation. A biological/chemical transformation can involve the action of at least one enzyme and/or a chemical reagent in a reaction. For example, a DNA sample can be digested into fragments by one or more restriction enzymes, or an exogenous molecule can be attached to a fragmented DNA sample with a ligase. In some embodiments of any of the aspects, a DNA sample can undergo enzymatic replication, e.g., by polymerase chain reaction (PCR) or isothermal amplification (e.g., RPA).

Transformation, measurement, and/or detection of a target molecule, e.g. a mRNA or polypeptide can comprise contacting a sample obtained from a subject with a reagent (e.g. a detection reagent) which is specific for the target, e.g., a target-specific reagent. In some embodiments of any of the aspects, the target-specific reagent is detectably labeled. In some embodiments of any of the aspects, the target-specific reagent is capable of generating a detectable signal. In some embodiments of any of the aspects, the target-specific reagent generates a detectable signal when the target molecule is present.

In certain embodiments, nucleic acids can be isolated, derived, or amplified from a biological sample, such as a sample from a food item. Techniques for the detection of a nucleic acid are known by persons skilled in the art, and can include but are not limited to, isothermal amplification (e.g., RPA), PCR procedures, RT-PCR, quantitative RT-PCR, Northern blot analysis, differential gene expression, RNase protection assay, microarray based analysis, next-generation sequencing; hybridization methods, etc.

In general, isothermal amplification (e.g., RPA) is comprised of (i) sequence-specific hybridization of primers to specific genes or sequences within a nucleic acid sample or library, (ii) subsequent amplification involving multiple rounds of primer annealing, elongation, and strand displacement (as a non-limiting example, using a combination of recombinase, single stranded binding proteins, and DNA polymerase), and (iii) detection of the product through such methods as sequencing to confirm the identity of the amplified product or general assays such as turbidity. In some types of isothermal amplification, turbidity results from pyrophosphate byproducts produced during the reaction; these byproducts form a white precipitate that increases the turbidity of the solution. The primers used in isothermal amplification are oligonucleotides of sufficient length and appropriate sequence to provide initiation of polymerization, i.e. each primer is specifically designed to be complementary to a strand of the template (e.g., genetic locus, genetic barcode element as described herein) to be amplified. In contrast to the polymerase chain reaction (PCR) technology in which the reaction is carried out with a series of alternating temperature steps or cycles, isothermal amplification is carried out at one temperature, and does not require a thermal cycler or thermostable enzymes.

In general, the PCR procedure is a method of gene amplification comprised of (i) sequence-specific hybridization of primers to specific genes or sequences within a nucleic acid sample or library, (ii) subsequent amplification involving multiple rounds of primer annealing, elongation, and thermal denaturation using a thermostable DNA polymerase, and (iii) analyzing the PCR products for a band of the correct size or sequence. The primers used in PCR are oligonucleotides of sufficient length and appropriate sequence to provide initiation of polymerization, i.e. each primer is specifically designed to be complementary to a strand of the (e.g., genetic locus, genetic barcode element as described herein) to be amplified. In an alternative embodiment, mRNA level of gene expression products described herein can be determined by reverse-transcription (RT) PCR or by quantitative RT-PCR (QRT-PCR) or real-time PCR methods. Methods of RT-PCR and QRT-PCR are well known in the art.

In some embodiments of any of the aspects, the level and/or sequence of a nucleic acid can be measured by a quantitative sequencing technology, e.g. a quantitative next-generation sequencing technology. Methods of sequencing a nucleic acid sequence are well known in the art. Briefly, a sample obtained from a subject can be contacted with one or more primers which specifically hybridize to a single-strand nucleic acid sequence (e.g., primer binding sequence) flanking the target sequence (e.g., a genetic barcode element; e.g., barcode region) and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing (i.e., dideoxy chain termination), high-throughput sequencing, next generation sequencing, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

Nucleic acid, e.g., deoxyribonucleic acid (DNA) and ribonucleic acid (RNA), molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials (Roiff, A et al. PCR: Clinical Diagnostics and Research, Springer (1994)).

In some embodiments of any of the aspects, one or more of the detection reagents (e.g. an antibody reagent and/or nucleic acid probe) can comprise a detectable label and/or comprise the ability to generate a detectable signal (e.g. by catalyzing a reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies and nucleic acid probes) are well known in the art.

In some embodiments of any of the aspects, detectable labels can include labels that can be detected by spectroscopic, photochemical, biochemical, immunochemical, electromagnetic, radiochemical, or chemical means, such as fluorescence, chemifluorescence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, a detection reagent is labeled with a fluorescent compound. When the fluorescently labeled reagent is exposed to light of the proper wavelength, its presence can then be detected due to fluorescence. In some embodiments of any of the aspects, a detectable label can be a fluorescent dye molecule, or fluorophore including, but not limited to fluorescein, phycoerythrin, phycocyanin, o-phthalaldehyde, fluorescamine, Cy3™, Cy5™, allophycocyanin, Texas Red, peridinin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetramethylrhodamine isothiocyanate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g., cyanine dyes such as Cy3, Cy5, etc.; BODIPY dyes and quinoline dyes. In some embodiments of any of the aspects, a detectable label can be a radiolabel including, but not limited to ³H, ¹²⁵I ³⁵S, ¹⁴C, ³²P, and ³³P. In some embodiments of any of the aspects, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments of any of the aspects, a detectable label is a chemiluminescent label, including, but not limited to lucigenin, luminol, luciferin, isoluminol, theromatic acridinium ester, imidazole, acridinium salt and oxalate ester. In some embodiments of any of the aspects, a detectable label can be a spectral colorimetric label including, but not limited to colloidal gold or colored glass or plastic (e.g., polystyrene, polypropylene, and latex) beads.

In some embodiments of any of the aspects, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromogenic substrate. Such streptavidin peroxidase detection kits are commercially available, e.g., from DAKO; Carpinteria, CA. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylenediaminetetraacetic acid (EDTA).

A level which is less than a reference level can be a level which is less by at least about 10%, at least about 20%, at least about 50%, at least about 60%, at least about 80%, at least about 90%, or less relative to the reference level. In some embodiments of any of the aspects, a level which is less than a reference level can be a level which is statistically significantly less than the reference level.

A level which is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments of any of the aspects, a level which is more than a reference level can be a level which is statistically significantly greater than the reference level.

In some embodiments of any of the aspects, the reference can also be a level of expression of the target molecule in a control sample, a pooled sample of control items or a numeric value or range of values based on the same. In some embodiments of any of the aspects, the reference can be the level of a target molecule in a sample obtained from the same item at an earlier point in time.

The term “sample” or “test sample” as used herein denotes a sample taken or isolated from an item marked or tracked as described herein. A “sample” or “test sample” also refers to a sample taken from an item for which one wishes to determine the provenance as described herein. The term “test sample” also includes untreated or pretreated (or pre-processed) samples.

The test sample can be obtained by removing a sample from an item, but can also be accomplished by using a previously isolated sample (e.g. isolated at a prior time point by the same or another person).

In some embodiments of any of the aspects, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of any of the aspects, the test sample can be a frozen test sample. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, homogenization, sonication, filtration, thawing, purification, and any combinations thereof. In some embodiments of any of the aspects, the test sample can be treated with a chemical and/or biological reagent. Chemical and/or biological reagents can be employed, for example, to protect and/or maintain the stability of the sample, including biomolecules (e.g., nucleic acid and protein) therein, during processing. The skilled artisan is well aware of methods and processes appropriate for pre-processing of biological samples required for detection of a nucleic acid as described herein.

In some embodiments of any of the aspects, the methods, assays, and systems described herein can further comprise a step of obtaining or having obtained a test sample from an item.

### Kits

Another aspect of the technology described herein relates to kits for marking or determining the provenance of an item, among others. Described herein are kit components that can be included in one or more of the kits described herein.

In some embodiments, the kit comprises an effective amount of an engineered microorganism as described herein. As will be appreciated by one of skill in the art, an engineered microorganism can be supplied in a lyophilized form or a concentrated form that can diluted or suspended in liquid prior to use. In some embodiments of any of the aspects, the engineered microorganism can be supplied in a liquid suspension or another carrier acceptable for consumption (e.g., human consumption).

Acceptable carriers and diluents include saline, aqueous buffer solutions, solvents and/or dispersion media. The use of such carriers and diluents is well known in the art. Some non-limiting examples of materials which can serve as acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) excipients, such as cocoa butter; (8) oils, such as peanut oil, cottonseed oil, safflower oil, sesame oil, olive oil, corn oil and soybean oil; (9) glycols, such as propylene glycol; (10) polyols, such as glycerin, sorbitol, mannitol and polyethylene glycol (PEG); (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (18) pH buffered solutions; (19) polyesters, polycarbonates and/or polyanhydrides; (20) bulking agents, such as polypeptides and amino acids and (21) other non-toxic compatible substances employed in formulations. Wetting agents, coloring agents, release agents, coating agents, sweetening agents, flavoring agents, perfuming agents, preservative and antioxidants can also be present in the formulation. The terms such as “excipient”, “carrier”, “acceptable carrier” or the like are used interchangeably herein. In some embodiments, the carrier inhibits the degradation of the active agent, e.g. the engineered microorganism as described herein.

Preferred formulations include those that are non-toxic to the engineered microorganisms described herein. In some embodiments of any of the aspects, the carrier does not comprise any of the essential gene product(s) (e.g., essential compounds, essential nutrients) for which the engineered microorganism comprises at least one inactivating modification. The engineered microorganism can be supplied in aliquots or in unit doses.

In some embodiments of any of the aspects, the kit comprises at least one set of primers for amplification (e.g., isothermal amplification). In some embodiments of any of the aspects, the set of amplification primers is specific to at least one genetic barcode element. In some embodiments of any of the aspects, the primers are provided at a sufficient concentration, e.g., 5 uM to 35 uM, to be added to reaction mixture. As a non-limiting example, the primers are provided at a concentration of at least 1 uM, at least 2 uM, at least 3 uM, at least 4 uM, at least 5 uM, at least 6 uM, at least 7 uM, at least 8 uM, at least 9 uM, at least 10 uM, at least 11 uM, at least 12 uM, at least 13 uM, at least 14 uM, at least 15 uM, at least 16 uM, at least 17 uM, at least 18 uM, at least 19 uM, at least 20 uM, at least 21 uM, at least 22 uM, at least 23 uM, at least 24 uM, at least 25 uM, at least 26 uM, at least 27 uM, at least 28 uM, at least 29 uM, at least 30 uM, at least 35 uM, at least 40 uM, at least 45 uM, at least or at least 50 uM. In some embodiments of any of the aspects, the primers comprise SEQ ID NOs: 1 and 4.

In some embodiments of any of the aspects, the kit further comprises a recombinase and single-stranded DNA binding (SSB) protein. In some embodiments of any of the aspects, the single-stranded DNA-binding protein is a gp32 SSB protein. In some embodiments of any of the aspects, the recombinase is a uvsX recombinase. In some embodiments of any of the aspects, the recombinase and single-stranded DNA binding proteins are provided at a sufficient amount to be added to the reaction mixture. In some embodiments of any of the aspects, the kit comprises RPA pellets comprising RPA reagents (e.g., DNA polymerase, helicase, SSB) at a sufficient concentration. See e.g., U.S. Pat. No. 7,666,598, the content of which is incorporated herein by reference in its entirety.

In some embodiments of any of the aspects, the kit further comprises at least one of the following: reaction buffer, diluent, water, magnesium acetate (or another magnesium compound such as magnesium chloride) dNTPs, DTT, and/or an RNase inhibitor.

In some embodiments of any of the aspects, the kit further comprises reagents for isolating nucleic acid from the sample. In some embodiments of any of the aspects, the kit further comprises reagents for isolating DNA from the sample. In some embodiments of any of the aspects, the kit further comprises reagents for isolating RNA from the sample. In some embodiments of any of the aspects, the kit further comprises detergent, e.g., for lysing the sample. In some embodiments of any of the aspects, the kit further comprises a sample collection device, such a swab. In some embodiments of any of the aspects, the kit further comprises a sample collection container, optionally containing transport media.

In some embodiments of any of the aspects, the kit further comprises reagents for detecting the amplification product(s), comprising reagents appropriate for a detection method selected from: lateral flow detection; hybridization with conjugated or unconjugated DNA; colorimetric assays; gel electrophoresis; Specific High-sensitivity Enzymatic Reporter unLOCKing (SHERLOCK); sequencing; and quantitative polymerase chain reaction (qPCR). In some embodiments of any of the aspects, the kit further comprises an additional set of primers and/or a detectable probe (e.g., for detection using qPCR, sequencing). In some embodiments of any of the aspects, the kit further comprises a light source, a light filter, and/or a detection device.

In some embodiments of any of the aspects, the kit further comprises a negative control (e.g., a sample not comprising a genetic barcode element) or positive control (e.g., a sample known to comprise a genetic barcode element). In some embodiments, the kit comprises an effective amount of the reagents as described herein. As will be appreciated by one of skill in the art, the reagents can be supplied in a lyophilized form or a concentrated form that can diluted or suspended in liquid prior to use. The kit reagents described herein can be supplied in aliquots or in unit doses.

In some embodiments, the components described herein can be provided singularly or in any combination as a kit. Such a kit includes the components described herein, e.g., a composition comprising an engineered microorganism, packaging materials thereof, and optionally a device or implement for applying the engineered microorganism to an item. Such kits can optionally include one or more agents that permit the detection of an engineered microorganism or a set thereof. In addition, a kit optionally comprises informational material.

In some embodiments, the compositions in a kit can be provided in a watertight or gas tight container which in some embodiments is substantially free of other components of the kit. For example, an engineered microorganism composition can be supplied in more than one container, e.g., it can be supplied in a container having sufficient reagent for a predetermined number of applications, e.g., 1, 2, 3 or greater. One or more components as described herein can be provided in any form, e.g., liquid, dried or lyophilized form. Liquids or components for suspension or solution of the engineered microorganism composition can be provided in sterile form and should not contain microorganisms (engineered or otherwise) other than those to be applied to a given object or item or product to be marked or tagged or tracked as described herein. When the components described herein are provided in a liquid solution, the liquid solution preferably is an aqueous solution.

The informational material can be descriptive, instructional, marketing or other material that relates to the methods described herein. The informational material of the kits is not limited in its form. In some embodiments, the informational material can include information about production of the engineered microorganism, concentration, date of expiration, batch or production site information, and so forth. In some embodiments, the informational material relates to methods for using or administering the components of the kit.

The kit can include a component for the detection of the engineered microorganism or the genetic barcode element of the engineered microorganism. In addition, the kit can include one or more antibodies that bind a cell marker, or primers for an isothermal amplification (e.g., RPA, LAMP, HDA, RAA, etc.), RT-PCR or PCR reaction, e.g., a semi-quantitative or quantitative RT-PCR or PCR reaction. The detection reagent can be linked to a label, e.g., a radiological, fluorescent (e.g., GFP) or colorimetric label for use in detection. If the detection reagent is a primer, it can be supplied in dry preparation, e.g., lyophilized, or in a solution. In one embodiment, primers and/or other reagents are present in an array or microarray format, e.g., on a solid support.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

In some embodiments of any of the aspects, the kit can further comprise a detection device. As a non-limiting example, a detection device can comprise a light-emitting diode (LED) light source and/or a filter (e.g., plastic filter specific for the emitting wavelength of a detectable marker). In some embodiments of any of the aspects, the kit and/or the detection device is field-deployable, i.e., transportable, non-refrigerated, and/or inexpensive. In some embodiments of any of the aspects, a detection device further comprises a wireless device (e.g., a cell phone, a personal digital assistant (PDA), a tablet).

### Systems

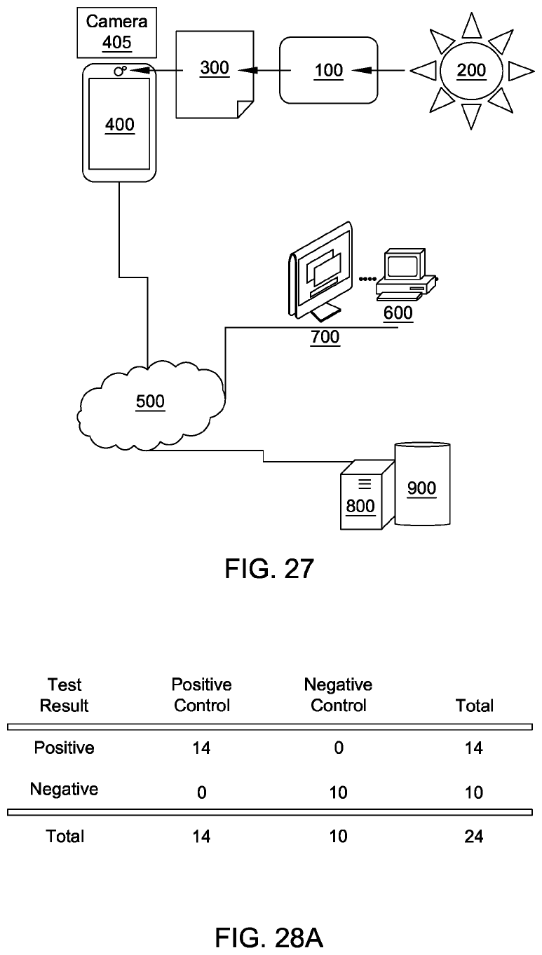

FIG. 27 shows an exemplary schematic of a system as described herein. As a non-limiting example, an engineered microorganism as described herein can be detected using an assay 100 as described herein. The results of the assay can be detected by exposing the detection assay 100 to a light source 200 (according to the specific excitation wavelength of a detection molecule in the assay) and a filter 300 (according to the specific emission wavelength of a detection molecule in the assay). The emitted wavelength of the detection molecule in the assay can be detected by the camera 405 of a portable computing device 400 (e.g., a mobile phone) or any other device comprising a camera 405. The portable computing device 400 can be connected to a network 500. In some embodiments, the network 500 can be connected to another computing device 600 and/or a server 800. The network 500 can be connected to various other devices, servers, or network equipment for implementing the present disclosure. A computing device 600 can be connected to a display 700. Computing device 400 or 600 can be any suitable computing device, including a desktop computer, server (including remote servers), mobile device, or any other suitable computing device. In some examples, programs for implementing the system can be stored in database 900 and run on server 800. Additionally, data and data processed or produced by said programs can be stored in database 900.

It should initially be understood that the methods and systems described herein can be implemented with any type of hardware and/or software, and can include use of a pre-programmed general purpose computing device. For example, the system (e.g., the detection system and/or the system for marking an item) can be implemented using a server, a personal computer, a portable computer, a thin client, or any suitable device or devices. The compositions, methods and/or components for the performance thereof can include the use of a single device at a single location, or multiple devices at a single, or multiple, locations that are connected together using any appropriate communication protocols over any communication medium such as electric cable, fiber optic cable, or in a wireless manner.

It should also be noted that the compositions, systems, and methods as described herein can be arranged or used in a format having a plurality of modules which perform particular functions. It should be understood that these modules are merely schematically illustrated based on their function for clarity purposes only, and do not necessary represent specific hardware or software. In this regard, these modules can be hardware and/or software implemented to substantially perform the particular functions discussed. Moreover, the modules can be combined together within the disclosure, or divided into additional modules based on the particular function desired. Thus, the disclosure should not be construed to limit the present technology as disclosed herein, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be performed in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively, or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of these. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program can, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA or an ASIC as noted above.

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from, or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, the term “spore” refers to a non-germinated endospore (e.g., of a spore-forming bacteria such as Bacillus species). It is generally understood that such spores are quiescent (e.g., non-dividing); have increased resilience to temperature, salinity, pH, and other harsh environmental factors compared to non-spore cells; and are able to persist in the environment for long periods of time. A spore carries and provides protection for nucleic acid comprising a genetic barcode element as described herein.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

A variant DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (e.g., mRNA) or antisense RNA derived from a nucleic acid fragment or fragments and/or to the translation of mRNA into a polypeptide.

“Expression products” include RNA transcribed from a gene, and polypeptides obtained by translation of mRNA transcribed from a gene. The term “gene” refers to the nucleic acid sequence which is transcribed (DNA) to RNA in vitro or in vivo when operably linked to appropriate regulatory sequences. The gene may or may not include regions preceding and following a coding region, e.g. 5′ untranslated (5′UTR) or “leader” sequences and 3′ UTR or “trailer” sequences, as well as intervening sequences (introns) between individual coding segments (exons).

“Marker” in the context of the present invention refers to an expression product, e.g., nucleic acid or polypeptide which is differentially present in a sample taken from an item having an engineered microorganism, as compared to a comparable sample taken from control items.

In some embodiments, a nucleic acid comprising a genetic barcode element as described herein is comprised by a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a genetic barcode element as described herein, a fusion protein or a nucleic acid encoding an expression product which is operably linked to at least one non-native (e.g., heterologous) genetic control element (e.g., a promoter, suppressor, activator, enhancer, response element, or the like).

As used herein, the term “viral vector” refers to a nucleic acid vector construct that includes at least one element of viral origin and has the capacity to be packaged into a viral vector particle. The viral vector can contain the genetic barcode element as described herein in place of non-essential viral genes. The vector and/or particle can be utilized for the purpose of transferring nucleic acids into cells either in vitro or in vivo.

It should be understood that the vectors described herein can, in some embodiments, be combined with other suitable compositions. In some embodiments, the vector is episomal. The use of a suitable episomal vector provides a means of maintaining the genetic barcode element as described herein in the subject in high copy number extra chromosomal DNA thereby eliminating potential effects of chromosomal integration.

As used herein, the term “hybridizing”, “hybridize”, “hybridization”, “annealing”, or “anneal” are used interchangeably in reference to the pairing of complementary nucleic acids using any process by which a strand of nucleic acid joins with a complementary strand through base pairing to form a hybridization complex. In other words, the term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation. Sequence determination, e.g., that indicates or confirms the presence of a given sequence element, e.g., a barcode element or region thereof, is a form of detecting.

In some embodiments of any of the aspects, a polypeptide, nucleic acid, cell, or microorganism as described herein can be engineered. As used herein, “engineered” refers to the aspect of having been manipulated by the hand of man. For example, a polynucleotide is considered to be “engineered” when at least one aspect of the polynucleotide, e.g., its sequence, has been manipulated by the hand of man to differ from the aspect as it exists in nature. A microorganism comprising an engineered polynucleotide sequence is considered to be an engineered microorganism. As is common practice and is understood by those in the art, progeny of an engineered cell are typically still referred to as “engineered” even though the actual manipulation was performed on a prior entity.

In some embodiments of any of the aspects, the engineered microorganism described herein is exogenous to the system in which it is used. In some embodiments of any of the aspects, the engineered microorganism described herein is ectopic. In some embodiments of any of the aspects, the engineered microorganism described herein is not endogenous.

The term “exogenous” refers to a substance present in a cell that is not encoded by such a cell as it occurs in nature. The term “exogenous” when used herein can refer to a nucleic acid or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In such instances, the increased level of expression is often achieved via introduction of an engineered construct that directs expression beyond that which normally occurs in the subject cell or organism. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

As used herein, “contacting” refers to any suitable means for delivering, or exposing, an agent to at least one cell. Exemplary delivery methods include, but are not limited to, direct delivery to spraying, dusting, stamping, or brushing with a liquid, suspension, emulsion or dry formulation of, e.g., an engineered microorganism as described herein. The term “contacting” also applies, for example, to the process used to introduce the modified nucleic acids to an organism or system, e.g., to an engineered microorganism as described herein. In this context, “contacting” can be by means of, e.g., cell culture medium, transfection, transduction, perfusion, injection, or other delivery method known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviations (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean+1%. In some embodiments of any of the aspects, the term “about” when used in connection with percentages can mean±5%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A microorganism engineered to comprise at least one genetic barcode element and at least one of:
   a) an inactivating modification of at least one essential gene; or
   b) an inactivating modification of at least one germination gene.
2. The engineered microorganism of paragraph 1, wherein the microorganism is engineered to comprise a genetic barcode element, an inactivating modification of at least one essential gene, and an inactivating modification of at least one germination gene.
3. The engineered microorganism of paragraph 1, wherein the microorganism is engineered to comprise a genetic barcode element and an inactivating modification of at least one essential gene.
4. The engineered microorganism any of paragraphs 1-3, wherein the microorganism is a yeast or a bacterium.
5. The engineered microorganism of any of paragraphs 1-4, wherein the microorganism is a Saccharomyces yeast or a Bacillus bacterium.
6. The engineered microorganism of any of paragraphs 1-5, wherein the microorganism is Saccharomyces cerevisiae, Bacillus subtilis, or Bacillus thuringiensis.
7. The engineered microorganism of any of paragraphs 1-6, wherein the microorganism is engineered from Saccharomyces cerevisiae strain BY4743, Bacillus subtilis strain 168, or Bacillus thuringiensis strain HD-73.
8. The engineered microorganism of any of paragraphs 1-7, wherein the genetic barcode element comprises:
   a) a first primer binding sequence;
   b) at least one barcode region;
   c) a Cas enzyme scaffold;
   d) a transcription initiation site; and
   e) a second primer binding sequence.
9. The engineered microorganism of any of paragraphs 1-7, wherein the genetic barcode element comprises:
   a) a first primer binding sequence;
   b) at least one barcode region;
   c) a transcription initiation site; and
   d) a second primer binding sequence.
10. The engineered microorganism of any of paragraphs 1-7, wherein the genetic barcode element comprises:
   a) a first primer binding sequence;
   b) at least one barcode region; and
   c) a second primer binding sequence.
11. The engineered microorganism of any of paragraphs 1-10, wherein the microorganism is engineered to comprise first and second barcode regions.
12. The engineered microorganism of paragraph 11, wherein the first barcode region indicates that an item on which the microorganism is detected is from one of a group of known sources, and the second barcode region indicates that an item on which the microorganism is detected is from a particular source of said group of sources.
13. The engineered microorganism of any of paragraphs 8-12, wherein the first primer binding sequence and second primer binding sequence comprise sites for binding of PCR or RPA primers.
14. The engineered microorganism of any of paragraphs 8-13, wherein the barcode region comprises 20-40 base pairs.
15. The engineered microorganism of any of paragraphs 8-14, wherein the barcode region comprises a Hamming distance of at least 5 base pairs relative to barcode regions comprised by other items marked with an engineered microorganism of any of paragraphs 1-14.
16. The engineered microorganism of any of paragraphs 8-15, wherein the barcode region is unique or distinguishable from at least one other barcode region comprised by other items marked with an engineered microorganism of any of paragraphs 1-15.
17. The engineered microorganism of any of paragraphs 8-16 wherein the Cas enzyme scaffold comprises a scaffold for Cas13.
18. The engineered microorganism of any of paragraphs 8-17, wherein the transcription initiation site comprises a T7 transcription initiation site.
19. The engineered microorganism of any of paragraphs 1-18, wherein the at least one essential gene comprises a conditional essential gene.
20. The engineered microorganism of any of paragraphs 1-19, wherein the at least one conditional essential gene comprises an essential compound synthesis gene.
21. The engineered microorganism of paragraph 20, wherein the at least one essential compound synthesis gene comprises an amino acid synthesis gene.
22. The engineered microorganism of paragraph 20, wherein the at least one essential compound synthesis gene comprises a nucleotide synthesis gene.
23. The engineered microorganism of paragraph 20, wherein the at least one essential compound synthesis gene comprises a synthesis gene for threonine, methionine, tryptophan, phenylalanine, histidine, leucine, lysine, or uracil.
24. The engineered microorganism of paragraph 20, wherein the at least one essential compound synthesis gene is selected from the group consisting of thrC, metA, trpC, pheA, HIS3, LEU2, LYS2, MET15, and URA3.
25. The engineered microorganism of any of paragraphs 20-24, comprising an inactivating modification of at least two or more essential compound synthesis genes.
26. The engineered microorganism of any of paragraphs 1-25, wherein the at least one germination gene is selected from the group consisting of cwlJ, sleB, gerAB, gerBB, and gerKB.
27. The engineered microorganism of any of paragraphs 1-26, comprising an inactivating modification of two or more germination genes.
28. The engineered microorganism of any of paragraphs 1-27, wherein the engineered microorganism is inactivated through boiling prior to use.
29. A method of determining the provenance of an item, the method comprising:
   a) contacting an item with at least one engineered microorganism of any one of paragraphs 1-28;
   b) isolating nucleic acid from the item;
   c) detecting the genetic barcode element of the at least one isolated engineered microorganism; and
   d) determining the provenance of the item based on the detected genetic barcode element of the at least one isolated engineered microorganism.
30. The method of paragraph 29, further comprising inactivating the at least one engineered microorganism prior to step (a).
31. The method of paragraph 29 or 30, further comprising distributing the item in between step (a) and step (b).
32. A method of determining the provenance of an item, the method comprising:
   a) isolating nucleic acid from the item; and
   b) detecting the presence of a genetic barcode element, wherein the presence of the genetic barcode element indicates the presence of at least one engineered microorganism comprising a genetic barcode element and an inactivating modification of at least one essential compound synthesis gene or an inactivating modification of at least one germination gene, wherein the presence of the at least one engineered microorganism determines the provenance of the item.
33. A method of marking the provenance of an item, the method comprising contacting the item with at least one engineered microorganism of any of paragraphs 1-28.
34. The method of any of paragraphs 32-33, wherein the microorganism comprises first and second barcode regions, wherein the first barcode region indicates that an item on which the microorganism is detected is from one of a group of known sources, and the second barcode region indicates that an item on which the microorganism is detected is from a particular source of said group of sources.
35. The method of paragraph 34, comprising detecting the presence of the first barcode region in a nucleic acid sample from an item, thereby determining that the item is from a group of known sources.
36. The method of paragraph 34 or 35, further comprising detecting the presence of the second barcode region in the same or different nucleic acid sample from the item, thereby determining that the item is from a particular member of said group of known sources.
37. The method of any of paragraphs 32-36, wherein the item is a food item.
38. The method of any of paragraphs 32-37, wherein the step of detecting the genetic barcode element comprises a method selected from the group consisting of sequencing, hybridization with fluorescent or colorimetric DNA, and SHERLOCK.
39. The method of any of paragraphs 32-38, wherein the sequence of the barcode region of the engineered microorganism is specific for the item or group of items.
40. The method of any of paragraphs 32-39, wherein the sequence of the barcode region of the engineered microorganism is specific for a point of origin of an item or group of items.
41. The method of any of paragraphs 32-40, wherein the step of detecting the genetic barcode element of the isolated nucleic acid comprises:
   a) detecting the first barcode region; and
   b) if the first barcode region is detected, then detecting the second barcode region; or if the first barcode region is not detected, then determining that no engineered microorganism is present on the item.
42. A method of determining the path of an item or an individual across a surface, comprising:
   a) contacting a surface with at least two engineered microorganisms of any one of paragraphs 1-28;
   b) allowing the item or individual to contact the surface in a continuous or discontinuous path;
   c) isolating nucleic acid from the item or individual;
   d) detecting the genetic barcode elements of the at least two isolated engineered microorganisms; and
   e) determining the path of the item or individual across the surface based on the detected genetic barcode element of the at least two isolated engineered microorganisms.
43. The method of paragraph 42, wherein the surface comprises sand, soil, carpet, or wood.
44. The method of paragraph 42 or 43, wherein the surface is divided into a grid comprising grid sections, wherein each grid section comprises at least one engineered microorganism that is distinguishable from all other engineered microorganism on the surface.
45. The method of paragraph 44, wherein each grid section comprises at least two distinguishable engineered microorganisms.
46. The method of paragraph 44, wherein each grid section comprises at least three distinguishable engineered microorganisms.
47. The method of paragraph 44, wherein each grid section comprises at least four distinguishable engineered microorganisms.
48. The method of paragraph 44, wherein the item or individual is determined to have contacted a specific grid section if at least one engineered microorganism originating from the specific grid section is detected on the item or individual.
49. The method of paragraph 48, wherein the path of the item or individual across the surface comprises the specific grid sections that the item or individual is determined to have contacted.
50. The method of paragraph 44, wherein the item or individual is determined to not have contacted a specific grid section if none of the engineered microorganisms originating from the specific grid section are detected on the item or individual.
51. The method of paragraph 50, wherein the path of the item or individual across the surface does not comprise the specific grid sections that the item or individual is determined to not have contacted.

### EXAMPLES

### Example 1: Barcoded Strain for Food Provenance

Described herein is a Bacillus subtilis that is both germination deficient and crippled for growth in natural environments and contains a sequence that allows for rapid tracking and identification.

Also described herein is a strain of Saccharomyces cerevisiae that contains a sequence that allows for rapid tracking and identification. This strain has a set of deletions which cripple it for growth in the wild. The strain is not germination deficient, but a production protocol (e.g., boiling) can be applied that kills all spores while keeping them structurally intact.

Both strains are safe for release and tracking in the environment. The primary envisioned use of these engineered strains is such that purchasers can determine the sources of their produce. This can be very useful for tracking back sources of contaminated food to a specific farm or processing plant, thereby minimizing the need for agricultural product recalls.

Engineered B. subtilis

B. subtilis strain 168 was modified as follows: ΔthrC::lox72, ΔmetA::lox72, ΔtrpC::lox72, ΔpheA::lox72, ΔsleB::lox72, ΔcwlJ::lox72, ΔgerAB::lox72, ΔgerBB::lox72, ΔgerKB::lox72, ycgO::UTS (lox72)

In ycgO::UTS, the UTS is a Unique Tracking Sequence (described below).

As used herein, “lox72” refers to the 150 bp scar left after Cre-lox-mediated excision of the antibiotic marker.

This engineered B. subtilis strain, denoted Δ9, is deficient for germination based on the deletion of the three germinate receptors (e.g., gerAB, gerBB, gerKB) and the two enzymes required for degrading the cell wall of the spore (e.g., sleB, cwlJ).

The “Δ9” strain is a quadruple auxotroph, comprising mutations or deletions in essential compound synthesis genes (e.g., thrC, metA, trpC, pheA). These deletions block the ability to grow unless media is supplemented with threonine, methionine, tryptophan, and phenylalanine.

CwlJ and SleB are enzymes that are needed to degrade the spore cell wall during germination. ΔcwlJ ΔsleB mutants are deficient in their ability or unable to degrade the spore cell wall during germination

GerA, GerB, and GerK germinant receptors that sense and respond to nutrients. ΔgerAB ΔgerBB ΔgerKB mutants thus has a decreased ability or in ability to sense and respond to nutrients for germination.

Δ9 is essentially a “pebble” with DNA inside them. Vegetative cells also cannot thrive in natural environments. 168 is a strain background of B. subtilis. The Δ9 strain comprises these germination mutants together with nutritional deficiency and barcodes.

Barcodes and mutations as described for B. subtilis can also be engineered into Bacillus thuringiensis (an agricultural biocide).

Engineered S. cerevisiae Strains

Saccharomyces cerevisiae strain BY4743 MATa/a was modified as follows: his3 Δ1/his3 Δ1 leu2 Δ0/leu2 Δ0 LYS2/lys2 Δ0 met15 Δ0/MET15 ura3 Δ0/ura3 Δ0 ho::UTS.

In ho::UTS, the UTS is a Unique Tracking Sequence (described below) his3, leu2, lys2, met15, and ura3 are common nutrition markers. Deletions of these genes make the strains inviable in the absence of the nutrient being supplemented back into the growth medium.

### Unique Tracking Sequence

The UTS comprises the following elements, e.g., in the following order: (a) RPA primer 1 (e.g., GATAAACACAGGAAACAGCTATGACCATGATTACG, SEQ ID NO: 1); (b) unique barcode region (see below); (c) Cas13 scaffold (e.g., GTTTTAGTCCCCTTCGTTTTTGGGGTAGTCTAAATC, SEQ ID NO: 2); (c) T7 transcription site (e.g., CCCTATAGTGAGTCGTATTAGAATT, SEQ ID NO: 3); (d) RPA primer 2 (e.g., GGGATCCTCTAGAAATATGGATTACTTGGTAGAACAG, SEQ ID NO: 4). See e.g., FIG. 1.

The two RPA primers permit amplification of the full UTS sequence. The RPA primers were chosen to be compatible for amplification by both qPCR (common amplification method used in labs) and RPA reagents (common amplification method used in the field).

### Unique Barcode Regions

A number of barcodes have been designed. The barcode can be 28 base pairs in length. All barcodes are designed to have a Hamming distance of at least 5 base pairs. This permits accurate detection and differentiation of the barcode sequence by multiple detection methods including sequencing, hybridization with fluorescent or colorimetric DNA, and SHERLOCK (CRISPR-Cas+RNAse alert).

Non-limiting examples of barcodes are included below.

Barcode_1
(SEQ ID NO: 5)
TCGTCAGTCGTAACTTGGGAACGCACAT
Barcode_2
(SEQ ID NO: 6)
CTTTGGGGTTGGATAAATCTGTCGTGGT
Barcode_3
(SEQ ID NO: 7)
TGAATAAGCGCGGTCCCTAATGTTGGTG
Barcode_4
(SEQ ID NO: 8)
ACCGGTTTATAGTTCGAACAGTCGCAGC
Barcode_5
(SEQ ID NO: 9)
CCCCGTGTGGTAACTACGCAAGCCTAAC
Barcode_6
(SEQ ID NO: 10)
ACGTAGGGGGGCGCGTAACCACTAGCTC
Barcode_7
(SEQ ID NO: 11)
AGTGTCCCTTATTCTACTTTGAATTATC
Barcode_8
(SEQ ID NO: 12)
GAGTTACGGGTCAGGATCATTGCGCAGG
Barcode_9
(SEQ ID NO: 13)
GTGAGTCCGGCCTATCACGTTTGGTAGG
Barcode_10
(SEQ ID NO: 14)
TCAGGGGAAACGAGTTAAGCAGAGGCAG
Barcode_11
(SEQ ID NO: 15)
CTTGGTCCAATCGTATGCTAAGAGTAGC
Barcode_12
(SEQ ID NO: 16)
TCGGTTGCACGCGCGTCCTGCACGCTCG
Barcode_13
(SEQ ID NO: 17)
GTTCAAAAGCGGGAGTCCCGGTGAAACC
Barcode_14
(SEQ ID NO: 18)
GAGGGCTTCTACGAAATTGCCTCACCAT
Barcode_15
(SEQ ID NO: 19)
GGGATGCCCTGATAACGTAGAGTCTCAG
Barcode_16
(SEQ ID NO: 20)
TAGTACGGGCCACGATCTAAGTCGGCGG
Barcode_17
(SEQ ID NO: 21)
GTATGTGGGTACGATTGTAGGCTAGAAA
Barcode_18
(SEQ ID NO: 22)
AGAACTACCTACGTGGCCACGCAAGCCC
Barcode_19
(SEQ ID NO: 23)
TGCCTCAGAATGGGGTTTAACGTTCAGG
Barcode_20
(SEQ ID NO: 24)
ATGTTGGGACGCCAGACTGACACGCAAA
Barcode_21
(SEQ ID NO: 25)
CCTACACCTTCACCGAGTGTGGAGCAAG
Barcode_22
(SEQ ID NO: 26)
GTTTAACATTCGGGTATCGTCCTTACGA
Barcode_23
(SEQ ID NO: 27)
GCAATATTACTAGTCCGTGTCAGCCAGA
Barcode_24
(SEQ ID NO: 28)
CCGACTAGGTCGGCGATACCATAGCACC
Barcode_25
(SEQ ID NO: 29)
GATGACGCCTCACGTCCGTTGCCCCCTA
Barcode_26
(SEQ ID NO: 30)
GCTCCGTAGCAATTGATAAACCGCTGAG
Barcode_27
(SEQ ID NO: 31)
CTATCGGATTTTTAGATAAATTACGAGG

### Universal Region of the UTS

The UTS comprises a universal region currently composed of a Cas13 scaffold and a T7 transcription site. This sequence allows for the rapid and generic detection of all UTS by fieldable assays. In addition, this universal sequence is compatible with multiple detection methods including sequencing, hybridization with fluorescent or colorimetric DNA, and SHERLOCK (CRISPR-Cas+RNAse alert).

### Group and Unique Barcode Regions

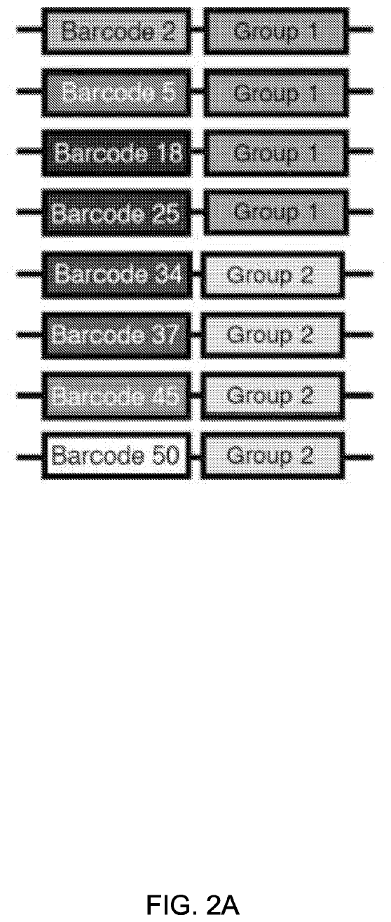

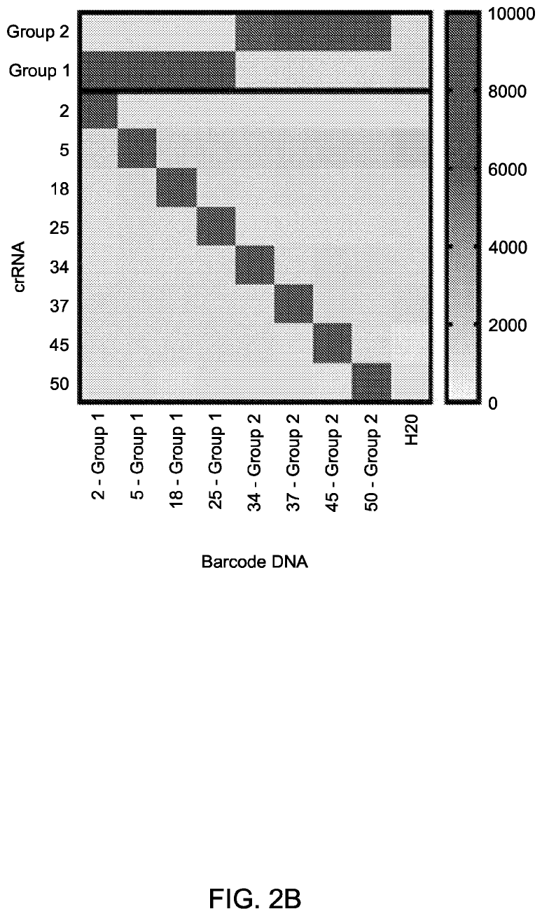

The system can also contain two tandem barcodes which can be called the group barcode and unique barcode. The group barcode has a similar sequence design to the universal barcode (e.g., unique tracking sequence) but is shared between multiple “products”. The unique barcode is unique to the sample. This allows for the final product to be made in groups. One can have all the UTS for one purpose share the group barcode but not the unique barcode. For example, this allows one to first test if any barcodes are present with the group barcode, and then follow with a second test to determine if the unique barcode is present (see e.g., FIG. 2A-2B).

### Example 2: Barcoded Microbial System for High Resolution Object Provenance

Globalization of supply chains has dramatically complicated the process of determining the origins of agricultural products and manufactured goods. Determining the origin of these objects is critical, e.g., in cases of foodborne illness, but current labeling technologies are prohibitively labor-intensive and are easy to remove, replace, or otherwise subvert (see e.g., Wognum et al. Advanced Engineering Informatics (2011), 25(1), 65-76, the content of which is incorporated herein by reference in its entirety). Similarly, tools that label unknown persons or objects passing through a location of interest can also be useful to law enforcement as a complement to fingerprinting and video surveillance (see e.g., Gooch et al. TrAC Trends in Analytical Chemistry (2016) 83(Part B), 49-54, the content of which is incorporated herein by reference in its entirety).

Microbial communities offer an alternative to standard labeling approaches. Any object (e.g., placed in and interacting with a particular environment) gradually adopts the naturally occurring microbes present in its environment (see e.g., Lax et al., Science 345, 1048-1052 (2014); Jiang et al. Cell 175, 277-291.e31 (2018); the contents of each of which are incorporated herein by reference in their entireties); thus, it has been suggested that the natural microbial composition of an object could be used to determine object provenance (see e.g., Lax et al., Microbiome 3, 21 (2015), the content of which is incorporated herein by reference in its entirety). Challenges with this approach include variability of microbial community composition between different locations areas (e.g., the microbial communities are not reliably large or stable enough to uniquely identify specific locations); moreover, using natural microbes requires extensive, expensive, and time-consuming mapping of natural environments.

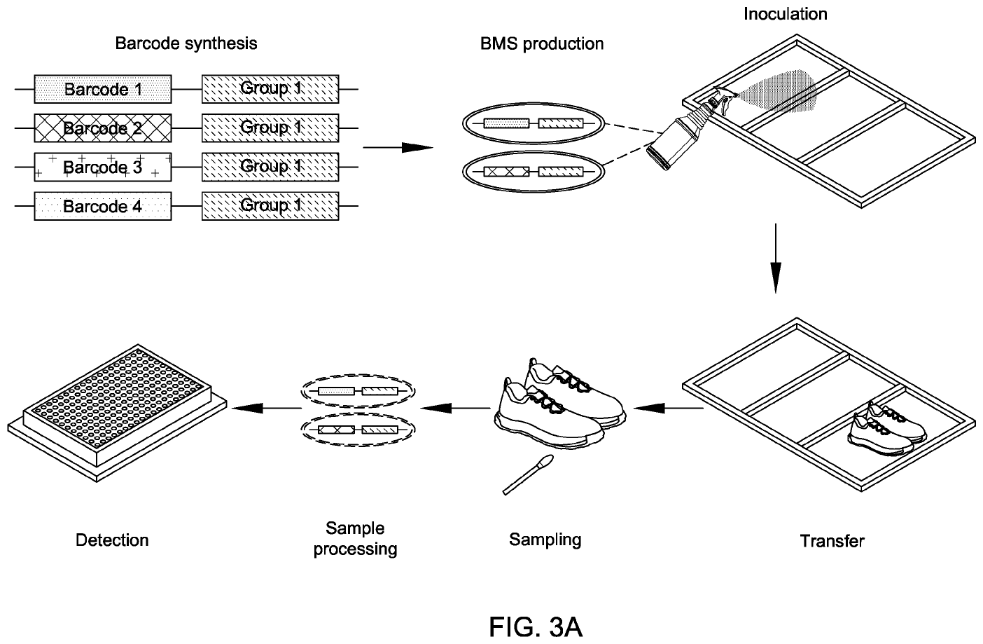

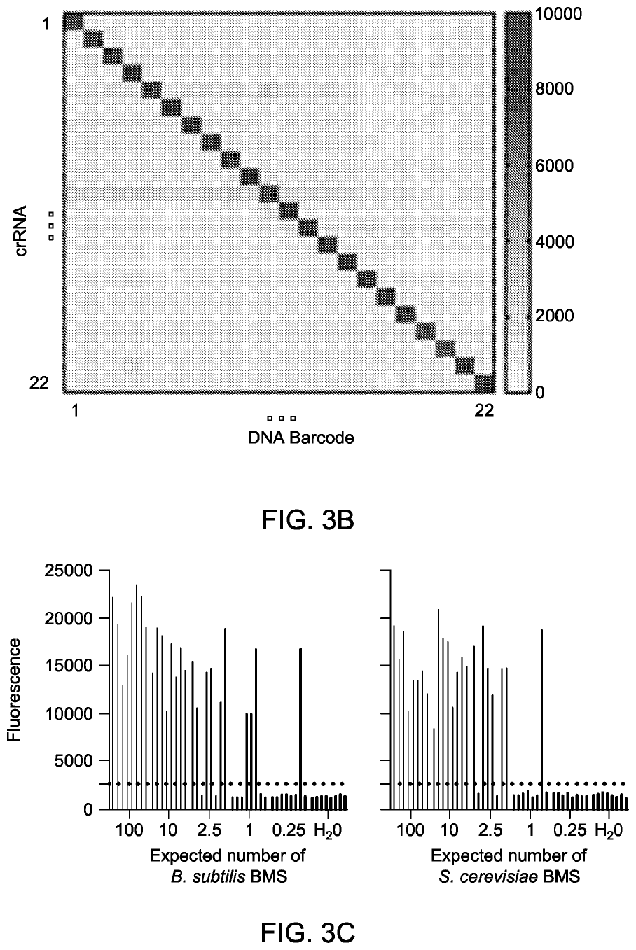

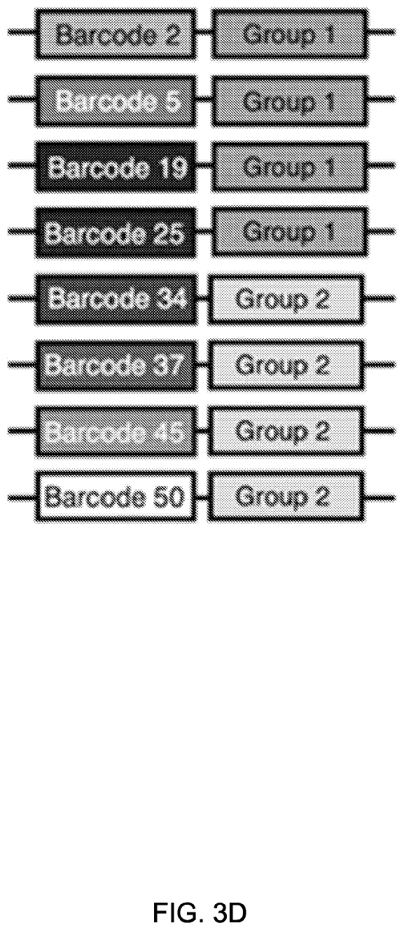

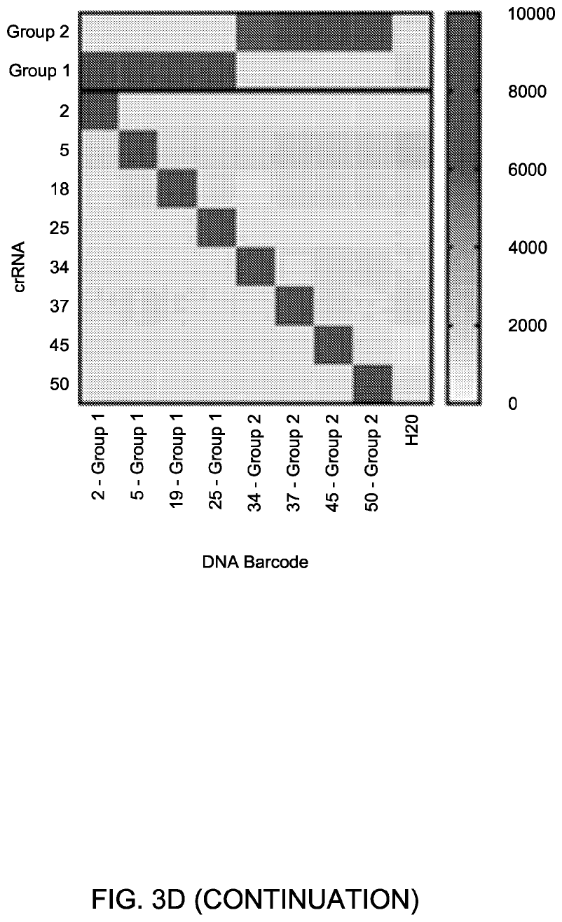

To circumvent these challenges, described herein is the deliberate introduction and use of synthetic microbes (e.g., non-viable microbial spores) harboring barcodes that uniquely identify particular locations of interest (e.g., food production areas). These microbes (e.g., synthetic spores) offer a sensitive, inexpensive, and safe way to map object provenance (e.g., food items) provided that several important criteria are met. These criteria include: 1) microbes must be compatible with growth at industrial scale; 2) the synthetic microbes must persist in the environment and reliably label objects that pass through it; 3) the microbes must be bio-contained and not viable in the wild to prevent adverse ecological effects or cross-contamination; and 4) the encoding and decoding of information regarding object provenance must be rapid, sensitive and specific. Barcoding approaches have been explored previously to model pathogen transmission, but did not explicitly address those challenges; see e.g., Buckley et al. App. and Env. Micro. 78, 8272-8280 (2012); Emanuel, et al., App. and Env. Micro. 78, 8281-8288 (2012); the contents of each of which are incorporated herein by reference in their entireties. Described herein is the BMS (Barcoded Microbial Spores; also referred to interchangeably as FMS (Forensic Microbial Spores)) system, a scalable, safe and sensitive system that uses DNA-barcoded microbial spore mixtures to permit the determination of object provenance (see e.g., FIG. 3A).

The BMS system leverages the natural ability of spores to persist for long periods in the environment without growth (see e.g., Ulrich et al., PLoS One. 2018 Dec. 4; 13(12):e0208425, the content of which is incorporated herein by reference in its entirety). Unique DNA barcodes were designed and integrated into the genomes of Bacillus subtilis (B. subtilis) and Saccharomyces cerevisiae (S. cerevisiae) spores, creating a set of BMS that can be used combinatorially to provide a nearly infinite set of unique identification codes. The BMS can be: 1) manufactured at scale using standard cloning and culturing techniques; 2) applied (i.e., inoculated) to surfaces by spraying; and 3) efficiently transferred to objects that come into contact with the inoculated surface. To identify barcodes, BMS sampled from the objects are lysed and can be decoded with a range of methods including but not limited to SHERLOCK, a recombinase polymerase amplification (RPA) method coupled with a Cas13a-based nucleic acid detection assay (see e.g., Gootenberg et al. Science, 356 (2017), pp. 438-442, the content of which is incorporated herein by reference in its entirety), PCR or qPCR, and sequencing (see e.g., FIGS. 3A and 7A).

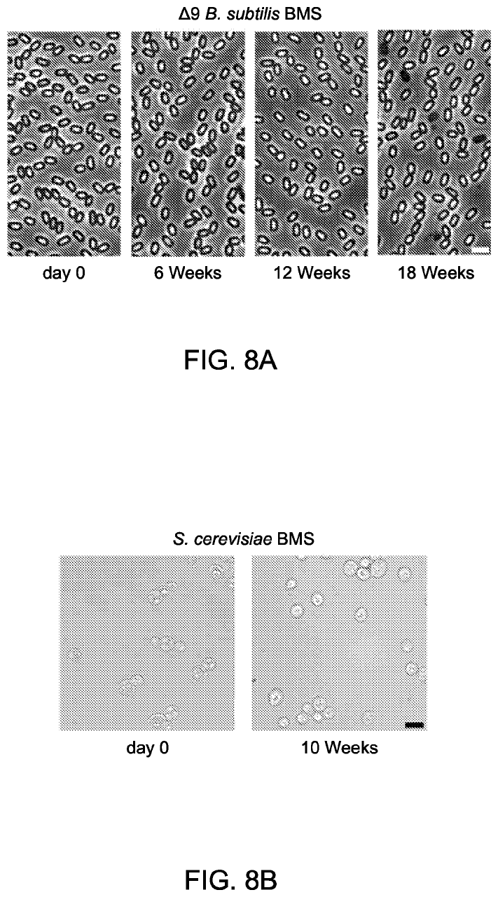

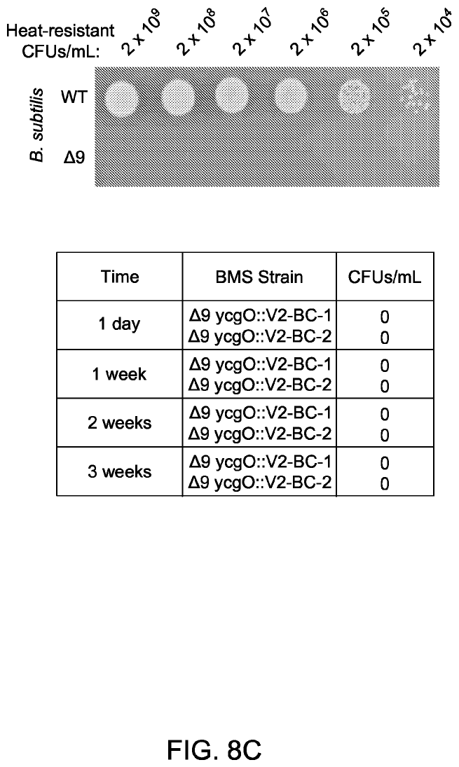

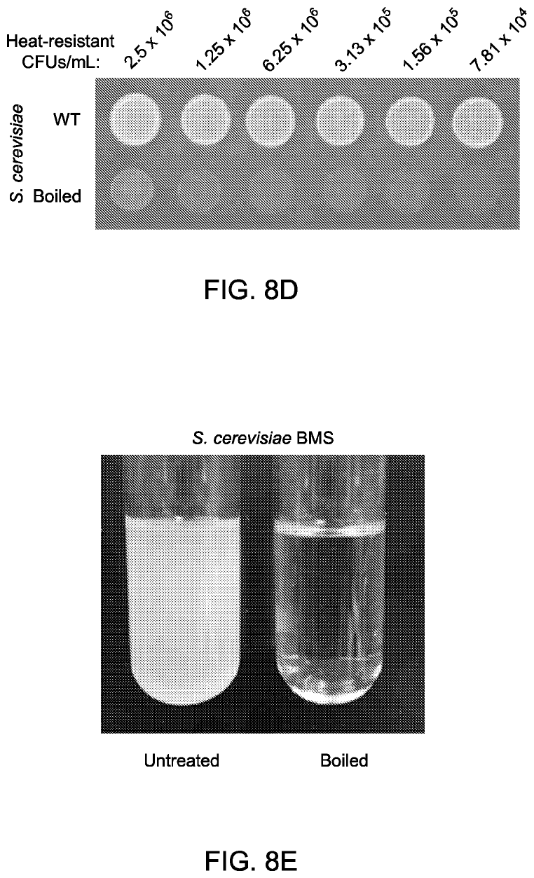

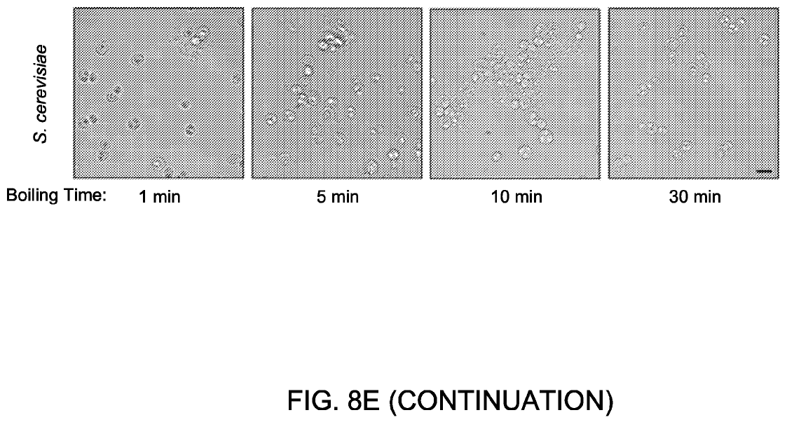

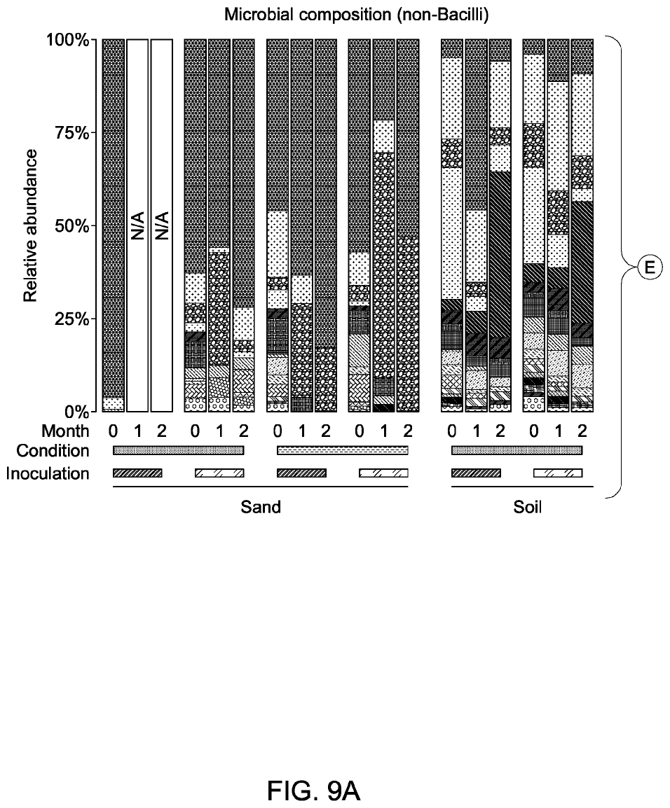

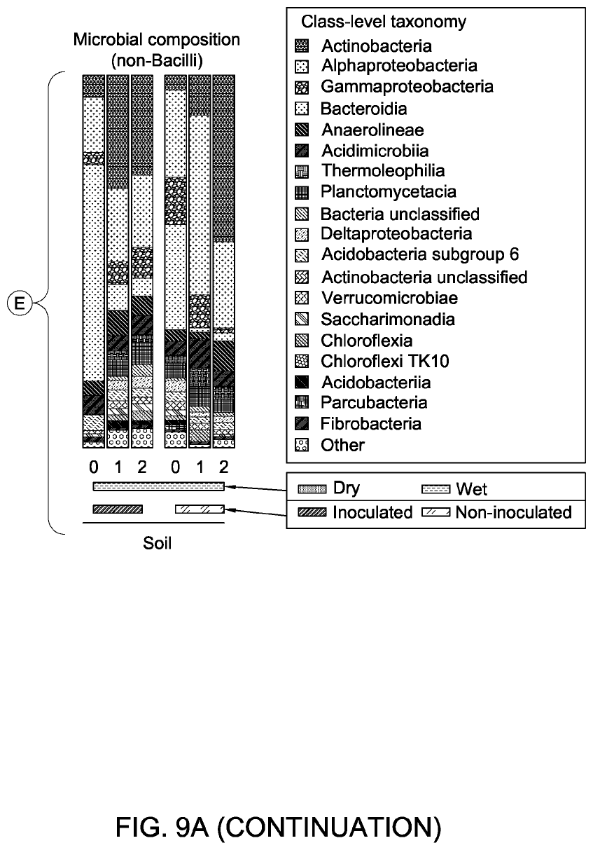

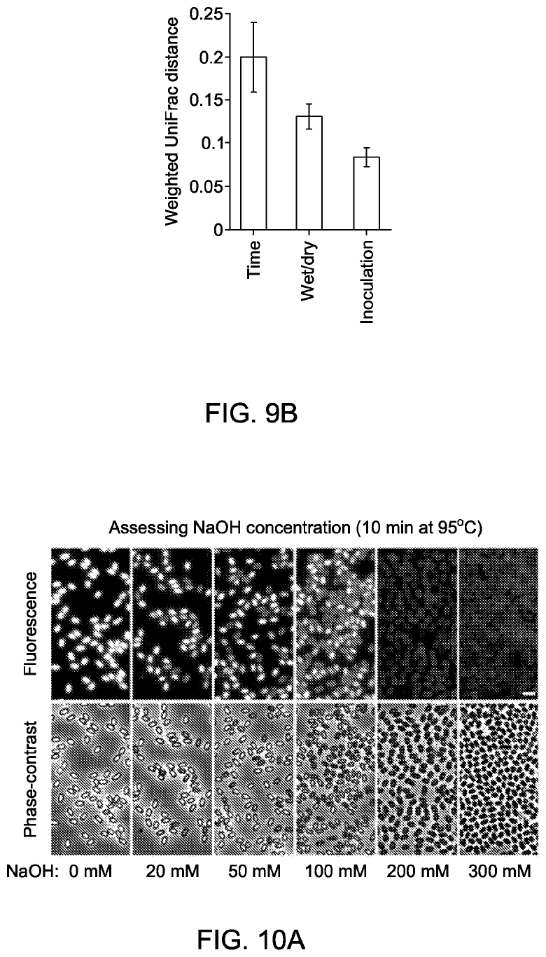

The BMS systems are designed to not impact the native environment into which they are applied (e.g., outside the laboratory), and BMS do not impact the native environment into which they are applied. First, auxotrophic strains were used that require amino acid supplementation for growth. Second, the cells were made to be germination deficient. For B. subtilis spores, the genes encoding the germinant receptors were deleted, and the genes that encode the cell wall lytic enzymes required to degrade the specialized spore cell wall were also deleted. Incubation of >10¹² spores generated from this mutant strain showed they were unable to form colonies or grow in rich medium, and remained stable and non-germinating at room temperature for >3 months (see e.g., FIG. 8A, FIG. 8C). For S. cerevisiae, spores were boiled for 30 minutes to heat-kill vegetative cells and spores prior to application. Incubation of >10⁸ boiled spores on rich medium yielded no colonies (see e.g., FIGS. 8B, 8D-8F). All antibiotic resistance cassettes used to generate the BMS were removed by site-specific recombination to prevent horizontal gene transfer of resistance genes to other organisms in the environment. Finally, the inserted sequence does not encode any gene and does not confer any fitness advantage if horizontally transferred. The microbiome of soil samples was tracked, and inoculation with BMS had insignificant effects on the microbiome compared to natural changes over time or in response to watering (see e.g., FIG. 9A-9B). Wild-type B. subtilis and S. cerevisiae are both commonly found in environmental and food samples.

Multiple BMS can be applied and then decoded simultaneously. A series of tandem (e.g., each 28 bp long) DNA barcodes were designed, each with a Hamming distance>5, allowing more than 10⁹ unique barcodes. To test the specificity of the barcode design in a field-deployable system, 22 barcodes were constructed and their matching crRNAs, and all permutations were assayed in vitro using SHERLOCK. All 22 crRNAs clearly distinguished the correct barcode target (see e.g., FIG. 3B). To scale the system, a rapid and facile method was devised to screen a large number of barcodes and crRNAs in parallel to eliminate those with high cross-reactivity or background; pooled n−1 RPA reactions were validated and performed in vitro with corresponding crRNA and water RPA controls testing 94 crRNA-barcode pairs, eliminating 17 for high background and 7 for cross-reactivity (see e.g., FIG. 7B, FIG. 7C). To test sensitivity and specificity in vivo, 57 barcodes were integrated into B. subtilis and 11 into S. cerevisiae. An efficient spore lysis protocol was developed using heat and sodium hydroxide (see e.g., FIG. 10A-10C), which allowed near single-spore resolution for detection using SHERLOCK (see e.g., FIG. 3C). In vivo and in vitro specificity screenings of crRNA-barcode pairs gave similar results (see e.g., FIG. 7C-7D). In addition, barcodes were tandemly designed with a unique sequence and a shared group sequence (see e.g., FIG. 7A) to aid in high-throughput detection settings where only a subset of samples contains the BMS of interest. In each tandem barcode, one barcode is unique and one is a group barcode that is shared with a subset of other tandem barcodes. The group barcode is compatible with field-deployable detections (e.g., SHERLOCK) (see e.g., FIG. 3D) and can be used to determine whether a BMS of interest is present or to classify groups of BMS, before using a second assay to uniquely identify the BMS (see e.g., FIG. 3D). This two-step process solves the throughput limitations of field-deployable detection and lowers the costs of sequencing. The multistep decoding approach can be used in highly parallelized settings.

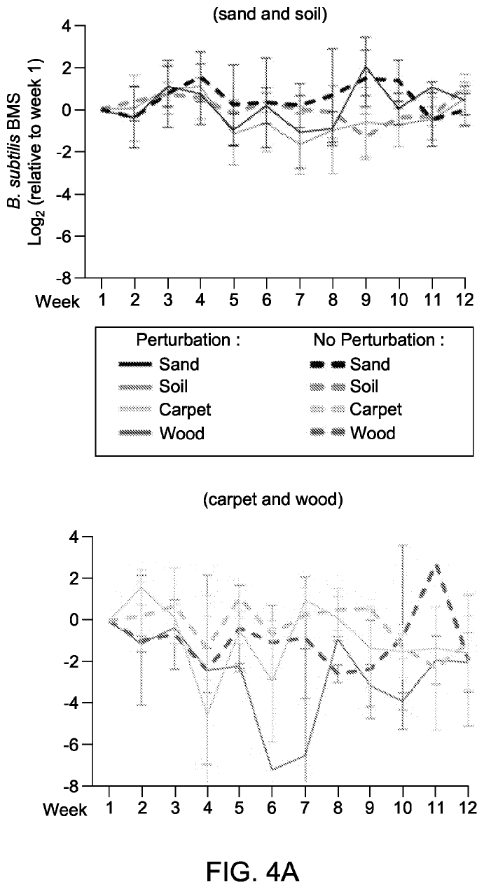

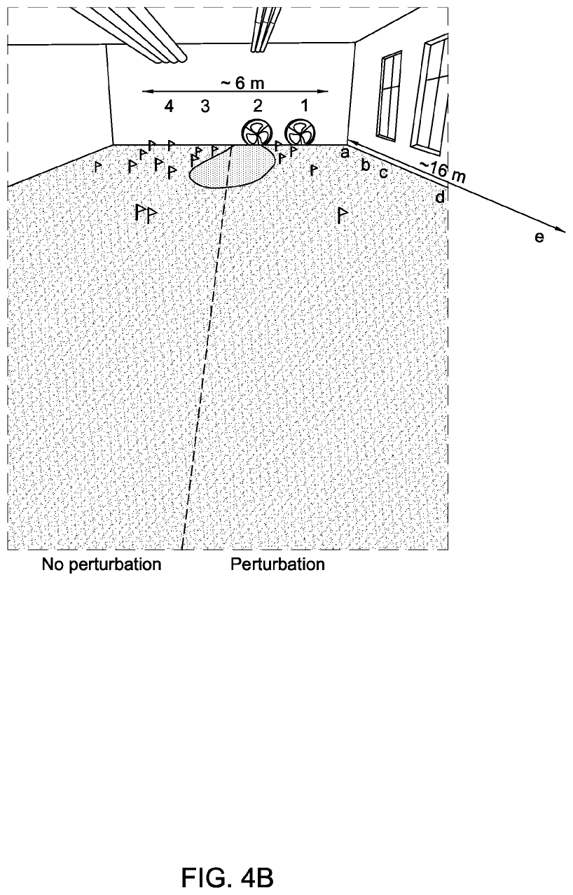

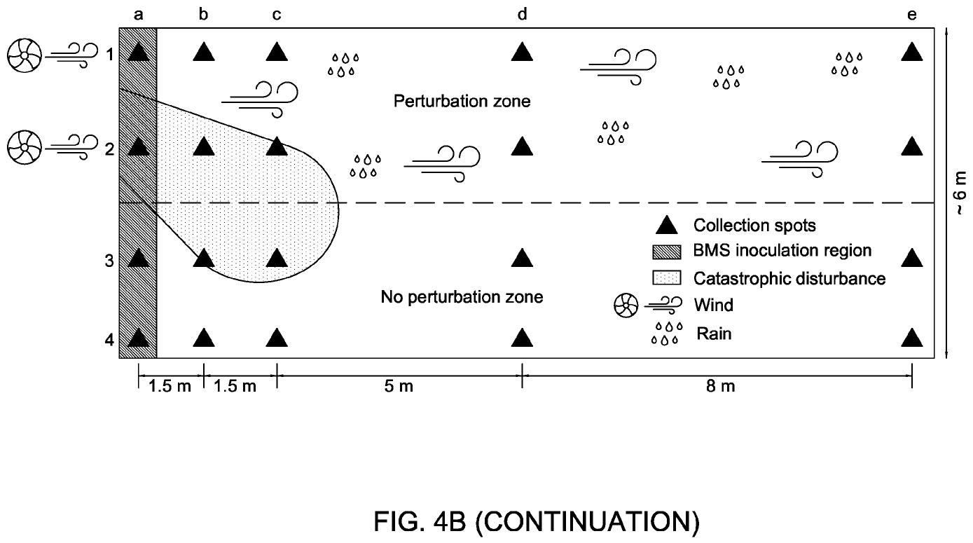

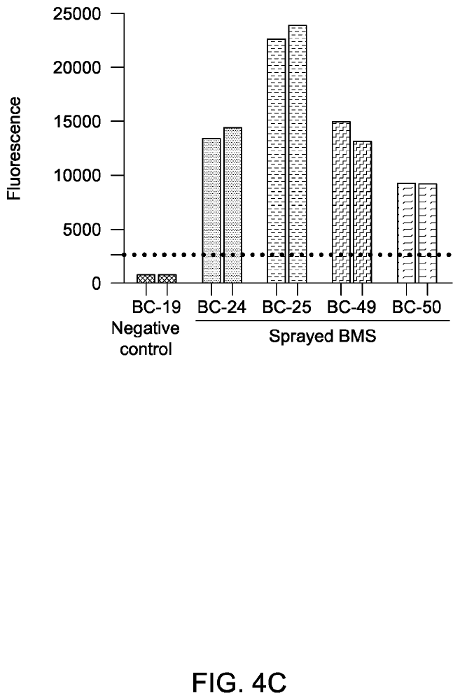

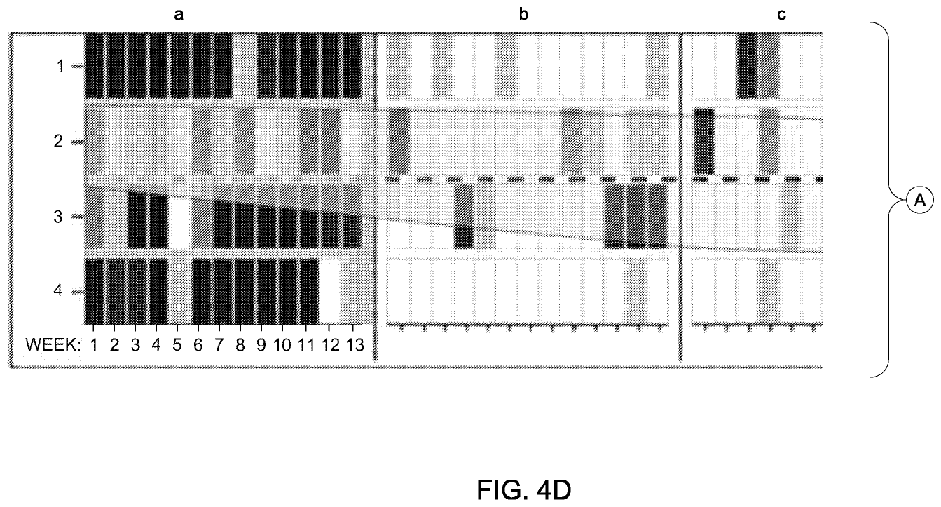

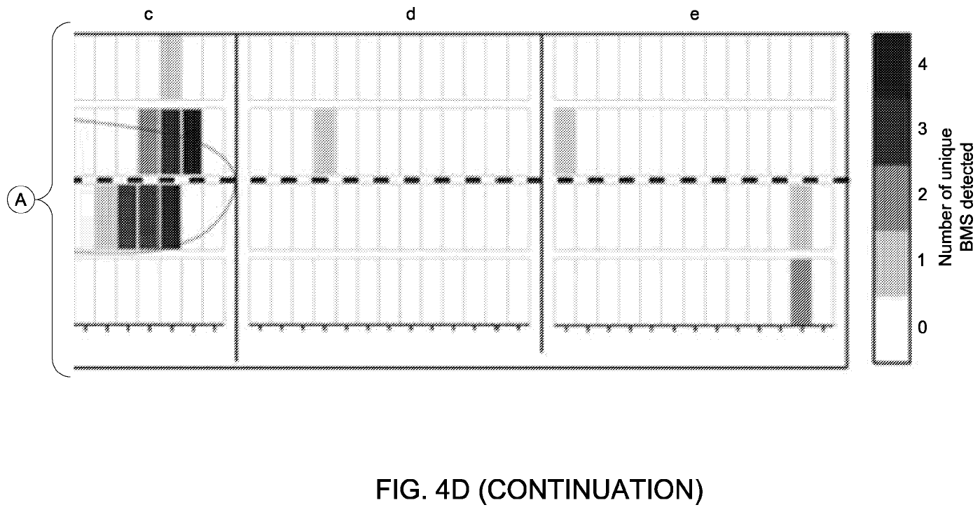

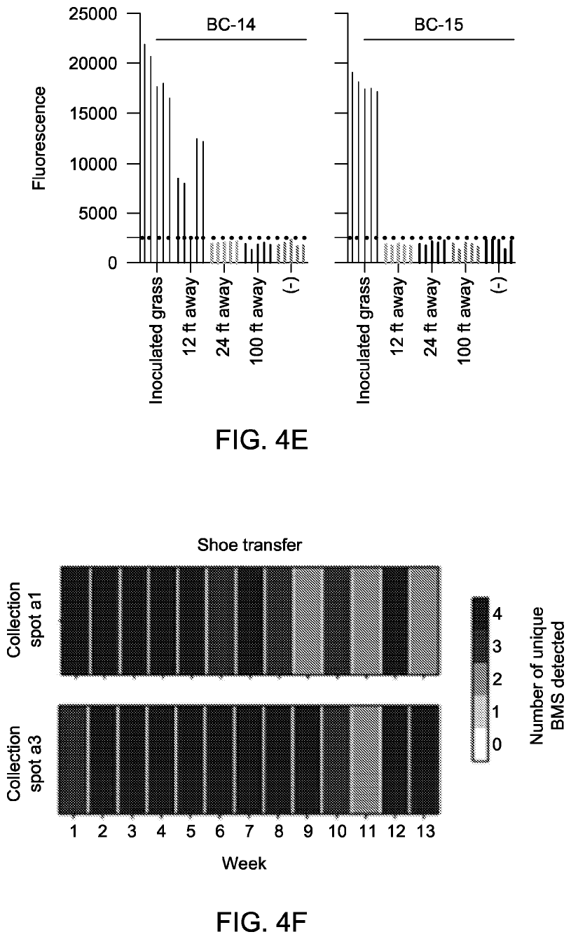

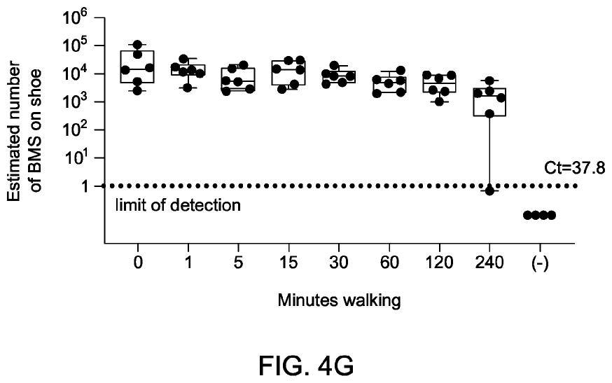

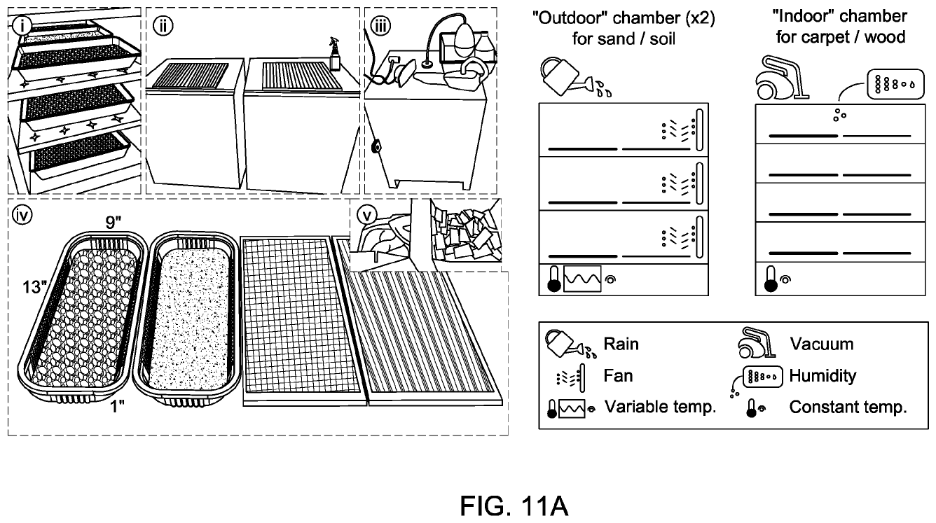

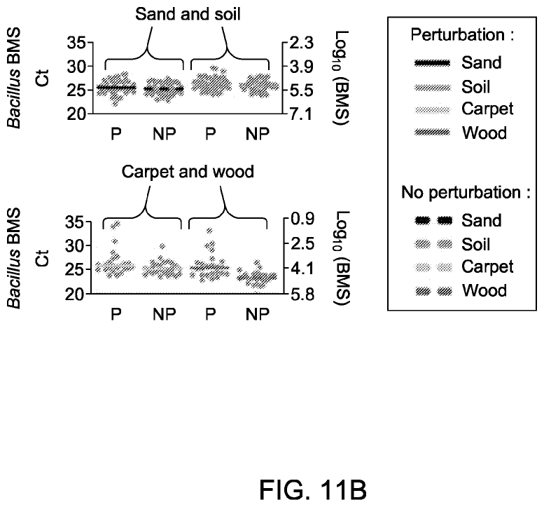

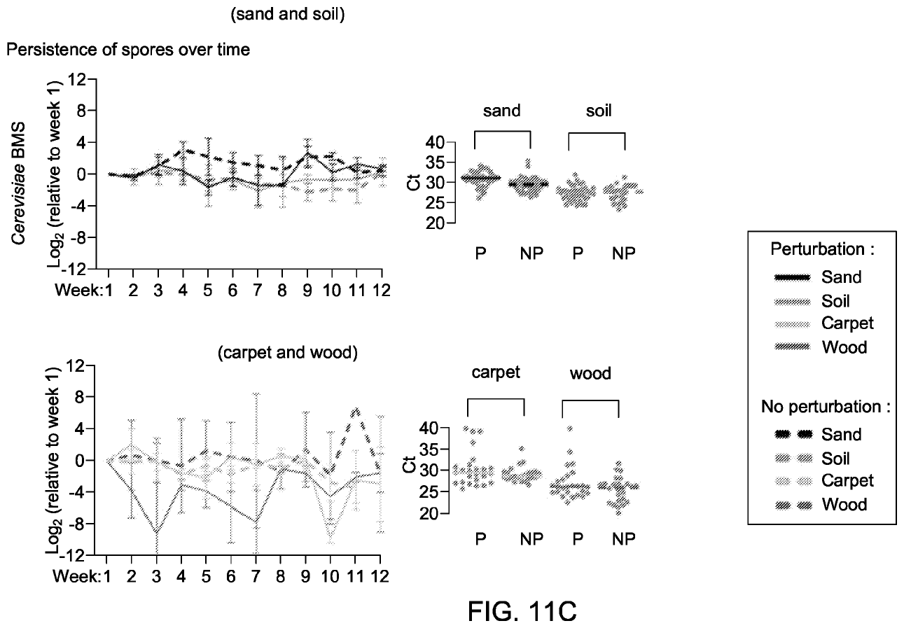

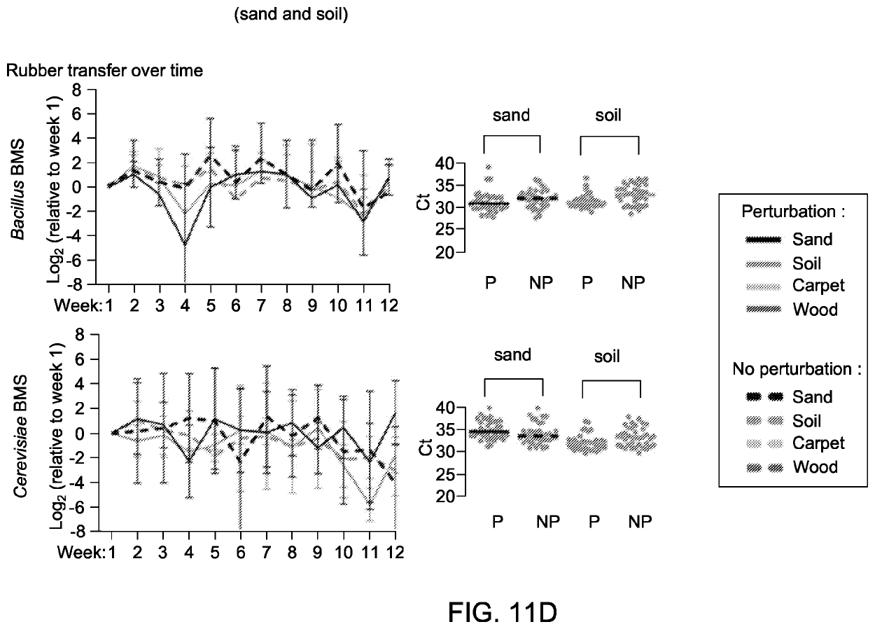

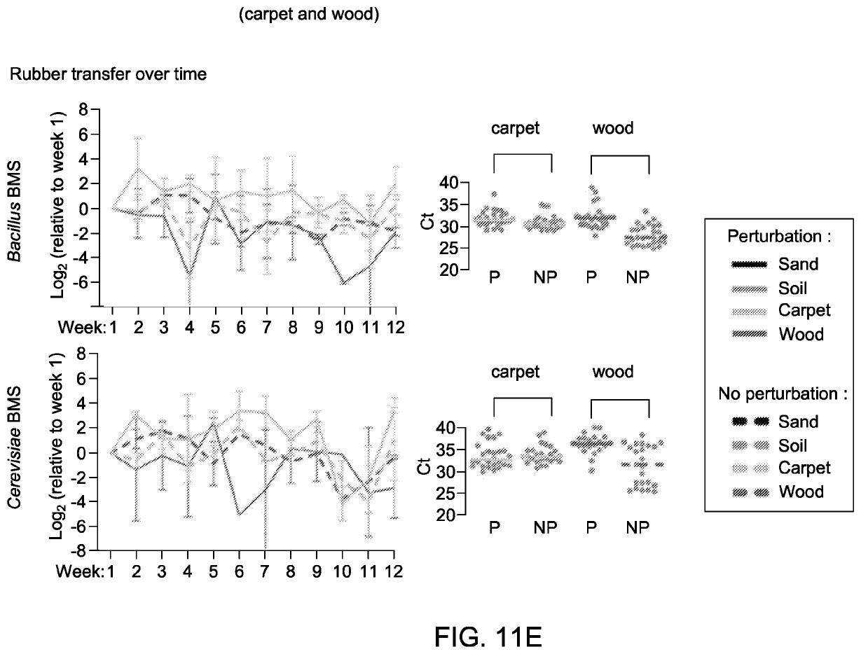

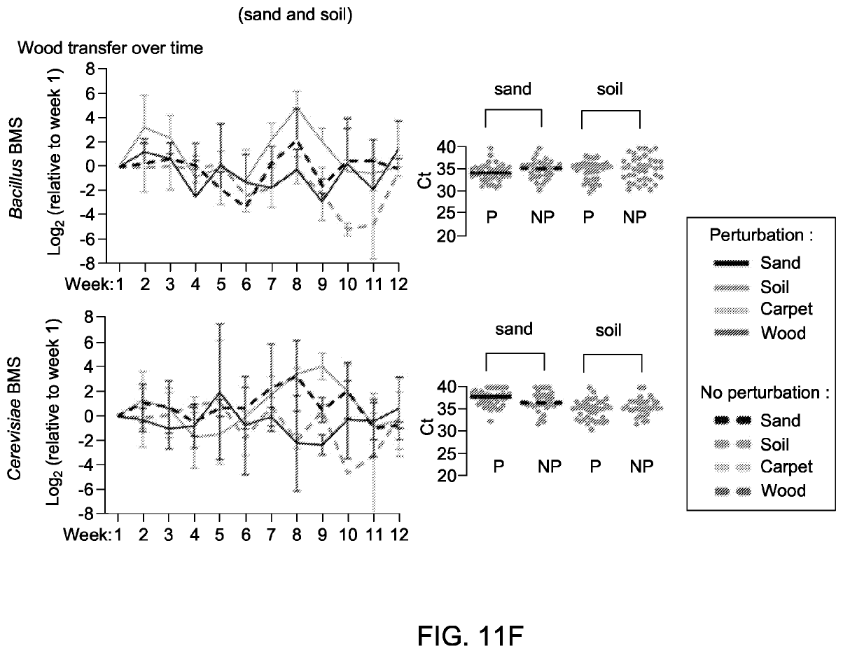

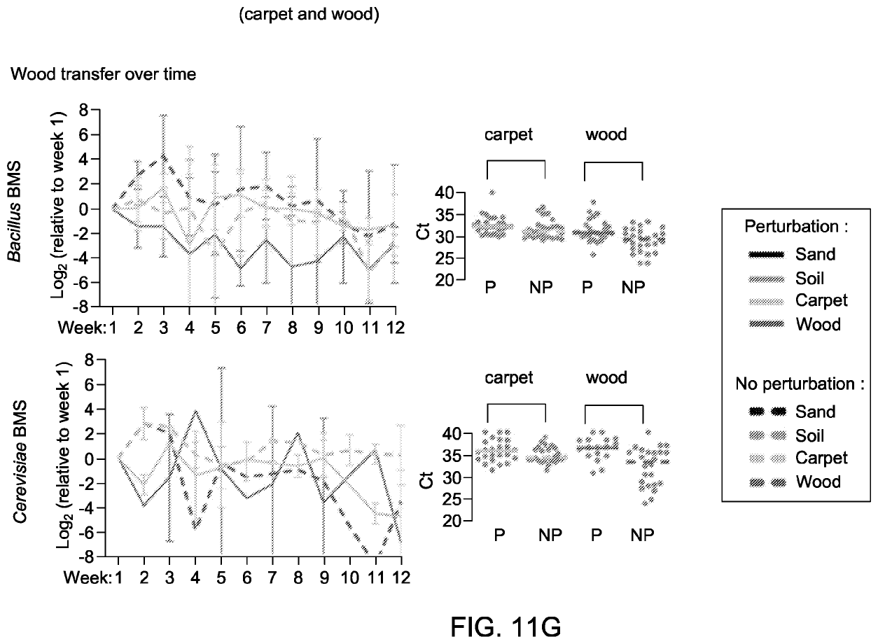

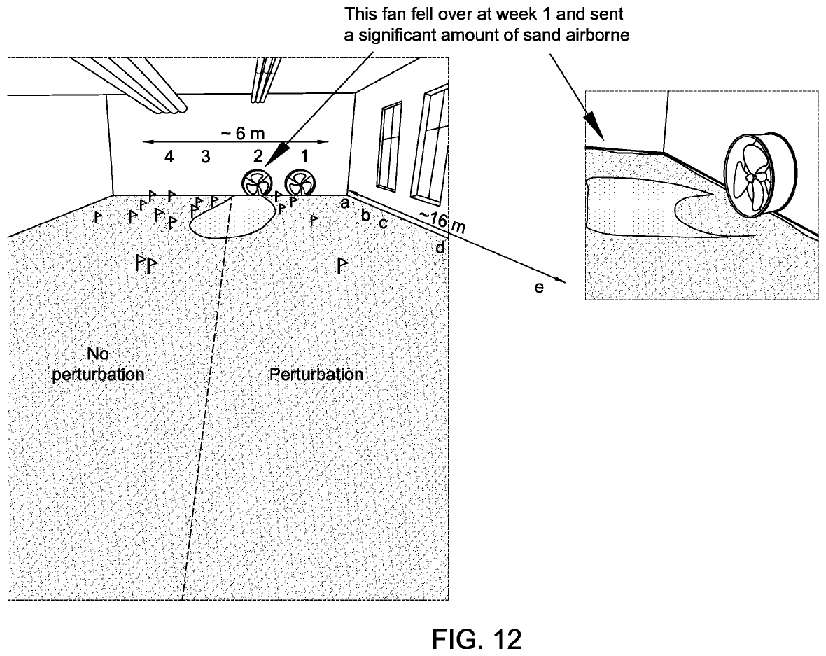

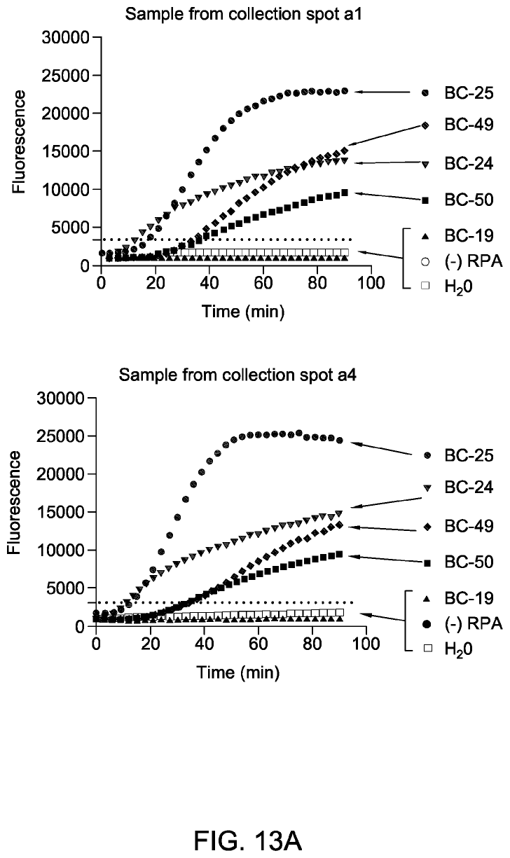

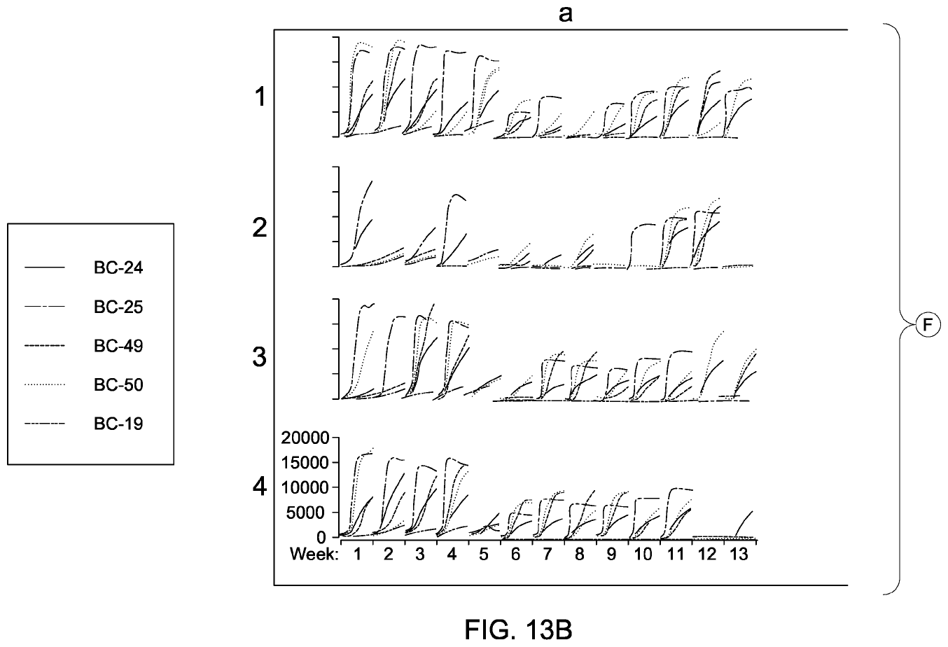

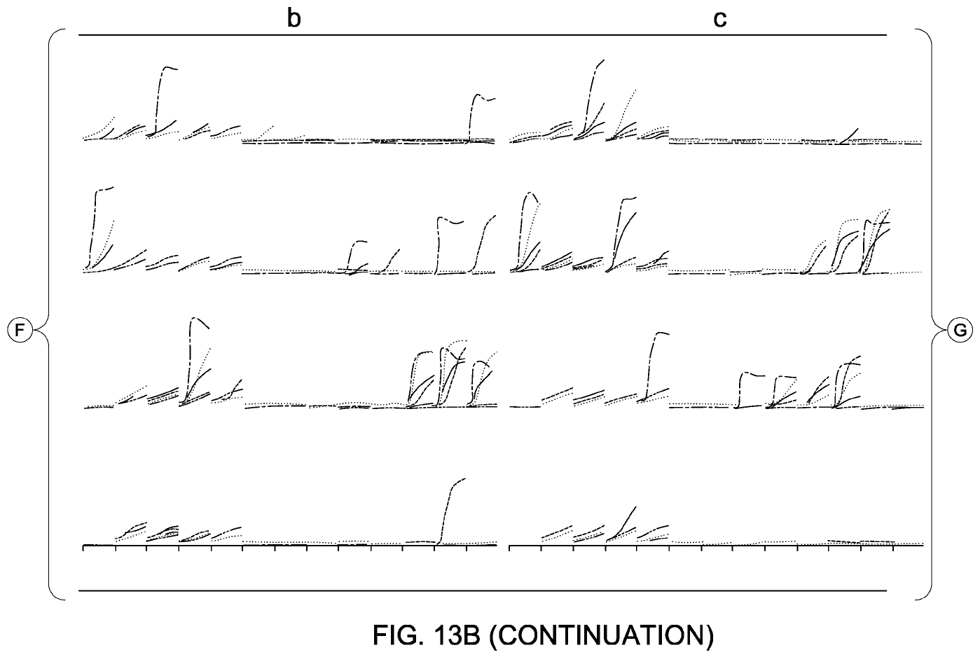

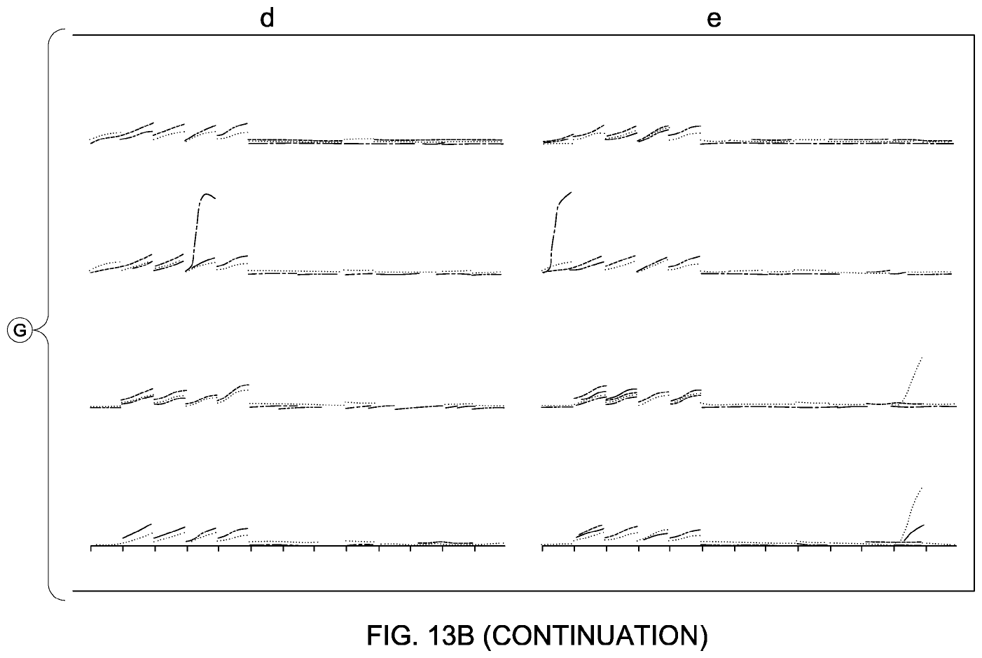

The BMS system is robust and can function on different surfaces in real-world environments (including simulated environments). First, in incubator-scale experiments (see e.g., FIG. 11A and Table 7), qPCR was used to detect and quantify BMS directly from surface samples or surface swabs. BMS persisted on sand, soil, carpet, and wood surfaces for at least three months with little to no loss overtime (see e.g., FIG. 4A, FIG. 11B-11C). Notably, multiple tested perturbations (e.g. simulated wind, rain, vacuuming, or sweeping in FIG. 11A) did not significantly reduce the ability to detect BMS from the surface. Second, a ˜100 m² indoor sandpit was constructed (see e.g., FIG. 4B and FIG. 12), one region was inoculated with BMS (see e.g., FIG. 4C), and the BMS could be readily detected for three months using SHERLOCK (see e.g., FIG. 4D and FIG. 13A-13B). Importantly, perturbations (e.g., simulated wind and rain) did not cause appreciable spreading to non-inoculated areas (see e.g., FIG. 4D and FIG. 13A-13B), even a catastrophic disturbance where a fan fell into the pit, displacing a significant amount of sand, only spread the BMS several meters (see e.g., FIG. 12). In an outdoor environment, BMS inoculated on grass was still detectable after 5 months of exposure to natural weather, with minimal spreading outside of the inoculated region (see e.g., FIG. 4E). This is consistent with low levels of re-aerosolization reported for other spore-forming Bacilli; see e.g., Bishop, et al., Lett. in App. Micro. 64, 364-369 (2017), the content of which is incorporated herein by reference in its entirety.

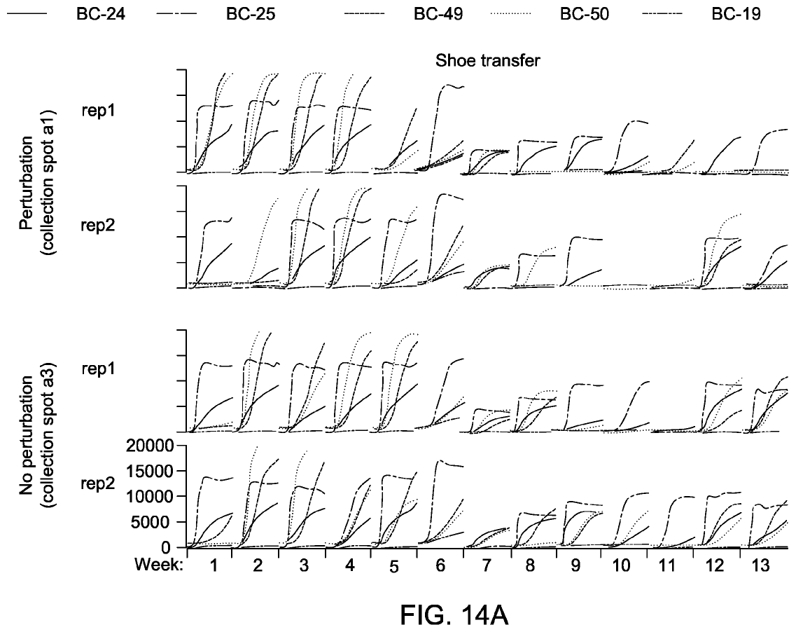

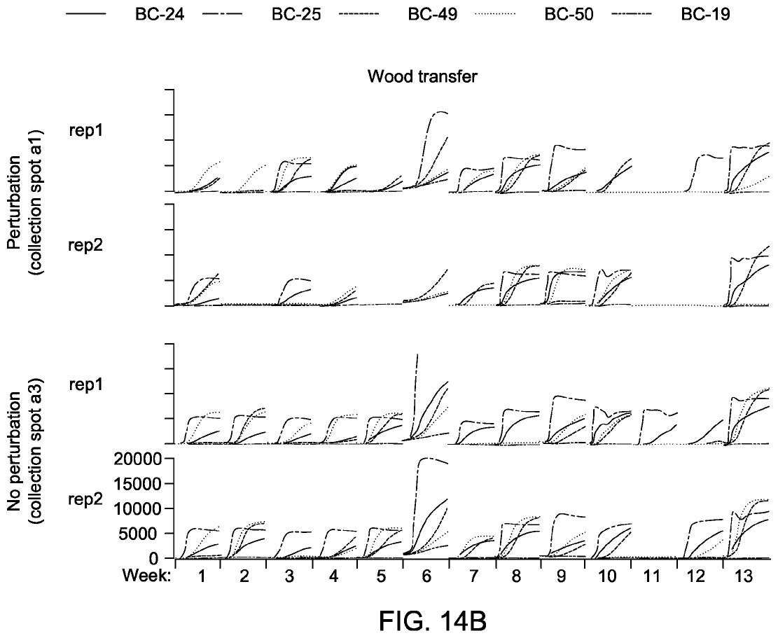

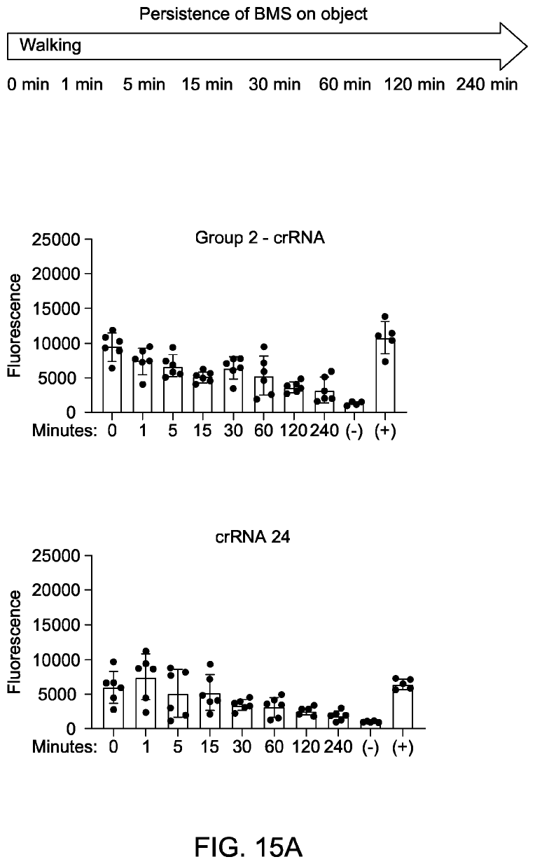

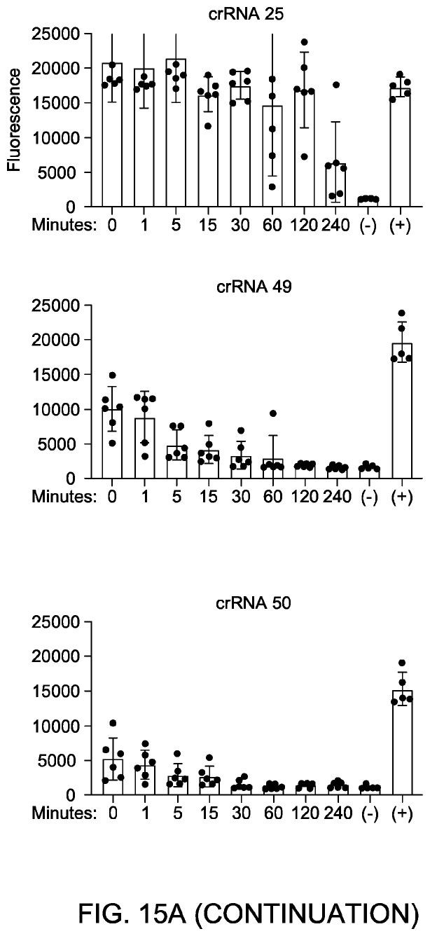

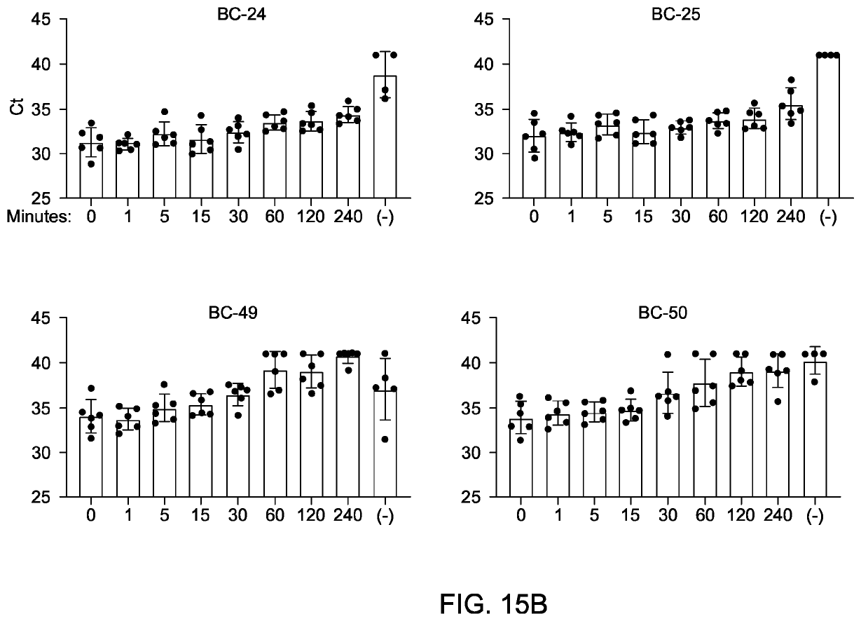

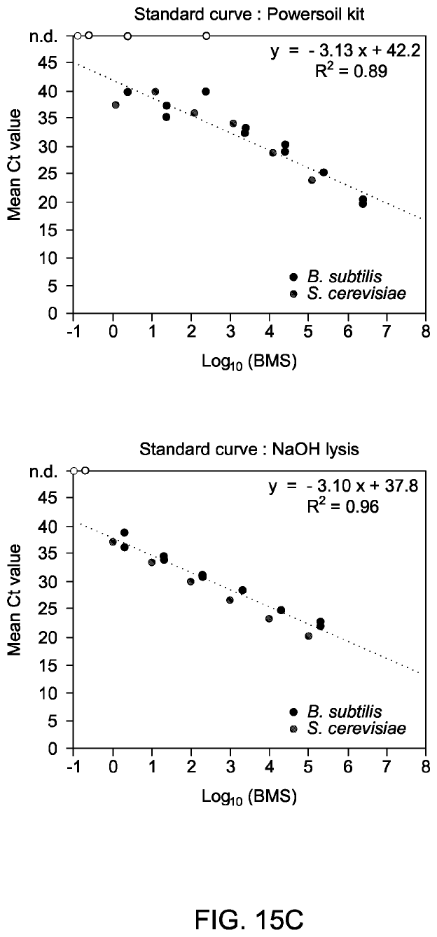

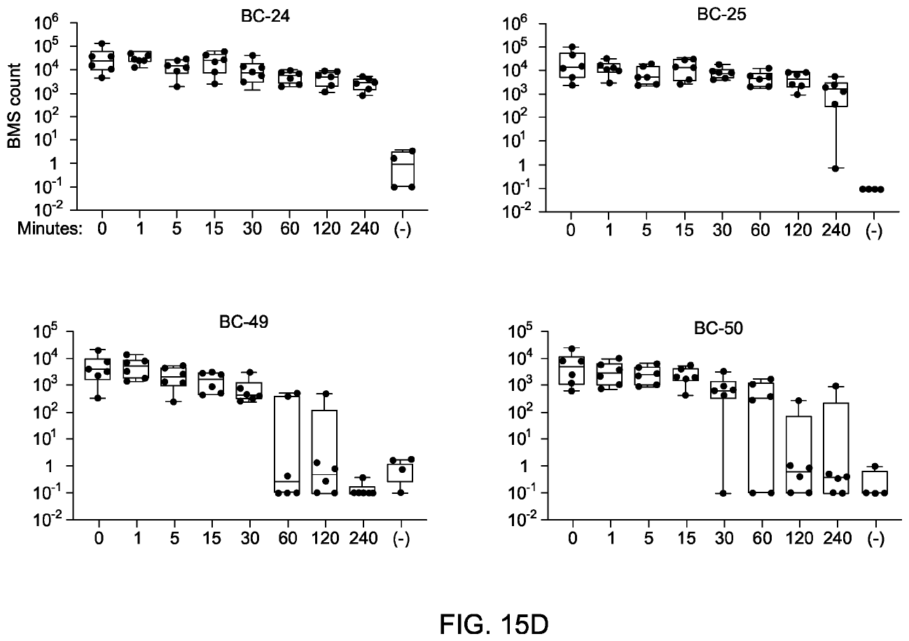

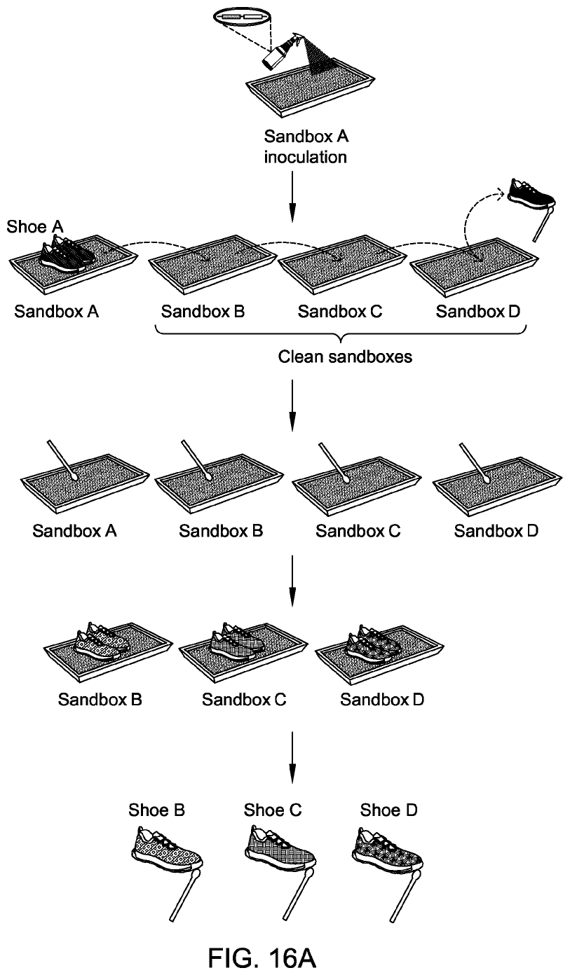

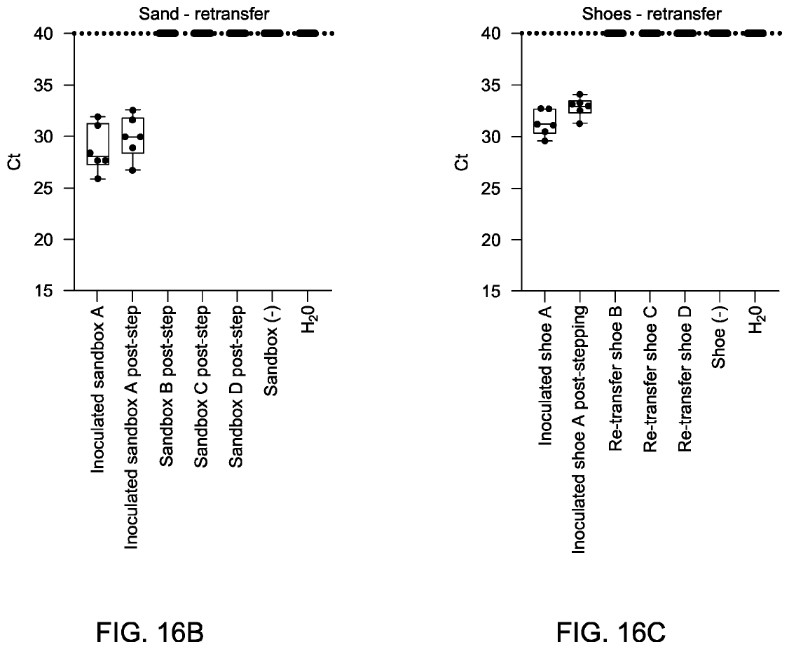

The BMS can be transferred onto objects that pass through test environments. In ˜1 m² scale, BMS could be transferred onto rubber or wooden objects simply by placing them on the BMS-inoculated surface (e.g., for several seconds), yielding up to ˜100 spores per microliter of reaction input (see e.g., FIG. 11D-11G). At larger scale (e.g., ˜100 m²), BMS were reliably transferred onto shoes worn in the BMS-inoculated sandpit (see e.g., FIG. 4F and FIG. 14A-14B). Furthermore, the BMS transferred onto shoes could still be detected (e.g., on the shoes) even after walking on non-inoculated surfaces for several hours, though BMS counts decreased by 2-fold with 2 hours of walking as quantified by qPCR (see e.g., FIG. 4G and FIG. 15A-15D). BMS could not be detected on non-inoculated surfaces after they were walked on with shoes that had traveled through BMS-inoculated regions (see e.g., FIG. 16A-16C). Thus, the BMS can persist in the environment without significant spreading; are transferable onto objects that pass through the environment; are retained on these objects; and can be sensitively and specifically detected (e.g., in <1 hours) using SHERLOCK.

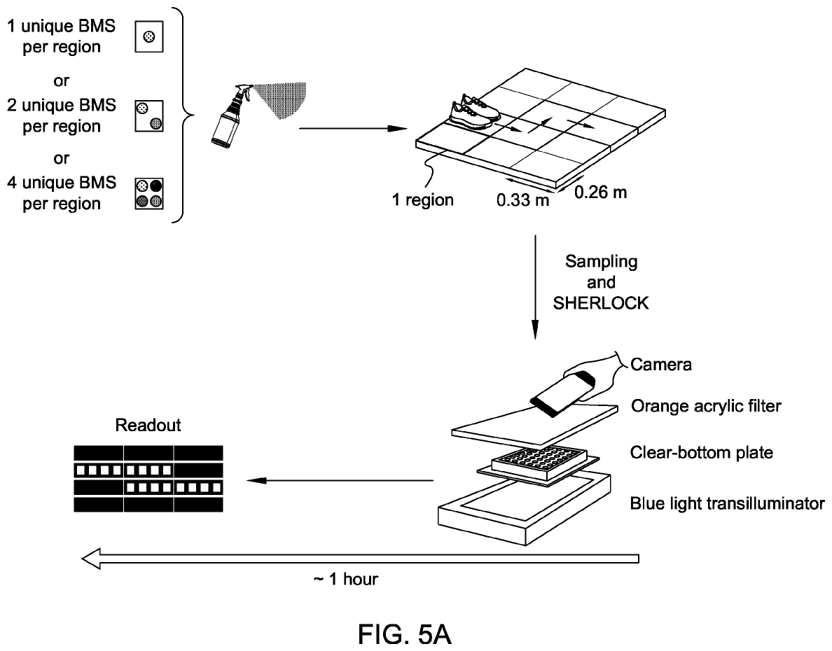

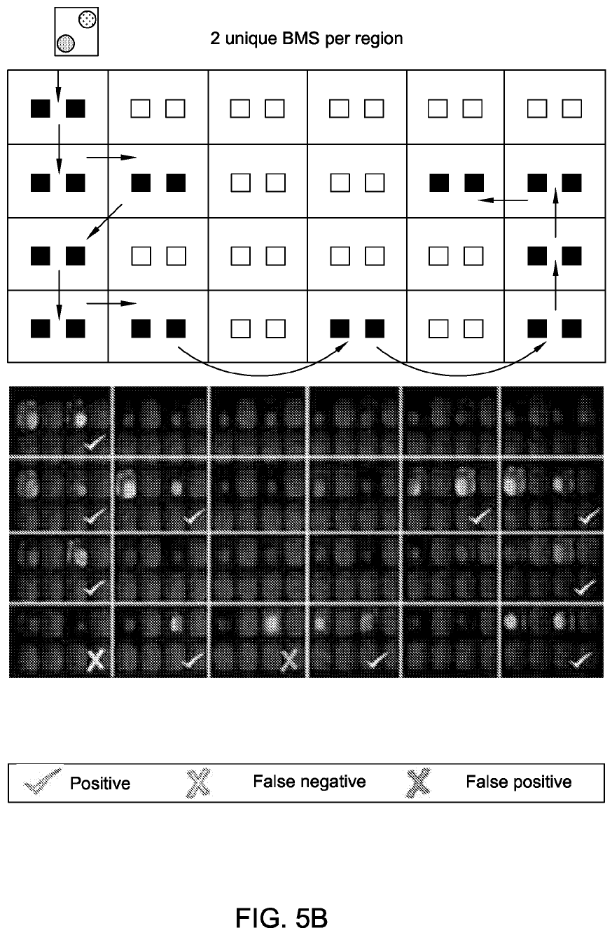

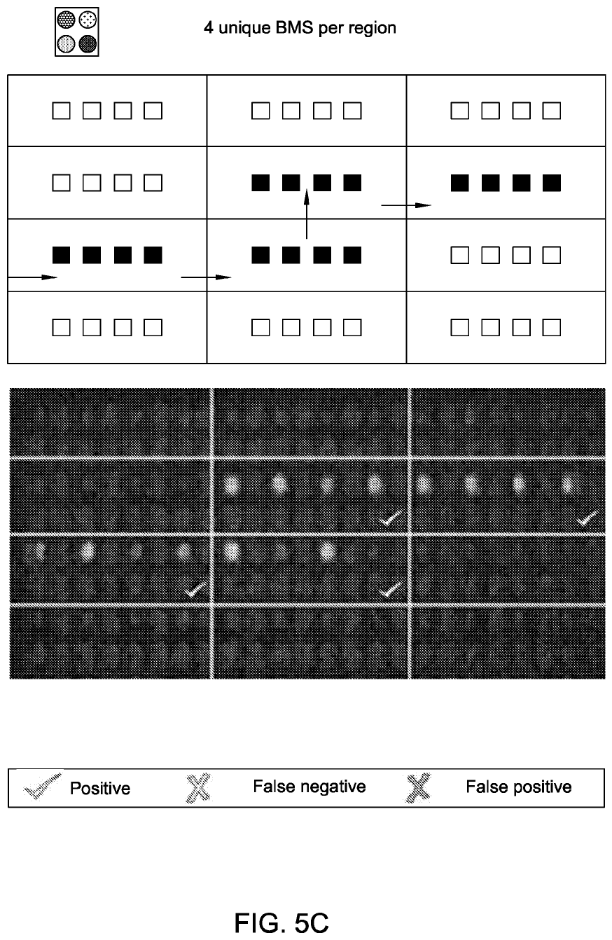

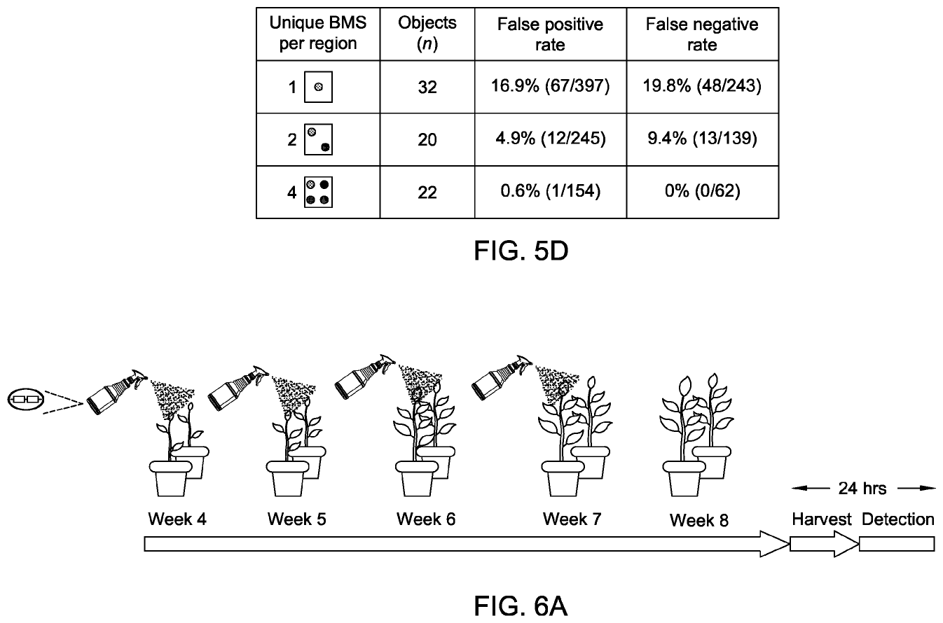

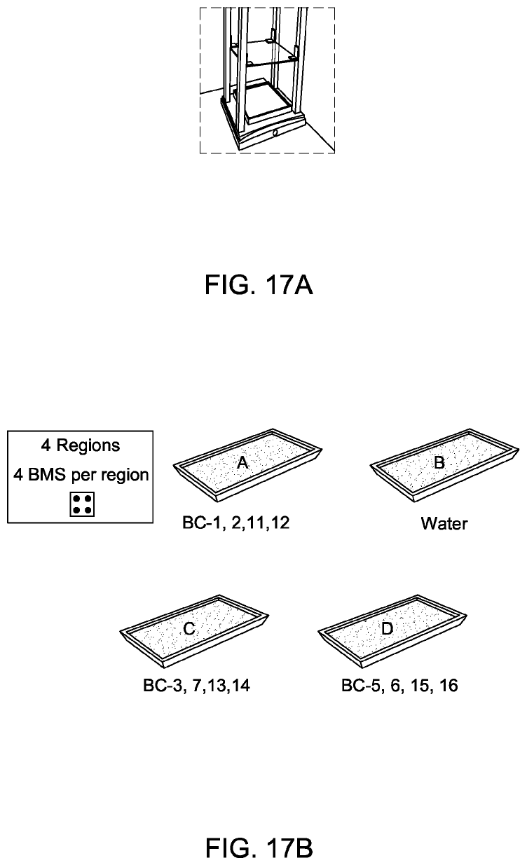

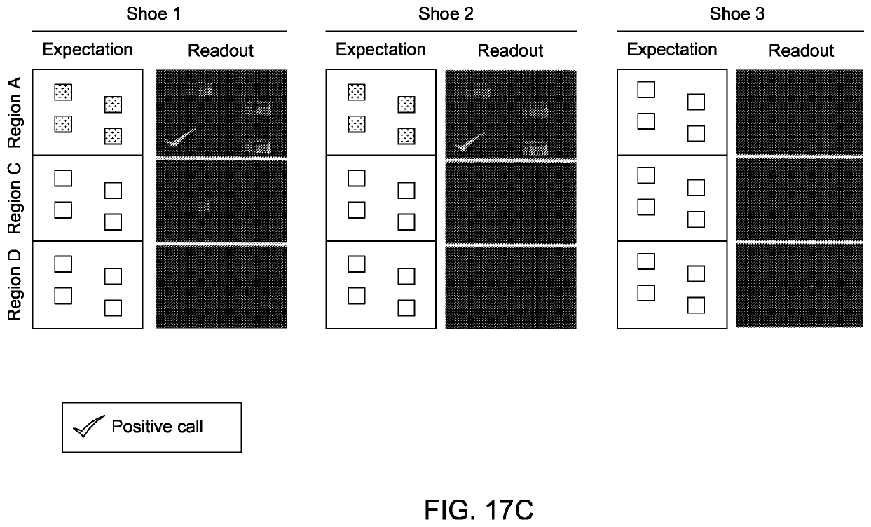

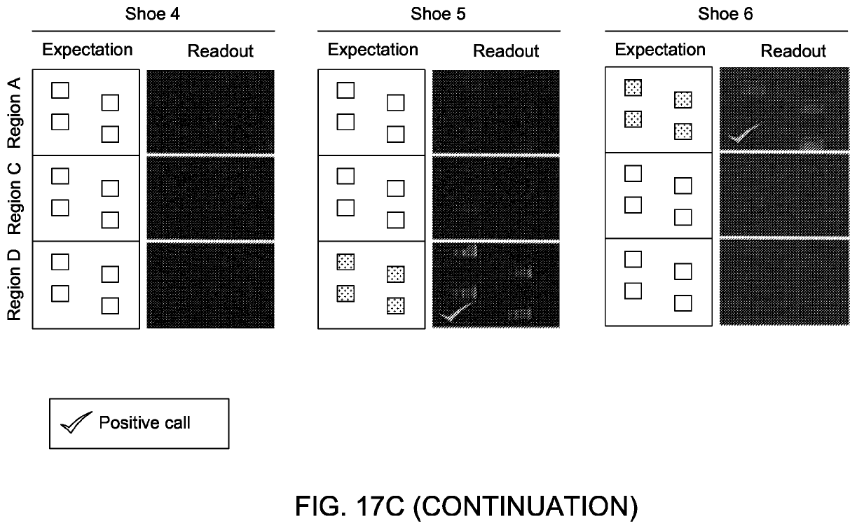

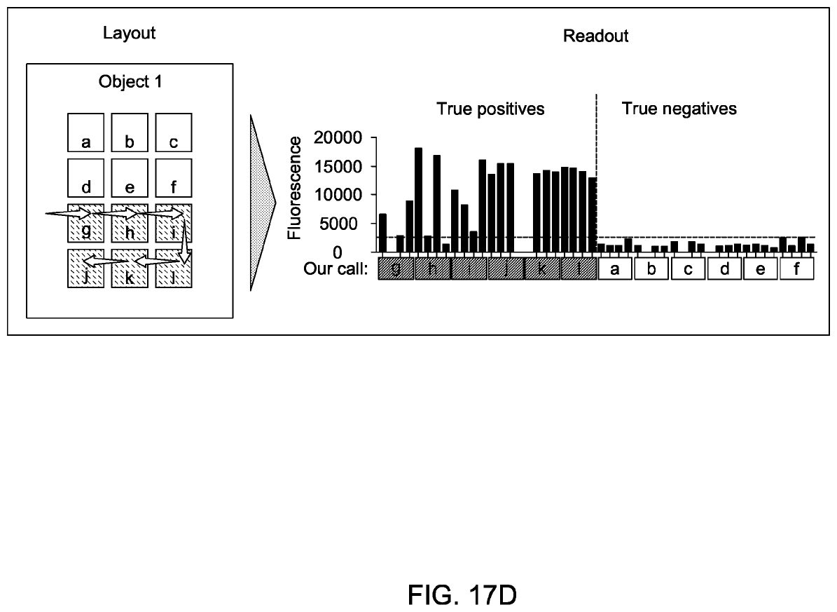

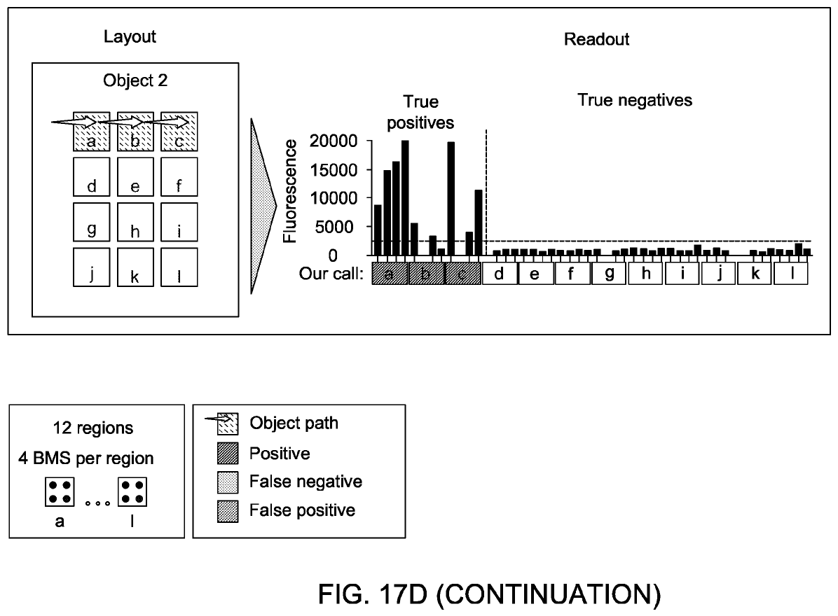

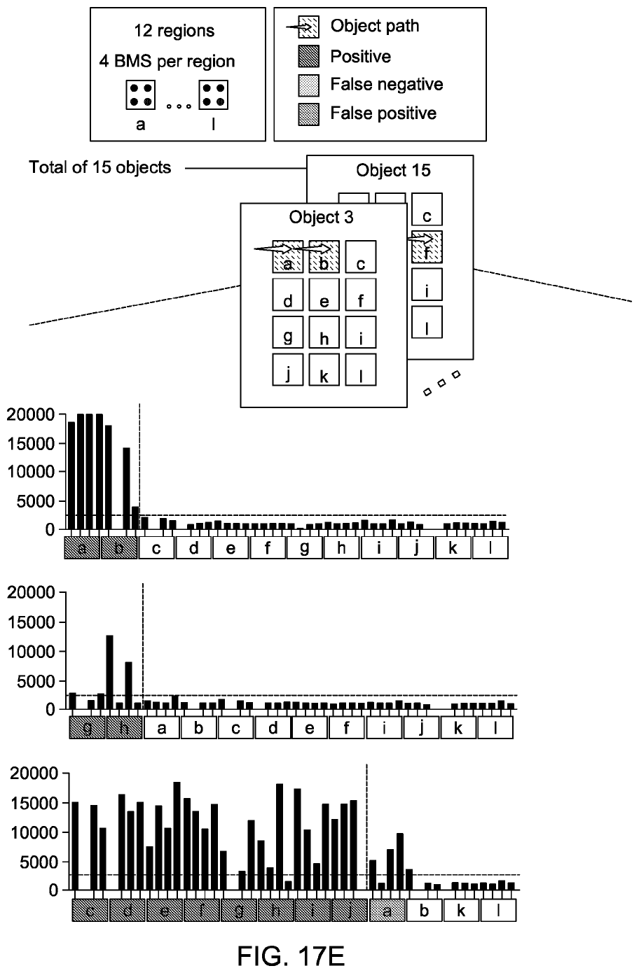

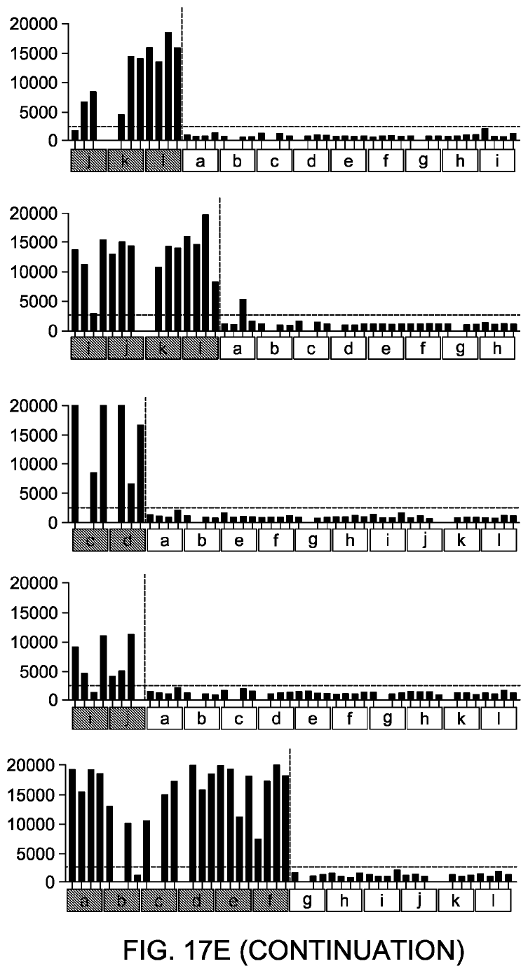

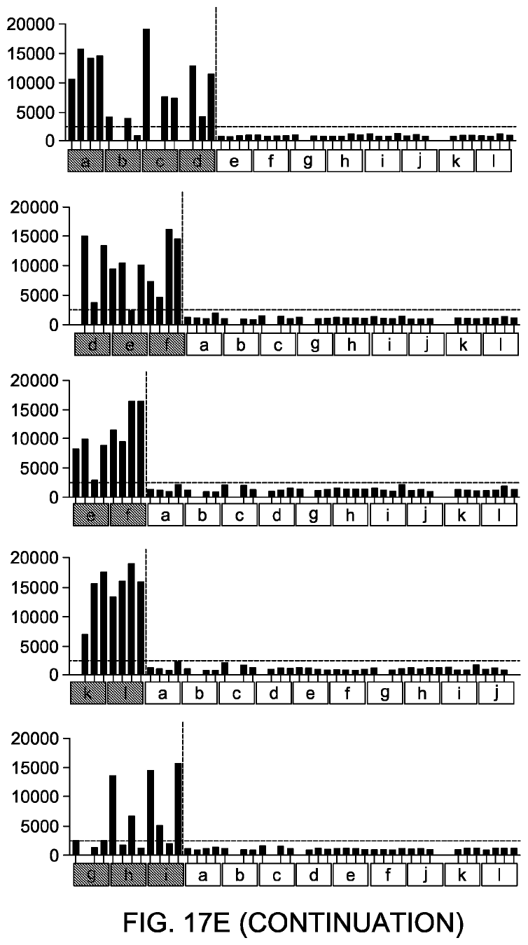

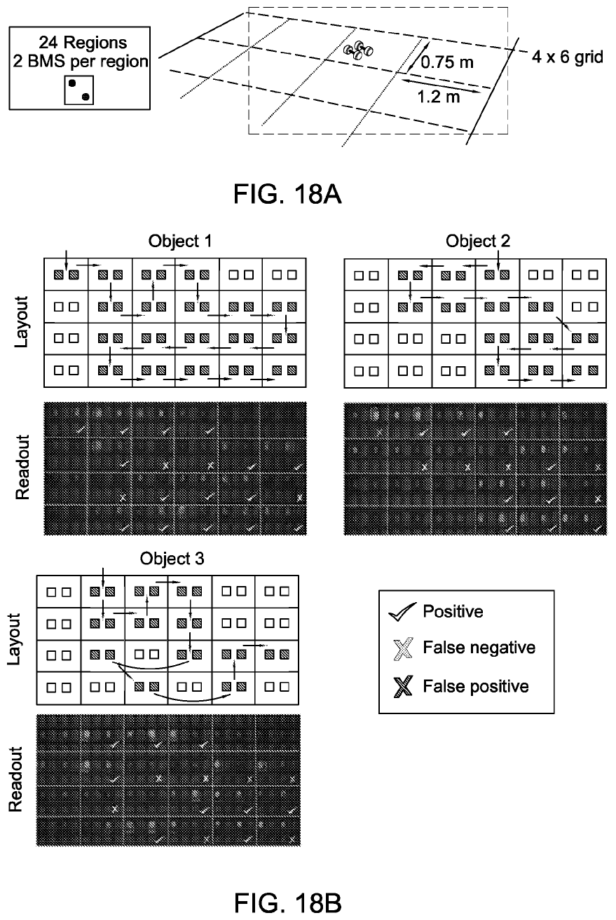

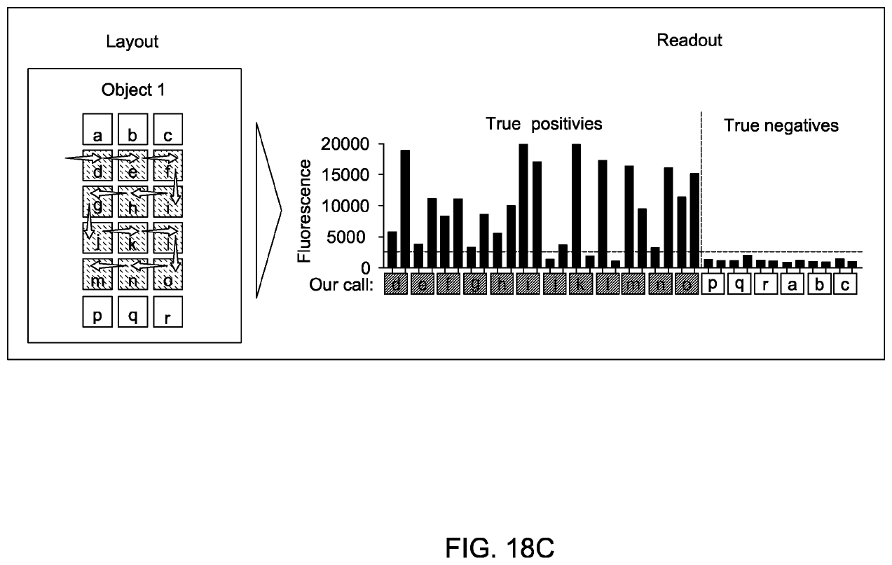

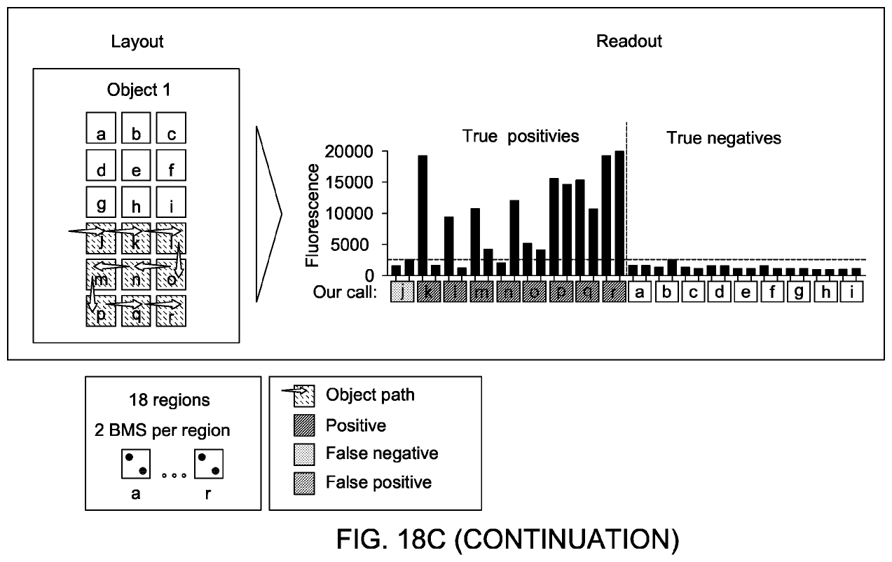

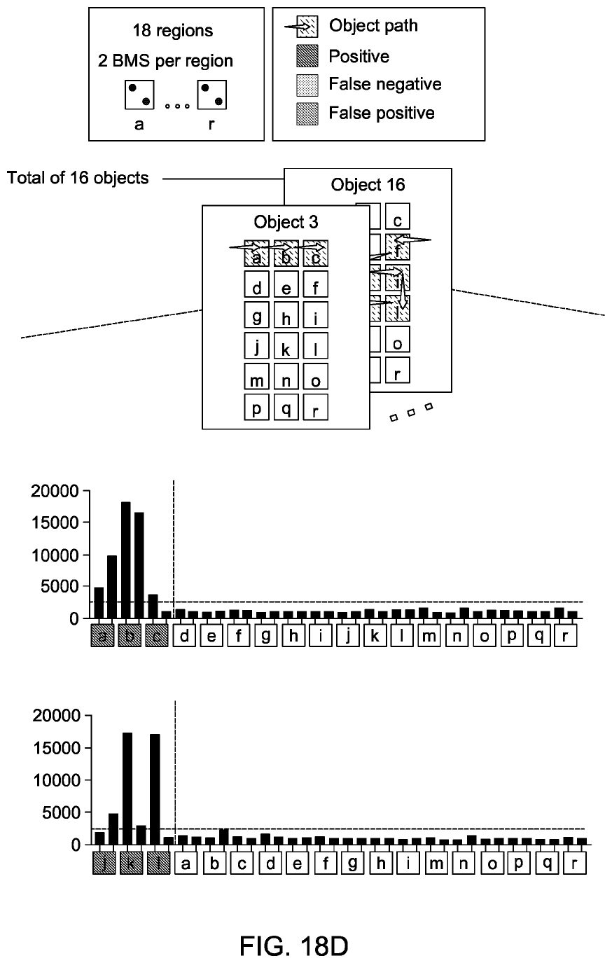

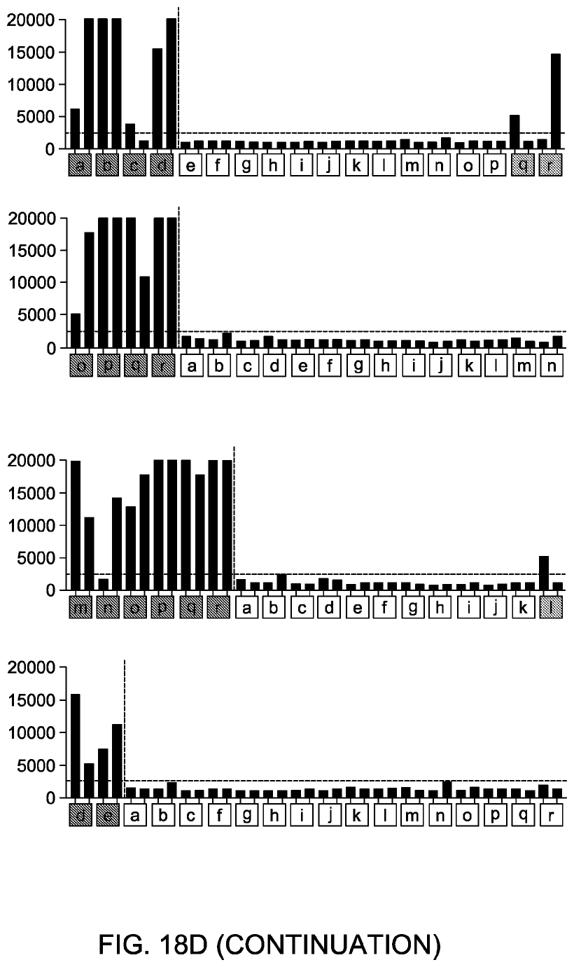

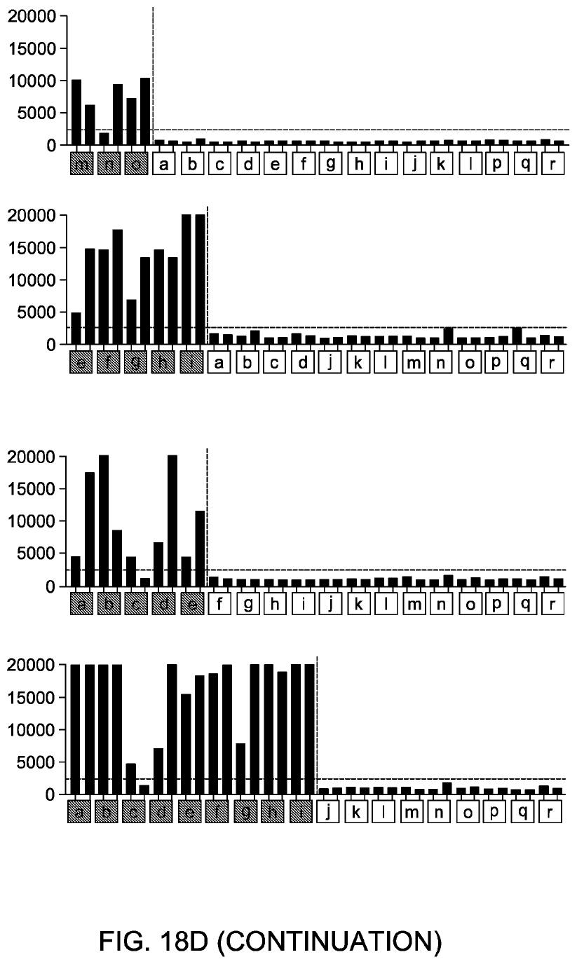

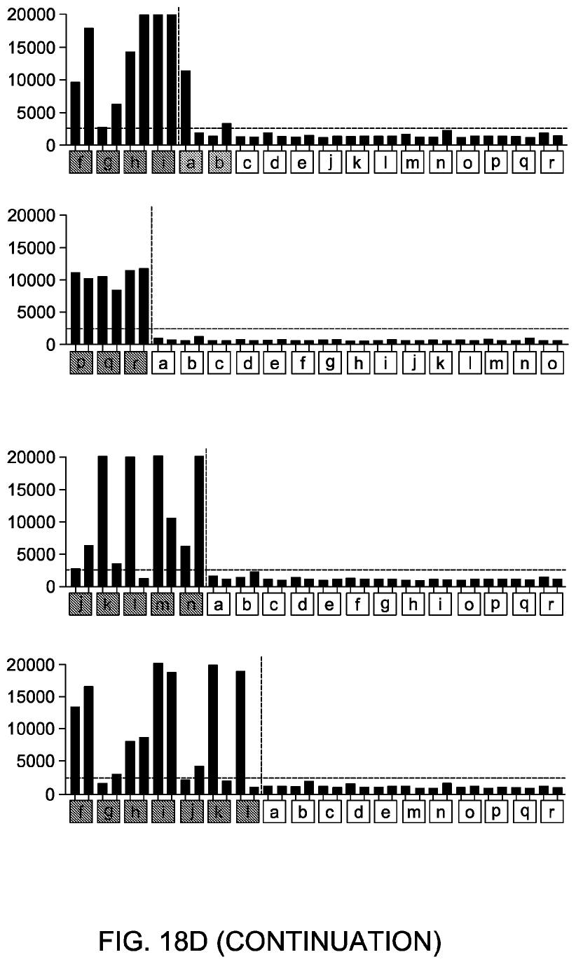

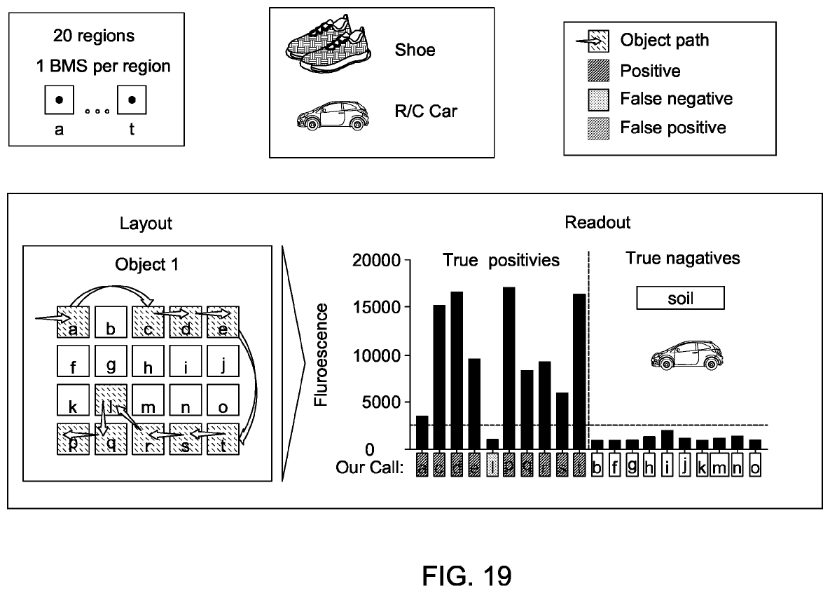

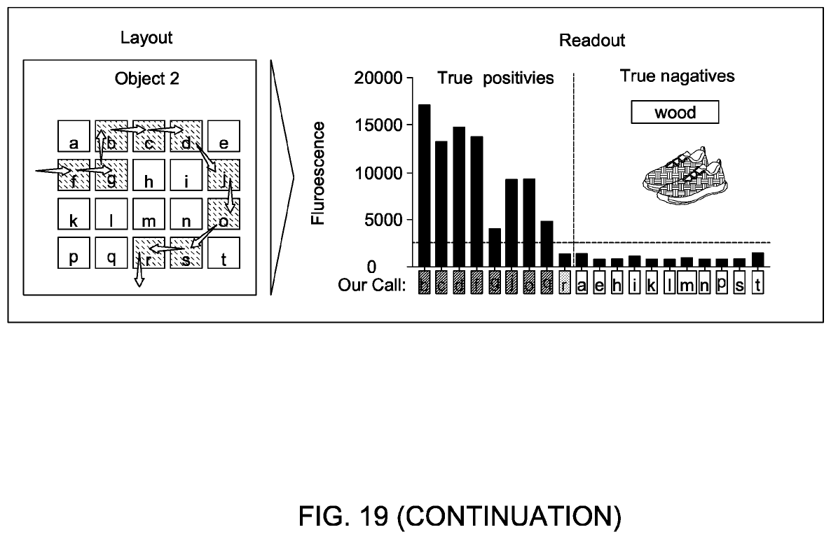

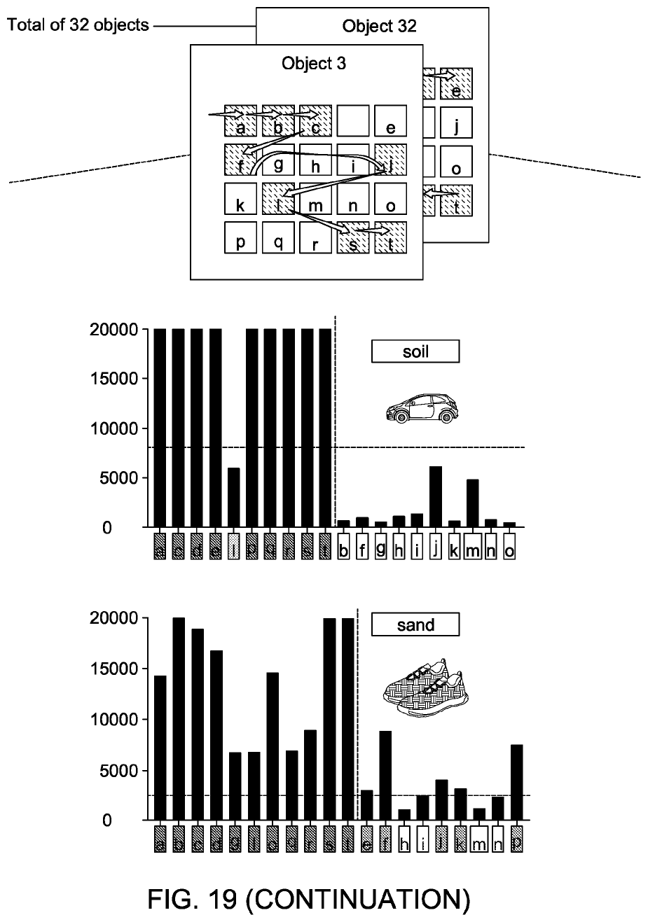

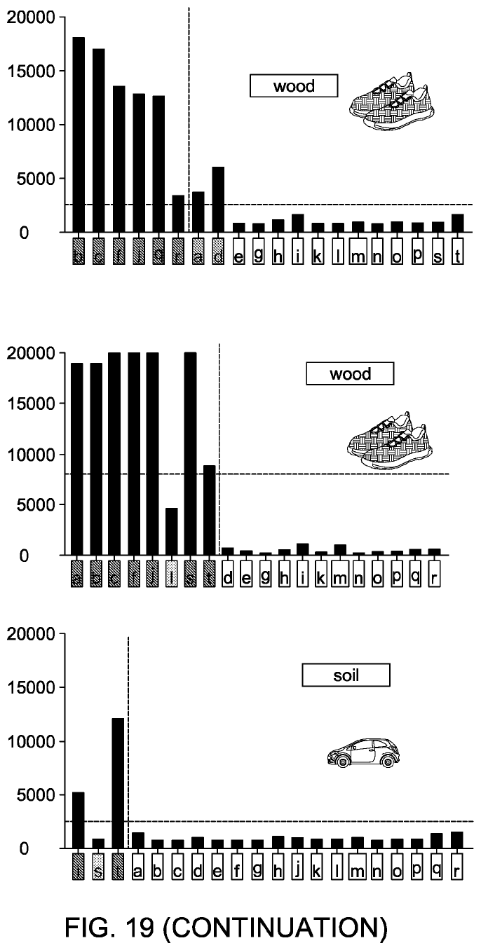

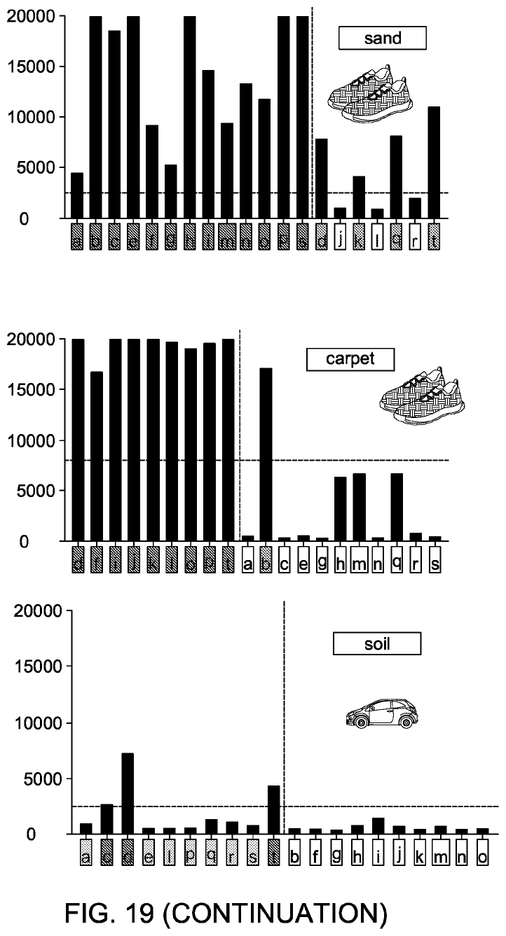

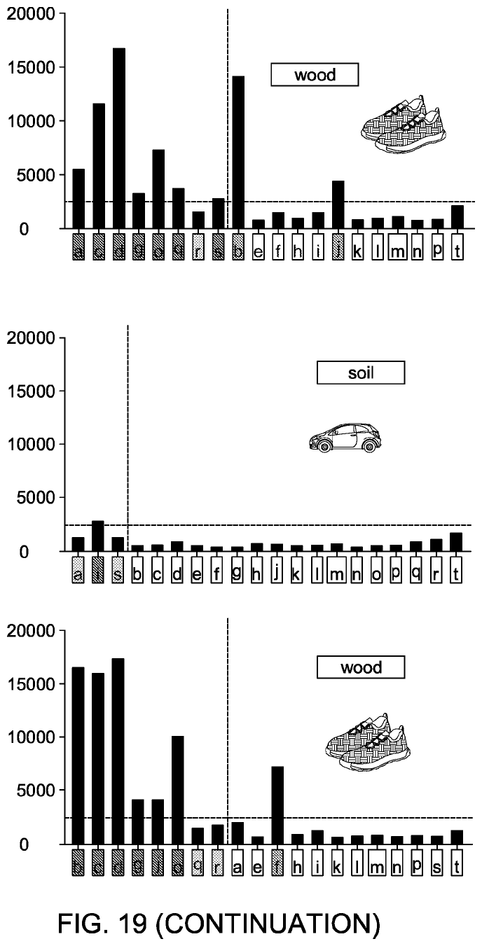

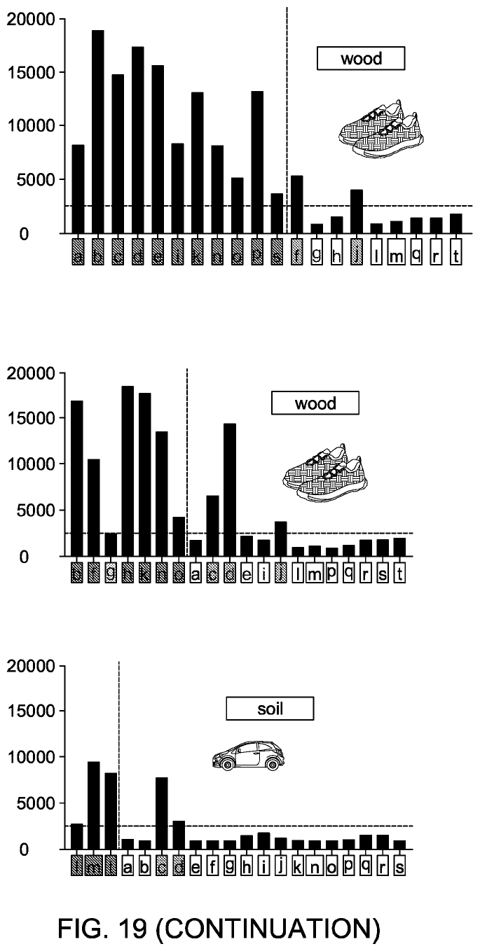

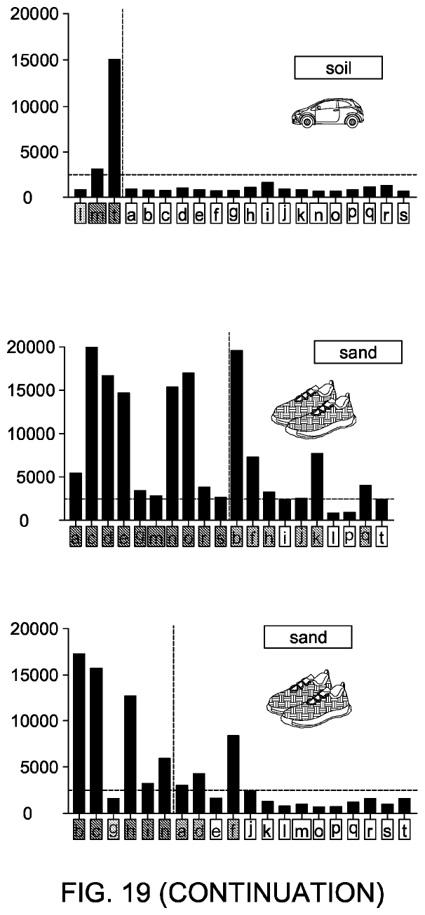

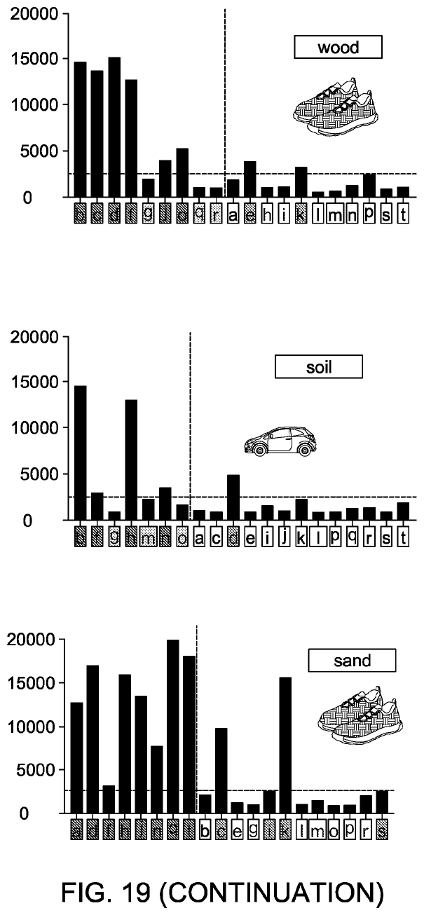

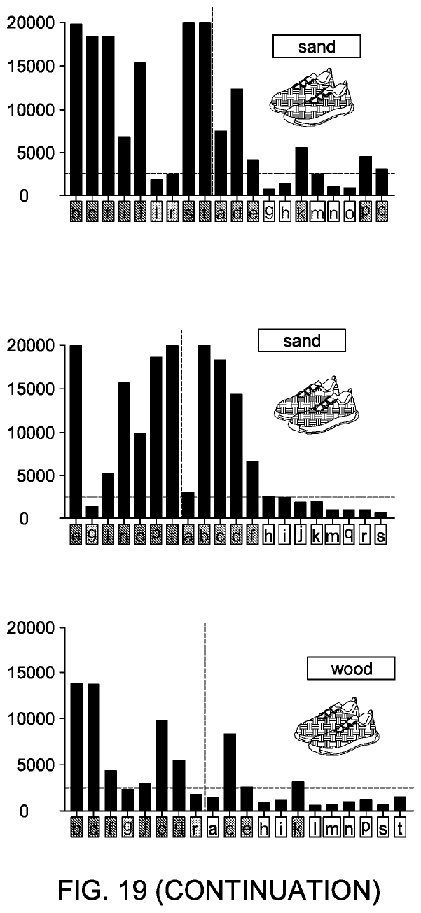

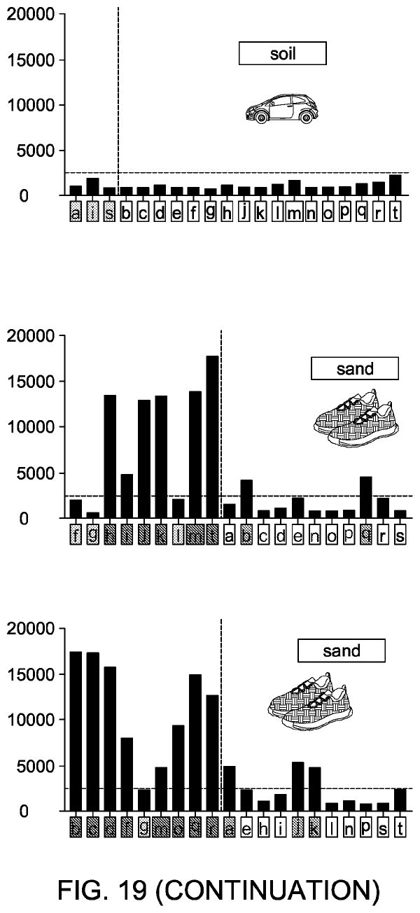

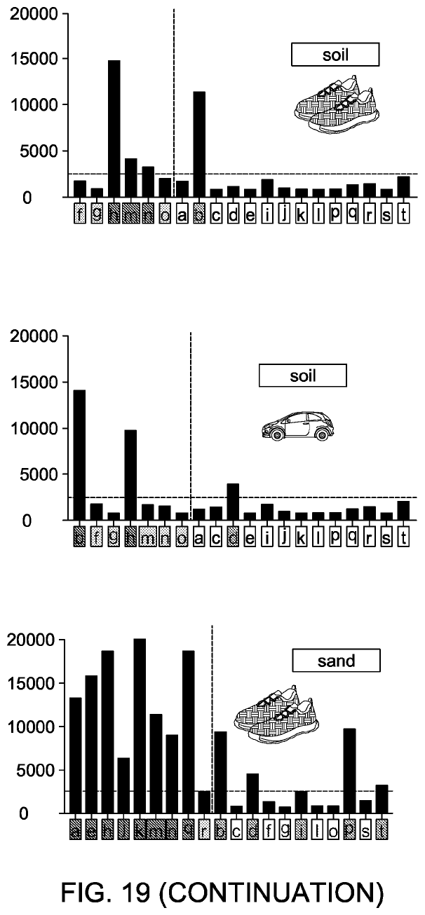

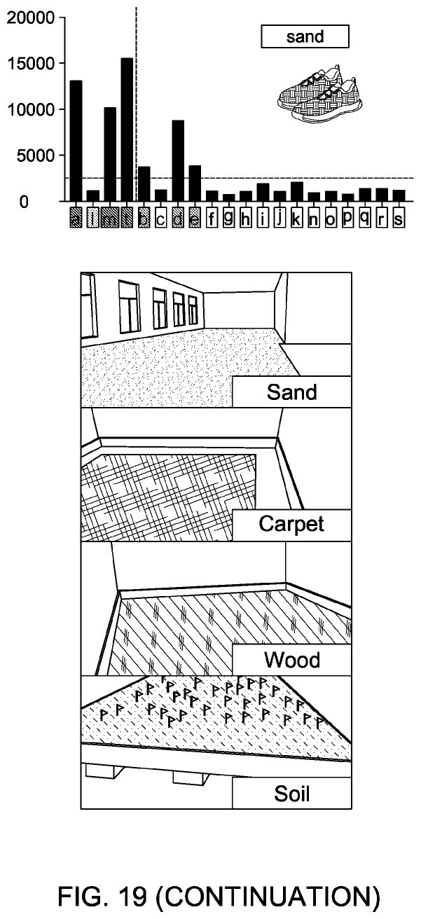

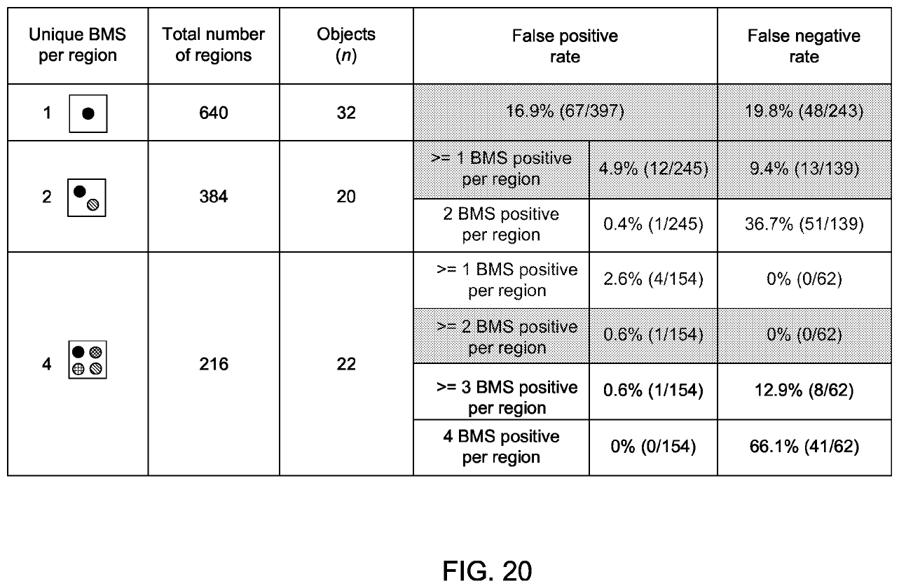

The BMS system can be used to label specific locations of interest to determine whether a person or object has passed through a specific environment. Different surfaces were divided into grids, each grid region was inoculated with 1, 2, or 4 unique BMS (see e.g., FIG. 5A), and a series of different test objects were traversed across them (e.g., shoes). To mimic in-field deployment, the following detection devices were used: a portable light source, an acrylic filter, and a mobile phone camera to image the SHERLOCK readout (see e.g., FIG. 5A, FIG. 17A) and to determine object provenance (see e.g., FIG. 5B and FIGS. 17-20). For example, object provenance was successfully determined in >20 tests with only a single false positive quadrant using 4 BMS per region (see e.g., FIG. 5C and FIG. 17A-17E). Notably, provenance could be determined in the field within ˜1 hour from sample collection. To evaluate the sensitivity and specificity of the system for determining an object's provenance or trajectory, more objects were tested, and the number of unique BMS inoculated in each quadrant, as well as the surface material (i.e. soil, carpet and wood) were changed (see e.g., FIG. 17A-17E, FIG. 18A-18D, FIG. 19). As expected, increasing the number of unique BMS used per quadrant improved the confidence levels of positive calls by adding redundancy to the decoding. Object provenance could be determined with a 0.6% (1/154) false positive rate and 0% (0/62) false negative rate if regions were inoculated with 4 unique BMS; inoculating with only 1 or 2 unique BMS per region still permitted object provenance to be determined, albeit at higher error rates. (see e.g., FIG. 5D, FIG. 20). Importantly, provenance could be determined on all 4 surface types tested (sand, soil, carpet, or wood) (see e.g., FIG. 19). More broadly, this experiment demonstrates that the BMS can be used to determine object provenance at meter-scale resolution, which would be extremely difficult to achieve using natural microbiome signatures; see e.g., Adams et al., Microbiome 3, 49 (2015), the content of which is incorporated herein by reference in its entirety.

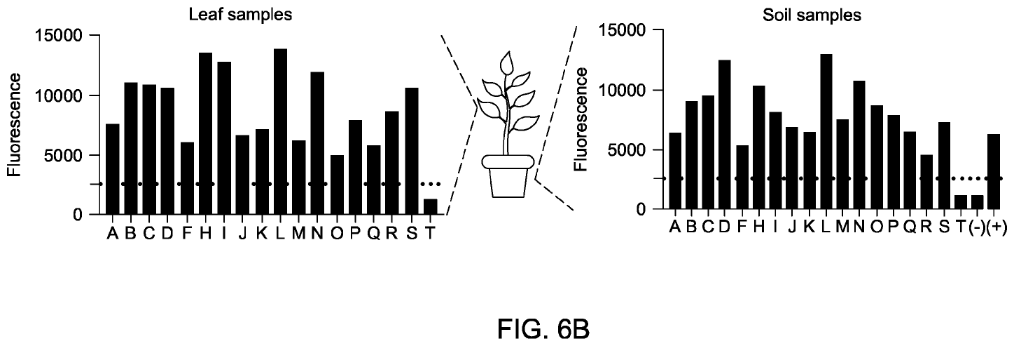

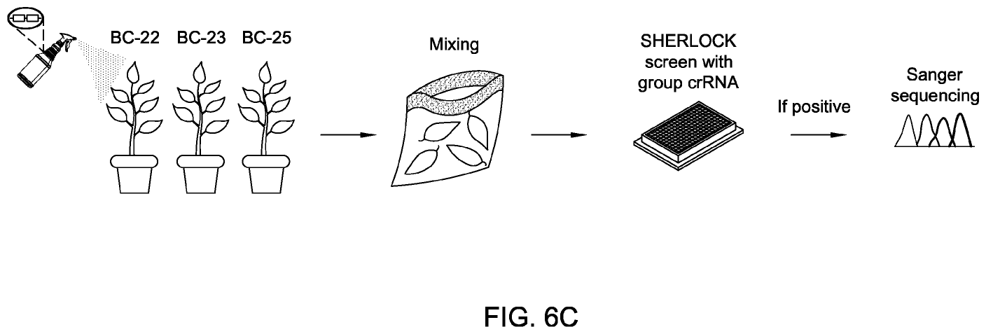

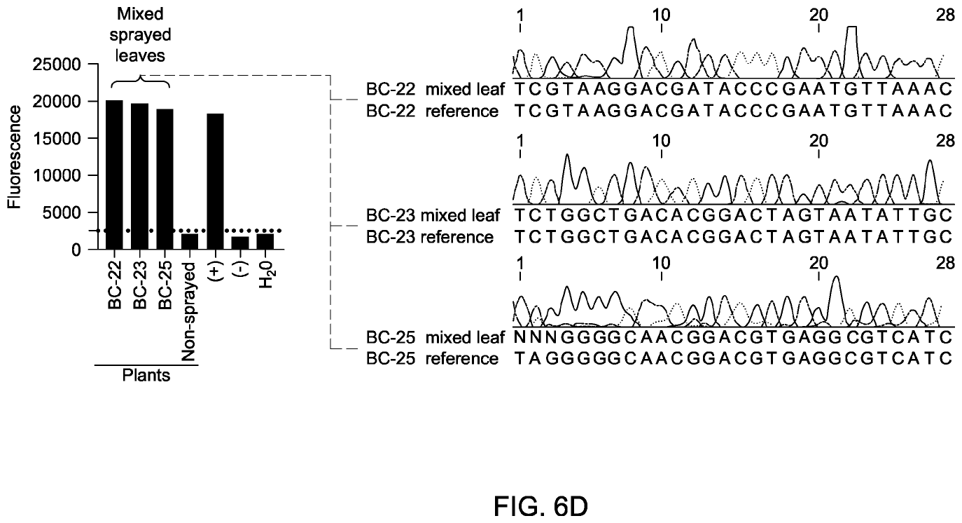

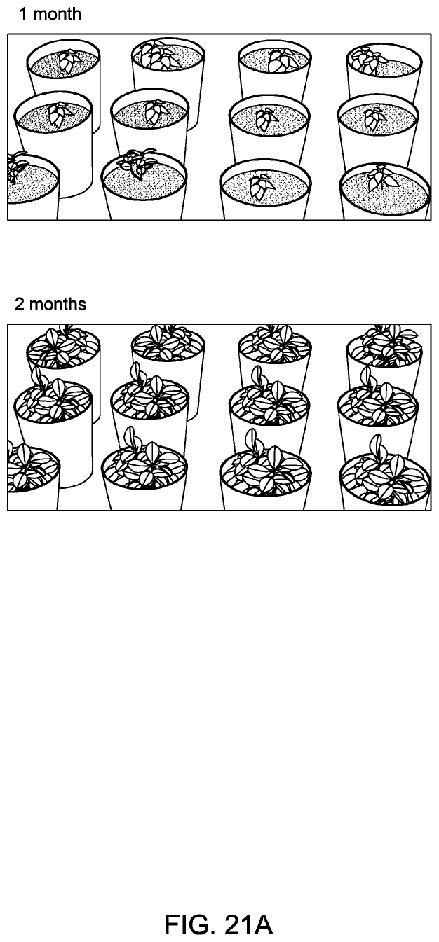

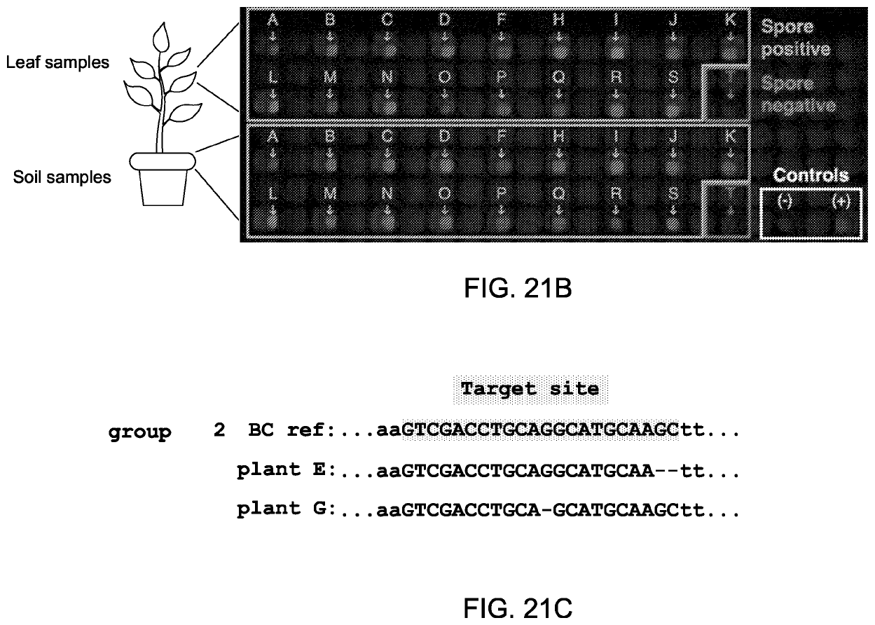

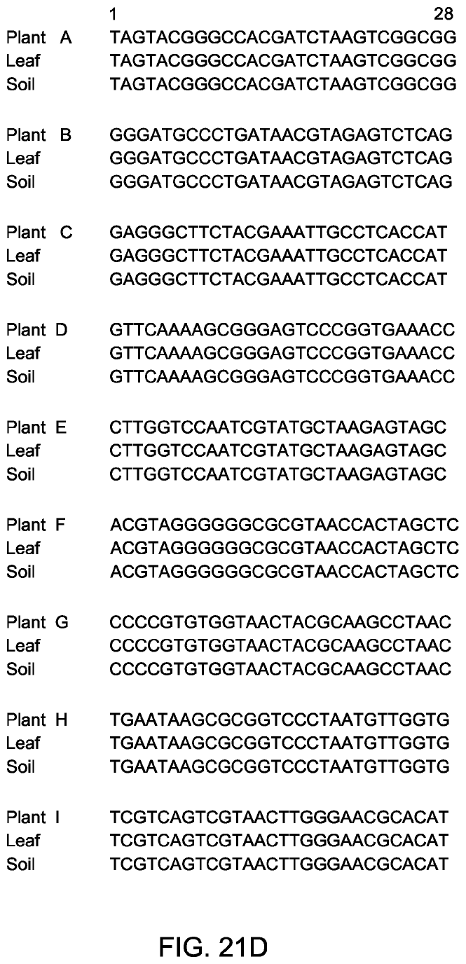

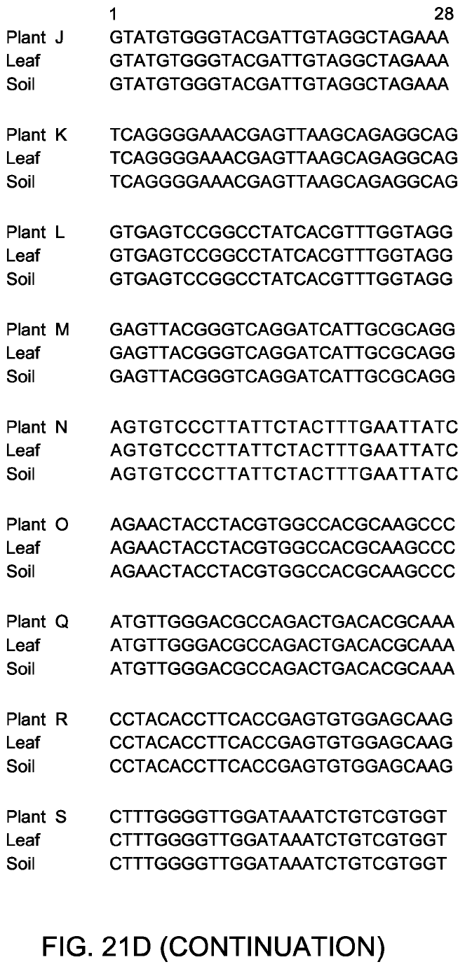

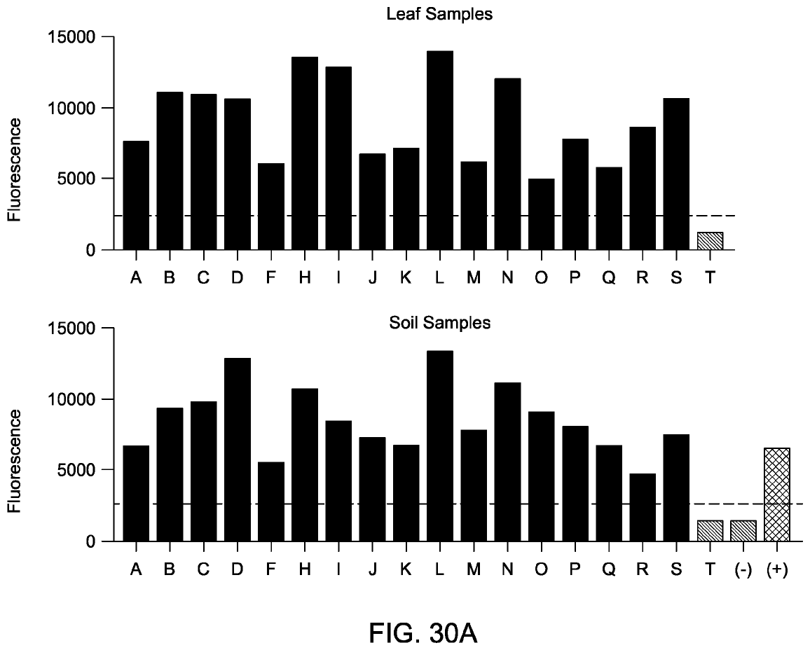

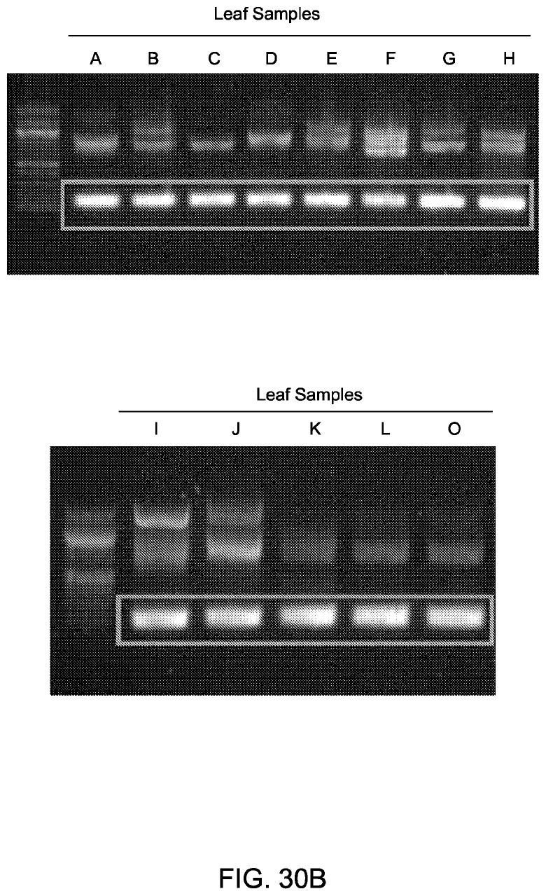

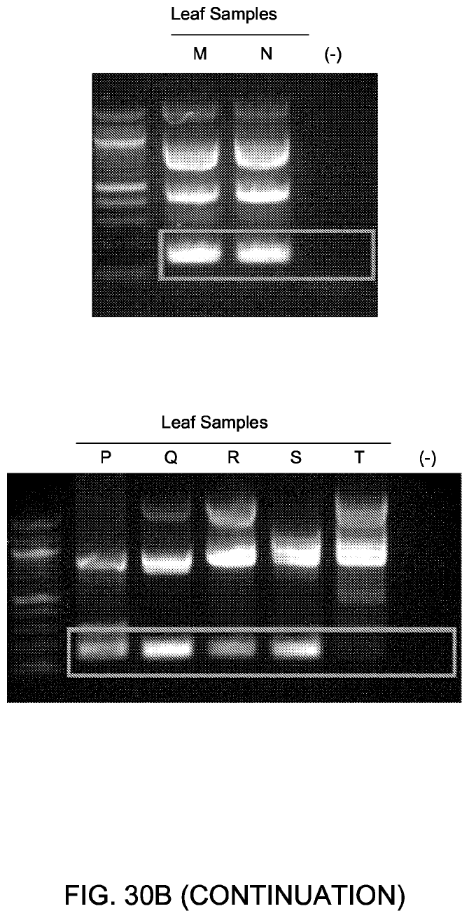

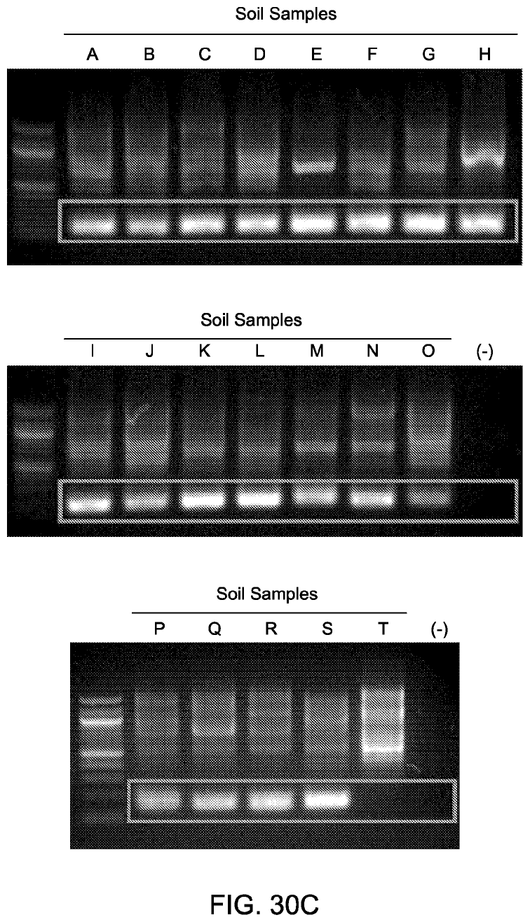

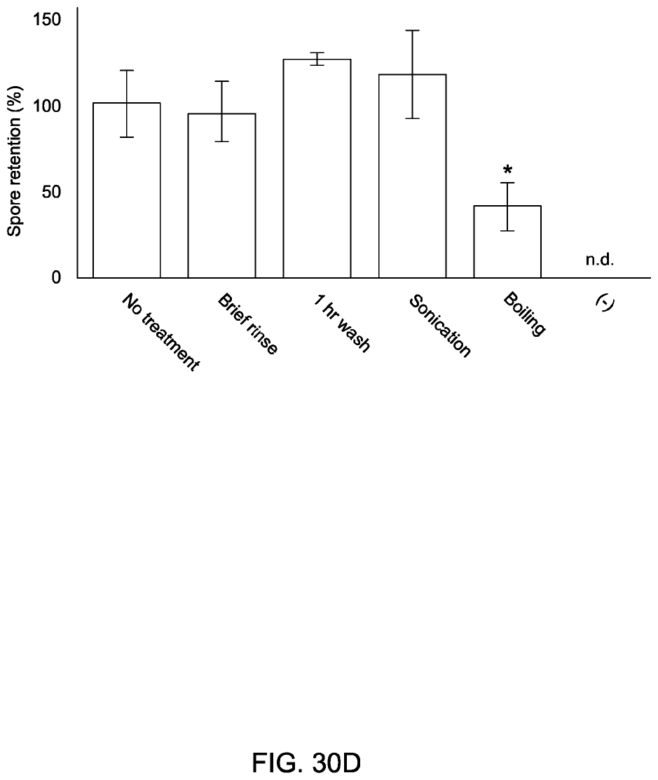

The BMS system offers a flexible and comprehensive approach to determining food provenance. Foodborne illness is a global health issue with 48 million cases reported every year in the US alone. There is an urgent need for rapid methods for identifying the source of food contamination; current foodborne illness tracking approaches often take weeks and are costly due to the complex modern market chain. Plants inoculated with B. subtilis BMS were used to map lab-grown leafy plants back to the specific pot in which they were grown (see e.g., FIGS. 6A, 6B, 21A-21D, FIG. 30A-30D). BMS were inoculated 4 times, beginning 1 week after the first set of leaves appeared, to match recommended inoculation protocols for Bacillus thuringiensis spores (Bt), an FDA-approved biocide that is widely used in agriculture; see e.g., Sanahuja et al., Plant Biotech. J. 9, 283-300 (2011), the content of which is incorporated herein by reference in its entirety. One week after the final BMS inoculation, a leaf and soil sample from each pot were harvested and tested using SHERLOCK. All the samples were positively detected except for the 2 plants that had received variant group barcode sequences, demonstrating the specificity of detection (see e.g., FIG. 6B and FIGS. 21B-21C). Using Sanger sequencing, the pot in which each plant was grown was then identified for all 18 BMS (see e.g., FIG. 21D). The process from DNA extraction to sequence identification took less than 24 hours. This time frame could likely be shortened to hours with massively parallelized hybridization-based detection; see e.g., Sarwat et al., Crit. Rev. in Biotech. 36, 191-203 (2016), the content of which is incorporated herein by reference in its entirety.

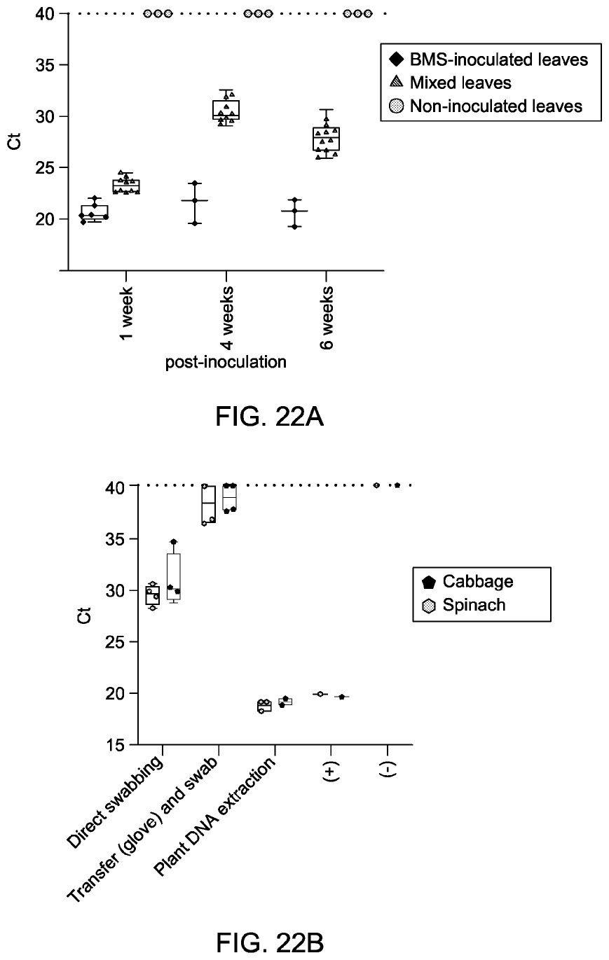

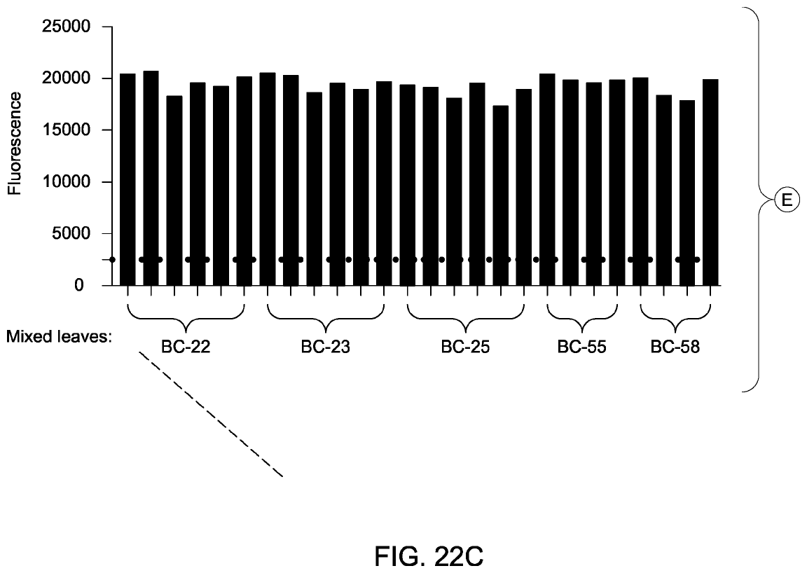

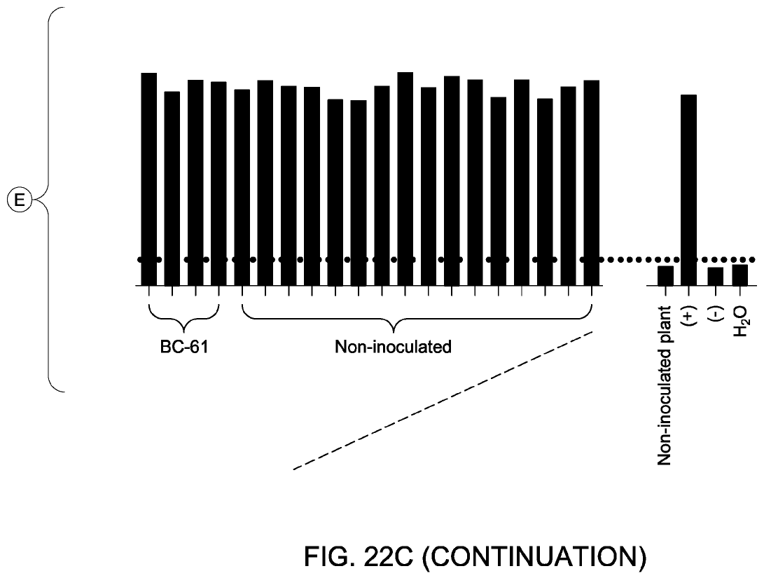

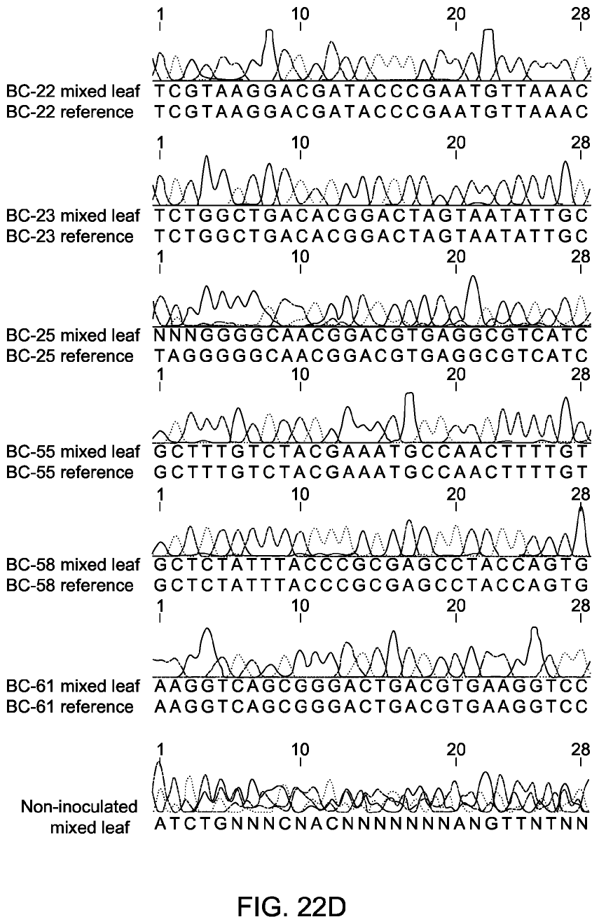

Cross-association of BMS-inoculated plants does not compromise the determination of provenance. To simulate cross-association that could occur during food processing, leaves from plants that were inoculated with unique BMS were mixed. Unlike the other inoculated surfaces, BMS-inoculated plants did not transfer as easily to objects that came into contact with the plants (see e.g., FIG. 11A-11G compared to FIG. 22A-22B). While there was detectable transfer between leaves, the amount of transfer still allowed Sanger sequencing to cleanly determine the origin of each leaf (see e.g., FIG. 6C-6E and FIG. 22C-22D).

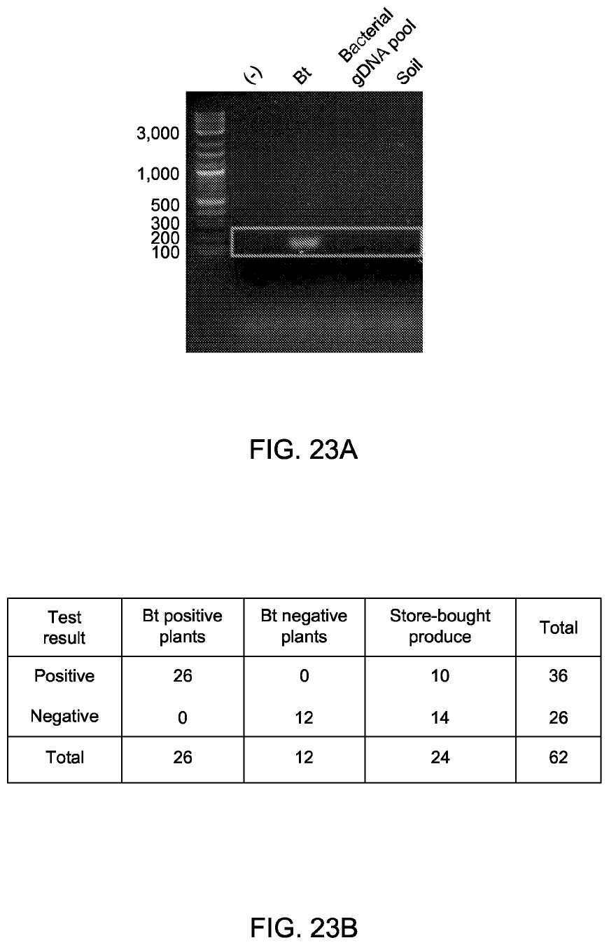

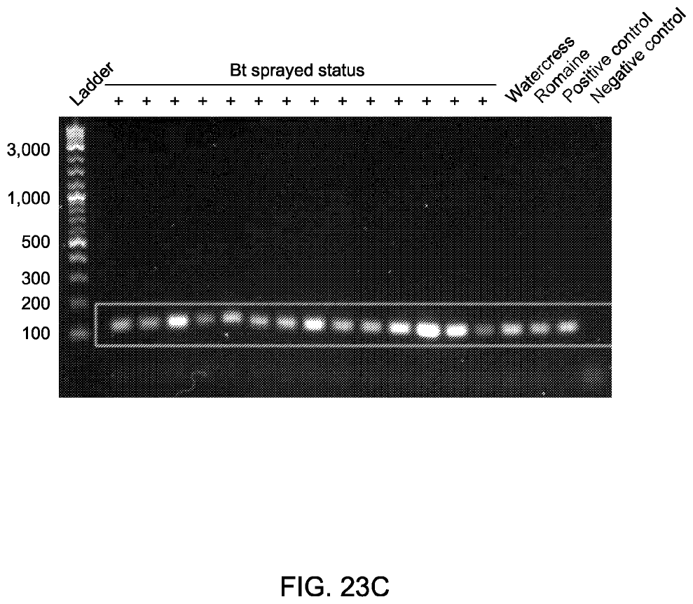

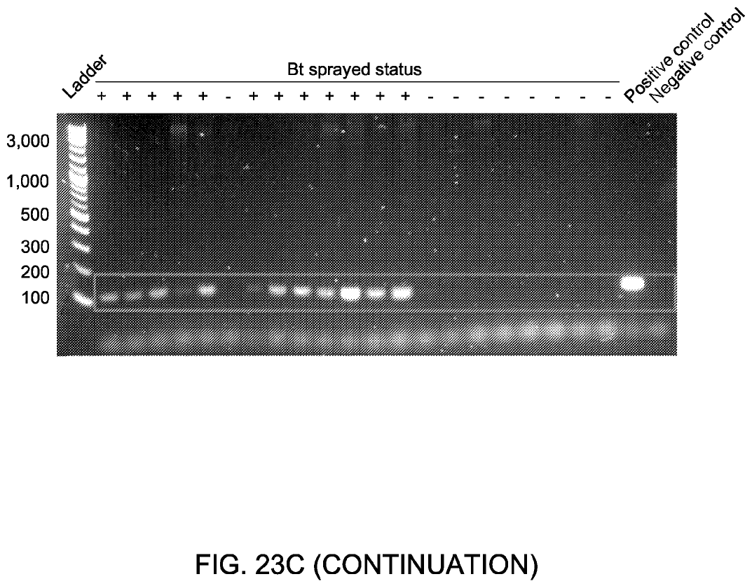

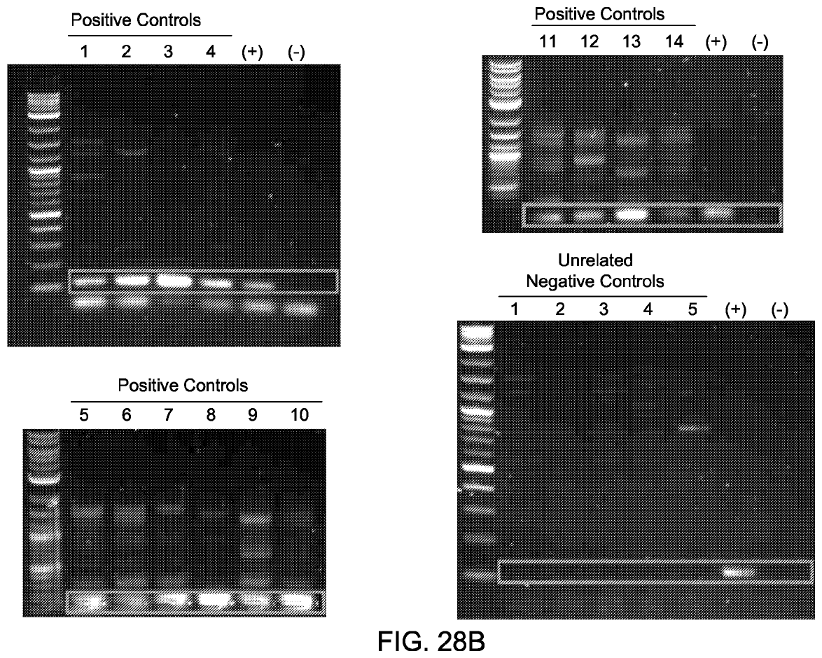

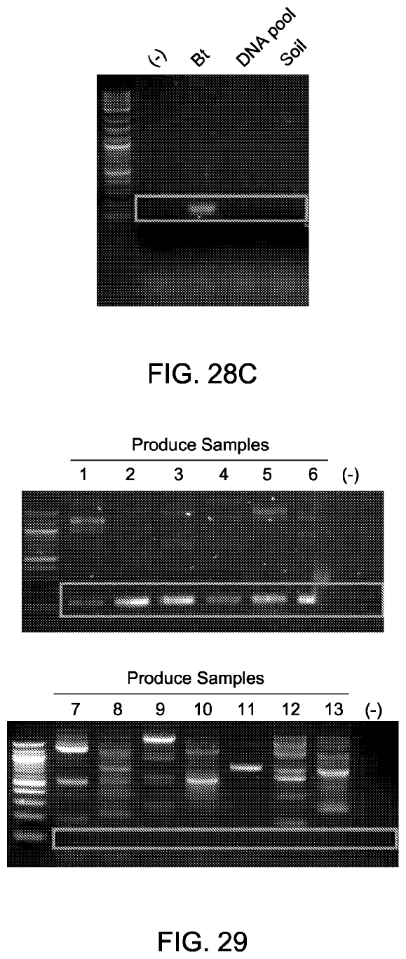

Bt can be used to determine food provenance. Bt spores were applied during farming as a surrogate to test whether BMS would persist through conditions of a real-world food supply chain. For plants of known Bt inoculation status, all Bt positive and negative plants were correctly identified (38 total plants) (see e.g., FIG. 23A-23C). To determine if spores applied during farming could be detected in produce purchased from a market, the Bt cry1A gene was targeted using qPCR. Bt was detected on all plants known to have been sprayed with Bt (14/14) and not detected on plants known to have not been treated with Bt (10/10) (see e.g., FIG. 28A-28C).

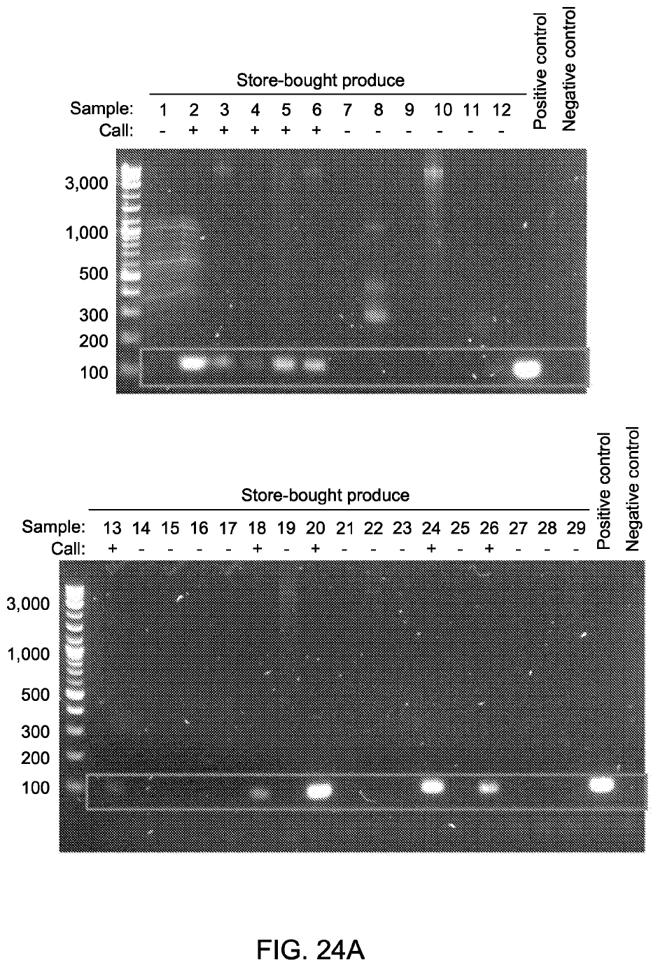

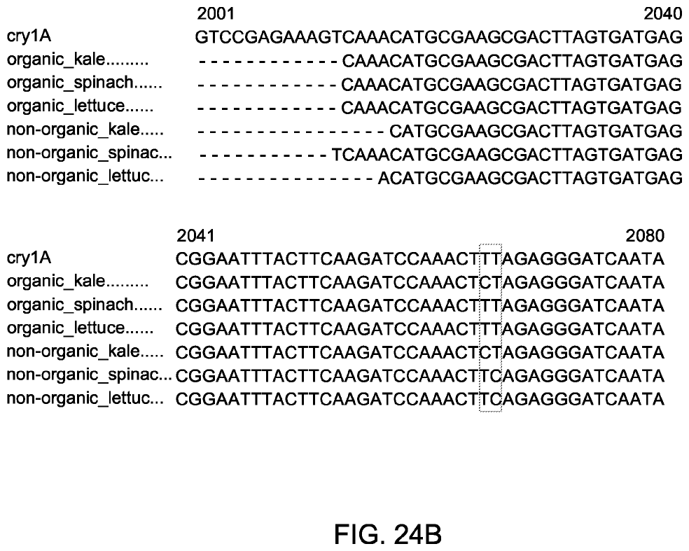

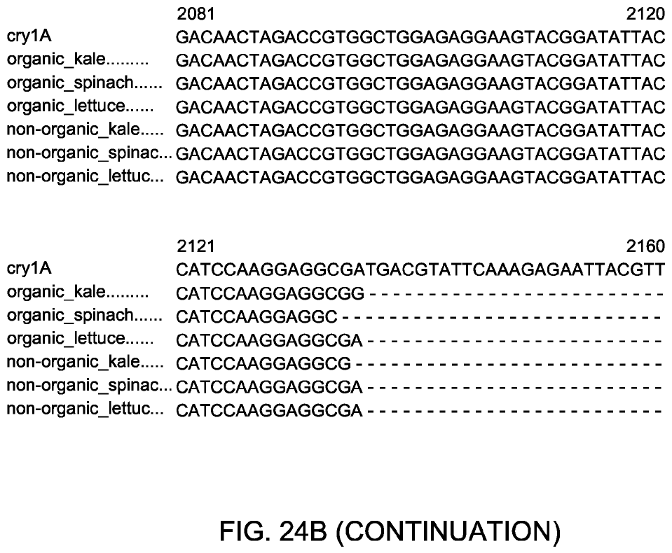

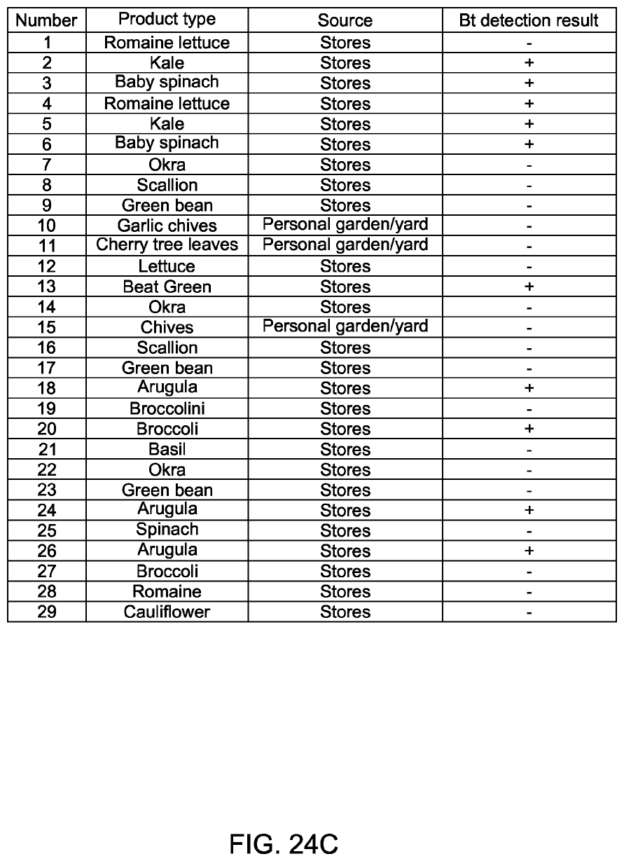

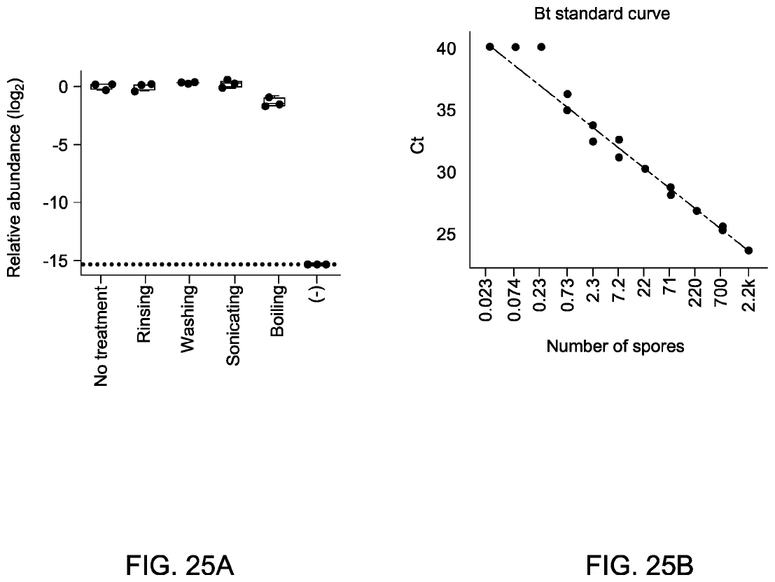

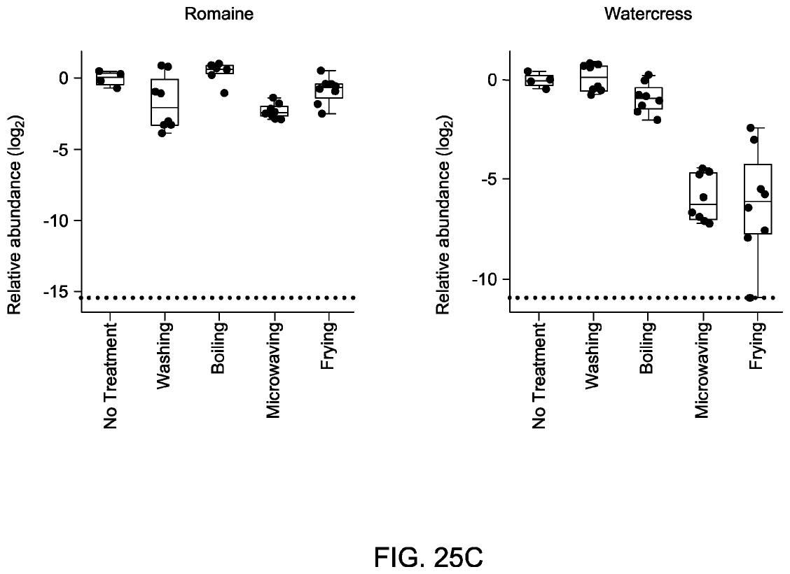

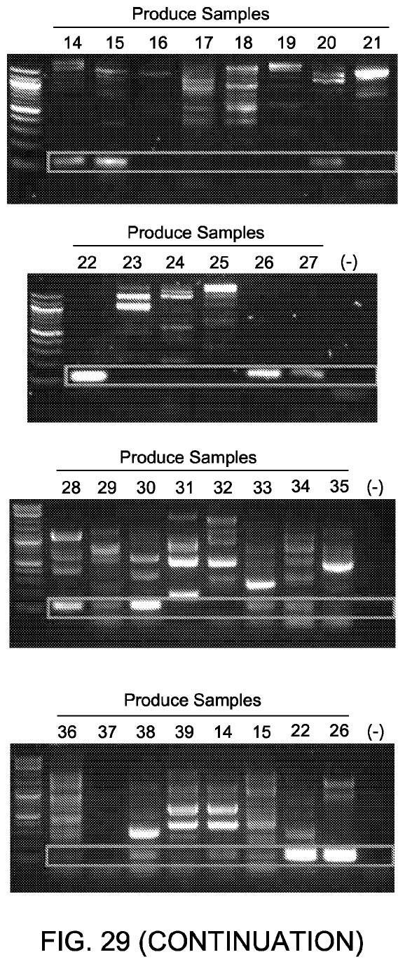

Furthermore, Bt was detected on 10 of 24 store bought produce of a priori unknown Bt status (see e.g., FIG. 23B, FIG. 24A-24C). In addition, Bt spores were detected in 19 out of 32 pieces of produce bought at stores (see e.g., FIG. 6A and FIG. 29, and Table 1). Strikingly, BMS and Bt spores remained detectable even after washing, boiling, frying, and microwaving (see e.g., FIG. 25A-25C), highlighting the ability to determine provenance from cooked foods. These results show the ability to use the BMS system to determine produce provenance.

As shown herein, rationally engineered microbial spores manufactured in a high-throughput manner provide a new solution to the object provenance problem. Collectively, these experiments demonstrate that the BMS: 1) persist in the environment; 2) do not spread out of the inoculation area; 3) transfer from soil, sand, wood, and carpet to contacting objects; and 4) permit sensitive and rapid readout using laboratory and field-deployable methods. The ability to rapidly label objects and determine their provenance or trajectories in real-world environments has a broad range of applications across agriculture, commerce, and forensics. Furthermore, BMS works across various environments. Without wishing to be bound by theory, BMS can be engineered for limited (e.g., self-limiting) propagation (e.g., a limited number of cell divisions), which can make the system compatible with signaling-based detection, thereby allowing for additional information about an object's trajectory and making the system more practical or actively contained for use in highly trafficked areas. Such limited propagation can also provide time-resolved information about location history, making the BMS system useful for an even wider range of applications.

### Materials and Methods

Barcode generation: A collection of 28-bp DNA barcodes with a hamming distance greater than 5 were bioinformatically generated (see e.g., Table 5). The collection of generated barcodes was screened against GenBank genome data using NCBI BLAST, and any barcodes found to align to genome sequences of Bacillus subtilis (B. subtilis) or Saccharomyces cerevisiae (S. cerevisiae) were eliminated from the collection.

Transformation and barcode insertion in bacteria: B. subtilis strains were derived from the wild-type strain 168 and are listed in Table 2. Insertion-deletion mutants were from the Bacillus knock-out (BKE) collection; see e.g., Koo et al. Cell Sys. 4, 291-305 (2017), the contents of which are incorporated herein by reference in their entirety. All BKE mutants were back-crossed twice into B. subtilis 168 before assaying and prior to antibiotic cassette removal. Antibiotic cassette removal was performed using a temperature-sensitive plasmid encoding the Cre recombinase.

DNA barcodes were produced by amplifying 164 bp synthetic megamers (see e.g., Table 6) using oligonucleotide primers oCB034 and oCB035 (see e.g., Table 3) in PCR. The barcode fragments were cloned using standard restriction digest cloning into the plasmid pCB018 (ycgO::lox66-kan-lox71), a vector for double-crossover integration at the ycgO locus.

Bacterial sporulation: For large scale spore production, B. subtilis strains were sporulated in 1 L supplemented Difco™ Sporulation Medium (DSM) by nutrient exhaustion at 37° C. in 4 L flasks. After 36 hours of growth and sporulation, the spores were pelleted by centrifugation at 7000 rpm for 30 min, washed 2 times with sterile distilled water, incubated at 80° C. for 40 min to kill non-sporulating cells and then washed 5 times with sterile distilled water. Spores were stored at 4° C. in phosphate-buffered saline.

Evaluating bacterial spore lysis by microscopy: To rapidly assess the efficacy of different spore lysis protocols, a fluorescent protein was expressed in the spore core and its release was monitored following lysis by fluorescence microscopy. The mScarlett gene was PCR amplified with oligonucleotide primers oCB049 and oCB050 (see e.g., Table 3) from plasmid pHCL147 (see e.g., Lim et al. PLOS Genet. 15, e1008284 (2019), the contents of which are incorporated herein by reference in their entirety), and inserted downstream of the strong sporulation promoter P_{sspB} in plasmid pCB137 (yycR::P_{sspB}-spec), a vector for double-crossover integration at the yycR locus.

Spores were immobilized on 2% agarose pads. Fluorescence and phase-contrast microscopy was performed using an Olympus™ BX61 microscope equipped with an UplanF1 100× phase-contrast objective and a CoolSnapHQ digital camera. Exposure time was 400 ms for mScarlett. Images were analyzed and processed using MetaMorph™ software.

Transformation and barcode insertion in yeast: Barcodes were introduced into S. cerevisiae yeast strain BY4743 with standard lithium acetate chemical transformation with a 15 min heat shock. Following overnight recovery in YPD media (10 g/L yeast extract, 20 g/L peptone, 20 g/L glucose), cultures were plated on YPD+G418 to select for transformants. Yeast were transformed with 1 μg of barcode oligos (see e.g., Table 6), and two linearized plasmids: 50 ng of Cas9 plasmid, F48V (2p-KanR-pRPL18B-Cas9-tPGK1-GapRepair) and 1 μg of gRNA plasmid, F51V, containing a single gRNA targeting HO and 200- to 300-bp sequences homologous to the GapRepair region in F48V. When both are transformed into yeast cells, the two linearized fragments assemble into a functional plasmid granting G418 resistance. Once the HO locus targeted by the gRNA was replaced with the barcode sequence, the assembled Cas9+gRNA plasmid was dispensable. Plasmids were cured by culturing cells in YPD for overnight followed by spreading cells on YPD plates and replica plated to YPD+G418 plates to select for colonies negative for plasmids.

Yeast sporulation: Yeast cells were cultured in 5 mL of YPD medium at 30° C. overnight, then transferred to 1 L of YPD medium and cultured for 24 hours. Cells were pelleted by centrifugation at 3000 g for 3 min and washed twice with sterile distilled water. Finally, cells were resuspended in 500 mL of sporulation medium (10 g/L potassium acetate, 1 g/L yeast extract, and 0.5 g/L dextrose anhydrate) and incubated at room temperature while shaking for 5 days. Presence of spores was confirmed by microscopy at 60× magnification.

Spores were pelleted and the supernatant was carefully removed. The spores were then washed once then resuspended in 25 mL of sterile distilled water and transferred to 50 mL conical tubes. These tubes were boiled at 100° C. for 1 hour to rupture any remaining vegetative cells. After boiling, spores were pelleted, washed twice then resuspended in 25 mL of distilled water.

Production of LsCas13a: LsCas13a was purified as described previously (see e.g., Gootenberg et al. Science 356, 438-442 (2017), the contents of which are incorporated herein by reference in their entirety), with some modifications. All buffers were made in UltraPure™ nuclease-free water and all labware used during purification were cleaned with RNaseZap™ before use. Purification of the expressed LsCas13a protein was performed in batch format using StrepTactin™ sepharose. The SUMO-protease cleaved LsCas13a was concentrated using an Amicon™ Ultra-0.5 centrifugal filter with a 100 kDa molecular weight cutoff filter. The protein was concentrated until the sample measured as 2 mg/mL using the BioRad™ Protein Assay. The LsCas13a was not purified or concentrated further, and instead stored as 2 mg/mL aliquots in lysis buffer supplemented with 1 mM DTT and 5% glycerol. The use of RNase free water for all buffers during the preparation of LaCas13a is critical to achieving low basal activity of LaCas13a. New batches of LaCas13a can be tested prior to use to ensure low basal activity in the absence of crRNA.

Recombinase polymerase amplification reaction and primer design: Recombinase Polymerase Amplification (RPA) reactions were performed as described previously (see e.g., Gootenberg et al., supra). The Twist-Dx™ Basic kit was used according to the manufacturer's instructions. RPA primers JQ24 and JQ42 (see e.g., Table 3) were used at 480 nM final concentration to amplify a DNA amplicon of 161-bp, containing the T7 promoter and barcode sequences. The T7 promoter sequence was designed in-between the forward RPA primer, JQ42, and the barcode sequences (see e.g., FIG. 7A). RPA reactions were run with 1 μL of template in a total reaction volume of 10 uL for 1-2 hours at 37° C., unless otherwise noted.

SHERLOCK detection reactions and crRNAs: Detection reaction reactions were performed as described previously (see e.g., Gootenberg et al., supra). crRNA preparation was performed as described previously (see e.g., Gootenberg et al., supra), except the in-vitro transcription reaction volume was scaled to 60 μL. All crRNA and barcode sequences used herein are available in Tables 4-6. BioTek™ readers were used for measuring fluorescence of reactions (Synergy™ H1 Plate Reader) at Excitation/Emission=485 nm/528 nm wavelength for 90 min. Positive threshold cutoff value of 2500 was determined by averaging the values of negative control fluorescence values plus 4σ.

Inoculating surfaces with spores: Spores were diluted to a final concentration≤1×10⁸ spores/mL in distilled water in order to reduce the viscosity of the solution. Spores were routinely stored at 4° C. for long periods of time, or at room temperature for short periods of time. Diluted spores were sprayed onto surfaces using handheld spray bottles (Fisher Scientific™). At this concentration, inoculation had no visible effect on most surfaces tried, though water stains with white residue did appear on hydrophobically-treated wood due to water beading up on the surfaces.

Swab collection and NaOH lysis protocol: Sterile nylon swabs (Becton Dickinson™) were dipped into sterile swab solution (0.15 M NaCl+0.1% Tween-20) and excess liquid was wiped away. The damp swab was rubbed over the object, covering each part of the surface, twice. The tip of the swab was clipped into a microcentrifuge tube, and 200 μL of freshly prepared 200 mM NaOH was pipetted onto the swab. The tube was heated to 95° C. for 10 min, then the base was neutralized with 20 μL 2 M HCl, and buffered with 20 μL 10× TE buffer (Tris-HCl 100 mM, EDTA 10 mM, pH 8.0). Lysate samples were optionally purified with 1× AMPure™ XP bead protocol (Beckman Coulter™).

Spore quantitation by qPCR: Quantitative Polymerase Chain Reactions (qPCR) were prepared in 10 μL reactions with SYBR Green I Master Mix (Roche), 1 μL of genomic extract as a template, 0.4 mg/mL Bovine Serum Albumin, and 1 μM of each primer (see e.g., Table 3). The reactions were carried out in a LightCycler 480 instrument (Roche™) with the following cycling conditions: (i) denaturation, 95° C./10 m (ii) amplification, 45 cycles 95° C./10 s, 60° C./5 s, 72° C./10 s.

Design of ˜1 m²-scale test surfaces and perturbations: Small-scale test surfaces were constructed in an incubator to simulate real world conditions in which the Barcoded Microbial Spores (BMS) can be deployed. 20 test surfaces were assembled, from 4 materials (sand, soil, carpet, and wood), which were then divided into control and perturbed conditions (see e.g., Table 7). Each surface was divided into a ˜0.2 m×0.3 m grid, denoting different locations for direct samples and transfer samples each week. The gridded area of each surface was inoculated with different pairs of barcoded strains, using a handheld spray bottles, to a final concentration of ˜1.2×10⁶ B. subtilis spores and 3.8×10⁴ S. cerevisiae spores per square cm.

For outdoor conditions, twelve ˜0.2 m×0.3 m trays were filled to a ˜2.5 cm depth with either sand or potting mix, and housed on shelved in one of two incubators (Shel Labs™) heated to 25° C. (see e.g., FIG. 11A). To simulate wind, one incubator was equipped with 140 mm computer fans, one directed at each of the 6 perturbed trays. Simulated rain was applied to perturbed surfaces each week, varying in intensity from handheld spray bottle (e.g., 200 mL/week) on weeks 1-6 to watering can (e.g., 500 mL/week) on weeks 7-12. The incubator housing the perturbed surfaces was also programmed to fluctuate in temperature between 25° C. and 35° C. with a period of one week.

For indoor conditions, 4 sections of carpet and 4 sections of laminate wood flooring were cut out and marked out ˜0.2 m×0.3 m sections on each for testing. All 8 surfaces were shelved in an incubator (Shel Labs™) heated to 25° C., and humidified to 40-50% RH (see e.g., FIG. 11A-11G). To simulate cleaning, perturbed carpet surfaces were cleaned using a handheld vacuum, while perturbed wood surfaces were cleaned with a hand broom.

Sampling from −1 m²-scale test surfaces over a three-month period: Each week for a 13-week period, 0.25 g of sand or soil was sampled from each surface from a different adjacent location (˜2.5 cm away) on the tray each week using a microcentrifuge tube. Samples were processed using a DNeasy Powersoil™ kit (Qiagen™) to isolate DNA. For carpet and wood samples, a different adjacent location on the surface was swabbed directly using the swab collection and NaOH lysis protocol to generate lysate for qPCR. For all surfaces, 2.5 cm×5 cm test objects (either rubber or plywood) were used to test transferability of spores; test objects were pressed onto the surface a single time, then processed with the swab collection and NaOH lysis protocol to generate lysate used for qPCR without AMPure™ XP bead cleanup.

Design of full-scale sandpit and perturbations: A 6 m×16 m×0.25 m indoor sandpit was built and equipped with drainage and a slight grade. A 1 m×6 m section along the top edge was inoculated with B. subtilis BC-24 & BC-25 spores with roughly 2.5×10¹¹ spores each, and S. cerevisiae BC-49 & BC-50 spores with roughly 1.25×10¹⁰ spores each. One half of the sand pit was designated for environmental perturbations, with 1 m diameter fans placed at the inoculation end, and hose simulated rain (1.27 cm/week) applied each week.

Sampling from full-scale sandpit over a three-month period: Each week for a 13-week period, 0.25 g of sand was sampled from 20 collection spots in the sandpit (see e.g., FIG. 4B and FIG. 12) and processed using the NaOH lysis protocol. Each week, sampling was performed at an adjacent location (<8 cm away) in the region each week using a microcentrifuge tube. For testing transferability, objects (e.g., shoes or wood) were pressed onto the surface a single time (see e.g., FIG. 14A-14B), then processed with the sample swab and NaOH lysis protocol. In this experiment, 2 μl of lysates were used for RPA reactions followed by SHERLOCK using crRNA 24 and 25 to detect B. subtilis BMS, and crRNA 49 and 50 to detect S. cerevisiae BMS. Threshold was determined as described above, using average values of negative control fluorescence values plus 4σ. A new Cas13a batch was prepared on week 6, so subsequent reactions have different baseline signal and threshold values accordingly (see e.g., FIG. 13A-13B).

Sampling from contained outdoor environment: A grass site was inoculated with 2 BMS (BC-14 and BC-15) in August in a contained outdoor environment at a government research facility. After 5 months of exposure to natural weather (sun, rain, snow, ice, hail, grass-cutting, and wildlife activities), 4 grass samples were obtained; 1 of which was from the inoculated grass site and the rest were from 12 feet, 24 feet or 100 feet away from the inoculated site. For each grass sample, DNA was isolated using DNeasy PowerSoil Pro™ kit (Qiagen™). 5 samples of DNA were isolated for each of the four grass samples resulting in a total of 20 samples. The extracted DNA was then amplified in 12 μL reactions of RPA using JQ24 and JQ42 primers at 37° C. for 1 hour (see e.g., Table 3). 1 μl of RPA product was then used for Cas13a detection. Each DNA sample was tested in triplicate for both BC-14 and BC-15 and the reaction was read on BioTek™ plate reader.

Measuring spore retainment on shoes: 24 pairs of shoes were worn in the inoculation region of the ˜100 m² sandpit (see e.g., FIG. 4A-4G), accumulating spores over a 1-min walking period. These shoes were then divided into 8 groups, and were worn while walking in non-inoculated areas for 0, 1, 5, 15, 30, 60, 120 or 240 min. Non-inoculated areas included city sidewalks, dirt paths, and grassy lawns. Shoes were processed with the swab collection and NaOH lysis protocol, followed by AMPure™ XP bead purification. 2 μL of purified DNA was used for RPA reactions followed by SHERLOCK using crRNA 24 and 25 to detect B. subtilis BMS, and crRNA 49 and 50 to detect S. cerevisiae BMS (see e.g., FIG. 15A). qPCR reactions were prepared in 10 μL volume with PowerUP™ SYBR Green Master Mix (ThermoFisher Scientific™), 2 μL of purified DNA as template, and 2.5 μM of each primer listed in Table 3, then ran on a QuantStudio™ 6 instrument (ThermoFisher Scientific™).

Measuring spore re-transfer on shoes: 3 sandboxes were inoculated by spraying 2 BMS per sandbox. BMS were transferred to shoes by stepping 5 times with two shoes. One shoe was sampled directly and used as a pre-stepping control; the other shoe was used to walk into 3 different non-inoculated sandboxes. Sand from all the sandboxes were sampled after this initial walk to determine if BMS was transferred to these non-inoculated sandboxes. To simulate re-transfer of spore from sand back onto another shoes, new shoes were stepped five times in the non-inoculated sandboxes. DNA from sand samples were isolated using DNeasy PowerSoil Pro™ kit (Qiagen™) DNA from swabbed shoes were processed with the sample swab and NaOH lysis protocol. BC-1, 2, 23, 25, 90 and 91 qPCR (see e.g., primers in Table 3) were used for qPCR.

Determining object provenance: Grids of varying sizes (averaging 0.25 m² per region (see e.g., FIGS. 17A-17E and 18C-18D), or averaging 1 m² per region (see e.g., FIGS. 18A and 19)) and materials (sand, soil, carpet, wood) were inoculated using approximately 2.5×10¹¹ B. subtilis spores and/or 1.25×10¹⁰ S. cerevisiae spores per region. Surfaces were inoculated by spraying then dried for at least 24 hours before shoes and remote-control cars were used as test objects, and were exposed to inoculated surfaces by walking or driving on the test grid surfaces. All surfaces of test objects were swabbed and processed with the NaOH lysis protocol. For 1 unique BMS per sand region, a region was called positive if SHERLOCK reaction was positive for the unique BMS of that region; for 2 unique BMS per sand region, a region was called positive if SHERLOCK reaction was positive for at least 1 of the 2 BMS of that region; for 4 unique BMS per sand region, a region was called positive if SHERLOCK reaction was positive for at least 2 BMS of that region. False positive and false negative rates were calculated using different threshold criteria for a positive call of a region (see e.g., FIG. 20).

Qualitative readout of SHERLOCK using mobile phone camera: To mimic in-field deployment, the setup included a portable blue light source and an orange acrylic filter for data collection (see e.g., FIG. 17A). In a dark room, a mobile phone, set at default setting (flash off), was used to photograph the SHERLOCK reaction plate after 30-60 min of reaction.

Barcode identification from a model farm: 20 garden pots were filled with potting mix and enclosed in canvas with 12 hours of daily blue light. One seedling was planted in each pot. Temperature was controlled to be around 23° C. Plants were watered every 2-3 days and exposed to blue light for 12 hours daily. Barcoded B. subtilis spores were inoculated by spraying on the plants after the first set of leaves appeared. Inoculation was done for each plant separately once a week for 4 weeks during the growth period. In total, ˜10⁸-10⁹ spores were inoculated onto each plant. One week after the final inoculation, plant samples were harvested and processed using DNeasy PowerSoil Pro™ Kit as described above to isolate DNA. Barcode DNA was amplified using BTv2-F and BCv2-R (see e.g., Table 3) using Kapa Biosystems™ HiFi HotStart ReadyMix, then Sanger sequencing was used to identify the barcode sequences (GENEWIZ).

Barcode identification of co-associated plants: 7 leafy plants were bought and each leaf was marked allowing identification of the source plant for each leaf after harvest. For 6 of the 7 plants, each plant was inoculated by spraying once with one unique BMS. One control plant was not inoculated with BMS. In total, ˜10⁸-10⁹ spores were inoculated onto each plant. At 1 week, 4 weeks, and 6 weeks after inoculation, one leaf from each of the 7 plants was harvested, mixed, and shaken together with other leaves for 5 min in a Ziploc™ bag to simulate co-association of produce during the food supply chain. Mixed leaves were then taken out of the bag and individually processed with DNeasy Powersoil™ kit (Qiagen™) for gDNA extraction. SHERLOCK was used to screen for leaf DNA samples that were positive with group 2 crRNA. For leaf DNA samples that were positive with group 2 crRNA, the amplified DNA was sent for Sanger sequencing for identification of plant provenance.

PCR of Bacillus thuringiensis on produce: Around 250 mg of each produce sample was cut into 1-3 mm pieces using a scalpel and then processed with the DNeasy Powersoil Pro™ kit (Qiagen™) to isolate 50 μL of eluted DNA. 1 μL of the DNA was used for PCR with primers BT-1F and BT-1R (see e.g., Table 3) using Phire HotStart II™ DNA polymerase (ThermoFisher Scientific™) with the following cycling conditions: (i) denaturation, 98° C./30 s (ii) amplification, 36 cycles 98° C./5 s, 60° C./5 s, 72° C./10 s (iii) extension, 72° C./4 min.

Robustness of Bacillus thuringiensis on produce: Produce were selected that were PCR-positive for Bacillus thuringiensis, and then these samples were treated with various cooking methods: washing, boiling, microwaving, or frying. For washing, the produce pieces were placed in a 50 mL conical tube covered with a screen with tap water running over the sample for 10 min then dried in paper towels. For boiling, a piece of produce was placed in an Eppendorfr^{M} tube filled with 1 mL of water and placed in a boiling beaker of water for 15 min then dried in paper towels. For microwaving, produce pieces were placed in a petri dish with the cover on at full power for 2 min. For frying, 1 mL of vegetable oil was added to a 250 mL or 400 mL beaker that was pre-heated for 1 min on hotplate set to 350° C. then the piece of produce was added and heated for 1 min with occasional stirring. Around 250 mg of cooked sample was processed with DNeasy PowerSoil Pro™ Kit (Qiagen™). qPCR was performed using primers BT-1F and BT-1R (see e.g., Table 3) and PowerUp™ SYBR Green Master Mix (ThermoFisher Scientific™).

Sampling and library preparation for microbiome analysis of inoculated surfaces: For incubator-scale experiments simulating outdoor conditions, 0.25 g of sand or soil was sampled each month from each tray, and extracted genomic DNA with a DNeasy Powersoil™ kit (Qiagen™) For each tray, two samples were taken from the same locations each month, one from a region that had been inoculated with BMS, the other from a non-inoculated region of the same tray. Sequencing libraries were created in two rounds as described elsewhere (see e.g., Gohl et al. Nat Biotechnol 34, 942-949 (2016), the contents of which are incorporated herein by reference in their entirety), first targeting the v3-v4 16S rRNA region with primers prCM509 and prCM510 (see e.g., Table 3) using Kapa Biosystems HiFi HotStart ReadyMix™ with the following cycling conditions: (i) denaturation, 95° C./5 min (ii) amplification, 20 cycles (or 30 cycles for low-biomass sand samples) 98° C./20 s, 55° C./15 s, 72° C./1 min (iii) extension, 72° C./5 min. Next, Illumina Nextera™ XT primers were used to add barcodes using Kapa Biosystems HiFi HotStart ReadyMix™ with the following cycling conditions: (i) denaturation, 95° C./5 min (ii) amplification, 8 cycles 98° C./20 s, 55° C./15 s, 72° C./1 min (iii) extension, 72° C./10 min. Following each round of PCR, samples were purified using AMPure™ XP beads (Beckman Coulter™). Sequencing was performed using a MiSeq™ v3 kit to collect 300 bp paired end reads.

Sequencing analysis for 16S metagenomics samples: Sample compositions were determined using QIIME2 v2018.4 (see e.g., Bolyen et al. Nat Biotechnol 37, 852-857 (2019), the contents of which are incorporated herein by reference in their entirety) on a shared computing cluster. Single end reads (from the v4 region) truncated to 150 bp were analyzed. Sample inference was performed with DADA2 (see e.g., Callahan et al. Nat Methods 13, 581-583 (2016), the contents of which are incorporated herein by reference in their entirety), then taxonomy was assigned according to the SILVA 132 database to the phylum level for coarse grained analysis, or genus level to determine BMS abundance. All reads with a genus-level taxonomic assignment of Bacillus were attributed to the B. subtilis BMS. Weighted UniFrac distance (see e.g., Chang et al. BMC Bioinformatics 12, 118 (2011), the contents of which are incorporated herein by reference in their entirety) calculations for soil samples were calculated from 10000 reads excluding Bacillus reads. The distance between two samples varying only in a single parameter was calculated for all month 2 samples. For example, to determine the effect of inoculation, weighted UniFrac distance was calculated between wet soil+/−inoculation at month 2, and averaged with the distance between dry soil+/−inoculation at month 2, etc.

Developing High-Throughput Methods to Screen Barcodes and crRNAs

In order to scale the BMS, a facile method was devised to quickly screen a large number of barcodes and crRNAs in parallel to eliminate those with high cross reactivity or background; Pooled n−1 barcode RPA reactions were performed with a corresponding crRNA and H₂O RPA controls. A pooled n−1 barcode RPA reaction denotes a single RPA reaction with all DNA barcodes pooled together except for one barcode that is left out, and this pooled reaction is screened against all crRNAs. Following the initial in vitro screen of 22 barcode crRNA pairs, 72 additional barcodes were made and all 94 were tested (see e.g., Table 6). Retesting the initial 22 crRNA validated the n−1 barcode assay; 19 of the 22 passed while crRNAs 7, 11, and 12 that were just below the cut-off in the pairwise test (see e.g., Methods) scored as cross-reactive in the pooled assay. In total, 17 of the 94 crRNAs were eliminated due to high background and 7 additional crRNAs were eliminated due to cross-reactivity with multiple barcodes (e.g., crRNAs: 7, 11, 12, 15, 26, 31, 33, 40, 41, 42, 52, 57, 59, 66, 67, 69, 70, 71, 79, 80, 82, 85, 86, 94; e.g., crRNAs: 7, 11, 15, 52, 82, 83). Cross-reactivity appeared to be caused by crRNAs instead of barcodes as no barcodes were cross-reactive with all crRNAs (see e.g., FIGS. 7B and 7C). To perform screening of crRNAs on n−1 and n BMS in vivo, one would need to mix spores at equimolar concentration to avoid amplification biases (see e.g., FIG. 7D).

### Feasibility Testing of BMS on Incubator-Scale Test Surfaces

Compared to in vitro tests, real-world environments often present challenges for enzyme-based analyte detection systems, both by sequestering analyte and inhibiting reactions. Real-world environments also present a challenge to the stability of forensic labels, by degrading or washing away the label over time. the feasibility of the forensic microbial spores was tested by constructing chambers to simulate different real-world environments and perturbations. Four material types were chosen: sand, soil, carpet, and wood, and the test surfaces of these materials were housed in modified incubators in order to simulate indoor and outdoor conditions. Perturbations (e.g. rain/wind/cleaning) were also devised that can be expected to remove spores from the surface.

Over a 3-month period, qPCR targeted to the forensic microbial spores demonstrated no significant loss of spores over time (see e.g., FIG. 4A). The perturbations did not significantly reduce detection compared to control surfaces for any of the 4 materials. Furthermore, in most cases, spores could be transferred to a rubber or wood test object after a single direct exposure to the inoculated surface, and subsequently detected by qPCR (see e.g., FIG. 11A-11G).

Additional factors that can be tested for their effect on BMS integrity over time include, but are not limited to: abiotic factors like sunlight radiation, pH, or chemical stresses (e.g. cleaning agents), or biotic factors like consumption or enzymatic degradation by other organisms. Without wishing to be bound by theory, extensive validation in real-world environments is expected to demonstrate the feasibility of BMS for different applications, and it is expected that such environmental factors have a limited impact on the detection of spores over time.

Sensitivity and Specificity of PCR-Based Detection of Bacillus thuringiensis

To validate the sensitivity of the primers used in detecting Bt, first a pool of non-Bt bacterial gDNA (Streptomyces Hygroscopicus, S. cerevisiae, B. subtilis, E. coli and Pseudomonas) along with Bt gDNA was tested, and only Bt gave rise to a PCR band (see e.g., FIG. 23A, 28C). To test the specificity of PCR based Bt detection 12 negative control produce samples were included either from personal gardens or a local farm that had not been sprayed with Bt and 26 positive controls from a local farm that were known to have been sprayed with Bt (see e.g., FIGS. 23B, 23C). Produce purchased from the grocery store was then tested, and 10 of 24 samples were positive for Bt (FIGS. 23B and 24A-24C). To further test the validity of the detected bands from produce, 6 PCR-positive samples were randomly selected and their PCR products were sent for Sanger sequencing. The sequences matched the Bt cry1A gene and belonged to 3 variants (see e.g., FIG. 24B). When the sequences were input into NCBI BLAST against the nr databases, the only organism that showed up in the top 100 hits was Bt (not shown). The same primers used in the PCR screens of produce (see e.g., FIGS. 23A-23C and 24A-24C) were used in a qPCR assay to assess the level of spores on produce after cooking. To validate the primers used in qPCR, a standard cure was made of purified Bt gDNA, which was sensitive enough to detect the amount of gDNA equivalent to as few as 10 spores (see e.g., FIG. S25B).

As BMS are grown in routine bacterial and yeast cell culture techniques, it is expected, without wishing to be bound by theory, that the BMS can be produced at an industrial scale. For example, Bacillus thuringiensis spores can be produced industrially for agricultural application at low cost.

### Tables

TABLE 1
Detection of Bacillus thuringiensis on produce samples
(+, positive; −, negative; NA, not available)

| Organic | Sample | Merchant/Source | BT |
|---|---|---|---|
| NA | Green beans | C-Mart ™ | − |
| NA | Okra | C-Mart ™ | − |
| NA | Scallion | C-Mart ™ | − |
| − | Baby spinach | Stop & Shop ™ | + |
| − | Broccolini | Stop & Shop ™ | − |
| − | Green beans | Stop & Shop ™ | − |
| − | Iceberg lettuce | Stop & Shop ™ | + |
| − | Kale | Stop & Shop ™ | + |
| − | Okra | Stop & Shop ™ | − |
| − | Romaine lettuce | Stop & Shop ™ | + |
| − | Scallion | Stop & Shop ™ | − |
| + | Baby spinach | Stop & Shop ™ | + |
| + | Kale | Stop & Shop ™ | + |
| + | Romaine lettuce | Stop & Shop ™ | + |
| − | Basil | Whole Food ™ | − |
| − | Green beans | Whole Food ™ | − |
| − | Okra | Whole Food ™ | − |
| + | Arugula | Whole Food ™ | + |
| + | Baby spinach | Whole Food ™ | + |
| + | Broccoli | Whole Food ™ | + |
| − | Watercress | Stop & Shop ™ | + |
| − | Baby spinach, brand B | Stop & Shop ™ | + |
| + | Romaine lettuce, brand B | Stop & Shop ™ | + |
| + | Cucumber | Stop & Shop ™ | − |
| + | Green beans, brand B | Stop & Shop ™ | − |
| − | Broccoli wokly ® | Stop & Shop ™ | + |
| − | Mini peppers | Stop & Shop ™ | + |
| − | Spinach, brand B | Stop & Shop ™ | + |
| − | Baby arugula | Stop & Shop ™ | + |
| − | Strawberries | Stop & Shop ™ | − |
| − | Brussels sprouts | Stop & Shop ™ | + |
| + | Iceberg lettuce, brand B | Stop & Shop ™ | + |

TABLE 2
Exemplary bacterial and yeast strains used herein. List includes wild type
strains and mutants generated herein. All unmarked mutations are in-frame
deletions generated by Cre-mediated recombination and contain a lox72 scar.

| | | | |
|---|---|---|---|
| Strain | Genotype | Alias | Source |

Saccharomyces cerevisiae strains

| | | | |
|---|---|---|---|
| BY4743 | MATa/MATα his3Δ1/his3Δ1 leu2Δ0/leu2Δ0 | WT | ATCC |
| | lys2Δ0/LYS2 met15Δ0/MET15 ura3Δ0/ura3Δ0 | | (American |
| | | | Type Culture |
| | | | Collection) |
| yBC1 | MATa/MATα his3Δ1/his3Δ1 leu2Δ0/leu2Δ0 | BC1 | Herein |
| | lys2Δ0/LYS2 met15Δ0/MET15 ura3Δ0/ura3Δ0 | | |
| | hoΔ::BC1 | | |
| yBC2 | MATa/MATα his3Δ1/his3Δ1 leu2Δ0/leu2Δ0 | BC2 | Herein |
| | lys2Δ0/LYS2 met15Δ0/MET15 ura3Δ0/ura3Δ0 | | |
| | hoΔ::BC2 | | |
| yBC3 | MATa/MATα his3Δ1/his3Δ1 leu2Δ0/leu2Δ0 | BC3 | Herein |
| | lys2Δ0/LYS2 met15Δ0/MET15 ura3Δ0/ura3Δ0 | | |
| | hoΔ::BC3 | | |
| yBC4 | MATa/MATα his3Δ1/his3Δ1 leu2Δ0/leu2Δ0 | BC4 | Herein |
| | lys2Δ0/LYS2 met15Δ0/MET15 ura3Δ0/ura3Δ0 | | |
| | hoΔ::BC4 | | |
| yBC5 | MATa/MATα his3Δ1/his3Δ1 leu2Δ0/leu2Δ0 | BC5 | Herein |
| | lys2Δ0/LYS2 met15Δ0/MET15 ura3Δ0/ura3Δ0 | | |
| | hoΔ::BC5 | | |
| yBC6 | MATa/MATα his3Δ1/his3Δ1 leu2Δ0/leu2Δ0 | BC6 | Herein |
| | lys2Δ0/LYS2 met15Δ0/MET15 ura3Δ0/ura3Δ0 | | |
| | hoΔ::BC6 | | |
| yBC7 | MATa/MATα his3Δ1/his3Δ1 leu2Δ0/leu2Δ0 | BC7 | Herein |
| | lys2Δ0/LYS2 met15Δ0/MET15 ura3Δ0/ura3Δ0 | | |
| | hoΔ::BC7 | | |
| yBC49 | MATa/MATα his3Δ1/his3Δ1 leu2Δ0/leu2Δ0 | BC49 | Herein |
| | lys2Δ0/LYS2 met15Δ0/MET15 ura3Δ0/ura3Δ0 | | |
| | hoΔ::BC49 | | |
| yBC50 | MATa/MATα his3Δ1/his3Δ1 leu2Δ0/leu2Δ0 | BC50 | Herein |
| | lys2Δ0/LYS2 met15Δ0/MET15 ura3Δ0/ura3Δ0 | | |
| | hoΔ::BC50 | | |

Bacillus subtilis strains

| | | | |
|---|---|---|---|
| 168 | Wild-type (trpC2) | WT | BGSC |
| | | | (Bacillus |
| | | | Genetic |
| | | | Stock |
| | | | Center) |
| BDR3897 | ΔthrC::lox72 ΔmetA::lox72 ΔtrpC::lox72 | Δ9 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| BDR3900 | ΔthrC::lox72 ΔmetA::lox72 ΔtrpC::lox72 | Δ9 ycgO::erm | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::erm | | |
| BDR3916 | ΔthrC::lox72 ΔmetA::lox72 ΔtrpC::lox72 | Δ9 yycR::cat | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | yycR::cat | | |
| BDR3916 | ΔthrC::lox72 ΔmetA::lox72 ΔtrpC::lox72 | Δ9 yycR::P_{sspB}- | Herein |
| | ΔpheA::lox72 ΔsleB::lox72 ΔcwlJ::lox72 | optRBS-mScarlett | |
| | ΔgerAB::lox72 ΔgerBB::lox72 ΔgerKB::lox72 | (Spec) | |
| | yycR::P_{sspB}-optRBS-mScarlett (Spec) | | |
| BDR4038 | ΔthrC::lox72 ΔmetA::lox72 ΔtrpC:lox72 | Δ9 ycgO::V2-BC1 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC1 (lox72) | | |
| BDR4039 | ΔthrC::lox72 ΔmetA::lox72 ΔtrpC::lox72 | Δ9 ycgO::V2-BC2 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC2 (lox72) | | |
| BDR4040 | ΔthrC::lox72 ΔmetA::lox72 ΔtrpC::lox72 | Δ9 ycgO::V2-BC3 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC3 (lox72) | | |
| BDR4041 | ΔthrC::lox72 ΔmetA::lox72 ΔtrpC::lox72 | Δ9 ycgO::V2-BC4 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC4 (lox72) | | |
| BDR4042 | ΔthrC::lox72 ΔmetA::lox72 ΔtrpC::lox72 | Δ9 ycgO::V2-BC5 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC5 (lox72) | | |
| BDR4043 | ΔthrC::lox72 ΔmetA::lox72 ΔtrpC::lox72 | Δ9 ycgO::V2-BC6 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC6 (lox72) | | |
| BDR4052 | ΔthrC::lox72 ΔmetA::lox72 ΔtrpC::lox72 | Δ9 ycgO::V2-BC7 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC7 (lox72) | | |
| BDR4053 | ΔthrC::lox72 ΔmetA::lox72 ΔtrpC::lox72 | Δ9 ycgO::V2-BC8 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC8 (lox72) | | |
| BDR4054 | ΔthrC::lox72 ΔmetA::lox72 ΔtrpC::lox72 | Δ9 ycgO::V2-BC9 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC9 (lox72) | | |
| BDR4055 | ΔthrC:lox72 ΔmetA:lox72 ΔtrpC::lox72 | Δ9 ycgO::V2-BC10 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC10 (lox72) | | |
| BDR4029 | ΔthrC::lox72 ΔmetA::lox72 ΔtrpC::lox72 | Δ9 ycgO::V2-BC11 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC11 (lox72) | | |
| BDR4030 | ΔthrC::lox72 ΔmetA::lox72 ΔtrpC::lox72 | Δ9 ycgO::V2-BC12 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC12 (lox72) | | |
| BDR4031 | ΔthrC::lox72 ΔmetA::lox72 ΔtrpC::lox72 | Δ9 ycgO::V2-BC13 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC13 (lox72) | | |
| BDR4032 | ΔthrC::lox72 ΔmetA::lox72 ΔtrpC::lox72 | Δ9 ycgO::V2-BC14 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC14 (lox72) | | |
| BDR4033 | ΔthrC::lox72 ΔmetA::lox72 ΔtrpC::lox72 | Δ9 ycgO::V2-BC15 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC15 (lox72) | | |
| BDR4034 | ΔthrC::lox72 ΔmetA::lox72 ΔtrpC::lox72 | Δ9 ycgO::V2-BC16 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC16 (lox72) | | |
| BDR4056 | ΔthrC::lox72 ΔmetA::lox72 ΔtrpC::lox72 | Δ9 ycgO::V2-BC17 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC17 (lox72) | | |
| BDR4057 | ΔthrC::lox72 ΔmetA::lox72 ΔtrpC::lox72 | Δ9 ycgO::V2-BC18 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC18 (lox72) | | |
| BDR4064 | ΔthrC::lox72 ΔmetA::lox72 ΔtrpC::lox72 | Δ9 ycgO::V2-BC19 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC19 (lox72) | | |
| BDR4065 | ΔthrC::lox72 ΔmetA::lox72 ΔtrpC::lox72 | Δ9 ycgO::V2-BC20 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC20 (lox72) | | |
| BDR4066 | ΔthrC::lox72 ΔmetA::lox72 ΔtrpC::lox72 | Δ9 ycgO::V2-BC21 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC21 (lox72) | | |
| BDR4067 | ΔthrC::lox72 ΔmetA::lox72 ΔtrpC::lox72 | Δ9 ycgO::V2-BC22 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC22 (lox72) | | |
| BDR4075 | ΔthrC::lox72 ΔmetA::lox72 ΔtrpC::lox72 | Δ9 ycgO::V2-BC23 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC23 (lox72) | | |
| BDR4068 | ΔthrC::lox72 ΔmetA::lox72 ΔtrpC::lox72 | Δ9 ycgO::V2-BC24 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC24 (lox72) | | |
| BDR4069 | ΔthrC::lox72 ΔmetA::lox72 ΔtrpC::lox72 | Δ9 ycgO::V2-BC25 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC25 (lox72) | | |
| BDR4070 | ΔthrC::lox72 ΔmetA::lox72 ΔtrpC::lox72 | Δ9 ycgO::V2-BC26 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC26 (lox72) | | |
| BDR4071 | ΔthrC::lox72 ΔmetA::lox72 ΔtrpC::lox72 | Δ9 ycgO::V2-BC27 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC27 (lox72) | | |
| BDR4232 | ΔthrC::lox72 ΔmetA::lox72 ΔtrpC::lox72 | Δ9 ycgO::V2-BC52 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC52 (lox72) | | |
| BDR4233 | ΔthrC::lox72 ΔmetA::lox72 ΔtrpC::lox72 | Δ9 ycgO::V2-BC53 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC53 (lox72) | | |
| BDR4234 | ΔthrC::lox72 ΔmetA::lox72 ΔtrpC::lox72 | Δ9 ycgO::V2-BC54 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC54 (lox72) | | |
| BDR4235 | ΔthrC::lox72 ΔmetA::lox72 ΔtrpC::lox72 | Δ9 ycgO::V2-BC55 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC55 (lox72) | | |
| BDR4236 | ΔthrC::lox72 ΔmetA::lox72 ΔtrpC::lox72 | Δ9 ycgO::V2-BC56 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC56 (lox72) | | |
| BDR4237 | ΔthrC::lox72 ΔmetA::lox72 ΔtrpC::lox72 | Δ9 ycgO::V2-BC57 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC57 (lox72) | | |
| BDR4238 | ΔthrC::lox72 ΔmetA::lox72 ΔtrpC::lox72 | Δ9 ycgO::V2-BC58 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC58 (lox72) | | |
| BDR4239 | ΔthrC::lox72 ΔmetA::lox72 ΔtrpC::lox72 | Δ9 ycgO::V2-BC59 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC59 (lox72) | | |
| BDR4240 | ΔthrC::lox72 ΔmetA::lox72 ΔtrpC::lox72 | Δ9 ycgO::V2-BC60 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC60 (lox72) | | |
| BDR4241 | ΔthrC::lox72 ΔmetA::lox72 ΔtrpC::lox72 | Δ9 ycgO::V2-BC61 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC61 (lox72) | | |
| BDR4242 | ΔthrC::lox72 ΔmetA::lox72 ΔtrpC::lox72 | Δ9 ycgO::V2-BC62 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC62 (lox72) | | |
| BDR4243 | ΔthrC::lox72 ΔmetA::lox72 ΔtrpC::lox72 | Δ9 ycgO::V2-BC63 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC63 (lox72) | | |
| BDR4244 | ΔthrC::lox72 ΔmetA::lox72 ΔtrpC::lox72 | Δ9 ycgO::V2-BC76 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC76 (lox72) | | |
| BDR4245 | ΔthrC::lox72 ΔmetA::lox72 ΔtrpC::lox72 | Δ9 ycgO::V2-BC77 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC77 (lox72) | | |
| BDR4246 | ΔthrC::lox72 ΔmetA::lox72 ΔtrpC::lox72 | Δ9 ycgO::V2-BC78 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC78 (lox72) | | |
| BDR4247 | ΔthrC::lox72 ΔmetA::lox72 ΔtrpC::lox72 | Δ9 ycgO::V2-BC79 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC79 (lox72) | | |
| BDR4248 | ΔthrC::lox72 ΔmetA::lox72 ΔtrpC::lox72 | Δ9 ycgO::V2-BC81 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC81 (lox72) | | |
| BDR4249 | ΔthrC::lox72 ΔmetA::lox72 ΔtrpC::lox72 | Δ9 ycgO::V2-BC82 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC82 (lox72) | | |
| BDR4250 | ΔthrC::lox72 ΔmetA::lox72 ΔtrpC::lox72 | Δ9 ycgO::V2-BC83 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC83 (lox72) | | |
| BDR4251 | ΔthrC::lox72 ΔmetA::lox72 ΔtrpC::lox72 | Δ9 ycgO::V2-BC84 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC84 (lox72) | | |
| BDR4252 | ΔthrC::lox72 ΔmetA::lox72 ΔtrpC::lox72 | Δ9 ycgO::V2-BC85 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC85 (lox72) | | |
| BDR4253 | ΔthrC::lox72 ΔmetA::lox72 ΔtrpC::lox72 | Δ9 ycgO::V2-BC86 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC86 (lox72) | | |
| BDR4254 | ΔthrC::lox72 ΔmetA::lox72 ΔtrpC::lox72 | Δ9 ycgO::V2-BC87 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC87 (lox72) | | |
| BDR4255 | ΔthrC::lox72 ΔmetA::lox72 ΔtrpC::lox72 | Δ9 ycgO::V2-BC88 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC88 (lox72) | | |
| BDR4256 | ΔthrC::lox72 ΔmetA::lox72 ΔtrpC::lox72 | Δ9 ycgO::V2-BC89 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC89 (lox72) | | |
| BDR4257 | ΔthrC::lox72 ΔmetA::lox72 ΔtrpC::lox72 | Δ9 ycgO::V2-BC90 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC90 (lox72) | | |
| BDR4258 | ΔthrC::lox72 ΔmetA::lox72 ΔtrpC::lox72 | Δ9 ycgO::V2-BC91 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC91 (lox72) | | |
| BDR4259 | ΔthrC::lox72 ΔmetA::lox72 ΔtrpC::lox72 | Δ9 ycgO::V2-BC92 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC92 (lox72) | | |
| BDR4260 | ΔthrC::lox72 ΔmetA::lox72 ΔtrpC::lox72 | Δ9 ycgO::V2-BC93 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC93 (lox72) | | |
| BDR4261 | ΔthrC::lox72 ΔmetA::lox72 ΔtrpC::lox72 | Δ9 ycgO::V2-BC94 | Herein |
| | ΔpheA::lox72 ΔsleB::lox72 ΔcwlJ::lox72 | | |
| | ΔgerAB::lox72 ΔgerBB::lox72 ΔgerKB::lox72 | | |
| | ycgO::V2-BC94 (lox72) | | |

While the number of different barcode sequences that can be used is very large, exemplary sequences for use in compositions and methods as described herein are shown in Tables 3-6. Each crRNA in Table 4 comprises a Cas13 scaffold (e.g., SEQ ID NO: 2) and a region that is complementary and/or hybridizes to a barcode region with the corresponding barcode number in Table 5. Each crRNA can be used to detect a barcode region of a genetic barcode element as described herein. Accordingly, described herein are systems comprising, for example, at least one crRNA selected from Table 4 (e.g., SEQ ID NOs: 59 and 153) and a genetic barcode element selected from Table 6 (e.g., SEQ ID NO: 22).

As a non-limiting example, SEQ ID NO: 59 can be used to detect a genetic barcode element comprising Barcode 1 (e.g., SEQ ID NO: 5); as a non-limiting example, SEQ ID NO: 59 can be used to detect the second barcode region of SEQ ID NO: 222. As a non-limiting example, SEQ ID NO: 59 is reproduced below, with bold nucleotides showing the region of the crRNA that hybridizes to the second barcode region of the genetic barcode element (e.g., Barcode 1, SEQ ID NO: 5) and italicized nucleotides showing the region of the crRNA comprising a Cas13 scaffold (e.g., SEQ ID NO: 2):

TCGTCAGTCGTAACTTGGGAACGCACATGTTTTAGTCC
CCTTCGTTTTTGGGGTAGTCTAAATCCCCTATAGTGAG
TCGTATTAATTTC

As a non-limiting example, SEQ ID NO: 153 can be used to detect a genetic barcode element comprising Group 2 barcode (e.g., SEQ ID NO: 221); as a non-limiting example, SEQ ID NO: 153 can be used to detect the first barcode region of SEQ ID NO: 222. As a non-limiting example, SEQ ID NO: 153 is reproduced below, with bold double-underlined nucleotides showing the region of the crRNA that hybridizes to the first barcode region of the genetic barcode element (e.g., Group 2, SEQ ID NO: 221) and italicized nucleotides showing the region of the crRNA comprising a Cas13 scaffold e.g., SEQ ID NO: 2):

[symbol]

GTTTTAGTCCCCTTCGTTTT
TGGGGTAGTCTAAATCCCCTATAGTGAGTCGTATTAATTTC

Each genetic barcode element in Table 6 comprises a first primer binding region, a transcription initiation site, a first barcode region, a second barcode region, and a second primer binding region. In some embodiments of any of the aspects, the first barcode region indicates that an item on which the microorganism is detected is from one of a group of known sources, and the second barcode region indicates that an item on which the microorganism is detected is from a particular source of said group of sources.

As a non-limiting example, SEQ ID NO: 222 is reproduced below, with italicized nucleotides showing the first and second primer binding regions (e.g., SEQ ID NO: 4 and reverse complement of SEQ ID NO: 1, respectively); bold italicized text shows the transcription initiation site (e.g., reverse complement of SEQ ID NO: 3); bold nucleotides showing the second barcode region of the genetic barcode element (e.g., Barcode 1, SEQ ID NO: 5, a “unique barcode”); and bold double underlined text shows the first barcode region (e.g., Group 2 barcode, SEQ ID NO: 221, a “group barcode):

CTGGGATCCTCTAGAAATATGGATTACTTGGTAGAACAG
AATTCTAATACGACTCACTATAGGGCTAGCTTCGTCAGT
CGTAACTTGGGAACGCACATTAGATCTCA [symbol] [symbol]
TTGGCGTAATCATGGTCATAGCTGTTTC
CTGTGTTTATCAG

TABLE 3
Primers used herein

| SEQ | | | |
|---|---|---|---|
| ID | Primer | | |
| NO: | Name | Sequence | Target |
| 32 | prCM488 | GATAAACACAGGAAACAGCTA | Barcode |
| | | TGACCATGATTACG | reverse |
| 33 | prCM515 | TCGTCAGTCGTAACTTGGGAA | Barcode 1 |
| | | CGCACAT | forward |
| 34 | prCM516 | CTTTGGGGTTGGATAAATCTG | Barcode 2 |
| | | TCGGTGT | forward |
| 35 | prCM517 | TGAATAAGCGCGGTCCCTAAT | Barcode 3 |
| | | GTTGGTG | forward |
| 36 | prCM518 | ACCGGTTTATAGTTCGAACAG | Barcode 4 |
| | | TCGCAGC | forward |
| 37 | prCM519 | CCCGTGTGGTAACTACGCAAG | Barcode 5 |
| | | CCTAAC | forward |
| 38 | prCM520 | GGGCGCGTAACCACTAGCTC | Barcode 6 |
| | | | forward |
| 39 | prCM521 | gggctagctAGTGTCCCTTAT | Barcode 7 |
| | | TCTACTTTGAATTATC | forward |
| 40 | prCM522 | GTTACGGGTCAGGATCATTGC | Barcode 8 |
| | | GCAGG | forward |
| 41 | prCM545 | CCGACTAGGTCGGCGATACC | Barcode 24 |
| | | ATAGCACC | forward |
| 42 | prCM546 | GATGACGCCTCACGTCCGTTG | Barcode 25 |
| | | CCCCCTA | forward |
| 43 | prCM547 | TGCCTCAGAATGGGGTTTAAC | Barcode 19 |
| | | GTTCAGG | forward |
| 44 | prCM548 | ACCGTCCTAGTGCGGATAGTG | Barcode 49 |
| | | GGACATG | forward |
| 45 | prCM549 | GTCGATACGGGTCGCTAGATAG | Barcode 50 |
| | | GTTGGG | forward |
| 46 | BCv2-F | GGGATCCTCTAGAAATATGG | universal |
| | | | barcode |
| | | | forward |
| 47 | BCV2-R | TGATAAACACAGGAAACAGC | universal |
| | | | barcode |
| | | | reverse |
| 48 | gRNA_ | ATCAACAACAGAGGACATAT | HO- |
| | gap_ | GCCC | targeting |
| | repair_ | | gRNA |
| | F | | |
| 49 | gRNA_ | ATCCTGCACTCATCTACTAC | HO- |
| | gap_ | CCG | targeting |
| | repair_ | | gRNA |
| | R | | |
| 50 | BT-1F | CAAACATGCGAAGCGACTTA | detects Bt |
| | | | cry1a |
| 51 | BT-1R | CGCCTCCTTGGATGGTAATA | detects Bt |
| | | | cry1a |
| 52 | JQ 39 | gaaatTAATACGACTCACTAT | forward |
| | | Aggg | primer |
| | | | to anneal |
| | | | to |
| | | | reverse |
| | | | crRNA |
| 53 | JQ 24 | GATAAACACAGGAAACAGCTAT | RPA reverse |
| | | GACCATGATTACG | primer |
| 54 | JQ 42 | GGGATCCTCTAGAAATATGGAT | RPA forward |
| | | TACTTGGTAGAACAG | primer |
| 55 | oCB034 | cgcGGATCCtctagaaatatgg | megaprimer |
| | | attacttg | forward |
| 56 | oCB035 | cggCTCGAGataaacacaggaa | megaprimer |
| | | acagctat | reverse |
| 57 | oCB049 | gcgAAGCTTacataaggaggaa | mScarlett |
| | | ctactatggtttctaaaggt | forward |
| 58 | oCB050 | GCGGGATCCttacttatacagt | mScarlett |
| | | tcatc | reverse |

TABLE 4
Exemplary crRNAs used herein (e.g., for SHERLOCK reactions)

| SEQ ID | | |
|---|---|---|
| NO: | Name | Sequence |
| 59 | Group 1 | TCGTCAGTCGTAACTTGGGAACGCACATGTTTTAGTCCCCTTCGTT |
| | crRNA | TTTGGGGTAGTCTAAATCCCCTATAGTGAGTCGTATTAATTTC |
| 60 | crRNA_ | CTTTGGGGTTGGATAAATCTGTCGTGGTGTTTTAGTCCCCTTCGTT |
| | Barcode_2 | TTTGGGGTAGTCTAAATCCCCTATAGTGAGTCGTATTAATTTC |
| 61 | crRNA_ | TGAATAAGCGCGGTCCCTAATGTTGGTGGTTTTAGTCCCCTTCGTT |
| | Barcode_3 | TTTGGGGTAGTCTAAATCCCCTATAGTGAGTCGTATTAATTTC |
| 62 | crRNA_ | ACCGGTTTATAGTTCGAACAGTCGCAGCGTTTTAGTCCCCTTCGTT |
| | Barcode_4 | TTTGGGGTAGTCTAAATCCCCTATAGTGAGTCGTATTAATTTC |
| 63 | crRNA_ | CCCCGTGTGGTAACTACGCAAGCCTAACGTTTTAGTCCCCTTCGTT |
| | Barcode_5 | TTTGGGGTAGTCTAAATCCCCTATAGTGAGTCGTATTAATTTC |
| 64 | crRNA_ | ACGTAGGGGGGCGCGTAACCACTAGCTCGTTTTAGTCCCCTTCGT |
| | Barcode_6 | TTTTGGGGTAGTCTAAATCCCCTATAGTGAGTCGTATTAATTTC |
| 65 | crRNA_ | AGTGTCCCTTATTCTACTTTGAATTATCGTTTTAGTCCCCTTCGTTT |
| | Barcode_7 | TTGGGGTAGTCTAAATCCCCTATAGTGAGTCGTATTAATTTC |
| 66 | crRNA_ | GAGTTACGGGTCAGGATCATTGCGCAGGGTTTTAGTCCCCTTCGTT |
| | Barcode_8 | TTTGGGGTAGTCTAAATCCCCTATAGTGAGTCGTATTAATTTC |
| 67 | crRNA_ | GTGAGTCCGGCCTATCACGTTTGGTAGGGTTTTAGTCCCCTTCGTT |
| | Barcode_9 | TTTGGGGTAGTCTAAATCCCCTATAGTGAGTCGTATTAATTTC |
| 68 | crRNA_ | TCAGGGGAAACGAGTTAAGCAGAGGCAGGTTTTAGTCCCCTTCGT |
| | Barcode_10 | TTTTGGGGTAGTCTAAATCCCCTATAGTGAGTCGTATTAATTTC |
| 69 | crRNA_ | CTTGGTCCAATCGTATGCTAAGAGTAGCGTTTTAGTCCCCTTCGTT |
| | Barcode_11 | TTTGGGGTAGTCTAAATCCCCTATAGTGAGTCGTATTAATTTC |
| 70 | crRNA_ | TCGGTTGCACGCGCGTCCTGCACGCTCGGTTTTAGTCCCCTTCGTT |
| | Barcode_12 | TTTGGGGTAGTCTAAATCCCCTATAGTGAGTCGTATTAATTTC |
| 71 | crRNA_ | GTTCAAAAGCGGGAGTCCCGGTGAAACCGTTTTAGTCCCCTTCGT |
| | Barcode_13 | TTTTGGGGTAGTCTAAATCCCCTATAGTGAGTCGTATTAATTTC |
| 72 | crRNA_ | GAGGGCTTCTACGAAATTGCCTCACCATGTTTTAGTCCCCTTCGTT |
| | Barcode_14 | TTTGGGGTAGTCTAAATCCCCTATAGTGAGTCGTATTAATTTC |
| 73 | crRNA_ | GGGATGCCCTGATAACGTAGAGTCTCAGGTTTTAGTCCCCTTCGTT |
| | Barcode_15 | TTTGGGGTAGTCTAAATCCCCTATAGTGAGTCGTATTAATTTC |
| 74 | crRNA_ | TAGTACGGGCCACGATCTAAGTCGGCGGGTTTTAGTCCCCTTCGTT |
| | Barcode_16 | TTTGGGGTAGTCTAAATCCCCTATAGTGAGTCGTATTAATTTC |
| 75 | crRNA_ | GTATGTGGGTACGATTGTAGGCTAGAAAGTTTTAGTCCCCTTCGTT |
| | Barcode_17 | TTTGGGGTAGTCTAAATCCCCTATAGTGAGTCGTATTAATTTC |
| 76 | crRNA_ | AGAACTACCTACGTGGCCACGCAAGCCCGTTTTAGTCCCCTTCGTT |
| | Barcode_18 | TTTGGGGTAGTCTAAATCCCCTATAGTGAGTCGTATTAATTTC |
| 77 | crRNA_ | TGCCTCAGAATGGGGTTTAACGTTCAGGGTTTTAGTCCCCTTCGTT |
| | Barcode_19 | TTTGGGGTAGTCTAAATCCCCTATAGTGAGTCGTATTAATTTC |
| 78 | crRNA_ | ATGTTGGGACGCCAGACTGACACGCAAAGTTTTAGTCCCCTTCGT |
| | Barcode_20 | TTTTGGGGTAGTCTAAATCCCCTATAGTGAGTCGTATTAATTTC |
| 79 | crRNA_ | CCTACACCTTCACCGAGTGTGGAGCAAGGTTTTAGTCCCCTTCGTT |
| | Barcode_21 | TTTGGGGTAGTCTAAATCCCCTATAGTGAGTCGTATTAATTTC |
| 80 | crRNA_ | GTTTAACATTCGGGTATCGTCCTTACGAGTTTTAGTCCCCTTCGTT |
| | Barcode_22 | TTTGGGGTAGTCTAAATCCCCTATAGTGAGTCGTATTAATTTC |
| 81 | crRNA_ | GCAATATTACTAGTCCGTGTCAGCCAGAGTTTTAGTCCCCTTCGTT |
| | Barcode_23 | TTTGGGGTAGTCTAAATCCCCTATAGTGAGTCGTATTAATTTC |
| 82 | crRNA_ | CCGACTAGGTCGGCGATACCATAGCACCGTTTTAGTCCCCTTCGTT |
| | Barcode_24 | TTTGGGGTAGTCTAAATCCCCTATAGTGAGTCGTATTAATTTC |
| 83 | crRNA_ | GATGACGCCTCACGTCCGTTGCCCCCTAGTTTTAGTCCCCTTCGTT |
| | Barcode_25 | TTTGGGGTAGTCTAAATCCCCTATAGTGAGTCGTATTAATTTC |
| 84 | crRNA_ | GCTCCGTAGCAATTGATAAACCGCTGAGGTTTTAGTCCCCTTCGTT |
| | Barcode_26 | TTTGGGGTAGTCTAAATCCCCTATAGTGAGTCGTATTAATTTC |
| 85 | crRNA_ | CTATCGGATTTTTAGATAAATTACGAGGGTTTTAGTCCCCTTCGTT |
| | Barcode_27 | TTTGGGGTAGTCTAAATCCCCTATAGTGAGTCGTATTAATTTC |
| 86 | crRNA_ | TTTTTAATCGGTAGCGACGATATATGACGTTTTAGTCCCCTTCGTT |
| | Barcode_28 | TTTGGGGTAGTCTAAATCCCCTATAGTGAGTCGTATTAATTTC |
| 87 | crRNA_ | TTGATTCGTGGGGATCCGCGGATAGAAGGTTTTAGTCCCCTTCGTT |
| | Barcode_29 | TTTGGGGTAGTCTAAATCCCCTATAGTGAGTCGTATTAATTTC |
| 88 | crRNA_ | CTAATATCTGATCCGTTATAACACCACAGTTTTAGTCCCCTTCGTT |
| | Barcode_30 | TTTGGGGTAGTCTAAATCCCCTATAGTGAGTCGTATTAATTTC |
| 89 | crRNA_ | TTGCGGGCCTGGACATTCAATCGCATACGTTTTAGTCCCCTTCGTT |
| | Barcode_31 | TTTGGGGTAGTCTAAATCCCCTATAGTGAGTCGTATTAATTTC |
| 90 | crRNA_ | GCCTAGACCAAAAGTCGCCTTCAGGGCAGTTTTAGTCCCCTTCGTT |
| | Barcode_32 | TTTGGGGTAGTCTAAATCCCCTATAGTGAGTCGTATTAATTTC |
| 91 | crRNA_ | ATAGGTAGGTAACGGAACTGACCGATAAGTTTTAGTCCCCTTCGT |
| | Barcode_33 | TTTTGGGGTAGTCTAAATCCCCTATAGTGAGTCGTATTAATTTC |
| 92 | crRNA_ | CGTACTCCTTACGCCTTACACGGACAATGTTTTAGTCCCCTTCGTT |
| | Barcode_34 | TTTGGGGTAGTCTAAATCCCCTATAGTGAGTCGTATTAATTTC |
| 93 | crRNA_ | GATTCTCTGGTCATGAGGGAAGATCGACGTTTTAGTCCCCTTCGTT |
| | Barcode_35 | TTTGGGGTAGTCTAAATCCCCTATAGTGAGTCGTATTAATTTC |
| 94 | crRNA_ | TTTGGACGGGGTCACTACTCTTTACAGCGTTTTAGTCCCCTTCGTT |
| | Barcode_36 | TTTGGGGTAGTCTAAATCCCCTATAGTGAGTCGTATTAATTTC |
| 95 | crRNA_ | TTCATGTATGCTGTCCTACCGCAGTAGCGTTTTAGTCCCCTTCGTT |
| | Barcode_37 | TTTGGGGTAGTCTAAATCCCCTATAGTGAGTCGTATTAATTTC |
| 96 | crRNA_ | TACATAAGGTCGTCCTCCGGTCACGCCCGTTTTAGTCCCCTTCGTT |
| | Barcode_38 | TTTGGGGTAGTCTAAATCCCCTATAGTGAGTCGTATTAATTTC |
| 97 | crRNA_ | CAGGCCGCTACTCAAGCGGGGATAGCTTGTTTTAGTCCCCTTCGTT |
| | Barcode_39 | TTTGGGGTAGTCTAAATCCCCTATAGTGAGTCGTATTAATTTC |
| 98 | crRNA_ | CGCTTAGCTAGATTGCCCCTGGAAGCGCGTTTTAGTCCCCTTCGTT |
| | Barcode_40 | TTTGGGGTAGTCTAAATCCCCTATAGTGAGTCGTATTAATTTC |
| 99 | crRNA_ | CGGAGTGTTTGCGAATCCCGAGCATTAGGTTTTAGTCCCCTTCGTT |
| | Barcode_41 | TTTGGGGTAGTCTAAATCCCCTATAGTGAGTCGTATTAATTTC |
| 100 | crRNA_ | GTCTTTCGAATCGTTCCTTATAACCCGCGTTTTAGTCCCCTTCGTTT |
| | Barcode_42 | TTGGGGTAGTCTAAATCCCCTATAGTGAGTCGTATTAATTTC |
| 101 | crRNA_ | AGGCTGAATCCTCCGGCGGCTGTTCTAAGTTTTAGTCCCCTTCGTT |
| | Barcode__43 | TTTGGGGTAGTCTAAATCCCCTATAGTGAGTCGTATTAATTTC |
| 102 | crRNA_ | CAGCCAAACGCTAGCAAAATAGGATATAGTTTTAGTCCCCTTCGT |
| | Barcode_44 | TTTTGGGGTAGTCTAAATCCCCTATAGTGAGTCGTATTAATTTC |
| 103 | crRNA_ | CAGACGCTGTCTACCTAGCAGAAGGGCTGTTTTAGTCCCCTTCGTT |
| | Barcode_45 | TTTGGGGTAGTCTAAATCCCCTATAGTGAGTCGTATTAATTTC |
| 104 | crRNA_ | AAGGTATAATGTTCAAGCGTGGTGGCTAGTTTTAGTCCCCTTCGTT |
| | Barcode_46 | TTTGGGGTAGTCTAAATCCCCTATAGTGAGTCGTATTAATTTC |
| 105 | crRNA_ | ACTGATCTCGGCGTAAATACACCGCAGAGTTTTAGTCCCCTTCGTT |
| | Barcode_47 | TTTGGGGTAGTCTAAATCCCCTATAGTGAGTCGTATTAATTTC |
| 106 | crRNA_ | TATGTGTATCCTAAGGTACCTTTACGGGGTTTTAGTCCCCTTCGTT |
| | Barcode_48 | TTTGGGGTAGTCTAAATCCCCTATAGTGAGTCGTATTAATTTC |
| 107 | crRNA_ | ACCGTCCTAGTGCGGATAGTGGGACATGGTTTTAGTCCCCTTCGTT |
| | Barcode_49 | TTTGGGGTAGTCTAAATCCCCTATAGTGAGTCGTATTAATTTC |
| 108 | crRNA_ | GTCGATACGGGTCGCTAGATAGGTTGGGGTTTTAGTCCCCTTCGTT |
| | Barcode_50 | TTTGGGGTAGTCTAAATCCCCTATAGTGAGTCGTATTAATTTC |
| 109 | crRNA_ | ACAGTCAGAGGCGCTTCAATCGTCGCCCGTTTTAGTCCCCTTCGTT |
| | Barcode_51 | TTTGGGGTAGTCTAAATCCCCTATAGTGAGTCGTATTAATTTC |
| 110 | crRNA_ | GCGACTCATTTAATTTCAACGGGTAGAAGTTTTAGTCCCCTTCGTT |
| | Barcode_52 | TTTGGGGTAGTCTAAATCCCCTATAGTGAGTCGTATTAATTTC |
| 111 | crRNA_ | TAGACAGAGGGAGCTATATTGCGCGGCGGTTTTAGTCCCCTTCGT |
| | Barcode_53 | TTTTGGGGTAGTCTAAATCCCCTATAGTGAGTCGTATTAATTTC |
| 112 | crRNA_ | GGCATTCAATAGCCAGTAACCTTAGACAGTTTTAGTCCCCTTCGTT |
| | Barcode_54 | TTTGGGGTAGTCTAAATCCCCTATAGTGAGTCGTATTAATTTC |
| 113 | crRNA_ | ACAAAAGTTGGCATTTCGTAGACAAAGCGTTTTAGTCCCCTTCGTT |
| | Barcode_55 | TTTGGGGTAGTCTAAATCCCCTATAGTGAGTCGTATTAATTTC |
| 114 | crRNA_ | TTGCATACTTTGCAGGTTAGGGCCGAGAGTTTTAGTCCCCTTCGTT |
| | Barcode_56 | TTTGGGGTAGTCTAAATCCCCTATAGTGAGTCGTATTAATTTC |
| 115 | crRNA_ | ACCGGAGTTCAAACGACTTTGGGTCCCTGTTTTAGTCCCCTTCGTT |
| | Barcode_57 | TTTGGGGTAGTCTAAATCCCCTATAGTGAGTCGTATTAATTTC |
| 116 | crRNA_ | CACTGGTAGGCTCGCGGGTAAATAGAGCGTTTTAGTCCCCTTCGT |
| | Barcode_58 | TTTTGGGGTAGTCTAAATCCCCTATAGTGAGTCGTATTAATTTC |
| 117 | crRNA_ | CCGAAGCTCAGTGTGATGTGCCTTCCGTGTTTTAGTCCCCTTCGTT |
| | Barcode_59 | TTTGGGGTAGTCTAAATCCCCTATAGTGAGTCGTATTAATTTC |
| 118 | crRNA_ | GTACGTATGACTGTGTCCCAGCTATGAAGTTTTAGTCCCCTTCGTT |
| | Barcode_60 | TTTGGGGTAGTCTAAATCCCCTATAGTGAGTCGTATTAATTTC |
| 119 | crRNA_ | GGACCTTCACGTCAGTCCCGCTGACCTTGTTTTAGTCCCCTTCGTT |
| | Barcode_61 | TTTGGGGTAGTCTAAATCCCCTATAGTGAGTCGTATTAATTTC |
| 120 | crRNA_ | GACCAATTATGTTGGCCACGCCACATTAGTTTTAGTCCCCTTCGTT |
| | Barcode_62 | TTTGGGGTAGTCTAAATCCCCTATAGTGAGTCGTATTAATTTC |
| 121 | crRNA_ | GTTTTCAAGGGCTCGACACAGCAGAACGGTTTTAGTCCCCTTCGTT |
| | Barcode_63 | TTTGGGGTAGTCTAAATCCCCTATAGTGAGTCGTATTAATTTC |
| 122 | crRNA_ | AACCCACTATAGAAGGGCCTTCGAGATTGTTTTAGTCCCCTTCGTT |
| | Barcode_64 | TTTGGGGTAGTCTAAATCCCCTATAGTGAGTCGTATTAATTTC |
| 123 | crRNA_ | GGAACCGTCAATTCCGCACTAATAATTTGTTTTAGTCCCCTTCGTT |
| | Barcode_65 | TTTGGGGTAGTCTAAATCCCCTATAGTGAGTCGTATTAATTTC |
| 124 | crRNA_ | CATGGCTCCGAAACGCCGTGCAAAGTGCGTTTTAGTCCCCTTCGTT |
| | Barcode_66 | TTTGGGGTAGTCTAAATCCCCTATAGTGAGTCGTATTAATTTC |
| 125 | crRNA_ | GCTTTCTCATTGTTACTCAGCGCACATTGTTTTAGTCCCCTTCGTTT |
| | Barcode_67 | TTGGGGTAGTCTAAATCCCCTATAGTGAGTCGTATTAATTTC |
| 126 | crRNA_ | TCAATCCTGAGGCCGATACTAACAGTCGGTTTTAGTCCCCTTCGTT |
| | Barcode_68 | TTTGGGGTAGTCTAAATCCCCTATAGTGAGTCGTATTAATTTC |
| 127 | crRNA_ | ATTCTGGCTATACCGAGCCCGGACTACTGTTTTAGTCCCCTTCGTT |
| | Barcode_69 | TTTGGGGTAGTCTAAATCCCCTATAGTGAGTCGTATTAATTTC |
| 128 | crRNA_ | CAACCATGCTTCGGATAGCAATACCACGGTTTTAGTCCCCTTCGTT |
| | Barcode_70 | TTTGGGGTAGTCTAAATCCCCTATAGTGAGTCGTATTAATTTC |
| 129 | crRNA_ | CTTGTCTCAAGAATAGTGCCCTATTCGTGTTTTAGTCCCCTTCGTT |
| | Barcode_71 | TTTGGGGTAGTCTAAATCCCCTATAGTGAGTCGTATTAATTTC |
| 130 | crRNA_ | CCGTAGGTAGAAACATGTATGGCGCAGAGTTTTAGTCCCCTTCGT |
| | Barcode_72 | TTTTGGGGTAGTCTAAATCCCCTATAGTGAGTCGTATTAATTTC |
| 131 | crRNA_ | CAGGGCCATGTCGTACTAGTAGCCTGCCGTTTTAGTCCCCTTCGTT |
| | Barcode_73 | TTTGGGGTAGTCTAAATCCCCTATAGTGAGTCGTATTAATTTC |
| 132 | crRNA_ | CGAGCCCCCCAAAAAGTCGGGCTACCGCGTTTTAGTCCCCTTCGT |
| | Barcode_74 | TTTTGGGGTAGTCTAAATCCCCTATAGTGAGTCGTATTAATTTC |
| 133 | crRNA_ | CATATAGGTACATGAAGCCCTACTGTATGTTTTAGTCCCCTTCGTT |
| | Barcode_75 | TTTGGGGTAGTCTAAATCCCCTATAGTGAGTCGTATTAATTTC |
| 134 | crRNA_ | CATACCGGATGGTAAACTCCGCGAACGCGTTTTAGTCCCCTTCGTT |
| | Barcode_76 | TTTGGGGTAGTCTAAATCCCCTATAGTGAGTCGTATTAATTTC |
| 135 | crRNA_ | TGCAATGACCAGGCGATACGAATCGTAGGTTTTAGTCCCCTTCGT |
| | Barcode_77 | TTTTGGGGTAGTCTAAATCCCCTATAGTGAGTCGTATTAATTTC |
| 136 | crRNA_ | CTCCCCCCCGCAGACACGCACGTGTTGCGTTTTAGTCCCCTTCGTT |
| | Barcode_78 | TTTGGGGTAGTCTAAATCCCCTATAGTGAGTCGTATTAATTTC |
| 137 | crRNA_ | TTTGTGGTTAGCCTTGTCCCGGAACAGAGTTTTAGTCCCCTTCGTT |
| | Barcode_79 | TTTGGGGTAGTCTAAATCCCCTATAGTGAGTCGTATTAATTTC |
| 138 | crRNA_ | CCCGGGTTGATGAGGAGTCACTCAACGAGTTTTAGTCCCCTTCGTT |
| | Barcode_80 | TTTGGGGTAGTCTAAATCCCCTATAGTGAGTCGTATTAATTTC |
| 139 | crRNA_ | ATCCATGTGCGGGGAGAAGTCTGGTGGAGTTTTAGTCCCCTTCGT |
| | Barcode_81 | TTTTGGGGTAGTCTAAATCCCCTATAGTGAGTCGTATTAATTTC |
| 140 | crRNA_ | CCCCGGTAATTTCGATGTTCGCCCCGGCGTTTTAGTCCCCTTCGTT |
| | Barcode_82 | TTTGGGGTAGTCTAAATCCCCTATAGTGAGTCGTATTAATTTC |
| 141 | crRNA_ | TGGGCTGCTAGTAAGTCGGGAGCCCTTAGTTTTAGTCCCCTTCGTT |
| | Barcode_83 | TTTGGGGTAGTCTAAATCCCCTATAGTGAGTCGTATTAATTTC |
| 142 | crRNA_ | CAGGCCGCTAACCTAAACTCGACCTCGAGTTTTAGTCCCCTTCGTT |
| | Barcode_84 | TTTGGGGTAGTCTAAATCCCCTATAGTGAGTCGTATTAATTTC |
| 143 | crRNA_ | CAACGCAAGCGCTCTTGGTTTAAGCTGTGTTTTAGTCCCCTTCGTT |
| | Barcode_85 | TTTGGGGTAGTCTAAATCCCCTATAGTGAGTCGTATTAATTTC |
| 144 | crRNA_ | GGCACACAACGATCTCCAAATGAACCAAGTTTTAGTCCCCTTCGT |
| | Barcode_86 | TTTTGGGGTAGTCTAAATCCCCTATAGTGAGTCGTATTAATTTC |
| 145 | crRNA_ | GTAGCGCGACCTCTTTAACTGTAGAAGGGTTTTAGTCCCCTTCGTT |
| | Barcode_87 | TTTGGGGTAGTCTAAATCCCCTATAGTGAGTCGTATTAATTTC |
| 146 | crRNA_ | GCTTATCGCATTCCGGGTGAAAACCTAGGTTTTAGTCCCCTTCGTT |
| | Barcode_88 | TTTGGGGTAGTCTAAATCCCCTATAGTGAGTCGTATTAATTTC |
| 147 | crRNA_ | AGGGTAATGAAGCCCCGGGATCGAACATGTTTTAGTCCCCTTCGT |
| | Barcode_89 | TTTTGGGGTAGTCTAAATCCCCTATAGTGAGTCGTATTAATTTC |
| 148 | crRNA_ | TAACTAGCGGCCTATACCCGCTGCACATGTTTTAGTCCCCTTCGTT |
| | Barcode_90 | TTTGGGGTAGTCTAAATCCCCTATAGTGAGTCGTATTAATTTC |
| 149 | crRNA_ | CAGAGATAGGTCTCGACACTTACGCGGTGTTTTAGTCCCCTTCGTT |
| | Barcode_91 | TTTGGGGTAGTCTAAATCCCCTATAGTGAGTCGTATTAATTTC |
| 150 | crRNA_ | GCCTAACGGCTGCAGCACGATTGCGCAAGTTTTAGTCCCCTTCGTT |
| | Barcode_92 | TTTGGGGTAGTCTAAATCCCCTATAGTGAGTCGTATTAATTTC |
| 151 | crRNA_ | GACGCCATTAAGGTGGGTCACCTTTTCAGTTTTAGTCCCCTTCGTT |
| | Barcode_93 | TTTGGGGTAGTCTAAATCCCCTATAGTGAGTCGTATTAATTTC |
| 152 | crRNA_ | GCATATGGCTAAATTCGCTAGTCGAGCGGTTTTAGTCCCCTTCGTT |
| | Barcode_94 | TTTGGGGTAGTCTAAATCCCCTATAGTGAGTCGTATTAATTTC |
| 153 | Group 2 | GTCGACCTGCAGGCATGCAAGCGTTTTAGTCCCCTTCGTTTTTGGG |
| | crRNA | GTAGTCTAAATCCCCTATAGTGAGTCGTATTAATTTC |

TABLE 5
Exemplary barcode regions used herein.
List of unique barcode sequences
used herein; see e.g., FIG. 7A for
more detailed barcode design.

| SEQ | | |
|---|---|---|
| ID | | |
| NO: | Name | Sequence |
| 5 | Barcode_1 | TCGTCAGTCGTAACTTGGGAACGCACAT |
| 6 | Barcode_2 | CTTTGGGGTTGGATAAATCTGTCGTGGT |
| 7 | Barcode_3 | TGAATAAGCGCGGTCCCTAATGTTGGTG |
| 8 | Barcode_4 | ACCGGTTTATAGTTCGAACAGTCGCAGC |
| 9 | Barcode_5 | CCCCGTGTGGTAACTACGCAAGCCTAAC |
| 10 | Barcode_6 | ACGTAGGGGGGCGCGTAACCACTAGCTC |
| 11 | Barcode_7 | AGTGTCCCTTATTCTACTTTGAATTATC |
| 12 | Barcode_8 | GAGTTACGGGTCAGGATCATTGCGCAGG |
| 13 | Barcode_9 | GTGAGTCCGGCCTATCACGTTTGGTAGG |
| 14 | Barcode_10 | TCAGGGGAAACGAGTTAAGCAGAGGCAG |
| 15 | Barcode_11 | CTTGGTCCAATCGTATGCTAAGAGTAGC |
| 16 | Barcode_12 | TCGGTTGCACGCGCGTCCTGCACGCTCG |
| 17 | Barcode_13 | GTTCAAAAGCGGGAGTCCCGGTGAAACC |
| 18 | Barcode_14 | GAGGGCTTCTACGAAATTGCCTCACCAT |
| 19 | Barcode_15 | GGGATGCCCTGATAACGTAGAGTCTCAG |
| 20 | Barcode_16 | TAGTACGGGCCACGATCTAAGTCGGCGG |
| 21 | Barcode_17 | GTATGTGGGTACGATTGTAGGCTAGAAA |
| 22 | Barcode_18 | AGAACTACCTACGTGGCCACGCAAGCCC |
| 23 | Barcode_19 | TGCCTCAGAATGGGGTTTAACGTTCAGG |
| 24 | Barcode_20 | ATGTTGGGACGCCAGACTGACACGCAAA |
| 25 | Barcode_21 | CCTACACCTTCACCGAGTGTGGAGCAAG |
| 26 | Barcode_22 | GTTTAACATTCGGGTATCGTCCTTACGA |
| 27 | Barcode_23 | GCAATATTACTAGTCCGTGTCAGCCAGA |
| 28 | Barcode_24 | CCGACTAGGTCGGCGATACCATAGCACC |
| 29 | Barcode_25 | GATGACGCCTCACGTCCGTTGCCCCCTA |
| 30 | Barcode_26 | GCTCCGTAGCAATTGATAAACCGCTGAG |
| 31 | Barcode_27 | CTATCGGATTTTTAGATAAATTACGAGG |
| 154 | Barcode_28 | TTTTTAATCGGTAGCGACGATATATGAC |
| 155 | Barcode_29 | TTGATTCGTGGGGATCCGCGGATAGAAG |
| 156 | Barcode_30 | CTAATATCTGATCCGTTATAACACCACA |
| 157 | Barcode_31 | TTGCGGGCCTGGACATTCAATCGCATAC |
| 158 | Barcode_32 | GCCTAGACCAAAAGTCGCCTTCAGGGCA |
| 159 | Barcode_33 | ATAGGTAGGTAACGGAACTGACCGATAA |
| 160 | Barcode_34 | CGTACTCCTTACGCCTTACACGGACAAT |
| 161 | Barcode_35 | GATTCTCTGGTCATGAGGGAAGATCGAC |
| 162 | Barcode_36 | TTTGGACGGGGTCACTACTCTTTACAGC |
| 163 | Barcode_37 | TTCATGTATGCTGTCCTACCGCAGTAGC |
| 164 | Barcode_38 | TACATAAGGTCGTCCTCCGGTCACGCCC |
| 165 | Barcode_39 | CAGGCCGCTACTCAAGCGGGGATAGCTT |
| 166 | Barcode_40 | CGCTTAGCTAGATTGCCCCTGGAAGCGC |
| 167 | Barcode_41 | CGGAGTGTTTGCGAATCCCGAGCATTAG |
| 168 | Barcode_42 | GTCTTTCGAATCGTTCCTTATAACCCGC |
| 169 | Barcode_43 | AGGCTGAATCCTCCGGCGGCTGTTCTAA |
| 170 | Barcode_44 | CAGCCAAACGCTAGCAAAATAGGATATA |
| 171 | Barcode_45 | CAGACGCTGTCTACCTAGCAGAAGGGCT |
| 172 | Barcode_46 | AAGGTATAATGTTCAAGCGTGGTGGCTA |
| 173 | Barcode_47 | ACTGATCTCGGCGTAAATACACCGCAGA |
| 174 | Barcode_48 | TATGTGTATCCTAAGGTACCTTTACGGG |
| 175 | Barcode_49 | ACCGTCCTAGTGCGGATAGTGGGACATG |
| 176 | Barcode_50 | GTCGATACGGGTCGCTAGATAGGTTGGG |
| 177 | Barcode_51 | ACAGTCAGAGGCGCTTCAATCGTCGCCC |
| 178 | Barcode_52 | GCGACTCATTTAATTTCAACGGGTAGAA |
| 179 | Barcode_53 | TAGACAGAGGGAGCTATATTGCGCGGCG |
| 180 | Barcode_54 | GGCATTCAATAGCCAGTAACCTTAGACA |
| 181 | Barcode_55 | ACAAAAGTTGGCATTTCGTAGACAAAGC |
| 182 | Barcode_56 | TTGCATACTTTGCAGGTTAGGGCCGAGA |
| 183 | Barcode_57 | ACCGGAGTTCAAACGACTTTGGGTCCCT |
| 184 | Barcode_58 | CACTGGTAGGCTCGCGGGTAAATAGAGC |
| 185 | Barcode_59 | CCGAAGCTCAGTGTGATGTGCCTTCCGT |
| 186 | Barcode_60 | GTACGTATGACTGTGTCCCAGCTATGAA |
| 187 | Barcode_61 | GGACCTTCACGTCAGTCCCGCTGACCTT |
| 188 | Barcode_62 | GACCAATTATGTTGGCCACGCCACATTA |
| 189 | Barcode_63 | GTTTTCAAGGGCTCGACACAGCAGAACG |
| 190 | Barcode_64 | AACCCACTATAGAAGGGCCTTCGAGATT |
| 191 | Barcode_65 | GGAACCGTCAATTCCGCACTAATAATTT |
| 192 | Barcode_66 | CATGGCTCCGAAACGCCGTGCAAAGTGC |
| 193 | Barcode_67 | GCTTTCTCATTGTTACTCAGCGCACATT |
| 194 | Barcode_68 | TCAATCCTGAGGCCGATACTAACAGTCG |
| 195 | Barcode_69 | ATTCTGGCTATACCGAGCCCGGACTACT |
| 196 | Barcode_70 | CAACCATGCTTCGGATAGCAATACCACG |
| 197 | Barcode_71 | CTTGTCTCAAGAATAGTGCCCTATTCGT |
| 198 | Barcode_72 | CCGTAGGTAGAAACATGTATGGCGCAGA |
| 199 | Barcode_73 | CAGGGCCATGTCGTACTAGTAGCCTGCC |
| 200 | Barcode_74 | CGAGCCCCCCAAAAAGTCGGGCTACCGC |
| 201 | Barcode_75 | CATATAGGTACATGAAGCCCTACTGTAT |
| 202 | Barcode_76 | CATACCGGATGGTAAACTCCGCGAACGC |
| 203 | Barcode_77 | TGCAATGACCAGGCGATACGAATCGTAG |
| 204 | Barcode_78 | CTCCCCCCCGCAGACACGCACGTGTTGC |
| 205 | Barcode_79 | TTTGTGGTTAGCCTTGTCCCGGAACAGA |
| 206 | Barcode_80 | CCCGGGTTGATGAGGAGTCACTCAACGA |
| 207 | Barcode_81 | ATCCATGTGCGGGGAGAAGTCTGGTGGA |
| 208 | Barcode_82 | CCCCGGTAATTTCGATGTTCGCCCCGGC |
| 209 | Barcode_83 | TGGGCTGCTAGTAAGTCGGGAGCCCTTA |
| 210 | Barcode_84 | CAGGCCGCTAACCTAAACTCGACCTCGA |
| 211 | Barcode_85 | CAACGCAAGCGCTCTTGGTTTAAGCTGT |
| 212 | Barcode_86 | GGCACACAACGATCTCCAAATGAACCAA |
| 213 | Barcode_87 | GTAGCGCGACCTCTTTAACTGTAGAAGG |
| 214 | Barcode_88 | GCTTATCGCATTCCGGGTGAAAACCTAG |
| 215 | Barcode_89 | AGGGTAATGAAGCCCCGGGATCGAACAT |
| 216 | Barcode_90 | TAACTAGCGGCCTATACCCGCTGCACAT |
| 217 | Barcode_91 | CAGAGATAGGTCTCGACACTTACGCGGT |
| 218 | Barcode_92 | GCCTAACGGCTGCAGCACGATTGCGCAA |
| 219 | Barcode_93 | GACGCCATTAAGGTGGGTCACCTTTTCA |
| 220 | Barcode_94 | GCATATGGCTAAATTCGCTAGTCGAGCG |
| 221 | Universal 2 | GTCGACCTGCAGGCATGCAAGC |

TABLE 6
Exemplary genetic barcode elements used herein. List of barcoded DNA
megamer sequences used for SHERLOCK reactions.

| SEQ | | |
|---|---|---|
| ID | | |
| NO: | Name | Sequence |
| 222 | F_ | CTGGGATCCTCTAGAAATATGGATTACTTGGTAGAACAGAATTCTAATA |
| | Barcode_ | CGACTCACTATAGGGCTAGCTTCGTCAGTCGTAACTTGGGAACGCACAT |
| | 1 | TAGATCTCAGTCGACCTGCAGGCATGCAAGCTTGGCGTAATCATGGTCA |
| | | TAGCTGTTTCCTGTGTTTATCAG |
| 223 | F_ | CTGGGATCCTCTAGAAATATGGATTACTTGGTAGAACAGAATTCTAATA |
| | Barcode_ | CGACTCACTATAGGGCTAGCTCTTTGGGGTTGGATAAATCTGTCGTGGTT |
| | 2 | AGATCTCAGTCGACCTGCAGGCATGCAAGCTTGGCGTAATCATGGTCAT |
| | | AGCTGTTTCCTGTGTTTATCAG |
| 224 | F_ | CTGGGATCCTCTAGAAATATGGATTACTTGGTAGAACAGAATTCTAATA |
| | Barcode_ | CGACTCACTATAGGGCTAGCTTGAATAAGCGCGGTCCCTAATGTTGGTG |
| | 3 | TAGATCTCAGTCGACCTGCAGGCATGCAAGCTTGGCGTAATCATGGTCA |
| | | TAGCTGTTTCCTGTGTTTATCAG |
| 225 | F_ | CTGGGATCCTCTAGAAATATGGATTACTTGGTAGAACAGAATTCTAATA |
| | Barcode_ | CGACTCACTATAGGGCTAGCTACCGGTTTATAGTTCGAACAGTCGCAGC |
| | 4 | TAGATCTCAGTCGACCTGCAGGCATGCAAGCTTGGCGTAATCATGGTCA |
| | | TAGCTGTTTCCTGTGTTTATCAG |
| 226 | F_ | CTGGGATCCTCTAGAAATATGGATTACTTGGTAGAACAGAATTCTAATA |
| | Barcode_ | CGACTCACTATAGGGCTAGCTCCCCGTGTGGTAACTACGCAAGCCTAAC |
| | 5 | TAGATCTCAGTCGACCTGCAGGCATGCAAGCTTGGCGTAATCATGGTCA |
| | | TAGCTGTTTCCTGTGTTTATCAG |
| 227 | F_ | CTGGGATCCTCTAGAAATATGGATTACTTGGTAGAACAGAATTCTAATA |
| | Barcode_ | CGACTCACTATAGGGCTAGCTACGTAGGGGGGCGCGTAACCACTAGCTC |
| | 6 | TAGATCTCAGTCGACCTGCAGGCATGCAAGCTTGGCGTAATCATGGTCA |
| | | TAGCTGTTTCCTGTGTTTATCAG |
| 228 | F_ | CTGGGATCCTCTAGAAATATGGATTACTTGGTAGAACAGAATTCTAATA |
| | Barcode_ | CGACTCACTATAGGGCTAGCTAGTGTCCCTTATTCTACTTTGAATTATCT |
| | 7 | AGATCTCAGTCGACCTGCAGGCATGCAAGCTTGGCGTAATCATGGTCAT |
| | | AGCTGTTTCCTGTGTTTATCAG |
| 229 | F_ | CTGGGATCCTCTAGAAATATGGATTACTTGGTAGAACAGAATTCTAATA |
| | Barcode_ | CGACTCACTATAGGGCTAGCTGAGTTACGGGTCAGGATCATTGCGCAGG |
| | 8 | TAGATCTCAGTCGACCTGCAGGCATGCAAGCTTGGCGTAATCATGGTCA |
| | | TAGCTGTTTCCTGTGTTTATCAG |
| 230 | F_ | CTGGGATCCTCTAGAAATATGGATTACTTGGTAGAACAGAATTCTAATA |
| | Barcode_ | CGACTCACTATAGGGCTAGCTGTGAGTCCGGCCTATCACGTTTGGTAGG |
| | 9 | TAGATCTCAGTCGACCTGCAGGCATGCAAGCTTGGCGTAATCATGGTCA |
| | | TAGCTGTTTCCTGTGTTTATCAG |
| 231 | F_ | CTGGGATCCTCTAGAAATATGGATTACTTGGTAGAACAGAATTCTAATA |
| | Barcode_ | CGACTCACTATAGGGCTAGCTTCAGGGGAAACGAGTTAAGCAGAGGCA |
| | 10 | GTAGATCTCAGTCGACCTGCAGGCATGCAAGCTTGGCGTAATCATGGTC |
| | | ATAGCTGTTTCCTGTGTTTATCAG |
| 232 | F_ | CTGGGATCCTCTAGAAATATGGATTACTTGGTAGAACAGAATTCTAATA |
| | Barcode_ | CGACTCACTATAGGGCTAGCTCTTGGTCCAATCGTATGCTAAGAGTAGC |
| | 11 | TAGATCTCAGTCGACCTGCAGGCATGCAAGCTTGGCGTAATCATGGTCA |
| | | TAGCTGTTTCCTGTGTTTATCAG |
| 233 | F_ | CTGGGATCCTCTAGAAATATGGATTACTTGGTAGAACAGAATTCTAATA |
| | Barcode_ | CGACTCACTATAGGGCTAGCTTCGGTTGCACGCGCGTCCTGCACGCTCG |
| | 12 | TAGATCTCAGTCGACCTGCAGGCATGCAAGCTTGGCGTAATCATGGTCA |
| | | TAGCTGTTTCCTGTGTTTATCAG |
| 234 | F_ | CTGGGATCCTCTAGAAATATGGATTACTTGGTAGAACAGAATTCTAATA |
| | Barcode_ | CGACTCACTATAGGGCTAGCTGTTCAAAAGCGGGAGTCCCGGTGAAACC |
| | 13 | TAGATCTCAGTCGACCTGCAGGCATGCAAGCTTGGCGTAATCATGGTCA |
| | | TAGCTGTTTCCTGTGTTTATCAG |
| 235 | F_ | CTGGGATCCTCTAGAAATATGGATTACTTGGTAGAACAGAATTCTAATA |
| | Barcode_ | CGACTCACTATAGGGCTAGCTGAGGGCTTCTACGAAATTGCCTCACCAT |
| | 14 | TAGATCTCAGTCGACCTGCAGGCATGCAAGCTTGGCGTAATCATGGTCA |
| | | TAGCTGTTTCCTGTGTTTATCAG |
| 236 | F_ | CTGGGATCCTCTAGAAATATGGATTACTTGGTAGAACAGAATTCTAATA |
| | Barcode_ | CGACTCACTATAGGGCTAGCTGGGATGCCCTGATAACGTAGAGTCTCAG |
| | 15 | TAGATCTCAGTCGACCTGCAGGCATGCAAGCTTGGCGTAATCATGGTCA |
| | | TAGCTGTTTCCTGTGTTTATCAG |
| 237 | F_ | CTGGGATCCTCTAGAAATATGGATTACTTGGTAGAACAGAATTCTAATA |
| | Barcode_ | CGACTCACTATAGGGCTAGCTTAGTACGGGCCACGATCTAAGTCGGCGG |
| | 16 | TAGATCTCAGTCGACCTGCAGGCATGCAAGCTTGGCGTAATCATGGTCA |
| | | TAGCTGTTTCCTGTGTTTATCAG |
| 238 | F_ | CTGGGATCCTCTAGAAATATGGATTACTTGGTAGAACAGAATTCTAATA |
| | Barcode_ | CGACTCACTATAGGGCTAGCTGTATGTGGGTACGATTGTAGGCTAGAAA |
| | 17 | TAGATCTCAGTCGACCTGCAGGCATGCAAGCTTGGCGTAATCATGGTCA |
| | | TAGCTGTTTCCTGTGTTTATCAG |
| 239 | F_ | CTGGGATCCTCTAGAAATATGGATTACTTGGTAGAACAGAATTCTAATA |
| | Barcode_ | CGACTCACTATAGGGCTAGCTAGAACTACCTACGTGGCCACGCAAGCCC |
| | 18 | TAGATCTCAGTCGACCTGCAGGCATGCAAGCTTGGCGTAATCATGGTCA |
| | | TAGCTGTTTCCTGTGTTTATCAG |
| 240 | F_ | CTGGGATCCTCTAGAAATATGGATTACTTGGTAGAACAGAATTCTAATA |
| | Barcode_ | CGACTCACTATAGGGCTAGCTTGCCTCAGAATGGGGTTTAACGTTCAGG |
| | 19 | TAGATCTCAGTCGACCTGCAGGCATGCAAGCTTGGCGTAATCATGGTCA |
| | | TAGCTGTTTCCTGTGTTTATCAG |
| 241 | F_ | CTGGGATCCTCTAGAAATATGGATTACTTGGTAGAACAGAATTCTAATA |
| | Barcode_ | CGACTCACTATAGGGCTAGCTATGTTGGGACGCCAGACTGACACGCAAA |
| | 20 | TAGATCTCAGTCGACCTGCAGGCATGCAAGCTTGGCGTAATCATGGTCA |
| | | TAGCTGTTTCCTGTGTTTATCAG |
| 242 | F_ | CTGGGATCCTCTAGAAATATGGATTACTTGGTAGAACAGAATTCTAATA |
| | Barcode_ | CGACTCACTATAGGGCTAGCTCCTACACCTTCACCGAGTGTGGAGCAAG |
| | 21 | TAGATCTCAGTCGACCTGCAGGCATGCAAGCTTGGCGTAATCATGGTCA |
| | | TAGCTGTTTCCTGTGTTTATCAG |
| 243 | F_ | CTGGGATCCTCTAGAAATATGGATTACTTGGTAGAACAGAATTCTAATA |
| | Barcode_ | CGACTCACTATAGGGCTAGCTGTTTAACATTCGGGTATCGTCCTTACGAT |
| | 22 | AGATCTCAGTCGACCTGCAGGCATGCAAGCTTGGCGTAATCATGGTCAT |
| | | AGCTGTTTCCTGTGTTTATCAG |
| 244 | F_ | CTGGGATCCTCTAGAAATATGGATTACTTGGTAGAACAGAATTCTAATA |
| | Barcode_ | CGACTCACTATAGGGCTAGCTGCAATATTACTAGTCCGTGTCAGCCAGA |
| | 23 | TAGATCTCAGTCGACCTGCAGGCATGCAAGCTTGGCGTAATCATGGTCA |
| | | TAGCTGTTTCCTGTGTTTATCAG |
| 245 | F_ | CTGGGATCCTCTAGAAATATGGATTACTTGGTAGAACAGAATTCTAATA |
| | Barcode_ | CGACTCACTATAGGGCTAGCTCCGACTAGGTCGGCGATACCATAGCACC |
| | 24 | TAGATCTCAGTCGACCTGCAGGCATGCAAGCTTGGCGTAATCATGGTCA |
| | | TAGCTGTTTCCTGTGTTTATCAG |
| 246 | F_ | CTGGGATCCTCTAGAAATATGGATTACTTGGTAGAACAGAATTCTAATA |
| | Barcode_ | CGACTCACTATAGGGCTAGCTGATGACGCCTCACGTCCGTTGCCCCCTA |
| | 25 | TAGATCTCAGTCGACCTGCAGGCATGCAAGCTTGGCGTAATCATGGTCA |
| | | TAGCTGTTTCCTGTGTTTATCAG |
| 247 | F_ | CTGGGATCCTCTAGAAATATGGATTACTTGGTAGAACAGAATTCTAATA |
| | Barcode_ | CGACTCACTATAGGGCTAGCTGCTCCGTAGCAATTGATAAACCGCTGAG |
| | 26 | TAGATCTCAGTCGACCTGCAGGCATGCAAGCTTGGCGTAATCATGGTCA |
| | | TAGCTGTTTCCTGTGTTTATCAG |
| 248 | F_ | CTGGGATCCTCTAGAAATATGGATTACTTGGTAGAACAGAATTCTAATA |
| | Barcode_ | CGACTCACTATAGGGCTAGCTCTATCGGATTTTTAGATAAATTACGAGG |
| | 27 | TAGATCTCAGTCGACCTGCAGGCATGCAAGCTTGGCGTAATCATGGTCA |
| | | TAGCTGTTTCCTGTGTTTATCAG |
| 249 | F_ | CTGGGATCCTCTAGAAATATGGATTACTTGGTAGAACAGAATTCTAATA |
| | Barcode_ | CGACTCACTATAGGGCTAGCTTTTTTAATCGGTAGCGACGATATATGAC |
| | 28 | TAGATCTCAGTCGACCTGCAGGCATGCAAGCTTGGCGTAATCATGGTCA |
| | | TAGCTGTTTCCTGTGTTTATCAG |
| 250 | F_ | CTGGGATCCTCTAGAAATATGGATTACTTGGTAGAACAGAATTCTAATA |
| | Barcode_ | CGACTCACTATAGGGCTAGCTTTGATTCGTGGGGATCCGCGGATAGAAG |
| | 29 | TAGATCTCAGTCGACCTGCAGGCATGCAAGCTTGGCGTAATCATGGTCA |
| | | TAGCTGTTTCCTGTGTTTATCAG |
| 251 | F_ | CTGGGATCCTCTAGAAATATGGATTACTTGGTAGAACAGAATTCTAATA |
| | Barcode_ | CGACTCACTATAGGGCTAGCTCTAATATCTGATCCGTTATAACACCACA |
| | 30 | TAGATCTCAGTCGACCTGCAGGCATGCAAGCTTGGCGTAATCATGGTCA |
| | | TAGCTGTTTCCTGTGTTTATCAG |
| 252 | F_ | CTGGGATCCTCTAGAAATATGGATTACTTGGTAGAACAGAATTCTAATA |
| | Barcode_ | CGACTCACTATAGGGCTAGCTTTGCGGGCCTGGACATTCAATCGCATAC |
| | 31 | TAGATCTCAGTCGACCTGCAGGCATGCAAGCTTGGCGTAATCATGGTCA |
| | | TAGCTGTTTCCTGTGTTTATCAG |
| 253 | F_ | CTGGGATCCTCTAGAAATATGGATTACTTGGTAGAACAGAATTCTAATA |
| | Barcode_ | CGACTCACTATAGGGCTAGCTGCCTAGACCAAAAGTCGCCTTCAGGGCA |
| | 32 | TAGATCTCAGTCGACCTGCAGGCATGCAAGCTTGGCGTAATCATGGTCA |
| | | TAGCTGTTTCCTGTGTTTATCAG |
| 254 | F_ | CTGGGATCCTCTAGAAATATGGATTACTTGGTAGAACAGAATTCTAATA |
| | Barcode_ | CGACTCACTATAGGGCTAGCTATAGGTAGGTAACGGAACTGACCGATAA |
| | 33 | TAGATCTCAGTCGACCTGCAGGCATGCAAGCTTGGCGTAATCATGGTCA |
| | | TAGCTGTTTCCTGTGTTTATCAG |
| 255 | F_ | CTGGGATCCTCTAGAAATATGGATTACTTGGTAGAACAGAATTCTAATA |
| | Barcode_ | CGACTCACTATAGGGCTAGCTCGTACTCCTTACGCCTTACACGGACAAT |
| | 34 | TAGATCTCAGTCGACCTGCAGGCATGCAAGCTTGGCGTAATCATGGTCA |
| | | TAGCTGTTTCCTGTGTTTATCAG |
| 256 | F_ | CTGGGATCCTCTAGAAATATGGATTACTTGGTAGAACAGAATTCTAATA |
| | Barcode_ | CGACTCACTATAGGGCTAGCTGATTCTCTGGTCATGAGGGAAGATCGAC |
| | 35 | TAGATCTCAGTCGACCTGCAGGCATGCAAGCTTGGCGTAATCATGGTCA |
| | | TAGCTGTTTCCTGTGTTTATCAG |
| 257 | F_ | CTGGGATCCTCTAGAAATATGGATTACTTGGTAGAACAGAATTCTAATA |
| | Barcode_ | CGACTCACTATAGGGCTAGCTTTTGGACGGGGTCACTACTCTTTACAGCT |
| | 36 | AGATCTCAGTCGACCTGCAGGCATGCAAGCTTGGCGTAATCATGGTCAT |
| | | AGCTGTTTCCTGTGTTTATCAG |
| 258 | F_ | CTGGGATCCTCTAGAAATATGGATTACTTGGTAGAACAGAATTCTAATA |
| | Barcode_ | CGACTCACTATAGGGCTAGCTTTCATGTATGCTGTCCTACCGCAGTAGCT |
| | 37 | AGATCTCAGTCGACCTGCAGGCATGCAAGCTTGGCGTAATCATGGTCAT |
| | | AGCTGTTTCCTGTGTTTATCAG |
| 259 | F_ | CTGGGATCCTCTAGAAATATGGATTACTTGGTAGAACAGAATTCTAATA |
| | Barcode_ | CGACTCACTATAGGGCTAGCTTACATAAGGTCGTCCTCCGGTCACGCCC |
| | 38 | TAGATCTCAGTCGACCTGCAGGCATGCAAGCTTGGCGTAATCATGGTCA |
| | | TAGCTGTTTCCTGTGTTTATCAG |
| 260 | F_ | CTGGGATCCTCTAGAAATATGGATTACTTGGTAGAACAGAATTCTAATA |
| | Barcode_ | CGACTCACTATAGGGCTAGCTCAGGCCGCTACTCAAGCGGGGATAGCTT |
| | 39 | TAGATCTCAGTCGACCTGCAGGCATGCAAGCTTGGCGTAATCATGGTCA |
| | | TAGCTGTTTCCTGTGTTTATCAG |
| 261 | F_ | CTGGGATCCTCTAGAAATATGGATTACTTGGTAGAACAGAATTCTAATA |
| | Barcode_ | CGACTCACTATAGGGCTAGCTCGCTTAGCTAGATTGCCCCTGGAAGCGC |
| | 40 | TAGATCTCAGTCGACCTGCAGGCATGCAAGCTTGGCGTAATCATGGTCA |
| | | TAGCTGTTTCCTGTGTTTATCAG |
| 262 | F_ | CTGGGATCCTCTAGAAATATGGATTACTTGGTAGAACAGAATTCTAATA |
| | Barcode_ | CGACTCACTATAGGGCTAGCTCGGAGTGTTTGCGAATCCCGAGCATTAG |
| | 41 | TAGATCTCAGTCGACCTGCAGGCATGCAAGCTTGGCGTAATCATGGTCA |
| | | TAGCTGTTTCCTGTGTTTATCAG |
| 263 | F_ | CTGGGATCCTCTAGAAATATGGATTACTTGGTAGAACAGAATTCTAATA |
| | Barcode_ | CGACTCACTATAGGGCTAGCTGTCTTTCGAATCGTTCCTTATAACCCGCT |
| | 42 | AGATCTCAGTCGACCTGCAGGCATGCAAGCTTGGCGTAATCATGGTCAT |
| | | AGCTGTTTCCTGTGTTTATCAG |
| 264 | F_ | CTGGGATCCTCTAGAAATATGGATTACTTGGTAGAACAGAATTCTAATA |
| | Barcode_ | CGACTCACTATAGGGCTAGCTAGGCTGAATCCTCCGGCGGCTGTTCTAA |
| | 43 | TAGATCTCAGTCGACCTGCAGGCATGCAAGCTTGGCGTAATCATGGTCA |
| | | TAGCTGTTTCCTGTGTTTATCAG |
| 265 | F_ | CTGGGATCCTCTAGAAATATGGATTACTTGGTAGAACAGAATTCTAATA |
| | Barcode_ | CGACTCACTATAGGGCTAGCTCAGCCAAACGCTAGCAAAATAGGATATA |
| | 44 | TAGATCTCAGTCGACCTGCAGGCATGCAAGCTTGGCGTAATCATGGTCA |
| | | TAGCTGTTTCCTGTGTTTATCAG |
| 266 | F_ | CTGGGATCCTCTAGAAATATGGATTACTTGGTAGAACAGAATTCTAATA |
| | Barcode_ | CGACTCACTATAGGGCTAGCTCAGACGCTGTCTACCTAGCAGAAGGGCT |
| | 45 | TAGATCTCAGTCGACCTGCAGGCATGCAAGCTTGGCGTAATCATGGTCA |
| | | TAGCTGTTTCCTGTGTTTATCAG |
| 267 | F_ | CTGGGATCCTCTAGAAATATGGATTACTTGGTAGAACAGAATTCTAATA |
| | Barcode_ | CGACTCACTATAGGGCTAGCTAAGGTATAATGTTCAAGCGTGGTGGCTA |
| | 46 | TAGATCTCAGTCGACCTGCAGGCATGCAAGCTTGGCGTAATCATGGTCA |
| | | TAGCTGTTTCCTGTGTTTATCAG |
| 268 | F_ | CTGGGATCCTCTAGAAATATGGATTACTTGGTAGAACAGAATTCTAATA |
| | Barcode_ | CGACTCACTATAGGGCTAGCTACTGATCTCGGCGTAAATACACCGCAGA |
| | 47 | TAGATCTCAGTCGACCTGCAGGCATGCAAGCTTGGCGTAATCATGGTCA |
| | | TAGCTGTTTCCTGTGTTTATCAG |
| 269 | F_ | CTGGGATCCTCTAGAAATATGGATTACTTGGTAGAACAGAATTCTAATA |
| | Barcode_ | CGACTCACTATAGGGCTAGCTTATGTGTATCCTAAGGTACCTTTACGGGT |
| | 48 | AGATCTCAGTCGACCTGCAGGCATGCAAGCTTGGCGTAATCATGGTCAT |
| | | AGCTGTTTCCTGTGTTTATCAG |
| 270 | F_ | CTGGGATCCTCTAGAAATATGGATTACTTGGTAGAACAGAATTCTAATA |
| | Barcode_ | CGACTCACTATAGGGCTAGCTACCGTCCTAGTGCGGATAGTGGGACATG |
| | 49 | TAGATCTCAGTCGACCTGCAGGCATGCAAGCTTGGCGTAATCATGGTCA |
| | | TAGCTGTTTCCTGTGTTTATCAG |
| 271 | F_ | CTGGGATCCTCTAGAAATATGGATTACTTGGTAGAACAGAATTCTAATA |
| | Barcode_ | CGACTCACTATAGGGCTAGCTGTCGATACGGGTCGCTAGATAGGTTGGG |
| | 50 | TAGATCTCAGTCGACCTGCAGGCATGCAAGCTTGGCGTAATCATGGTCA |
| | | TAGCTGTTTCCTGTGTTTATCAG |
| 272 | F_ | CTGGGATCCTCTAGAAATATGGATTACTTGGTAGAACAGAATTCTAATA |
| | Barcode_ | CGACTCACTATAGGGCTAGCTACAGTCAGAGGCGCTTCAATCGTCGCCC |
| | 51 | TAGATCTCAGTCGACCTGCAGGCATGCAAGCTTGGCGTAATCATGGTCA |
| | | TAGCTGTTTCCTGTGTTTATCAG |
| 273 | F_ | CTGGGATCCTCTAGAAATATGGATTACTTGGTAGAACAGAATTCTAATA |
| | Barcode_ | CGACTCACTATAGGGCTAGCTGCGACTCATTTAATTTCAACGGGTAGAA |
| | 52 | TAGATCTCAGTCGACCTGCAGGCATGCAAGCTTGGCGTAATCATGGTCA |
| | | TAGCTGTTTCCTGTGTTTATCAG |
| 274 | F_ | CTGGGATCCTCTAGAAATATGGATTACTTGGTAGAACAGAATTCTAATA |
| | Barcode_ | CGACTCACTATAGGGCTAGCTTAGACAGAGGGAGCTATATTGCGCGGCG |
| | 53 | TAGATCTCAGTCGACCTGCAGGCATGCAAGCTTGGCGTAATCATGGTCA |
| | | TAGCTGTTTCCTGTGTTTATCAG |
| 275 | F_ | CTGGGATCCTCTAGAAATATGGATTACTTGGTAGAACAGAATTCTAATA |
| | Barcode_ | CGACTCACTATAGGGCTAGCTGGCATTCAATAGCCAGTAACCTTAGACA |
| | 54 | TAGATCTCAGTCGACCTGCAGGCATGCAAGCTTGGCGTAATCATGGTCA |
| | | TAGCTGTTTCCTGTGTTTATCAG |
| 276 | F_ | CTGGGATCCTCTAGAAATATGGATTACTTGGTAGAACAGAATTCTAATA |
| | Barcode_ | CGACTCACTATAGGGCTAGCTACAAAAGTTGGCATTTCGTAGACAAAGC |
| | 55 | TAGATCTCAGTCGACCTGCAGGCATGCAAGCTTGGCGTAATCATGGTCA |
| | | TAGCTGTTTCCTGTGTTTATCAG |
| 277 | F_ | CTGGGATCCTCTAGAAATATGGATTACTTGGTAGAACAGAATTCTAATA |
| | Barcode_ | CGACTCACTATAGGGCTAGCTTTGCATACTTTGCAGGTTAGGGCCGAGA |
| | 56 | TAGATCTCAGTCGACCTGCAGGCATGCAAGCTTGGCGTAATCATGGTCA |
| | | TAGCTGTTTCCTGTGTTTATCAG |
| 278 | F_ | CTGGGATCCTCTAGAAATATGGATTACTTGGTAGAACAGAATTCTAATA |
| | Barcode_ | CGACTCACTATAGGGCTAGCTACCGGAGTTCAAACGACTTTGGGTCCCT |
| | 57 | TAGATCTCAGTCGACCTGCAGGCATGCAAGCTTGGCGTAATCATGGTCA |
| | | TAGCTGTTTCCTGTGTTTATCAG |
| 279 | F_ | CTGGGATCCTCTAGAAATATGGATTACTTGGTAGAACAGAATTCTAATA |
| | Barcode_ | CGACTCACTATAGGGCTAGCTCACTGGTAGGCTCGCGGGTAAATAGAGC |
| | 58 | TAGATCTCAGTCGACCTGCAGGCATGCAAGCTTGGCGTAATCATGGTCA |
| | | TAGCTGTTTCCTGTGTTTATCAG |
| 280 | F_ | CTGGGATCCTCTAGAAATATGGATTACTTGGTAGAACAGAATTCTAATA |
| | Barcode_ | CGACTCACTATAGGGCTAGCTCCGAAGCTCAGTGTGATGTGCCTTCCGT |
| | 59 | TAGATCTCAGTCGACCTGCAGGCATGCAAGCTTGGCGTAATCATGGTCA |
| | | TAGCTGTTTCCTGTGTTTATCAG |
| 281 | F_ | CTGGGATCCTCTAGAAATATGGATTACTTGGTAGAACAGAATTCTAATA |
| | Barcode_ | CGACTCACTATAGGGCTAGCTGTACGTATGACTGTGTCCCAGCTATGAA |
| | 60 | TAGATCTCAGTCGACCTGCAGGCATGCAAGCTTGGCGTAATCATGGTCA |
| | | TAGCTGTTTCCTGTGTTTATCAG |
| 282 | F_ | CTGGGATCCTCTAGAAATATGGATTACTTGGTAGAACAGAATTCTAATA |
| | Barcode_ | CGACTCACTATAGGGCTAGCTGGACCTTCACGTCAGTCCCGCTGACCTTT |
| | 61 | AGATCTCAGTCGACCTGCAGGCATGCAAGCTTGGCGTAATCATGGTCAT |
| | | AGCTGTTTCCTGTGTTTATCAG |
| 283 | F_ | CTGGGATCCTCTAGAAATATGGATTACTTGGTAGAACAGAATTCTAATA |
| | Barcode_ | CGACTCACTATAGGGCTAGCTGACCAATTATGTTGGCCACGCCACATTA |
| | 62 | TAGATCTCAGTCGACCTGCAGGCATGCAAGCTTGGCGTAATCATGGTCA |
| | | TAGCTGTTTCCTGTGTTTATCAG |
| 284 | F_ | CTGGGATCCTCTAGAAATATGGATTACTTGGTAGAACAGAATTCTAATA |
| | Barcode_ | CGACTCACTATAGGGCTAGCTGTTTTCAAGGGCTCGACACAGCAGAACG |
| | 63 | TAGATCTCAGTCGACCTGCAGGCATGCAAGCTTGGCGTAATCATGGTCA |
| | | TAGCTGTTTCCTGTGTTTATCAG |
| 285 | F_ | CTGGGATCCTCTAGAAATATGGATTACTTGGTAGAACAGAATTCTAATA |
| | Barcode_ | CGACTCACTATAGGGCTAGCTAACCCACTATAGAAGGGCCTTCGAGATT |
| | 64 | TAGATCTCAGTCGACCTGCAGGCATGCAAGCTTGGCGTAATCATGGTCA |
| | | TAGCTGTTTCCTGTGTTTATCAG |
| 286 | F_ | CTGGGATCCTCTAGAAATATGGATTACTTGGTAGAACAGAATTCTAATA |
| | Barcode_ | CGACTCACTATAGGGCTAGCTGGAACCGTCAATTCCGCACTAATAATTT |
| | 65 | TAGATCTCAGTCGACCTGCAGGCATGCAAGCTTGGCGTAATCATGGTCA |
| | | TAGCTGTTTCCTGTGTTTATCAG |
| 287 | F_ | CTGGGATCCTCTAGAAATATGGATTACTTGGTAGAACAGAATTCTAATA |
| | Barcode_ | CGACTCACTATAGGGCTAGCTCATGGCTCCGAAACGCCGTGCAAAGTGC |
| | 66 | TAGATCTCAGTCGACCTGCAGGCATGCAAGCTTGGCGTAATCATGGTCA |
| | | TAGCTGTTTCCTGTGTTTATCAG |
| 288 | F_ | CTGGGATCCTCTAGAAATATGGATTACTTGGTAGAACAGAATTCTAATA |
| | Barcode_ | CGACTCACTATAGGGCTAGCTGCTTTCTCATTGTTACTCAGCGCACATTT |
| | 67 | AGATCTCAGTCGACCTGCAGGCATGCAAGCTTGGCGTAATCATGGTCAT |
| | | AGCTGTTTCCTGTGTTTATCAG |
| 289 | F_ | CTGGGATCCTCTAGAAATATGGATTACTTGGTAGAACAGAATTCTAATA |
| | Barcode_ | CGACTCACTATAGGGCTAGCTTCAATCCTGAGGCCGATACTAACAGTCG |
| | 68 | TAGATCTCAGTCGACCTGCAGGCATGCAAGCTTGGCGTAATCATGGTCA |
| | | TAGCTGTTTCCTGTGTTTATCAG |
| 290 | F_ | CTGGGATCCTCTAGAAATATGGATTACTTGGTAGAACAGAATTCTAATA |
| | Barcode_ | CGACTCACTATAGGGCTAGCTATTCTGGCTATACCGAGCCCGGACTACT |
| | 69 | TAGATCTCAGTCGACCTGCAGGCATGCAAGCTTGGCGTAATCATGGTCA |
| | | TAGCTGTTTCCTGTGTTTATCAG |
| 291 | F_ | CTGGGATCCTCTAGAAATATGGATTACTTGGTAGAACAGAATTCTAATA |
| | Barcode_ | CGACTCACTATAGGGCTAGCTCAACCATGCTTCGGATAGCAATACCACG |
| | 70 | TAGATCTCAGTCGACCTGCAGGCATGCAAGCTTGGCGTAATCATGGTCA |
| | | TAGCTGTTTCCTGTGTTTATCAG |
| 292 | F_ | CTGGGATCCTCTAGAAATATGGATTACTTGGTAGAACAGAATTCTAATA |
| | Barcode_ | CGACTCACTATAGGGCTAGCTCTTGTCTCAAGAATAGTGCCCTATTCGTT |
| | 71 | AGATCTCAGTCGACCTGCAGGCATGCAAGCTTGGCGTAATCATGGTCAT |
| | | AGCTGTTTCCTGTGTTTATCAG |
| 293 | F_ | CTGGGATCCTCTAGAAATATGGATTACTTGGTAGAACAGAATTCTAATA |
| | Barcode_ | CGACTCACTATAGGGCTAGCTCCGTAGGTAGAAACATGTATGGCGCAGA |
| | 72 | TAGATCTCAGTCGACCTGCAGGCATGCAAGCTTGGCGTAATCATGGTCA |
| | | TAGCTGTTTCCTGTGTTTATCAG |
| 294 | F_ | CTGGGATCCTCTAGAAATATGGATTACTTGGTAGAACAGAATTCTAATA |
| | Barcode_ | CGACTCACTATAGGGCTAGCTCAGGGCCATGTCGTACTAGTAGCCTGCC |
| | 73 | TAGATCTCAGTCGACCTGCAGGCATGCAAGCTTGGCGTAATCATGGTCA |
| | | TAGCTGTTTCCTGTGTTTATCAG |
| 295 | F_ | CTGGGATCCTCTAGAAATATGGATTACTTGGTAGAACAGAATTCTAATA |
| | Barcode_ | CGACTCACTATAGGGCTAGCTCGAGCCCCCCAAAAAGTCGGGCTACCGC |
| | 74 | TAGATCTCAGTCGACCTGCAGGCATGCAAGCTTGGCGTAATCATGGTCA |
| | | TAGCTGTTTCCTGTGTTTATCAG |
| 296 | F_ | CTGGGATCCTCTAGAAATATGGATTACTTGGTAGAACAGAATTCTAATA |
| | Barcode_ | CGACTCACTATAGGGCTAGCTCATATAGGTACATGAAGCCCTACTGTAT |
| | 75 | TAGATCTCAGTCGACCTGCAGGCATGCAAGCTTGGCGTAATCATGGTCA |
| | | TAGCTGTTTCCTGTGTTTATCAG |
| 297 | F_ | CTGGGATCCTCTAGAAATATGGATTACTTGGTAGAACAGAATTCTAATA |
| | Barcode_ | CGACTCACTATAGGGCTAGCTCATACCGGATGGTAAACTCCGCGAACGC |
| | 76 | TAGATCTCAGTCGACCTGCAGGCATGCAAGCTTGGCGTAATCATGGTCA |
| | | TAGCTGTTTCCTGTGTTTATCAG |
| 298 | F_ | CTGGGATCCTCTAGAAATATGGATTACTTGGTAGAACAGAATTCTAATA |
| | Barcode_ | CGACTCACTATAGGGCTAGCTTGCAATGACCAGGCGATACGAATCGTAG |
| | 77 | TAGATCTCAGTCGACCTGCAGGCATGCAAGCTTGGCGTAATCATGGTCA |
| | | TAGCTGTTTCCTGTGTTTATCAG |
| 299 | F_ | CTGGGATCCTCTAGAAATATGGATTACTTGGTAGAACAGAATTCTAATA |
| | Barcode_ | CGACTCACTATAGGGCTAGCTCTCCCCCCCGCAGACACGCACGTGTTGC |
| | 78 | TAGATCTCAGTCGACCTGCAGGCATGCAAGCTTGGCGTAATCATGGTCA |
| | | TAGCTGTTTCCTGTGTTTATCAG |
| 300 | F_ | CTGGGATCCTCTAGAAATATGGATTACTTGGTAGAACAGAATTCTAATA |
| | Barcode_ | CGACTCACTATAGGGCTAGCTTTTGTGGTTAGCCTTGTCCCGGAACAGA |
| | 79 | TAGATCTCAGTCGACCTGCAGGCATGCAAGCTTGGCGTAATCATGGTCA |
| | | TAGCTGTTTCCTGTGTTTATCAG |
| 301 | F_ | CTGGGATCCTCTAGAAATATGGATTACTTGGTAGAACAGAATTCTAATA |
| | Barcode_ | CGACTCACTATAGGGCTAGCTCCCGGGTTGATGAGGAGTCACTCAACGA |
| | 80 | TAGATCTCAGTCGACCTGCAGGCATGCAAGCTTGGCGTAATCATGGTCA |
| | | TAGCTGTTTCCTGTGTTTATCAG |
| 302 | F_ | CTGGGATCCTCTAGAAATATGGATTACTTGGTAGAACAGAATTCTAATA |
| | Barcode_ | CGACTCACTATAGGGCTAGCTATCCATGTGCGGGGAGAAGTCTGGTGGA |
| | 81 | TAGATCTCAGTCGACCTGCAGGCATGCAAGCTTGGCGTAATCATGGTCA |
| | | TAGCTGTTTCCTGTGTTTATCAG |
| 303 | F_ | CTGGGATCCTCTAGAAATATGGATTACTTGGTAGAACAGAATTCTAATA |
| | Barcode_ | CGACTCACTATAGGGCTAGCTCCCCGGTAATTTCGATGTTCGCCCCGGCT |
| | 82 | AGATCTCAGTCGACCTGCAGGCATGCAAGCTTGGCGTAATCATGGTCAT |
| | | AGCTGTTTCCTGTGTTTATCAG |
| 304 | F_ | CTGGGATCCTCTAGAAATATGGATTACTTGGTAGAACAGAATTCTAATA |
| | Barcode_ | CGACTCACTATAGGGCTAGCTTGGGCTGCTAGTAAGTCGGGAGCCCTTA |
| | 83 | TAGATCTCAGTCGACCTGCAGGCATGCAAGCTTGGCGTAATCATGGTCA |
| | | TAGCTGTTTCCTGTGTTTATCAG |
| 305 | F_ | CTGGGATCCTCTAGAAATATGGATTACTTGGTAGAACAGAATTCTAATA |
| | Barcode_ | CGACTCACTATAGGGCTAGCTCAGGCCGCTAACCTAAACTCGACCTCGA |
| | 84 | TAGATCTCAGTCGACCTGCAGGCATGCAAGCTTGGCGTAATCATGGTCA |
| | | TAGCTGTTTCCTGTGTTTATCAG |
| 306 | F_ | CTGGGATCCTCTAGAAATATGGATTACTTGGTAGAACAGAATTCTAATA |
| | Barcode_ | CGACTCACTATAGGGCTAGCTCAACGCAAGCGCTCTTGGTTTAAGCTGT |
| | 85 | TAGATCTCAGTCGACCTGCAGGCATGCAAGCTTGGCGTAATCATGGTCA |
| | | TAGCTGTTTCCTGTGTTTATCAG |
| 307 | F_ | CTGGGATCCTCTAGAAATATGGATTACTTGGTAGAACAGAATTCTAATA |
| | Barcode_ | CGACTCACTATAGGGCTAGCTGGCACACAACGATCTCCAAATGAACCAA |
| | 86 | TAGATCTCAGTCGACCTGCAGGCATGCAAGCTTGGCGTAATCATGGTCA |
| | | TAGCTGTTTCCTGTGTTTATCAG |
| 308 | F_ | CTGGGATCCTCTAGAAATATGGATTACTTGGTAGAACAGAATTCTAATA |
| | Barcode_ | CGACTCACTATAGGGCTAGCTGTAGCGCGACCTCTTTAACTGTAGAAGG |
| | 87 | TAGATCTCAGTCGACCTGCAGGCATGCAAGCTTGGCGTAATCATGGTCA |
| | | TAGCTGTTTCCTGTGTTTATCAG |
| 309 | F_ | CTGGGATCCTCTAGAAATATGGATTACTTGGTAGAACAGAATTCTAATA |
| | Barcode_ | CGACTCACTATAGGGCTAGCTGCTTATCGCATTCCGGGTGAAAACCTAG |
| | 88 | TAGATCTCAGTCGACCTGCAGGCATGCAAGCTTGGCGTAATCATGGTCA |
| | | TAGCTGTTTCCTGTGTTTATCAG |
| 310 1 | F_ | CTGGGATCCTCTAGAAATATGGATTACTTGGTAGAACAGAATTCTAATA |
| | Barcode_ | CGACTCACTATAGGGCTAGCTAGGGTAATGAAGCCCCGGGATCGAACAT |
| | 89 | TAGATCTCAGTCGACCTGCAGGCATGCAAGCTTGGCGTAATCATGGTCA |
| | | TAGCTGTTTCCTGTGTTTATCAG |
| 311 | F_ | CTGGGATCCTCTAGAAATATGGATTACTTGGTAGAACAGAATTCTAATA |
| | Barcode_ | CGACTCACTATAGGGCTAGCTTAACTAGCGGCCTATACCCGCTGCACAT |
| | 90 | TAGATCTCAGTCGACCTGCAGGCATGCAAGCTTGGCGTAATCATGGTCA |
| | | TAGCTGTTTCCTGTGTTTATCAG |
| 312 | F_ | CTGGGATCCTCTAGAAATATGGATTACTTGGTAGAACAGAATTCTAATA |
| | Barcode_ | CGACTCACTATAGGGCTAGCTCAGAGATAGGTCTCGACACTTACGCGGT |
| | 9111 | TAGATCTCAGTCGACCTGCAGGCATGCAAGCTTGGCGTAATCATGGTCA |
| | | TAGCTGTTTCCTGTGTTTATCAG |
| 313 | F_ | CTGGGATCCTCTAGAAATATGGATTACTTGGTAGAACAGAATTCTAATA |
| | Barcode_ | CGACTCACTATAGGGCTAGCTGCCTAACGGCTGCAGCACGATTGCGCAA |
| | 92 | TAGATCTCAGTCGACCTGCAGGCATGCAAGCTTGGCGTAATCATGGTCA |
| | | TAGCTGTTTCCTGTGTTTATCAG |
| 314 | F_ | CTGGGATCCTCTAGAAATATGGATTACTTGGTAGAACAGAATTCTAATA |
| | Barcode_ | CGACTCACTATAGGGCTAGCTGACGCCATTAAGGTGGGTCACCTTTTCA |
| | 93 | TAGATCTCAGTCGACCTGCAGGCATGCAAGCTTGGCGTAATCATGGTCA |
| | | TAGCTGTTTCCTGTGTTTATCAG |
| 315 | F_ | CTGGGATCCTCTAGAAATATGGATTACTTGGTAGAACAGAATTCTAATA |
| | Barcode_ | CGACTCACTATAGGGCTAGCTGCATATGGCTAAATTCGCTAGTCGAGCG |
| | 94 | TAGATCTCAGTCGACCTGCAGGCATGCAAGCTTGGCGTAATCATGGTCA |
| | | TAGCTGTTTCCTGTGTTTATCAG |

TABLE 7
Conditions for incubator scale experiments to test spore persistence.
Description matrix of conditions used in the incubator experiments,
including control conditions, perturbations, and sampling techniques.

| | | | | Direct | Transfer |
|---|---|---|---|---|---|
| Surface | | Control | Perturbation | Sample | Sample |
| Material | Count | Conditions | Conditions | Technique | Technique |
| Sand | 3 control | Temperature: 25 C. | Temperature: | Powersoil ™ | Object Swab, |
| | & 3 | Fixed | Weekly oscillation | | NaOH lysis |
| | perturbed | | (25-35 C.) | | |
| | | Wind: None | Wind: Fans, fixed | | |
| | | | speed | | |
| | | Rain: None | Rain: weekly, | | |
| | | | varying intensity | | |
| | | | from spraying to | | |
| | | | watering can | | |
| Soil | 3 control | Temperature: 25 C. | Temperature: | Powersoil ™ | Object Swab, |
| | & 3 | Fixed | Weekly oscillation | | NaOH lysis |
| | perturbed | | (25-35 C.) | | |
| | | Wind: None | Wind: Fans, fixed | | |
| | | | speed | | |
| | | Rain: None | Rain: weekly, | | |
| | | | varying intensity | | |
| | | | from spraying to | | |
| | | | watering can | | |
| Carpet | 2 control | Temperature: 25 C. | Temperature: 25 C. | Surface | Object Swab, |
| | & 2 | Fixed | Fixed | Swab, | NaOH lysis |
| | perturbed | Humidity: 40-50% | Humidity: 40-50% | NaOH lysis | |
| | | Controlled | Controlled | | |
| | | Cleaning: None | Cleaning: | | |
| | | | Vacuuming | | |
| Wood | 2 control | Temperature: 25 C. | Temperature: 25 C. | Surface | Object Swab, |
| | & 2 | Fixed | Fixed | Swab, | NaOH lysis |
| | perturbed | Humidity: 40-50% | Humidity: 40-50% | NaOH lysis | |
| | | Controlled | Controlled | | |
| | | Cleaning: None | Cleaning: Sweeping | | |

TABLE 10
Summary of Barcode SEQ ID NOs

| | Barcode | Genetic barcode | |
|---|---|---|---|
| Barcode # | region | element | crRNA |
| (BC) | SEQ ID NO | SEQ ID NO | SEQ ID NO |
| 1 | 5 | 222 | 59 |
| 2 | 6 | 223 | 60 |
| 3 | 7 | 224 | 61 |
| 4 | 8 | 225 | 62 |
| 5 | 9 | 226 | 63 |
| 6 | 10 | 227 | 64 |
| 7 | 11 | 228 | 65 |
| 8 | 12 | 229 | 66 |
| 9 | 13 | 230 | 67 |
| 10 | 14 | 231 | 68 |
| 11 | 15 | 232 | 69 |
| 12 | 16 | 233 | 70 |
| 13 | 17 | 234 | 71 |
| 14 | 18 | 235 | 72 |
| 15 | 19 | 236 | 73 |
| 16 | 20 | 237 | 74 |
| 17 | 21 | 238 | 75 |
| 18 | 22 | 239 | 76 |
| 19 | 23 | 240 | 77 |
| 20 | 24 | 241 | 78 |
| 21 | 25 | 242 | 79 |
| 22 | 26 | 243 | 80 |
| 23 | 27 | 244 | 81 |
| 24 | 28 | 245 | 82 |
| 25 | 29 | 246 | 83 |
| 26 | 30 | 247 | 84 |
| 27 | 31 | 248 | 85 |
| 28 | 154 | 249 | 86 |
| 29 | 155 | 250 | 87 |
| 30 | 156 | 251 | 88 |
| 31 | 157 | 252 | 89 |
| 32 | 158 | 253 | 90 |
| 33 | 159 | 254 | 91 |
| 34 | 160 | 255 | 92 |
| 35 | 161 | 256 | 93 |
| 36 | 162 | 257 | 94 |
| 37 | 163 | 258 | 95 |
| 38 | 164 | 259 | 96 |
| 39 | 165 | 260 | 97 |
| 40 | 166 | 261 | 98 |
| 41 | 167 | 262 | 99 |
| 42 | 168 | 263 | 100 |
| 43 | 169 | 264 | 101 |
| 44 | 170 | 265 | 102 |
| 45 | 171 | 266 | 103 |
| 46 | 172 | 267 | 104 |
| 47 | 173 | 268 | 105 |
| 48 | 174 | 269 | 106 |
| 49 | 175 | 270 | 107 |
| 50 | 176 | 271 | 108 |
| 51 | 177 | 272 | 109 |
| 52 | 178 | 273 | 110 |
| 53 | 179 | 274 | 111 |
| 54 | 180 | 275 | 112 |
| 55 | 181 | 276 | 113 |
| 56 | 182 | 277 | 114 |
| 57 | 183 | 278 | 115 |
| 58 | 184 | 279 | 116 |
| 59 | 185 | 280 | 117 |
| 60 | 186 | 281 | 118 |
| 61 | 187 | 282 | 119 |
| 62 | 188 | 283 | 120 |
| 63 | 189 | 284 | 121 |
| 64 | 190 | 285 | 122 |
| 65 | 191 | 286 | 123 |
| 66 | 192 | 287 | 124 |
| 67 | 193 | 288 | 125 |
| 68 | 194 | 289 | 126 |
| 69 | 195 | 290 | 127 |
| 70 | 196 | 291 | 128 |
| 71 | 197 | 292 | 129 |
| 72 | 198 | 293 | 130 |
| 73 | 199 | 294 | 131 |
| 74 | 200 | 295 | 132 |
| 75 | 201 | 296 | 133 |
| 76 | 202 | 297 | 134 |
| 77 | 203 | 298 | 135 |
| 78 | 204 | 299 | 136 |
| 79 | 205 | 300 | 137 |
| 80 | 206 | 301 | 138 |
| 81 | 207 | 302 | 139 |
| 82 | 208 | 303 | 140 |
| 83 | 209 | 304 | 141 |
| 84 | 210 | 305 | 142 |
| 85 | 211 | 306 | 143 |
| 86 | 212 | 307 | 144 |
| 87 | 213 | 308 | 145 |
| 88 | 214 | 309 | 146 |
| 89 | 215 | 310 | 147 |
| 90 | 216 | 311 | 148 |
| 91 | 217 | 312 | 149 |
| 92 | 218 | 313 | 150 |
| 93 | 219 | 314 | 151 |
| 94 | 220 | 315 | 152 |
| Universal 2 | 221 | Each element | 153 |
| | | above comprises | |
| | | Universal BC-2 | |

### Example 3: DNA Barcodes in Bacillus thuringiensis HD-73

The general strategy for introducing DNA barcodes in Bacillus thuringiensis HD-73 includes the following. 1) Find a neutral locus in B. thuringiensis HD-73. This gene must not be essential and nor involved in sporulation. 2) Design a plasmid that allows transformation of B. thuringiensis HD-73. A modified version of pMiniMAD can be used, which is a vector that allows a rapid gene inactivation in naturally non-transformable Gram (+) bacteria. This modified plasmid contains a mCherry gene under the control of a constitutive strong promoter (Pveg) and comprises the barcode, an antibiotic marker and the homologous regions to the neutral locus that allow recombination. 3) Transform B. thuringiensis HD-73. B. thuringiensis HD-73 is transformed by electroporation. Once transformants appear on the selection plates, a screen can also be conducted for a specific phenotype (e.g., pink colonies, e.g., from mCherry expression). In a second step, these positive transformants are grown and incubated at a restrictive temperature (42° C.) in the presence of the antibiotic overnight and finally plated on LB agar. Those colonies that no longer display a red coloration and/or no longer display plasmid-specific antibiotic resistance represent candidate clones resulting from a double crossover event and loss of the vector. Finally, a molecular assay allows for confirmation of the deletion of the gene and the integration of the desired elements in the bacterial chromosome.

(1) Finding a neutral locus in B. thuringiensis HD-73 to allow integration of exogenous DNA sequences. In one embodiment, the neutral site is HD73_5011, which encodes a Type I pullulanase (see e.g., FIG. 31). The Type I pullulanase encoded by HD73_5011 cleaves α-(1→6) bonds in amylopectines, dextrins and pullulan (a polysaccharide polymer consisting of maltriose units). This locus can be used as its gene product is not essential nor involved in sporulation. This redundant enzyme is important when the growth medium contains the aforementioned substrates. Sporulation media does not comprise any of these compounds (e.g., amylopectines, dextrins, or pullulan). Therefore, without wishing to be bound by theory, no negative impact on the biology of B. thuringiensis HD-73 is expected by integration into the HD73_5011 locus. Another advantage of the HD73_5011 locus is its localization near to the replication origin (see e.g., FIG. 32). In some embodiments of any of the aspects, any locus in B. thuringiensis that is not essential nor involved in sporulation can be used to integrate a genetic barcode element as described herein.

(2) Designing a plasmid that for transformation of B. thuringiensis HD-73. First flanking regions of HD73_5011 (e.g., a region that is about 1 kbp long and upstream of HD73_5011 and can include a portion of HD73_5012, and a region that is about 1 kbp long and downstream of HD73_5011 and can include a portion of HD73_5010) were generated by PCR (see e.g., FIG. 33A). Gibson assembly was then used to join the 3 pieces (i.e., the upstream and downstream regions generated by PCR, and the barcode DNA linked to a resistance cassette such as kanamycin resistance) to the vector (e.g., a modified pMiniMAD plasmid comprising a constitutive strong promoter, such as the Pveg promoter, and a detectable marker, such as mCherry), resulting in a double crossover recombination plasmid (see e.g., FIG. 33B).

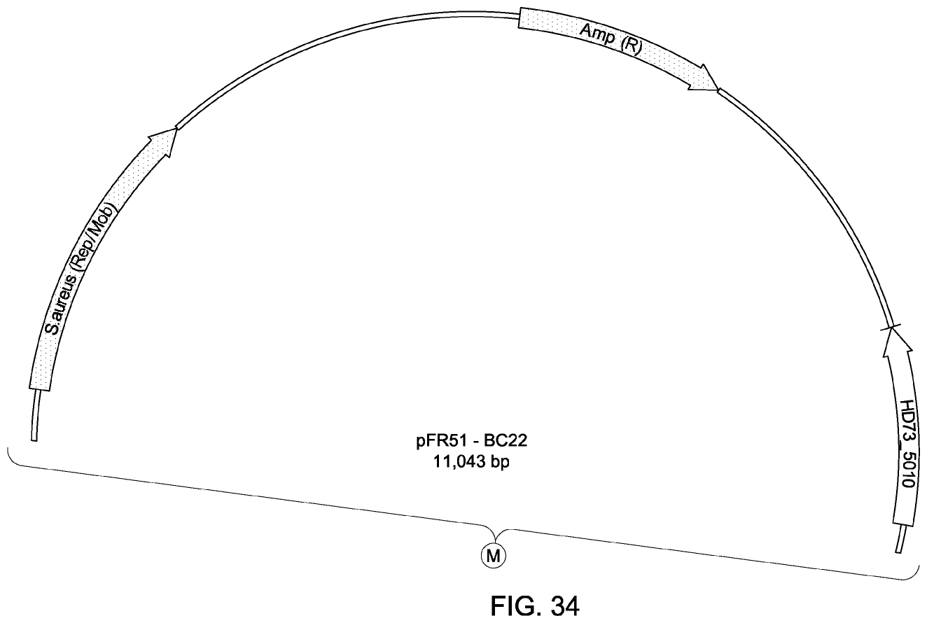

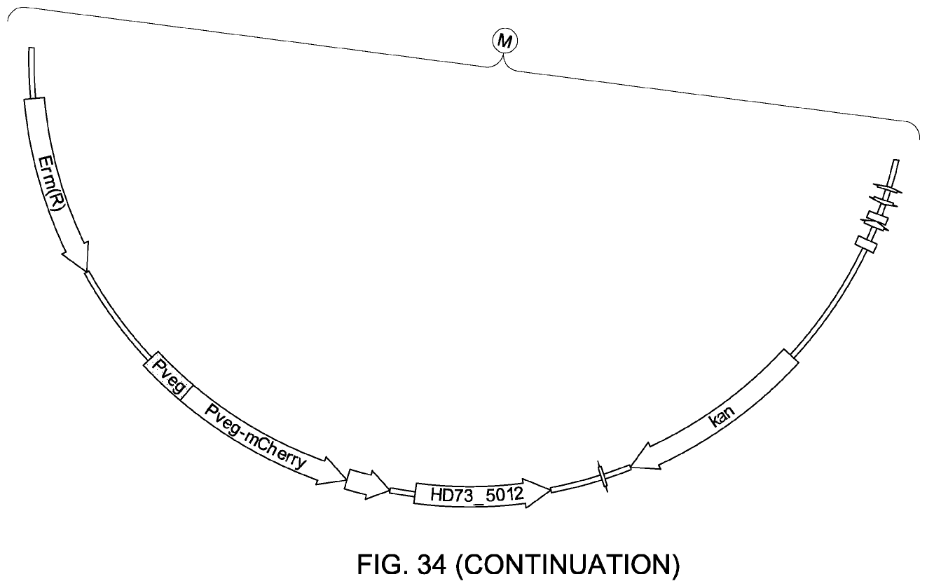

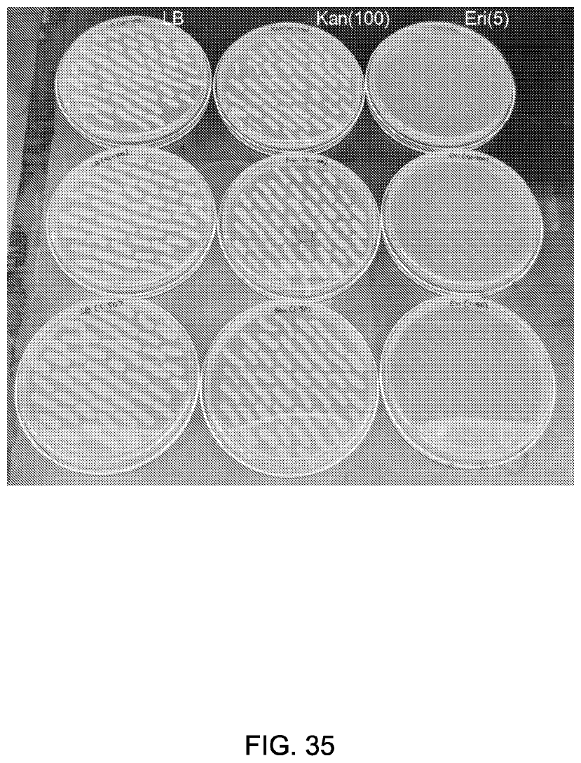

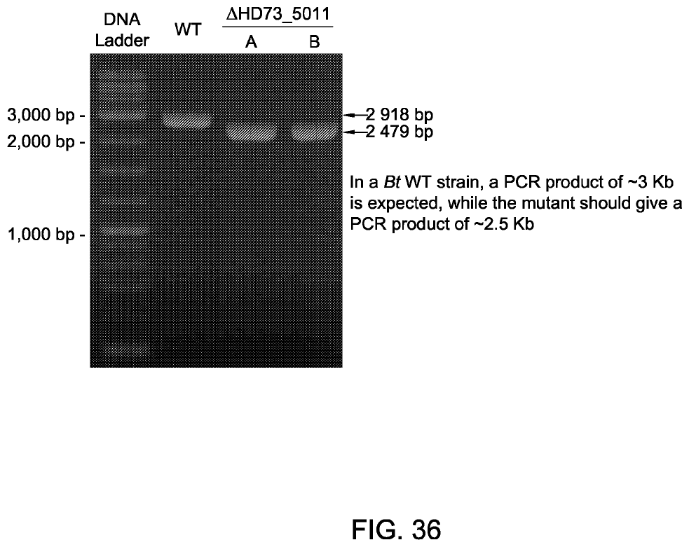

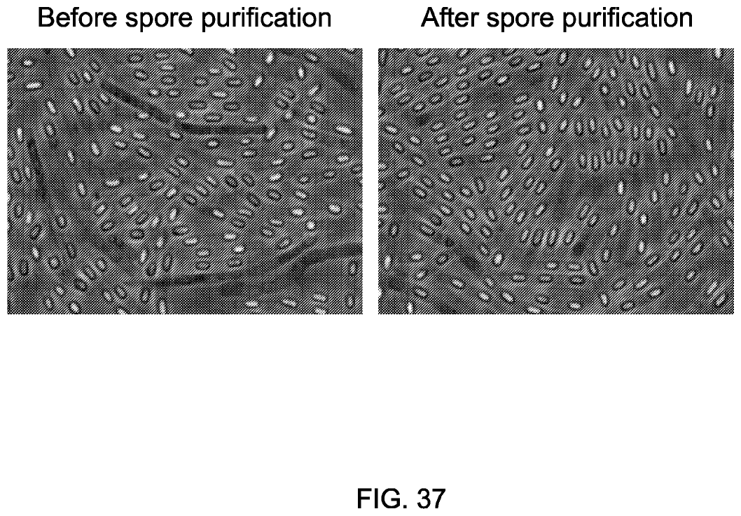

A pMiniMAD plasmid was used to transform B. thuringiensis HD-73 (e.g., by “looping in and out” or double crossover recombination). FIG. 34 shows the pFR51 plasmid, which comprises BC-22 (see e.g., SEQ ID NO: 26, SEQ ID NO: 243). While BC-22 is used in this example, it is contemplated that any barcode described herein or barcode designed according to the methods described herein can be used in Bt. The backbone of the pFR51 plasmid is pMiniMAD. This plasmid was prepared in both dam^{-/+}/dcm^{-/+} E. coli strains. 2 μg of pFR51 were used to transform B. thuringiensis kurstaki HD-73 by electroporation and selected on Erm (e.g., 5 ug/mL erythromycin) at 30° C. After 36h, ˜30 colonies appeared only from the dam⁻/dcm⁻ E. coli preparation. A pool of 5 individual colonies were used to inoculate 5 mL LB in a glass tube. This culture was grown for ˜5h at 30° C. 100 μL of this culture were used to inoculate 4.9 mL of LB-Kan (e.g., 100 ug/mL kanamycin) in a glass tube. This culture was grown overnight at 42° C. Early the next morning, the culture was serial diluted (on LB) and plated on LB plates. These plates were incubated at 37° C. for ˜10h. The same day, late in the afternoon, 150 colonies were patched from a serial dilution on LB-Eri (e.g., 5 ug/mL erythromycin), LB-Kan (e.g., 100 ug/mL kanamycin), and LB plates (in that order). These plates were incubated overnight at 30° C. 149 out of the 150 clones were Eri(5)-Sensitive and Kan(100)-Resistant (see e.g., FIG. 35), indicating that they have lost the vector (e.g., losing the erythromycin resistance) and are the result from a double cross-over recombination event (e.g., retaining the kanamycin resistance of the BC-DNA-kan insertion). Only 1 clone was Eri(5)-Sensitive and Kan(100)-Sensitive (see e.g., box denoted missing patch on the Kan(100) plate in FIG. 35); this patch likely lost the plasmid early, e.g., before integration of BC-DNA-kan. Two clones were picked to make single colonies, to extract gDNA to confirm the mutant (see e.g., FIG. 36), and to store a glycerol stock.

A fresh single-colony from one of the mutant strains was used to inoculate 50 mL of DSM_{complete}. The culture was incubated with aeration for 48h at 37° C. to induce sporulation. After this time, a picture of the culture was taken before and after spore purification (see e.g., FIG. 37). Heat-resistant colonies (e.g., spores) were also determined by plating aliquots of a serial dilution (e.g., 1×10⁹ heat-resistant CFUs/mL). Such a barcoded Bt strain can be used to determine the provenance of an item (e.g., food), as described herein. In some embodiments of any of the aspects, the barcoded Bt strain further comprises an inactivating modification of at least one essential gene (e.g., thrC, metA, trpC, and/or pheA) and/or an inactivating modification of at least one germination gene (e.g., cwlJ, sleB, gerAB, and/or gerKB).

1. A microorganism engineered to comprise at least one genetic barcode element and at least one of:
a) an inactivating modification of at least one essential gene; or
b) an inactivating modification of at least one germination gene.

2.-3. (canceled)

4. The engineered microorganism of claim 1, wherein the microorganism is a yeast or a bacterium.

5. The engineered microorganism of claim 1, wherein the microorganism is a Saccharomyces yeast or a Bacillus bacterium.

6. The engineered microorganism of claim 1, wherein the microorganism is Saccharomyces cerevisiae, Bacillus subtilis, or Bacillus thuringiensis.

7. (canceled)

8. The engineered microorganism of claim 1, wherein the genetic barcode element comprises:
a) a first primer binding sequence;
b) at least one barcode region;
c) a Cas enzyme scaffold;
d) a transcription initiation site; and
e) a second primer binding sequence; or
the genetic barcode element comprises:
f) a first primer binding sequence;
g) at least one barcode region;
h) a transcription initiation site; and
i) a second primer binding sequence; or
the genetic barcode element comprises:
j) a first primer binding sequence;
k) at least one barcode region; and
l) a second primer binding sequence.

9.-10. (canceled)

11. The engineered microorganism of claim 1, wherein the microorganism is engineered to comprise first and second barcode regions, wherein the first barcode region indicates that an item on which the microorganism is detected is from one of a group of known sources, and the second barcode region indicates that an item on which the microorganism is detected is from a particular source of said group of sources.

12. (canceled)

13. The engineered microorganism claim 8, wherein the first primer binding sequence and second primer binding sequence comprise sites for binding of PCR or RPA primers.

14. The engineered microorganism of claim 8, wherein the barcode region:
a) comprises 20-40 base pairs,
b) comprises a Hamming distance of at least 5 base pairs relative to barcode regions comprised by other items marked with an engineered microorganism of claim 8; or
c) is unique or distinguishable from at least one other barcode region comprised by other items marked with an engineered microorganism of claim 8.

15.-16. (canceled)

17. The engineered microorganism of claim 8, wherein the Cas enzyme scaffold comprises a scaffold for Cas13.

18. The engineered microorganism of claim 8, wherein the transcription initiation site comprises a T7 transcription initiation site.

19. The engineered microorganism of claim 1, wherein the at least one essential gene comprises an essential compound synthesis gene selected from an amino acid synthesis gene or a nucleotide synthesis gene.

20.-22. (canceled)

23. The engineered microorganism of claim 19, wherein the at least one essential compound synthesis gene comprises a synthesis gene for threonine, methionine, tryptophan, phenylalanine, histidine, leucine, lysine, or uracil, or is selected from the group consisting of thrC, metA, trpC, pheA, HIS3, LEU2, LYS2, MET15, and URA3.

24. (canceled)

25. The engineered microorganism of claim 19, comprising an inactivating modification of at least two or more essential compound synthesis genes.

26. The engineered microorganism of claim 1, wherein the at least one germination gene is selected from the group consisting of cwlJ, sleB, gerAB, gerBB, and gerKB.

27. The engineered microorganism of claim 1, comprising an inactivating modification of two or more germination genes.

28. The engineered microorganism of claim 1, wherein the engineered microorganism is inactivated through boiling prior to use.

29. A method of determining the provenance of an item, the method comprising:
a) contacting an item with at least one engineered microorganism of claim 1;
b) isolating nucleic acid from the item;
c) detecting the genetic barcode element of the at least one isolated engineered microorganism; and
d) determining the provenance of the item based on the detected genetic barcode element of the at least one isolated engineered microorganism.

30.-31. (canceled)

32. A method of determining the provenance of an item, the method comprising:
a) isolating nucleic acid from the item; and
b) detecting the presence of a genetic barcode element, wherein the presence of the genetic barcode element indicates the presence of at least one engineered microorganism comprising a genetic barcode element and an inactivating modification of at least one essential compound synthesis gene or an inactivating modification of at least one germination gene, wherein the presence of the at least one engineered microorganism determines the provenance of the item.

33. A method of marking the provenance of an item, the method comprising contacting the item with at least one engineered microorganism of claim 1.

34.-41. (canceled)

42. A method of determining the path of an item or an individual across a surface, comprising:
a) contacting a surface with at least two engineered microorganisms of claim 1;
b) allowing the item or individual to contact the surface in a continuous or discontinuous path;
c) isolating nucleic acid from the item or individual;
d) detecting the genetic barcode elements of the at least two isolated engineered microorganisms; and
e) determining the path of the item or individual across the surface based on the detected genetic barcode element of the at least two isolated engineered microorganisms.

43.-51. (canceled)


SEQUENCE LISTING (358 sequences)

SEQ ID NO: 1 - DNA, 35 bp, Artificial Sequence
  Description of Artificial Sequence Synthetic primer
gataaacacaggaaacagctatgaccatgattacg

SEQ ID NO: 2 - DNA, 36 bp, Artificial Sequence
  Description of Artificial Sequence Synthetic oligonucleotide
gttttagtccccttcgtttttggggtagtctaaatc

SEQ ID NO: 3 - DNA, 25 bp, Artificial Sequence
  Description of Artificial Sequence Synthetic oligonucleotide
ccctatagtgagtcgtattagaatt

SEQ ID NO: 4 - DNA, 37 bp, Artificial Sequence
  Description of Artificial Sequence Synthetic primer
gggatcctctagaaatatggattacttggtagaacag

SEQ ID NO: 5 - DNA, 28 bp, Artificial Sequence
  Description of Artificial Sequence Synthetic oligonucleotide
tcgtcagtcgtaacttgggaacgcacat

SEQ ID NO: 6 - DNA, 28 bp, Artificial Sequence
  Description of Artificial Sequence Synthetic oligonucleotide
ctttggggttggataaatctgtcgtggt

SEQ ID NO: 7 - DNA, 28 bp, Artificial Sequence
  Description of Artificial Sequence Synthetic oligonucleotide
tgaataagcgcggtccctaatgttggtg

SEQ ID NO: 8 - DNA, 28 bp, Artificial Sequence
  Description of Artificial Sequence Synthetic oligonucleotide
accggtttatagttcgaacagtcgcagc

SEQ ID NO: 9 - DNA, 28 bp, Artificial Sequence
  Description of Artificial Sequence Synthetic oligonucleotide
ccccgtgtggtaactacgcaagcctaac

SEQ ID NO: 10 - DNA, 28 bp, Artificial Sequence
  Description of Artificial Sequence Synthetic oligonucleotide
acgtaggggggcgcgtaaccactagctc

SEQ ID NO: 11 - DNA, 28 bp, Artificial Sequence
  Description of Artificial Sequence Synthetic oligonucleotide
agtgtcccttattctactttgaattatc

SEQ ID NO: 12 - DNA, 28 bp, Artificial Sequence
  Description of Artificial Sequence Synthetic oligonucleotide
gagttacgggtcaggatcattgcgcagg

SEQ ID NO: 13 - DNA, 28 bp, Artificial Sequence
  Description of Artificial Sequence Synthetic oligonucleotide
gtgagtccggcctatcacgtttggtagg

SEQ ID NO: 14 - DNA, 28 bp, Artificial Sequence
  Description of Artificial Sequence Synthetic oligonucleotide
tcaggggaaacgagttaagcagaggcag

SEQ ID NO: 15 - DNA, 28 bp, Artificial Sequence
  Description of Artificial Sequence Synthetic oligonucleotide
cttggtccaatcgtatgctaagagtagc

SEQ ID NO: 16 - DNA, 28 bp, Artificial Sequence
  Description of Artificial Sequence Synthetic oligonucleotide
tcggttgcacgcgcgtcctgcacgctcg

SEQ ID NO: 17 - DNA, 28 bp, Artificial Sequence
  Description of Artificial Sequence Synthetic oligonucleotide
gttcaaaagcgggagtcccggtgaaacc

SEQ ID NO: 18 - DNA, 28 bp, Artificial Sequence
  Description of Artificial Sequence Synthetic oligonucleotide
gagggcttctacgaaattgcctcaccat

SEQ ID NO: 19 - DNA, 28 bp, Artificial Sequence
  Description of Artificial Sequence Synthetic oligonucleotide
gggatgccctgataacgtagagtctcag

SEQ ID NO: 20 - DNA, 28 bp, Artificial Sequence
  Description of Artificial Sequence Synthetic oligonucleotide
tagtacgggccacgatctaagtcggcgg

SEQ ID NO: 21 - DNA, 28 bp, Artificial Sequence
  Description of Artificial Sequence Synthetic oligonucleotide
gtatgtgggtacgattgtaggctagaaa

SEQ ID NO: 22 - DNA, 28 bp, Artificial Sequence
  Description of Artificial Sequence Synthetic oligonucleotide
agaactacctacgtggccacgcaagccc

SEQ ID NO: 23 - DNA, 28 bp, Artificial Sequence
  Description of Artificial Sequence Synthetic oligonucleotide
tgcctcagaatggggtttaacgttcagg

SEQ ID NO: 24 - DNA, 28 bp, Artificial Sequence
  Description of Artificial Sequence Synthetic oligonucleotide
atgttgggacgccagactgacacgcaaa

SEQ ID NO: 25 - DNA, 28 bp, Artificial Sequence
  Description of Artificial Sequence Synthetic oligonucleotide
cctacaccttcaccgagtgtggagcaag

SEQ ID NO: 26 - DNA, 28 bp, Artificial Sequence
  Description of Artificial Sequence Synthetic oligonucleotide
gtttaacattcgggtatcgtccttacga

SEQ ID NO: 27 - DNA, 28 bp, Artificial Sequence
  Description of Artificial Sequence Synthetic oligonucleotide
gcaatattactagtccgtgtcagccaga

SEQ ID NO: 28 - DNA, 28 bp, Artificial Sequence
  Description of Artificial Sequence Synthetic oligonucleotide
ccgactaggtcggcgataccatagcacc

SEQ ID NO: 29 - DNA, 28 bp, Artificial Sequence
  Description of Artificial Sequence Synthetic oligonucleotide
gatgacgcctcacgtccgttgcccccta

SEQ ID NO: 30 - DNA, 28 bp, Artificial Sequence
  Description of Artificial Sequence Synthetic oligonucleotide
gctccgtagcaattgataaaccgctgag

SEQ ID NO: 31 - DNA, 28 bp, Artificial Sequence
  Description of Artificial Sequence Synthetic oligonucleotide
ctatcggatttttagataaattacgagg

SEQ ID NO: 32 - DNA, 35 bp, Artificial Sequence; identical to SEQ ID NO: 1
  Description of Artificial Sequence Synthetic primer

SEQ ID NO: 33 - DNA, 28 bp, Artificial Sequence
  Description of Artificial Sequence Synthetic primer
tcgtcagtcgtaacttgggaacgcacat

SEQ ID NO: 34 - DNA, 28 bp, Artificial Sequence
  Description of Artificial Sequence Synthetic primer
ctttggggttggataaatctgtcgtggt

SEQ ID NO: 35 - DNA, 28 bp, Artificial Sequence
  Description of Artificial Sequence Synthetic primer
tgaataagcgcggtccctaatgttggtg

SEQ ID NO: 36 - DNA, 28 bp, Artificial Sequence
  Description of Artificial Sequence Synthetic primer
accggtttatagttcgaacagtcgcagc

SEQ ID NO: 37 - DNA, 27 bp, Artificial Sequence
  Description of Artificial Sequence Synthetic primer
cccgtgtggtaactacgcaagcctaac

SEQ ID NO: 38 - DNA, 20 bp, Artificial Sequence
  Description of Artificial Sequence Synthetic primer
gggcgcgtaaccactagctc

SEQ ID NO: 39 - DNA, 37 bp, Artificial Sequence
  Description of Artificial Sequence Synthetic primer
gggctagctagtgtcccttattctactttgaattatc

SEQ ID NO: 40 - DNA, 26 bp, Artificial Sequence
  Description of Artificial Sequence Synthetic primer
gttacgggtcaggatcattgcgcagg

SEQ ID NO: 41 - DNA, 28 bp, Artificial Sequence
  Description of Artificial Sequence Synthetic primer
ccgactaggtcggcgataccatagcacc

SEQ ID NO: 42 - DNA, 28 bp, Artificial Sequence
  Description of Artificial Sequence Synthetic primer
gatgacgcctcacgtccgttgcccccta

SEQ ID NO: 43 - DNA, 28 bp, Artificial Sequence
  Description of Artificial Sequence Synthetic primer
tgcctcagaatggggtttaacgttcagg

SEQ ID NO: 44 - DNA, 28 bp, Artificial Sequence
  Description of Artificial Sequence Synthetic primer
accgtcctagtgcggatagtgggacatg

SEQ ID NO: 45 - DNA, 28 bp, Artificial Sequence
  Description of Artificial Sequence Synthetic primer
gtcgatacgggtcgctagataggttggg

SEQ ID NO: 46 - DNA, 20 bp, Artificial Sequence
  Description of Artificial Sequence Synthetic primer
gggatcctctagaaatatgg

SEQ ID NO: 47 - DNA, 20 bp, Artificial Sequence
  Description of Artificial Sequence Synthetic primer
tgataaacacaggaaacagc

SEQ ID NO: 48 - DNA, 24 bp, Artificial Sequence
  Description of Artificial Sequence Synthetic primer
atcaacaacagaggacatatgccc

SEQ ID NO: 49 - DNA, 23 bp, Artificial Sequence
  Description of Artificial Sequence Synthetic primer
atcctgcactcatctactacccg

SEQ ID NO: 50 - DNA, 20 bp, Artificial Sequence
  Description of Artificial Sequence Synthetic primer
caaacatgcgaagcgactta

SEQ ID NO: 51 - DNA, 20 bp, Artificial Sequence
  Description of Artificial Sequence Synthetic primer
cgcctccttggatggtaata

SEQ ID NO: 52 - DNA, 25 bp, Artificial Sequence
  Description of Artificial Sequence Synthetic primer
gaaattaatacgactcactataggg

SEQ ID NO: 53 - DNA, 35 bp, Artificial Sequence; identical to SEQ ID NO: 1
  Description of Artificial Sequence Synthetic primer

SEQ ID NO: 54 - DNA, 37 bp, Artificial Sequence; identical to SEQ ID NO: 4
  Description of Artificial Sequence Synthetic primer

SEQ ID NO: 55 - DNA, 30 bp, Artificial Sequence
  Description of Artificial Sequence Synthetic primer
cgcggatcctctagaaatatggattacttg

SEQ ID NO: 56 - DNA, 30 bp, Artificial Sequence
  Description of Artificial Sequence Synthetic primer
cggctcgagataaacacaggaaacagctat

SEQ ID NO: 57 - DNA, 42 bp, Artificial Sequence
  Description of Artificial Sequence Synthetic primer
gcgaagcttacataaggaggaactactatggtttctaaaggt

SEQ ID NO: 58 - DNA, 27 bp, Artificial Sequence
  Description of Artificial Sequence Synthetic primer
gcgggatccttacttatacagttcatc

SEQ ID NO: 59 - DNA, 89 bp, Artificial Sequence
  Description of Artificial Sequence Synthetic oligonucleotide
tcgtcagtcgtaacttgggaacgcacatgttttagtccccttcgtttttggggtagtctaaatcccctatagtgagtcgtattaatttc

SEQ ID NO: 60 - DNA, 89 bp, Artificial Sequence
  Description of Artificial Sequence Synthetic oligonucleotide
ctttggggttggataaatctgtcgtggtgttttagtccccttcgtttttggggtagtctaaatcccctatagtgagtcgtattaatttc

SEQ ID NO: 61 - DNA, 89 bp, Artificial Sequence
  Description of Artificial Sequence Synthetic oligonucleotide
tgaataagcgcggtccctaatgttggtggttttagtccccttcgtttttggggtagtctaaatcccctatagtgagtcgtattaatttc

SEQ ID NO: 62 - DNA, 89 bp, Artificial Sequence
  Description of Artificial Sequence Synthetic oligonucleotide
accggtttatagttcgaacagtcgcagcgttttagtccccttcgtttttggggtagtctaaatcccctatagtgagtcgtattaatttc

SEQ ID NO: 63 - DNA, 89 bp, Artificial Sequence
  Description of Artificial Sequence Synthetic oligonucleotide
ccccgtgtggtaactacgcaagcctaacgttttagtccccttcgtttttggggtagtctaaatcccctatagtgagtcgtattaatttc

SEQ ID NO: 64 - DNA, 89 bp, Artificial Sequence
  Description of Artificial Sequence Synthetic oligonucleotide
acgtaggggggcgcgtaaccactagctcgttttagtccccttcgtttttggggtagtctaaatcccctatagtgagtcgtattaatttc

SEQ ID NO: 65 - DNA, 89 bp, Artificial Sequence
  Description of Artificial Sequence Synthetic oligonucleotide
agtgtcccttattctactttgaattatcgttttagtccccttcgtttttggggtagtctaaatcccctatagtgagtcgtattaatttc

SEQ ID NO: 66 - DNA, 89 bp, Artificial Sequence
  Description of Artificial Sequence Synthetic oligonucleotide
gagttacgggtcaggatcattgcgcagggttttagtccccttcgtttttggggtagtctaaatcccctatagtgagtcgtattaatttc

SEQ ID NO: 67 - DNA, 89 bp, Artificial Sequence
  Description of Artificial Sequence Synthetic oligonucleotide
gtgagtccggcctatcacgtttggtagggttttagtccccttcgtttttggggtagtctaaatcccctatagtgagtcgtattaatttc

SEQ ID NO: 68 - DNA, 89 bp, Artificial Sequence
  Description of Artificial Sequence Synthetic oligonucleotide
tcaggggaaacgagttaagcagaggcaggttttagtccccttcgtttttggggtagtctaaatcccctatagtgagtcgtattaatttc

SEQ ID NO: 69 - DNA, 89 bp, Artificial Sequence
  Description of Artificial Sequence Synthetic oligonucleotide
cttggtccaatcgtatgctaagagtagcgttttagtccccttcgtttttggggtagtctaaatcccctatagtgagtcgtattaatttc

SEQ ID NO: 70 - DNA, 89 bp, Artificial Sequence
  Description of Artificial Sequence Synthetic oligonucleotide
tcggttgcacgcgcgtcctgcacgctcggttttagtccccttcgtttttggggtagtctaaatcccctatagtgagtcgtattaatttc

SEQ ID NO: 71 - DNA, 89 bp, Artificial Sequence
  Description of Artificial Sequence Synthetic oligonucleotide
gttcaaaagcgggagtcccggtgaaaccgttttagtccccttcgtttttggggtagtctaaatcccctatagtgagtcgtattaatttc

SEQ ID NO: 72 - DNA, 89 bp, Artificial Sequence
  Description of Artificial Sequence Synthetic oligonucleotide
gagggcttctacgaaattgcctcaccatgttttagtccccttcgtttttggggtagtctaaatcccctatagtgagtcgtattaatttc

SEQ ID NO: 73 - DNA, 89 bp, Artificial Sequence
  Description of Artificial Sequence Synthetic oligonucleotide
gggatgccctgataacgtagagtctcaggttttagtccccttcgtttttggggtagtctaaatcccctatagtgagtcgtattaatttc

SEQ ID NO: 74 - DNA, 89 bp, Artificial Sequence
  Description of Artificial Sequence Synthetic oligonucleotide
tagtacgggccacgatctaagtcggcgggttttagtccccttcgtttttggggtagtctaaatcccctatagtgagtcgtattaatttc

SEQ ID NO: 75 - DNA, 89 bp, Artificial Sequence
  Description of Artificial Sequence Synthetic oligonucleotide
gtatgtgggtacgattgtaggctagaaagttttagtccccttcgtttttggggtagtctaaatcccctatagtgagtcgtattaatttc

SEQ ID NO: 76 - DNA, 89 bp, Artificial Sequence
  Description of Artificial Sequence Synthetic oligonucleotide
agaactacctacgtggccacgcaagcccgttttagtccccttcgtttttggggtagtctaaatcccctatagtgagtcgtattaatttc

SEQ ID NO: 77 - DNA, 89 bp, Artificial Sequence
  Description of Artificial Sequence Synthetic oligonucleotide
tgcctcagaatggggtttaacgttcagggttttagtccccttcgtttttggggtagtctaaatcccctatagtgagtcgtattaatttc

SEQ ID NO: 78 - DNA, 89 bp, Artificial Sequence
  Description of Artificial Sequence Synthetic oligonucleotide
atgttgggacgccagactgacacgcaaagttttagtccccttcgtttttggggtagtctaaatcccctatagtgagtcgtattaatttc

SEQ ID NO: 79 - DNA, 89 bp, Artificial Sequence
  Description of Artificial Sequence Synthetic oligonucleotide
cctacaccttcaccgagtgtggagcaaggttttagtccccttcgtttttggggtagtctaaatcccctatagtgagtcgtattaatttc

SEQ ID NO: 80 - DNA, 89 bp, Artificial Sequence
  Description of Artificial Sequence Synthetic oligonucleotide
gtttaacattcgggtatcgtccttacgagttttagtccccttcgtttttggggtagtctaaatcccctatagtgagtcgtattaatttc

SEQ ID NO: 81 - DNA, 89 bp, Artificial Sequence
  Description of Artificial Sequence Synthetic oligonucleotide
gcaatattactagtccgtgtcagccagagttttagtccccttcgtttttggggtagtctaaatcccctatagtgagtcgtattaatttc

SEQ ID NO: 82 - DNA, 89 bp, Artificial Sequence
  Description of Artificial Sequence Synthetic oligonucleotide
ccgactaggtcggcgataccatagcaccgttttagtccccttcgtttttggggtagtctaaatcccctatagtgagtcgtattaatttc

SEQ ID NO: 83 - DNA, 89 bp, Artificial Sequence
  Description of Artificial Sequence Synthetic oligonucleotide
gatgacgcctcacgtccgttgccccctagttttagtccccttcgtttttggggtagtctaaatcccctatagtgagtcgtattaatttc

SEQ ID NO: 84 - DNA, 89 bp, Artificial Sequence
  Description of Artificial Sequence Synthetic oligonucleotide
gctccgtagcaattgataaaccgctgaggttttagtccccttcgtttttggggtagtctaaatcccctatagtgagtcgtattaatttc

SEQ ID NO: 85 - DNA, 89 bp, Artificial Sequence
  Description of Artificial Sequence Synthetic oligonucleotide
ctatcggatttttagataaattacgagggttttagtccccttcgtttttggggtagtctaaatcccctatagtgagtcgtattaatttc

SEQ ID NO: 86 - DNA, 89 bp, Artificial Sequence
  Description of Artificial Sequence Synthetic oligonucleotide
tttttaatcggtagcgacgatatatgacgttttagtccccttcgtttttggggtagtctaaatcccctatagtgagtcgtattaatttc

SEQ ID NO: 87 - DNA, 89 bp, Artificial Sequence
  Description of Artificial Sequence Synthetic oligonucleotide
ttgattcgtggggatccgcggatagaaggttttagtccccttcgtttttggggtagtctaaatcccctatagtgagtcgtattaatttc

SEQ ID NO: 88 - DNA, 89 bp, Artificial Sequence
  Description of Artificial Sequence Synthetic oligonucleotide
ctaatatctgatccgttataacaccacagttttagtccccttcgtttttggggtagtctaaatcccctatagtgagtcgtattaatttc

SEQ ID NO: 89 - DNA, 89 bp, Artificial Sequence
  Description of Artificial Sequence Synthetic oligonucleotide
ttgcgggcctggacattcaatcgcatacgttttagtccccttcgtttttggggtagtctaaatcccctatagtgagtcgtattaatttc

SEQ ID NO: 90 - DNA, 89 bp, Artificial Sequence
  Description of Artificial Sequence Synthetic oligonucleotide
gcctagaccaaaagtcgccttcagggcagttttagtccccttcgtttttggggtagtctaaatcccctatagtgagtcgtattaatttc

SEQ ID NO: 91 - DNA, 89 bp, Artificial Sequence
  Description of Artificial Sequence Synthetic oligonucleotide
ataggtaggtaacggaactgaccgataagttttagtccccttcgtttttggggtagtctaaatcccctatagtgagtcgtattaatttc

SEQ ID NO: 92 - DNA, 89 bp, Artificial Sequence
  Description of Artificial Sequence Synthetic oligonucleotide
cgtactccttacgccttacacggacaatgttttagtccccttcgtttttggggtagtctaaatcccctatagtgagtcgtattaatttc

SEQ ID NO: 93 - DNA, 89 bp, Artificial Sequence
  Description of Artificial Sequence Synthetic oligonucleotide
gattctctggtcatgagggaagatcgacgttttagtccccttcgtttttggggtagtctaaatcccctatagtgagtcgtattaatttc

SEQ ID NO: 94 - DNA, 89 bp, Artificial Sequence
  Description of Artificial Sequence Synthetic oligonucleotide
tttggacggggtcactactctttacagcgttttagtccccttcgtttttggggtagtctaaatcccctatagtgagtcgtattaatttc

SEQ ID NO: 95 - DNA, 89 bp, Artificial Sequence
  Description of Artificial Sequence Synthetic oligonucleotide
ttcatgtatgctgtcctaccgcagtagcgttttagtccccttcgtttttggggtagtctaaatcccctatagtgagtcgtattaatttc

SEQ ID NO: 96 - DNA, 89 bp, Artificial Sequence
  Description of Artificial Sequence Synthetic oligonucleotide
tacataaggtcgtcctccggtcacgcccgttttagtccccttcgtttttggggtagtctaaatcccctatagtgagtcgtattaatttc

SEQ ID NO: 97 - DNA, 89 bp, Artificial Sequence
  Description of Artificial Sequence Synthetic oligonucleotide
caggccgctactcaagcggggatagcttgttttagtccccttcgtttttggggtagtctaaatcccctatagtgagtcgtattaatttc

SEQ ID NO: 98 - DNA, 89 bp, Artificial Sequence
  Description of Artificial Sequence Synthetic oligonucleotide
cgcttagctagattgcccctggaagcgcgttttagtccccttcgtttttggggtagtctaaatcccctatagtgagtcgtattaatttc

SEQ ID NO: 99 - DNA, 89 bp, Artificial Sequence
  Description of Artificial Sequence Synthetic oligonucleotide
cggagtgtttgcgaatcccgagcattaggttttagtccccttcgtttttggggtagtctaaatcccctatagtgagtcgtattaatttc

SEQ ID NO: 100 - DNA, 89 bp, Artificial Sequence
  Description of Artificial Sequence Synthetic oligonucleotide
gtctttcgaatcgttccttataacccgcgttttagtccccttcgtttttggggtagtctaaatcccctatagtgagtcgtattaatttc

SEQ ID NO: 101 - DNA, 89 bp, Artificial Sequence
  Description of Artificial Sequence Synthetic oligonucleotide
aggctgaatcctccggcggctgttctaagttttagtccccttcgtttttggggtagtctaaatcccctatagtgagtcgtattaatttc

SEQ ID NO: 102 - DNA, 89 bp, Artificial Sequence
  Description of Artificial Sequence Synthetic oligonucleotide
cagccaaacgctagcaaaataggatatagttttagtccccttcgtttttggggtagtctaaatcccctatagtgagtcgtattaatttc

SEQ ID NO: 103 - DNA, 89 bp, Artificial Sequence
  Description of Artificial Sequence Synthetic oligonucleotide
cagacgctgtctacctagcagaagggctgttttagtccccttcgtttttggggtagtctaaatcccctatagtgagtcgtattaatttc

SEQ ID NO: 104 - DNA, 89 bp, Artificial Sequence
  Description of Artificial Sequence Synthetic oligonucleotide
aaggtataatgttcaagcgtggtggctagttttagtccccttcgtttttggggtagtctaaatcccctatagtgagtcgtattaatttc

SEQ ID NO: 105 - DNA, 89 bp, Artificial Sequence
  Description of Artificial Sequence Synthetic oligonucleotide
actgatctcggcgtaaatacaccgcagagttttagtccccttcgtttttggggtagtctaaatcccctatagtgagtcgtattaatttc

SEQ ID NO: 106 - DNA, 89 bp, Artificial Sequence
  Description of Artificial Sequence Synthetic oligonucleotide
tatgtgtatcctaaggtacctttacggggttttagtccccttcgtttttggggtagtctaaatcccctatagtgagtcgtattaatttc

SEQ ID NO: 107 - DNA, 89 bp, Artificial Sequence
  Description of Artificial Sequence Synthetic oligonucleotide
accgtcctagtgcggatagtgggacatggttttagtccccttcgtttttggggtagtctaaatcccctatagtgagtcgtattaatttc

SEQ ID NO: 108 - DNA, 89 bp, Artificial Sequence
  Description of Artificial Sequence Synthetic oligonucleotide
gtcgatacgggtcgctagataggttggggttttagtccccttcgtttttggggtagtctaaatcccctatagtgagtcgtattaatttc

SEQ ID NO: 109 - DNA, 89 bp, Artificial Sequence
  Description of Artificial Sequence Synthetic oligonucleotide
acagtcagaggcgcttcaatcgtcgcccgttttagtccccttcgtttttggggtagtctaaatcccctatagtgagtcgtattaatttc

SEQ ID NO: 110 - DNA, 89 bp, Artificial Sequence
  Description of Artificial Sequence Synthetic oligonucleotide
gcgactcatttaatttcaacgggtagaagttttagtccccttcgtttttggggtagtctaaatcccctatagtgagtcgtattaatttc

SEQ ID NO: 111 - DNA, 89 bp, Artificial Sequence
  Description of Artificial Sequence Synthetic oligonucleotide
tagacagagggagctatattgcgcggcggttttagtccccttcgtttttggggtagtctaaatcccctatagtgagtcgtattaatttc

SEQ ID NO: 112 - DNA, 89 bp, Artificial Sequence
  Description of Artificial Sequence Synthetic oligonucleotide
ggcattcaatagccagtaaccttagacagttttagtccccttcgtttttggggtagtctaaatcccctatagtgagtcgtattaatttc

SEQ ID NO: 113 - DNA, 89 bp, Artificial Sequence
  Description of Artificial Sequence Synthetic oligonucleotide
acaaaagttggcatttcgtagacaaagcgttttagtccccttcgtttttggggtagtctaaatcccctatagtgagtcgtattaatttc

SEQ ID NO: 114 - DNA, 89 bp, Artificial Sequence
  Description of Artificial Sequence Synthetic oligonucleotide
ttgcatactttgcaggttagggccgagagttttagtccccttcgtttttggggtagtctaaatcccctatagtgagtcgtattaatttc

SEQ ID NO: 115 - DNA, 89 bp, Artificial Sequence
  Description of Artificial Sequence Synthetic oligonucleotide
accggagttcaaacgactttgggtccctgttttagtccccttcgtttttggggtagtctaaatcccctatagtgagtcgtattaatttc

SEQ ID NO: 116 - DNA, 89 bp, Artificial Sequence
  Description of Artificial Sequence Synthetic oligonucleotide
cactggtaggctcgcgggtaaatagagcgttttagtccccttcgtttttggggtagtctaaatcccctatagtgagtcgtattaatttc

SEQ ID NO: 117 - DNA, 89 bp, Artificial Sequence
  Description of Artificial Sequence Synthetic oligonucleotide
ccgaagctcagtgtgatgtgccttccgtgttttagtccccttcgtttttggggtagtctaaatcccctatagtgagtcgtattaatttc

SEQ ID NO: 118 - DNA, 89 bp, Artificial Sequence
  Description of Artificial Sequence Synthetic oligonucleotide
gtacgtatgactgtgtcccagctatgaagttttagtccccttcgtttttggggtagtctaaatcccctatagtgagtcgtattaatttc

SEQ ID NO: 119 - DNA, 89 bp, Artificial Sequence
  Description of Artificial Sequence Synthetic oligonucleotide
ggaccttcacgtcagtcccgctgaccttgttttagtccccttcgtttttggggtagtctaaatcccctatagtgagtcgtattaatttc

SEQ ID NO: 120 - DNA, 89 bp, Artificial Sequence
  Description of Artificial Sequence Synthetic oligonucleotide
gaccaattatgttggccacgccacattagttttagtccccttcgtttttggggtagtctaaatcccctatagtgagtcgtattaatttc

SEQ ID NO: 121 - DNA, 89 bp, Artificial Sequence
  Description of Artificial Sequence Synthetic oligonucleotide
gttttcaagggctcgacacagcagaacggttttagtccccttcgtttttggggtagtctaaatcccctatagtgagtcgtattaatttc

SEQ ID NO: 122 - DNA, 89 bp, Artificial Sequence
  Description of Artificial Sequence Synthetic oligonucleotide
aacccactatagaagggccttcgagattgttttagtccccttcgtttttggggtagtctaaatcccctatagtgagtcgtattaatttc

SEQ ID NO: 123 - DNA, 89 bp, Artificial Sequence
  Description of Artificial Sequence Synthetic oligonucleotide
ggaaccgtcaattccgcactaataatttgttttagtccccttcgtttttggggtagtctaaatcccctatagtgagtcgtattaatttc

SEQ ID NO: 124 - DNA, 89 bp, Artificial Sequence
  Description of Artificial Sequence Synthetic oligonucleotide
catggctccgaaacgccgtgcaaagtgcgttttagtccccttcgtttttggggtagtctaaatcccctatagtgagtcgtattaatttc

SEQ ID NO: 125 - DNA, 89 bp, Artificial Sequence
  Description of Artificial Sequence Synthetic oligonucleotide
gctttctcattgttactcagcgcacattgttttagtccccttcgtttttggggtagtctaaatcccctatagtgagtcgtattaatttc

SEQ ID NO: 126 - DNA, 89 bp, Artificial Sequence
  Description of Artificial Sequence Synthetic oligonucleotide
tcaatcctgaggccgatactaacagtcggttttagtccccttcgtttttggggtagtctaaatcccctatagtgagtcgtattaatttc

SEQ ID NO: 127 - DNA, 89 bp, Artificial Sequence
  Description of Artificial Sequence Synthetic oligonucleotide
attctggctataccgagcccggactactgttttagtccccttcgtttttggggtagtctaaatcccctatagtgagtcgtattaatttc

SEQ ID NO: 128 - DNA, 89 bp, Artificial Sequence
  Description of Artificial Sequence Synthetic oligonucleotide
caaccatgcttcggatagcaataccacggttttagtccccttcgtttttggggtagtctaaatcccctatagtgagtcgtattaatttc

SEQ ID NO: 129 - DNA, 89 bp, Artificial Sequence
  Description of Artificial Sequence Synthetic oligonucleotide
cttgtctcaagaatagtgccctattcgtgttttagtccccttcgtttttggggtagtctaaatcccctatagtgagtcgtattaatttc

SEQ ID NO: 130 - DNA, 89 bp, Artificial Sequence
  Description of Artificial Sequence Synthetic oligonucleotide
ccgtaggtagaaacatgtatggcgcagagttttagtccccttcgtttttggggtagtctaaatcccctatagtgagtcgtattaatttc

SEQ ID NO: 131 - DNA, 89 bp, Artificial Sequence
  Description of Artificial Sequence Synthetic oligonucleotide
cagggccatgtcgtactagtagcctgccgttttagtccccttcgtttttggggtagtctaaatcccctatagtgagtcgtattaatttc

SEQ ID NO: 132 - DNA, 89 bp, Artificial Sequence
  Description of Artificial Sequence Synthetic oligonucleotide
cgagccccccaaaaagtcgggctaccgcgttttagtccccttcgtttttggggtagtctaaatcccctatagtgagtcgtattaatttc

SEQ ID NO: 133 - DNA, 89 bp, Artificial Sequence
  Description of Artificial Sequence Synthetic oligonucleotide
catataggtacatgaagccctactgtatgttttagtccccttcgtttttggggtagtctaaatcccctatagtgagtcgtattaatttc

SEQ ID NO: 134 - DNA, 89 bp, Artificial Sequence
  Description of Artificial Sequence Synthetic oligonucleotide
cataccggatggtaaactccgcgaacgcgttttagtccccttcgtttttggggtagtctaaatcccctatagtgagtcgtattaatttc

SEQ ID NO: 135 - DNA, 89 bp, Artificial Sequence
  Description of Artificial Sequence Synthetic oligonucleotide
tgcaatgaccaggcgatacgaatcgtaggttttagtccccttcgtttttggggtagtctaaatcccctatagtgagtcgtattaatttc

SEQ ID NO: 136 - DNA, 89 bp, Artificial Sequence
  Description of Artificial Sequence Synthetic oligonucleotide
ctcccccccgcagacacgcacgtgttgcgttttagtccccttcgtttttggggtagtctaaatcccctatagtgagtcgtattaatttc

SEQ ID NO: 137 - DNA, 89 bp, Artificial Sequence
  Description of Artificial Sequence Synthetic oligonucleotide
tttgtggttagccttgtcccggaacagagttttagtccccttcgtttttggggtagtctaaatcccctatagtgagtcgtattaatttc

SEQ ID NO: 138 - DNA, 89 bp, Artificial Sequence
  Description of Artificial Sequence Synthetic oligonucleotide
cccgggttgatgaggagtcactcaacgagttttagtccccttcgtttttggggtagtctaaatcccctatagtgagtcgtattaatttc

SEQ ID NO: 139 - DNA, 89 bp, Artificial Sequence
  Description of Artificial Sequence Synthetic oligonucleotide
atccatgtgcggggagaagtctggtggagttttagtccccttcgtttttggggtagtctaaatcccctatagtgagtcgtattaatttc

SEQ ID NO: 140 - DNA, 89 bp, Artificial Sequence
  Description of Artificial Sequence Synthetic oligonucleotide
ccccggtaatttcgatgttcgccccggcgttttagtccccttcgtttttggggtagtctaaatcccctatagtgagtcgtattaatttc

SEQ ID NO: 141 - DNA, 89 bp, Artificial Sequence
  Description of Artificial Sequence Synthetic oligonucleotide
tgggctgctagtaagtcgggagcccttagttttagtccccttcgtttttggggtagtctaaatcccctatagtgagtcgtattaatttc

SEQ ID NO: 142 - DNA, 89 bp, Artificial Sequence
  Description of Artificial Sequence Synthetic oligonucleotide
caggccgctaacctaaactcgacctcgagttttagtccccttcgtttttggggtagtctaaatcccctatagtgagtcgtattaatttc

SEQ ID NO: 143 - DNA, 89 bp, Artificial Sequence
  Description of Artificial Sequence Synthetic oligonucleotide
caacgcaagcgctcttggtttaagctgtgttttagtccccttcgtttttggggtagtctaaatcccctatagtgagtcgtattaatttc

SEQ ID NO: 144 - DNA, 89 bp, Artificial Sequence
  Description of Artificial Sequence Synthetic oligonucleotide
ggcacacaacgatctccaaatgaaccaagttttagtccccttcgtttttggggtagtctaaatcccctatagtgagtcgtattaatttc

SEQ ID NO: 145 - DNA, 89 bp, Artificial Sequence
  Description of Artificial Sequence Synthetic oligonucleotide
gtagcgcgacctctttaactgtagaagggttttagtccccttcgtttttggggtagtctaaatcccctatagtgagtcgtattaatttc

SEQ ID NO: 146 - DNA, 89 bp, Artificial Sequence
  Description of Artificial Sequence Synthetic oligonucleotide
gcttatcgcattccgggtgaaaacctaggttttagtccccttcgtttttggggtagtctaaatcccctatagtgagtcgtattaatttc

SEQ ID NO: 147 - DNA, 89 bp, Artificial Sequence
  Description of Artificial Sequence Synthetic oligonucleotide
agggtaatgaagccccgggatcgaacatgttttagtccccttcgtttttggggtagtctaaatcccctatagtgagtcgtattaatttc

SEQ ID NO: 148 - DNA, 89 bp, Artificial Sequence
  Description of Artificial Sequence Synthetic oligonucleotide
taactagcggcctatacccgctgcacatgttttagtccccttcgtttttggggtagtctaaatcccctatagtgagtcgtattaatttc

SEQ ID NO: 149 - DNA, 89 bp, Artificial Sequence
  Description of Artificial Sequence Synthetic oligonucleotide
cagagataggtctcgacacttacgcggtgttttagtccccttcgtttttggggtagtctaaatcccctatagtgagtcgtattaatttc

SEQ ID NO: 150 - DNA, 89 bp, Artificial Sequence
  Description of Artificial Sequence Synthetic oligonucleotide
gcctaacggctgcagcacgattgcgcaagttttagtccccttcgtttttggggtagtctaaatcccctatagtgagtcgtattaatttc

SEQ ID NO: 151 - DNA, 89 bp, Artificial Sequence
  Description of Artificial Sequence Synthetic oligonucleotide
gacgccattaaggtgggtcaccttttcagttttagtccccttcgtttttggggtagtctaaatcccctatagtgagtcgtattaatttc

SEQ ID NO: 152 - DNA, 89 bp, Artificial Sequence
  Description of Artificial Sequence Synthetic oligonucleotide
gcatatggctaaattcgctagtcgagcggttttagtccccttcgtttttggggtagtctaaatcccctatagtgagtcgtattaatttc

SEQ ID NO: 153 - DNA, 83 bp, Artificial Sequence
  Description of Artificial Sequence Synthetic oligonucleotide
gtcgacctgcaggcatgcaagcgttttagtccccttcgtttttggggtagtctaaatcccctatagtgagtcgtattaatttc

SEQ ID NO: 154 - DNA, 28 bp, Artificial Sequence
  Description of Artificial Sequence Synthetic oligonucleotide
tttttaatcggtagcgacgatatatgac

SEQ ID NO: 155 - DNA, 28 bp, Artificial Sequence
  Description of Artificial Sequence Synthetic oligonucleotide
ttgattcgtggggatccgcggatagaag

SEQ ID NO: 156 - DNA, 28 bp, Artificial Sequence
  Description of Artificial Sequence Synthetic oligonucleotide
ctaatatctgatccgttataacaccaca

SEQ ID NO: 157 - DNA, 28 bp, Artificial Sequence
  Description of Artificial Sequence Synthetic oligonucleotide
ttgcgggcctggacattcaatcgcatac

SEQ ID NO: 158 - DNA, 28 bp, Artificial Sequence
  Description of Artificial Sequence Synthetic oligonucleotide
gcctagaccaaaagtcgccttcagggca

SEQ ID NO: 159 - DNA, 28 bp, Artificial Sequence
  Description of Artificial Sequence Synthetic oligonucleotide
ataggtaggtaacggaactgaccgataa

SEQ ID NO: 160 - DNA, 28 bp, Artificial Sequence
  Description of Artificial Sequence Synthetic oligonucleotide
cgtactccttacgccttacacggacaat

SEQ ID NO: 161 - DNA, 28 bp, Artificial Sequence
  Description of Artificial Sequence Synthetic oligonucleotide
gattctctggtcatgagggaagatcgac

SEQ ID NO: 162 - DNA, 28 bp, Artificial Sequence
  Description of Artificial Sequence Synthetic oligonucleotide
tttggacggggtcactactctttacagc

SEQ ID NO: 163 - DNA, 28 bp, Artificial Sequence
  Description of Artificial Sequence Synthetic oligonucleotide
ttcatgtatgctgtcctaccgcagtagc

SEQ ID NO: 164 - DNA, 28 bp, Artificial Sequence
  Description of Artificial Sequence Synthetic oligonucleotide
tacataaggtcgtcctccggtcacgccc

SEQ ID NO: 165 - DNA, 28 bp, Artificial Sequence
  Description of Artificial Sequence Synthetic oligonucleotide
caggccgctactcaagcggggatagctt

SEQ ID NO: 166 - DNA, 28 bp, Artificial Sequence
  Description of Artificial Sequence Synthetic oligonucleotide
cgcttagctagattgcccctggaagcgc

SEQ ID NO: 167 - DNA, 28 bp, Artificial Sequence
  Description of Artificial Sequence Synthetic oligonucleotide
cggagtgtttgcgaatcccgagcattag

SEQ ID NO: 168 - DNA, 28 bp, Artificial Sequence
  Description of Artificial Sequence Synthetic oligonucleotide
gtctttcgaatcgttccttataacccgc

SEQ ID NO: 169 - DNA, 28 bp, Artificial Sequence
  Description of Artificial Sequence Synthetic oligonucleotide
aggctgaatcctccggcggctgttctaa

SEQ ID NO: 170 - DNA, 28 bp, Artificial Sequence
  Description of Artificial Sequence Synthetic oligonucleotide
cagccaaacgctagcaaaataggatata

SEQ ID NO: 171 - DNA, 28 bp, Artificial Sequence
  Description of Artificial Sequence Synthetic oligonucleotide
cagacgctgtctacctagcagaagggct

SEQ ID NO: 172 - DNA, 28 bp, Artificial Sequence
  Description of Artificial Sequence Synthetic oligonucleotide
aaggtataatgttcaagcgtggtggcta

SEQ ID NO: 173 - DNA, 28 bp, Artificial Sequence
  Description of Artificial Sequence Synthetic oligonucleotide
actgatctcggcgtaaatacaccgcaga

SEQ ID NO: 174 - DNA, 28 bp, Artificial Sequence
  Description of Artificial Sequence Synthetic oligonucleotide
tatgtgtatcctaaggtacctttacggg

SEQ ID NO: 175 - DNA, 28 bp, Artificial Sequence
  Description of Artificial Sequence Synthetic oligonucleotide
accgtcctagtgcggatagtgggacatg

SEQ ID NO: 176 - DNA, 28 bp, Artificial Sequence
  Description of Artificial Sequence Synthetic oligonucleotide
gtcgatacgggtcgctagataggttggg

SEQ ID NO: 177 - DNA, 28 bp, Artificial Sequence
  Description of Artificial Sequence Synthetic oligonucleotide
acagtcagaggcgcttcaatcgtcgccc

SEQ ID NO: 178 - DNA, 28 bp, Artificial Sequence
  Description of Artificial Sequence Synthetic oligonucleotide
gcgactcatttaatttcaacgggtagaa

SEQ ID NO: 179 - DNA, 28 bp, Artificial Sequence
  Description of Artificial Sequence Synthetic oligonucleotide
tagacagagggagctatattgcgcggcg

SEQ ID NO: 180 - DNA, 28 bp, Artificial Sequence
  Description of Artificial Sequence Synthetic oligonucleotide
ggcattcaatagccagtaaccttagaca

SEQ ID NO: 181 - DNA, 28 bp, Artificial Sequence
  Description of Artificial Sequence Synthetic oligonucleotide
acaaaagttggcatttcgtagacaaagc

SEQ ID NO: 182 - DNA, 28 bp, Artificial Sequence
  Description of Artificial Sequence Synthetic oligonucleotide
ttgcatactttgcaggttagggccgaga

SEQ ID NO: 183 - DNA, 28 bp, Artificial Sequence
  Description of Artificial Sequence Synthetic oligonucleotide
accggagttcaaacgactttgggtccct

SEQ ID NO: 184 - DNA, 28 bp, Artificial Sequence
  Description of Artificial Sequence Synthetic oligonucleotide
cactggtaggctcgcgggtaaatagagc

SEQ ID NO: 185 - DNA, 28 bp, Artificial Sequence
  Description of Artificial Sequence Synthetic oligonucleotide
ccgaagctcagtgtgatgtgccttccgt

SEQ ID NO: 186 - DNA, 28 bp, Artificial Sequence
  Description of Artificial Sequence Synthetic oligonucleotide
gtacgtatgactgtgtcccagctatgaa

SEQ ID NO: 187 - DNA, 28 bp, Artificial Sequence
  Description of Artificial Sequence Synthetic oligonucleotide
ggaccttcacgtcagtcccgctgacctt

SEQ ID NO: 188 - DNA, 28 bp, Artificial Sequence
  Description of Artificial Sequence Synthetic oligonucleotide
gaccaattatgttggccacgccacatta

SEQ ID NO: 189 - DNA, 28 bp, Artificial Sequence
  Description of Artificial Sequence Synthetic oligonucleotide
gttttcaagggctcgacacagcagaacg

SEQ ID NO: 190 - DNA, 28 bp, Artificial Sequence
  Description of Artificial Sequence Synthetic oligonucleotide
aacccactatagaagggccttcgagatt

SEQ ID NO: 191 - DNA, 28 bp, Artificial Sequence
  Description of Artificial Sequence Synthetic oligonucleotide
ggaaccgtcaattccgcactaataattt

SEQ ID NO: 192 - DNA, 28 bp, Artificial Sequence
  Description of Artificial Sequence Synthetic oligonucleotide
catggctccgaaacgccgtgcaaagtgc

SEQ ID NO: 193 - DNA, 28 bp, Artificial Sequence
  Description of Artificial Sequence Synthetic oligonucleotide
gctttctcattgttactcagcgcacatt

SEQ ID NO: 194 - DNA, 28 bp, Artificial Sequence
  Description of Artificial Sequence Synthetic oligonucleotide
tcaatcctgaggccgatactaacagtcg

SEQ ID NO: 195 - DNA, 28 bp, Artificial Sequence
  Description of Artificial Sequence Synthetic oligonucleotide
attctggctataccgagcccggactact

SEQ ID NO: 196 - DNA, 28 bp, Artificial Sequence
  Description of Artificial Sequence Synthetic oligonucleotide
caaccatgcttcggatagcaataccacg

SEQ ID NO: 197 - DNA, 28 bp, Artificial Sequence
  Description of Artificial Sequence Synthetic oligonucleotide
cttgtctcaagaatagtgccctattcgt

SEQ ID NO: 198 - DNA, 28 bp, Artificial Sequence
  Description of Artificial Sequence Synthetic oligonucleotide
ccgtaggtagaaacatgtatggcgcaga

SEQ ID NO: 199 - DNA, 28 bp, Artificial Sequence
  Description of Artificial Sequence Synthetic oligonucleotide
cagggccatgtcgtactagtagcctgcc

SEQ ID NO: 200 - DNA, 28 bp, Artificial Sequence
  Description of Artificial Sequence Synthetic oligonucleotide
cgagccccccaaaaagtcgggctaccgc

SEQ ID NO: 201 - DNA, 28 bp, Artificial Sequence
  Description of Artificial Sequence Synthetic oligonucleotide
catataggtacatgaagccctactgtat

SEQ ID NO: 202 - DNA, 28 bp, Artificial Sequence
  Description of Artificial Sequence Synthetic oligonucleotide
cataccggatggtaaactccgcgaacgc

SEQ ID NO: 203 - DNA, 28 bp, Artificial Sequence
  Description of Artificial Sequence Synthetic oligonucleotide
tgcaatgaccaggcgatacgaatcgtag

SEQ ID NO: 204 - DNA, 28 bp, Artificial Sequence
  Description of Artificial Sequence Synthetic oligonucleotide
ctcccccccgcagacacgcacgtgttgc

SEQ ID NO: 205 - DNA, 28 bp, Artificial Sequence
  Description of Artificial Sequence Synthetic oligonucleotide
tttgtggttagccttgtcccggaacaga

SEQ ID NO: 206 - DNA, 28 bp, Artificial Sequence
  Description of Artificial Sequence Synthetic oligonucleotide
cccgggttgatgaggagtcactcaacga

SEQ ID NO: 207 - DNA, 28 bp, Artificial Sequence
  Description of Artificial Sequence Synthetic oligonucleotide
atccatgtgcggggagaagtctggtgga

SEQ ID NO: 208 - DNA, 28 bp, Artificial Sequence
  Description of Artificial Sequence Synthetic oligonucleotide
ccccggtaatttcgatgttcgccccggc

SEQ ID NO: 209 - DNA, 28 bp, Artificial Sequence
  Description of Artificial Sequence Synthetic oligonucleotide
tgggctgctagtaagtcgggagccctta

SEQ ID NO: 210 - DNA, 28 bp, Artificial Sequence
  Description of Artificial Sequence Synthetic oligonucleotide
caggccgctaacctaaactcgacctcga

SEQ ID NO: 211 - DNA, 28 bp, Artificial Sequence
  Description of Artificial Sequence Synthetic oligonucleotide
caacgcaagcgctcttggtttaagctgt

SEQ ID NO: 212 - DNA, 28 bp, Artificial Sequence
  Description of Artificial Sequence Synthetic oligonucleotide
ggcacacaacgatctccaaatgaaccaa

SEQ ID NO: 213 - DNA, 28 bp, Artificial Sequence
  Description of Artificial Sequence Synthetic oligonucleotide
gtagcgcgacctctttaactgtagaagg

SEQ ID NO: 214 - DNA, 28 bp, Artificial Sequence
  Description of Artificial Sequence Synthetic oligonucleotide
gcttatcgcattccgggtgaaaacctag

SEQ ID NO: 215 - DNA, 28 bp, Artificial Sequence
  Description of Artificial Sequence Synthetic oligonucleotide
agggtaatgaagccccgggatcgaacat

SEQ ID NO: 216 - DNA, 28 bp, Artificial Sequence
  Description of Artificial Sequence Synthetic oligonucleotide
taactagcggcctatacccgctgcacat

SEQ ID NO: 217 - DNA, 28 bp, Artificial Sequence
  Description of Artificial Sequence Synthetic oligonucleotide
cagagataggtctcgacacttacgcggt

SEQ ID NO: 218 - DNA, 28 bp, Artificial Sequence
  Description of Artificial Sequence Synthetic oligonucleotide
gcctaacggctgcagcacgattgcgcaa

SEQ ID NO: 219 - DNA, 28 bp, Artificial Sequence
  Description of Artificial Sequence Synthetic oligonucleotide
gacgccattaaggtgggtcaccttttca

SEQ ID NO: 220 - DNA, 28 bp, Artificial Sequence
  Description of Artificial Sequence Synthetic oligonucleotide
gcatatggctaaattcgctagtcgagcg

SEQ ID NO: 221 - DNA, 22 bp, Artificial Sequence
  Description of Artificial Sequence Synthetic oligonucleotide
gtcgacctgcaggcatgcaagc

SEQ ID NO: 222 - DNA, 170 bp, Artificial Sequence
  Description of Artificial Sequence Synthetic polynucleotide
ctgggatcctctagaaatatggattacttggtagaacagaattctaatacgactcactatagggctagcttcgtcagtcgtaacttgggaacgcacattagatctcagtcgacctgcaggcatgcaagcttggcgtaatcatggtcatagctgtttcctgtgtttatcag

SEQ ID NO: 223 - DNA, 170 bp, Artificial Sequence
  Description of Artificial Sequence Synthetic polynucleotide
ctgggatcctctagaaatatggattacttggtagaacagaattctaatacgactcactatagggctagctctttggggttggataaatctgtcgtggttagatctcagtcgacctgcaggcatgcaagcttggcgtaatcatggtcatagctgtttcctgtgtttatcag

SEQ ID NO: 224 - DNA, 170 bp, Artificial Sequence
  Description of Artificial Sequence Synthetic polynucleotide
ctgggatcctctagaaatatggattacttggtagaacagaattctaatacgactcactatagggctagcttgaataagcgcggtccctaatgttggtgtagatctcagtcgacctgcaggcatgcaagcttggcgtaatcatggtcatagctgtttcctgtgtttatcag

SEQ ID NO: 225 - DNA, 170 bp, Artificial Sequence
  Description of Artificial Sequence Synthetic polynucleotide
ctgggatcctctagaaatatggattacttggtagaacagaattctaatacgactcactatagggctagctaccggtttatagttcgaacagtcgcagctagatctcagtcgacctgcaggcatgcaagcttggcgtaatcatggtcatagctgtttcctgtgtttatcag

SEQ ID NO: 226 - DNA, 170 bp, Artificial Sequence
  Description of Artificial Sequence Synthetic polynucleotide
ctgggatcctctagaaatatggattacttggtagaacagaattctaatacgactcactatagggctagctccccgtgtggtaactacgcaagcctaactagatctcagtcgacctgcaggcatgcaagcttggcgtaatcatggtcatagctgtttcctgtgtttatcag

SEQ ID NO: 227 - DNA, 170 bp, Artificial Sequence
  Description of Artificial Sequence Synthetic polynucleotide
ctgggatcctctagaaatatggattacttggtagaacagaattctaatacgactcactatagggctagctacgtaggggggcgcgtaaccactagctctagatctcagtcgacctgcaggcatgcaagcttggcgtaatcatggtcatagctgtttcctgtgtttatcag

SEQ ID NO: 228 - DNA, 170 bp, Artificial Sequence
  Description of Artificial Sequence Synthetic polynucleotide
ctgggatcctctagaaatatggattacttggtagaacagaattctaatacgactcactatagggctagctagtgtcccttattctactttgaattatctagatctcagtcgacctgcaggcatgcaagcttggcgtaatcatggtcatagctgtttcctgtgtttatcag

SEQ ID NO: 229 - DNA, 170 bp, Artificial Sequence
  Description of Artificial Sequence Synthetic polynucleotide
ctgggatcctctagaaatatggattacttggtagaacagaattctaatacgactcactatagggctagctgagttacgggtcaggatcattgcgcaggtagatctcagtcgacctgcaggcatgcaagcttggcgtaatcatggtcatagctgtttcctgtgtttatcag

SEQ ID NO: 230 - DNA, 170 bp, Artificial Sequence
  Description of Artificial Sequence Synthetic polynucleotide
ctgggatcctctagaaatatggattacttggtagaacagaattctaatacgactcactatagggctagctgtgagtccggcctatcacgtttggtaggtagatctcagtcgacctgcaggcatgcaagcttggcgtaatcatggtcatagctgtttcctgtgtttatcag

SEQ ID NO: 231 - DNA, 170 bp, Artificial Sequence
  Description of Artificial Sequence Synthetic polynucleotide
ctgggatcctctagaaatatggattacttggtagaacagaattctaatacgactcactatagggctagcttcaggggaaacgagttaagcagaggcagtagatctcagtcgacctgcaggcatgcaagcttggcgtaatcatggtcatagctgtttcctgtgtttatcag

SEQ ID NO: 232 - DNA, 170 bp, Artificial Sequence
  Description of Artificial Sequence Synthetic polynucleotide
ctgggatcctctagaaatatggattacttggtagaacagaattctaatacgactcactatagggctagctcttggtccaatcgtatgctaagagtagctagatctcagtcgacctgcaggcatgcaagcttggcgtaatcatggtcatagctgtttcctgtgtttatcag

SEQ ID NO: 233 - DNA, 170 bp, Artificial Sequence
  Description of Artificial Sequence Synthetic polynucleotide
ctgggatcctctagaaatatggattacttggtagaacagaattctaatacgactcactatagggctagcttcggttgcacgcgcgtcctgcacgctcgtagatctcagtcgacctgcaggcatgcaagcttggcgtaatcatggtcatagctgtttcctgtgtttatcag

SEQ ID NO: 234 - DNA, 170 bp, Artificial Sequence
  Description of Artificial Sequence Synthetic polynucleotide
ctgggatcctctagaaatatggattacttggtagaacagaattctaatacgactcactatagggctagctgttcaaaagcgggagtcccggtgaaacctagatctcagtcgacctgcaggcatgcaagcttggcgtaatcatggtcatagctgtttcctgtgtttatcag

SEQ ID NO: 235 - DNA, 170 bp, Artificial Sequence
  Description of Artificial Sequence Synthetic polynucleotide
ctgggatcctctagaaatatggattacttggtagaacagaattctaatacgactcactatagggctagctgagggcttctacgaaattgcctcaccattagatctcagtcgacctgcaggcatgcaagcttggcgtaatcatggtcatagctgtttcctgtgtttatcag

SEQ ID NO: 236 - DNA, 170 bp, Artificial Sequence
  Description of Artificial Sequence Synthetic polynucleotide
ctgggatcctctagaaatatggattacttggtagaacagaattctaatacgactcactatagggctagctgggatgccctgataacgtagagtctcagtagatctcagtcgacctgcaggcatgcaagcttggcgtaatcatggtcatagctgtttcctgtgtttatcag

SEQ ID NO: 237 - DNA, 170 bp, Artificial Sequence
  Description of Artificial Sequence Synthetic polynucleotide
ctgggatcctctagaaatatggattacttggtagaacagaattctaatacgactcactatagggctagcttagtacgggccacgatctaagtcggcggtagatctcagtcgacctgcaggcatgcaagcttggcgtaatcatggtcatagctgtttcctgtgtttatcag

SEQ ID NO: 238 - DNA, 170 bp, Artificial Sequence
  Description of Artificial Sequence Synthetic polynucleotide
ctgggatcctctagaaatatggattacttggtagaacagaattctaatacgactcactatagggctagctgtatgtgggtacgattgtaggctagaaatagatctcagtcgacctgcaggcatgcaagcttggcgtaatcatggtcatagctgtttcctgtgtttatcag

SEQ ID NO: 239 - DNA, 170 bp, Artificial Sequence
  Description of Artificial Sequence Synthetic polynucleotide
ctgggatcctctagaaatatggattacttggtagaacagaattctaatacgactcactatagggctagctagaactacctacgtggccacgcaagccctagatctcagtcgacctgcaggcatgcaagcttggcgtaatcatggtcatagctgtttcctgtgtttatcag

SEQ ID NO: 240 - DNA, 170 bp, Artificial Sequence
  Description of Artificial Sequence Synthetic polynucleotide
ctgggatcctctagaaatatggattacttggtagaacagaattctaatacgactcactatagggctagcttgcctcagaatggggtttaacgttcaggtagatctcagtcgacctgcaggcatgcaagcttggcgtaatcatggtcatagctgtttcctgtgtttatcag

SEQ ID NO: 241 - DNA, 170 bp, Artificial Sequence
  Description of Artificial Sequence Synthetic polynucleotide
ctgggatcctctagaaatatggattacttggtagaacagaattctaatacgactcactatagggctagctatgttgggacgccagactgacacgcaaatagatctcagtcgacctgcaggcatgcaagcttggcgtaatcatggtcatagctgtttcctgtgtttatcag

SEQ ID NO: 242 - DNA, 170 bp, Artificial Sequence
  Description of Artificial Sequence Synthetic polynucleotide
ctgggatcctctagaaatatggattacttggtagaacagaattctaatacgactcactatagggctagctcctacaccttcaccgagtgtggagcaagtagatctcagtcgacctgcaggcatgcaagcttggcgtaatcatggtcatagctgtttcctgtgtttatcag

SEQ ID NO: 243 - DNA, 170 bp, Artificial Sequence
  Description of Artificial Sequence Synthetic polynucleotide
ctgggatcctctagaaatatggattacttggtagaacagaattctaatacgactcactatagggctagctgtttaacattcgggtatcgtccttacgatagatctcagtcgacctgcaggcatgcaagcttggcgtaatcatggtcatagctgtttcctgtgtttatcag

SEQ ID NO: 244 - DNA, 170 bp, Artificial Sequence
  Description of Artificial Sequence Synthetic polynucleotide
ctgggatcctctagaaatatggattacttggtagaacagaattctaatacgactcactatagggctagctgcaatattactagtccgtgtcagccagatagatctcagtcgacctgcaggcatgcaagcttggcgtaatcatggtcatagctgtttcctgtgtttatcag

SEQ ID NO: 245 - DNA, 170 bp, Artificial Sequence
  Description of Artificial Sequence Synthetic polynucleotide
ctgggatcctctagaaatatggattacttggtagaacagaattctaatacgactcactatagggctagctccgactaggtcggcgataccatagcacctagatctcagtcgacctgcaggcatgcaagcttggcgtaatcatggtcatagctgtttcctgtgtttatcag

SEQ ID NO: 246 - DNA, 170 bp, Artificial Sequence
  Description of Artificial Sequence Synthetic polynucleotide
ctgggatcctctagaaatatggattacttggtagaacagaattctaatacgactcactatagggctagctgatgacgcctcacgtccgttgccccctatagatctcagtcgacctgcaggcatgcaagcttggcgtaatcatggtcatagctgtttcctgtgtttatcag

SEQ ID NO: 247 - DNA, 170 bp, Artificial Sequence
  Description of Artificial Sequence Synthetic polynucleotide
ctgggatcctctagaaatatggattacttggtagaacagaattctaatacgactcactatagggctagctgctccgtagcaattgataaaccgctgagtagatctcagtcgacctgcaggcatgcaagcttggcgtaatcatggtcatagctgtttcctgtgtttatcag

SEQ ID NO: 248 - DNA, 170 bp, Artificial Sequence
  Description of Artificial Sequence Synthetic polynucleotide
ctgggatcctctagaaatatggattacttggtagaacagaattctaatacgactcactatagggctagctctatcggatttttagataaattacgaggtagatctcagtcgacctgcaggcatgcaagcttggcgtaatcatggtcatagctgtttcctgtgtttatcag

SEQ ID NO: 249 - DNA, 170 bp, Artificial Sequence
  Description of Artificial Sequence Synthetic polynucleotide
ctgggatcctctagaaatatggattacttggtagaacagaattctaatacgactcactatagggctagcttttttaatcggtagcgacgatatatgactagatctcagtcgacctgcaggcatgcaagcttggcgtaatcatggtcatagctgtttcctgtgtttatcag

SEQ ID NO: 250 - DNA, 170 bp, Artificial Sequence
  Description of Artificial Sequence Synthetic polynucleotide
ctgggatcctctagaaatatggattacttggtagaacagaattctaatacgactcactatagggctagctttgattcgtggggatccgcggatagaagtagatctcagtcgacctgcaggcatgcaagcttggcgtaatcatggtcatagctgtttcctgtgtttatcag

SEQ ID NO: 251 - DNA, 170 bp, Artificial Sequence
  Description of Artificial Sequence Synthetic polynucleotide
ctgggatcctctagaaatatggattacttggtagaacagaattctaatacgactcactatagggctagctctaatatctgatccgttataacaccacatagatctcagtcgacctgcaggcatgcaagcttggcgtaatcatggtcatagctgtttcctgtgtttatcag

SEQ ID NO: 252 - DNA, 170 bp, Artificial Sequence
  Description of Artificial Sequence Synthetic polynucleotide
ctgggatcctctagaaatatggattacttggtagaacagaattctaatacgactcactatagggctagctttgcgggcctggacattcaatcgcatactagatctcagtcgacctgcaggcatgcaagcttggcgtaatcatggtcatagctgtttcctgtgtttatcag

SEQ ID NO: 253 - DNA, 170 bp, Artificial Sequence
  Description of Artificial Sequence Synthetic polynucleotide
ctgggatcctctagaaatatggattacttggtagaacagaattctaatacgactcactatagggctagctgcctagaccaaaagtcgccttcagggcatagatctcagtcgacctgcaggcatgcaagcttggcgtaatcatggtcatagctgtttcctgtgtttatcag

SEQ ID NO: 254 - DNA, 170 bp, Artificial Sequence
  Description of Artificial Sequence Synthetic polynucleotide
ctgggatcctctagaaatatggattacttggtagaacagaattctaatacgactcactatagggctagctataggtaggtaacggaactgaccgataatagatctcagtcgacctgcaggcatgcaagcttggcgtaatcatggtcatagctgtttcctgtgtttatcag

SEQ ID NO: 255 - DNA, 170 bp, Artificial Sequence
  Description of Artificial Sequence Synthetic polynucleotide
ctgggatcctctagaaatatggattacttggtagaacagaattctaatacgactcactatagggctagctcgtactccttacgccttacacggacaattagatctcagtcgacctgcaggcatgcaagcttggcgtaatcatggtcatagctgtttcctgtgtttatcag

SEQ ID NO: 256 - DNA, 170 bp, Artificial Sequence
  Description of Artificial Sequence Synthetic polynucleotide
ctgggatcctctagaaatatggattacttggtagaacagaattctaatacgactcactatagggctagctgattctctggtcatgagggaagatcgactagatctcagtcgacctgcaggcatgcaagcttggcgtaatcatggtcatagctgtttcctgtgtttatcag

SEQ ID NO: 257 - DNA, 170 bp, Artificial Sequence
  Description of Artificial Sequence Synthetic polynucleotide
ctgggatcctctagaaatatggattacttggtagaacagaattctaatacgactcactatagggctagcttttggacggggtcactactctttacagctagatctcagtcgacctgcaggcatgcaagcttggcgtaatcatggtcatagctgtttcctgtgtttatcag

SEQ ID NO: 258 - DNA, 170 bp, Artificial Sequence
  Description of Artificial Sequence Synthetic polynucleotide
ctgggatcctctagaaatatggattacttggtagaacagaattctaatacgactcactatagggctagctttcatgtatgctgtcctaccgcagtagctagatctcagtcgacctgcaggcatgcaagcttggcgtaatcatggtcatagctgtttcctgtgtttatcag

SEQ ID NO: 259 - DNA, 170 bp, Artificial Sequence
  Description of Artificial Sequence Synthetic polynucleotide
ctgggatcctctagaaatatggattacttggtagaacagaattctaatacgactcactatagggctagcttacataaggtcgtcctccggtcacgccctagatctcagtcgacctgcaggcatgcaagcttggcgtaatcatggtcatagctgtttcctgtgtttatcag

SEQ ID NO: 260 - DNA, 170 bp, Artificial Sequence
  Description of Artificial Sequence Synthetic polynucleotide
ctgggatcctctagaaatatggattacttggtagaacagaattctaatacgactcactatagggctagctcaggccgctactcaagcggggatagctttagatctcagtcgacctgcaggcatgcaagcttggcgtaatcatggtcatagctgtttcctgtgtttatcag

SEQ ID NO: 261 - DNA, 170 bp, Artificial Sequence
  Description of Artificial Sequence Synthetic polynucleotide
ctgggatcctctagaaatatggattacttggtagaacagaattctaatacgactcactatagggctagctcgcttagctagattgcccctggaagcgctagatctcagtcgacctgcaggcatgcaagcttggcgtaatcatggtcatagctgtttcctgtgtttatcag

SEQ ID NO: 262 - DNA, 170 bp, Artificial Sequence
  Description of Artificial Sequence Synthetic polynucleotide
ctgggatcctctagaaatatggattacttggtagaacagaattctaatacgactcactatagggctagctcggagtgtttgcgaatcccgagcattagtagatctcagtcgacctgcaggcatgcaagcttggcgtaatcatggtcatagctgtttcctgtgtttatcag

SEQ ID NO: 263 - DNA, 170 bp, Artificial Sequence
  Description of Artificial Sequence Synthetic polynucleotide
ctgggatcctctagaaatatggattacttggtagaacagaattctaatacgactcactatagggctagctgtctttcgaatcgttccttataacccgctagatctcagtcgacctgcaggcatgcaagcttggcgtaatcatggtcatagctgtttcctgtgtttatcag

SEQ ID NO: 264 - DNA, 170 bp, Artificial Sequence
  Description of Artificial Sequence Synthetic polynucleotide
ctgggatcctctagaaatatggattacttggtagaacagaattctaatacgactcactatagggctagctaggctgaatcctccggcggctgttctaatagatctcagtcgacctgcaggcatgcaagcttggcgtaatcatggtcatagctgtttcctgtgtttatcag

SEQ ID NO: 265 - DNA, 170 bp, Artificial Sequence
  Description of Artificial Sequence Synthetic polynucleotide
ctgggatcctctagaaatatggattacttggtagaacagaattctaatacgactcactatagggctagctcagccaaacgctagcaaaataggatatatagatctcagtcgacctgcaggcatgcaagcttggcgtaatcatggtcatagctgtttcctgtgtttatcag

SEQ ID NO: 266 - DNA, 170 bp, Artificial Sequence
  Description of Artificial Sequence Synthetic polynucleotide
ctgggatcctctagaaatatggattacttggtagaacagaattctaatacgactcactatagggctagctcagacgctgtctacctagcagaagggcttagatctcagtcgacctgcaggcatgcaagcttggcgtaatcatggtcatagctgtttcctgtgtttatcag

SEQ ID NO: 267 - DNA, 170 bp, Artificial Sequence
  Description of Artificial Sequence Synthetic polynucleotide
ctgggatcctctagaaatatggattacttggtagaacagaattctaatacgactcactatagggctagctaaggtataatgttcaagcgtggtggctatagatctcagtcgacctgcaggcatgcaagcttggcgtaatcatggtcatagctgtttcctgtgtttatcag

SEQ ID NO: 268 - DNA, 170 bp, Artificial Sequence
  Description of Artificial Sequence Synthetic polynucleotide
ctgggatcctctagaaatatggattacttggtagaacagaattctaatacgactcactatagggctagctactgatctcggcgtaaatacaccgcagatagatctcagtcgacctgcaggcatgcaagcttggcgtaatcatggtcatagctgtttcctgtgtttatcag

SEQ ID NO: 269 - DNA, 170 bp, Artificial Sequence
  Description of Artificial Sequence Synthetic polynucleotide
ctgggatcctctagaaatatggattacttggtagaacagaattctaatacgactcactatagggctagcttatgtgtatcctaaggtacctttacgggtagatctcagtcgacctgcaggcatgcaagcttggcgtaatcatggtcatagctgtttcctgtgtttatcag

SEQ ID NO: 270 - DNA, 170 bp, Artificial Sequence
  Description of Artificial Sequence Synthetic polynucleotide
ctgggatcctctagaaatatggattacttggtagaacagaattctaatacgactcactatagggctagctaccgtcctagtgcggatagtgggacatgtagatctcagtcgacctgcaggcatgcaagcttggcgtaatcatggtcatagctgtttcctgtgtttatcag

SEQ ID NO: 271 - DNA, 170 bp, Artificial Sequence
  Description of Artificial Sequence Synthetic polynucleotide
ctgggatcctctagaaatatggattacttggtagaacagaattctaatacgactcactatagggctagctgtcgatacgggtcgctagataggttgggtagatctcagtcgacctgcaggcatgcaagcttggcgtaatcatggtcatagctgtttcctgtgtttatcag

SEQ ID NO: 272 - DNA, 170 bp, Artificial Sequence
  Description of Artificial Sequence Synthetic polynucleotide
ctgggatcctctagaaatatggattacttggtagaacagaattctaatacgactcactatagggctagctacagtcagaggcgcttcaatcgtcgccctagatctcagtcgacctgcaggcatgcaagcttggcgtaatcatggtcatagctgtttcctgtgtttatcag

SEQ ID NO: 273 - DNA, 170 bp, Artificial Sequence
  Description of Artificial Sequence Synthetic polynucleotide
ctgggatcctctagaaatatggattacttggtagaacagaattctaatacgactcactatagggctagctgcgactcatttaatttcaacgggtagaatagatctcagtcgacctgcaggcatgcaagcttggcgtaatcatggtcatagctgtttcctgtgtttatcag

SEQ ID NO: 274 - DNA, 170 bp, Artificial Sequence
  Description of Artificial Sequence Synthetic polynucleotide
ctgggatcctctagaaatatggattacttggtagaacagaattctaatacgactcactatagggctagcttagacagagggagctatattgcgcggcgtagatctcagtcgacctgcaggcatgcaagcttggcgtaatcatggtcatagctgtttcctgtgtttatcag

SEQ ID NO: 275 - DNA, 170 bp, Artificial Sequence
  Description of Artificial Sequence Synthetic polynucleotide
ctgggatcctctagaaatatggattacttggtagaacagaattctaatacgactcactatagggctagctggcattcaatagccagtaaccttagacatagatctcagtcgacctgcaggcatgcaagcttggcgtaatcatggtcatagctgtttcctgtgtttatcag

SEQ ID NO: 276 - DNA, 170 bp, Artificial Sequence
  Description of Artificial Sequence Synthetic polynucleotide
ctgggatcctctagaaatatggattacttggtagaacagaattctaatacgactcactatagggctagctacaaaagttggcatttcgtagacaaagctagatctcagtcgacctgcaggcatgcaagcttggcgtaatcatggtcatagctgtttcctgtgtttatcag

SEQ ID NO: 277 - DNA, 170 bp, Artificial Sequence
  Description of Artificial Sequence Synthetic polynucleotide
ctgggatcctctagaaatatggattacttggtagaacagaattctaatacgactcactatagggctagctttgcatactttgcaggttagggccgagatagatctcagtcgacctgcaggcatgcaagcttggcgtaatcatggtcatagctgtttcctgtgtttatcag

SEQ ID NO: 278 - DNA, 170 bp, Artificial Sequence
  Description of Artificial Sequence Synthetic polynucleotide
ctgggatcctctagaaatatggattacttggtagaacagaattctaatacgactcactatagggctagctaccggagttcaaacgactttgggtcccttagatctcagtcgacctgcaggcatgcaagcttggcgtaatcatggtcatagctgtttcctgtgtttatcag

SEQ ID NO: 279 - DNA, 170 bp, Artificial Sequence
  Description of Artificial Sequence Synthetic polynucleotide
ctgggatcctctagaaatatggattacttggtagaacagaattctaatacgactcactatagggctagctcactggtaggctcgcgggtaaatagagctagatctcagtcgacctgcaggcatgcaagcttggcgtaatcatggtcatagctgtttcctgtgtttatcag

SEQ ID NO: 280 - DNA, 170 bp, Artificial Sequence
  Description of Artificial Sequence Synthetic polynucleotide
ctgggatcctctagaaatatggattacttggtagaacagaattctaatacgactcactatagggctagctccgaagctcagtgtgatgtgccttccgttagatctcagtcgacctgcaggcatgcaagcttggcgtaatcatggtcatagctgtttcctgtgtttatcag

SEQ ID NO: 281 - DNA, 170 bp, Artificial Sequence
  Description of Artificial Sequence Synthetic polynucleotide
ctgggatcctctagaaatatggattacttggtagaacagaattctaatacgactcactatagggctagctgtacgtatgactgtgtcccagctatgaatagatctcagtcgacctgcaggcatgcaagcttggcgtaatcatggtcatagctgtttcctgtgtttatcag

SEQ ID NO: 282 - DNA, 170 bp, Artificial Sequence
  Description of Artificial Sequence Synthetic polynucleotide
ctgggatcctctagaaatatggattacttggtagaacagaattctaatacgactcactatagggctagctggaccttcacgtcagtcccgctgacctttagatctcagtcgacctgcaggcatgcaagcttggcgtaatcatggtcatagctgtttcctgtgtttatcag

SEQ ID NO: 283 - DNA, 170 bp, Artificial Sequence
  Description of Artificial Sequence Synthetic polynucleotide
ctgggatcctctagaaatatggattacttggtagaacagaattctaatacgactcactatagggctagctgaccaattatgttggccacgccacattatagatctcagtcgacctgcaggcatgcaagcttggcgtaatcatggtcatagctgtttcctgtgtttatcag

SEQ ID NO: 284 - DNA, 170 bp, Artificial Sequence
  Description of Artificial Sequence Synthetic polynucleotide
ctgggatcctctagaaatatggattacttggtagaacagaattctaatacgactcactatagggctagctgttttcaagggctcgacacagcagaacgtagatctcagtcgacctgcaggcatgcaagcttggcgtaatcatggtcatagctgtttcctgtgtttatcag

SEQ ID NO: 285 - DNA, 170 bp, Artificial Sequence
  Description of Artificial Sequence Synthetic polynucleotide
ctgggatcctctagaaatatggattacttggtagaacagaattctaatacgactcactatagggctagctaacccactatagaagggccttcgagatttagatctcagtcgacctgcaggcatgcaagcttggcgtaatcatggtcatagctgtttcctgtgtttatcag

SEQ ID NO: 286 - DNA, 170 bp, Artificial Sequence
  Description of Artificial Sequence Synthetic polynucleotide
ctgggatcctctagaaatatggattacttggtagaacagaattctaatacgactcactatagggctagctggaaccgtcaattccgcactaataattttagatctcagtcgacctgcaggcatgcaagcttggcgtaatcatggtcatagctgtttcctgtgtttatcag

SEQ ID NO: 287 - DNA, 170 bp, Artificial Sequence
  Description of Artificial Sequence Synthetic polynucleotide
ctgggatcctctagaaatatggattacttggtagaacagaattctaatacgactcactatagggctagctcatggctccgaaacgccgtgcaaagtgctagatctcagtcgacctgcaggcatgcaagcttggcgtaatcatggtcatagctgtttcctgtgtttatcag

SEQ ID NO: 288 - DNA, 170 bp, Artificial Sequence
  Description of Artificial Sequence Synthetic polynucleotide
ctgggatcctctagaaatatggattacttggtagaacagaattctaatacgactcactatagggctagctgctttctcattgttactcagcgcacatttagatctcagtcgacctgcaggcatgcaagcttggcgtaatcatggtcatagctgtttcctgtgtttatcag

SEQ ID NO: 289 - DNA, 170 bp, Artificial Sequence
  Description of Artificial Sequence Synthetic polynucleotide
ctgggatcctctagaaatatggattacttggtagaacagaattctaatacgactcactatagggctagcttcaatcctgaggccgatactaacagtcgtagatctcagtcgacctgcaggcatgcaagcttggcgtaatcatggtcatagctgtttcctgtgtttatcag

SEQ ID NO: 290 - DNA, 170 bp, Artificial Sequence
  Description of Artificial Sequence Synthetic polynucleotide
ctgggatcctctagaaatatggattacttggtagaacagaattctaatacgactcactatagggctagctattctggctataccgagcccggactacttagatctcagtcgacctgcaggcatgcaagcttggcgtaatcatggtcatagctgtttcctgtgtttatcag

SEQ ID NO: 291 - DNA, 170 bp, Artificial Sequence
  Description of Artificial Sequence Synthetic polynucleotide
ctgggatcctctagaaatatggattacttggtagaacagaattctaatacgactcactatagggctagctcaaccatgcttcggatagcaataccacgtagatctcagtcgacctgcaggcatgcaagcttggcgtaatcatggtcatagctgtttcctgtgtttatcag

SEQ ID NO: 292 - DNA, 170 bp, Artificial Sequence
  Description of Artificial Sequence Synthetic polynucleotide
ctgggatcctctagaaatatggattacttggtagaacagaattctaatacgactcactatagggctagctcttgtctcaagaatagtgccctattcgttagatctcagtcgacctgcaggcatgcaagcttggcgtaatcatggtcatagctgtttcctgtgtttatcag

SEQ ID NO: 293 - DNA, 170 bp, Artificial Sequence
  Description of Artificial Sequence Synthetic polynucleotide
ctgggatcctctagaaatatggattacttggtagaacagaattctaatacgactcactatagggctagctccgtaggtagaaacatgtatggcgcagatagatctcagtcgacctgcaggcatgcaagcttggcgtaatcatggtcatagctgtttcctgtgtttatcag

SEQ ID NO: 294 - DNA, 170 bp, Artificial Sequence
  Description of Artificial Sequence Synthetic polynucleotide
ctgggatcctctagaaatatggattacttggtagaacagaattctaatacgactcactatagggctagctcagggccatgtcgtactagtagcctgcctagatctcagtcgacctgcaggcatgcaagcttggcgtaatcatggtcatagctgtttcctgtgtttatcag

SEQ ID NO: 295 - DNA, 170 bp, Artificial Sequence
  Description of Artificial Sequence Synthetic polynucleotide
ctgggatcctctagaaatatggattacttggtagaacagaattctaatacgactcactatagggctagctcgagccccccaaaaagtcgggctaccgctagatctcagtcgacctgcaggcatgcaagcttggcgtaatcatggtcatagctgtttcctgtgtttatcag

SEQ ID NO: 296 - DNA, 170 bp, Artificial Sequence
  Description of Artificial Sequence Synthetic polynucleotide
ctgggatcctctagaaatatggattacttggtagaacagaattctaatacgactcactatagggctagctcatataggtacatgaagccctactgtattagatctcagtcgacctgcaggcatgcaagcttggcgtaatcatggtcatagctgtttcctgtgtttatcag

SEQ ID NO: 297 - DNA, 170 bp, Artificial Sequence
  Description of Artificial Sequence Synthetic polynucleotide
ctgggatcctctagaaatatggattacttggtagaacagaattctaatacgactcactatagggctagctcataccggatggtaaactccgcgaacgctagatctcagtcgacctgcaggcatgcaagcttggcgtaatcatggtcatagctgtttcctgtgtttatcag

SEQ ID NO: 298 - DNA, 170 bp, Artificial Sequence
  Description of Artificial Sequence Synthetic polynucleotide
ctgggatcctctagaaatatggattacttggtagaacagaattctaatacgactcactatagggctagcttgcaatgaccaggcgatacgaatcgtagtagatctcagtcgacctgcaggcatgcaagcttggcgtaatcatggtcatagctgtttcctgtgtttatcag

SEQ ID NO: 299 - DNA, 170 bp, Artificial Sequence
  Description of Artificial Sequence Synthetic polynucleotide
ctgggatcctctagaaatatggattacttggtagaacagaattctaatacgactcactatagggctagctctcccccccgcagacacgcacgtgttgctagatctcagtcgacctgcaggcatgcaagcttggcgtaatcatggtcatagctgtttcctgtgtttatcag

SEQ ID NO: 300 - DNA, 170 bp, Artificial Sequence
  Description of Artificial Sequence Synthetic polynucleotide
ctgggatcctctagaaatatggattacttggtagaacagaattctaatacgactcactatagggctagcttttgtggttagccttgtcccggaacagatagatctcagtcgacctgcaggcatgcaagcttggcgtaatcatggtcatagctgtttcctgtgtttatcag

SEQ ID NO: 301 - DNA, 170 bp, Artificial Sequence
  Description of Artificial Sequence Synthetic polynucleotide
ctgggatcctctagaaatatggattacttggtagaacagaattctaatacgactcactatagggctagctcccgggttgatgaggagtcactcaacgatagatctcagtcgacctgcaggcatgcaagcttggcgtaatcatggtcatagctgtttcctgtgtttatcag

SEQ ID NO: 302 - DNA, 170 bp, Artificial Sequence
  Description of Artificial Sequence Synthetic polynucleotide
ctgggatcctctagaaatatggattacttggtagaacagaattctaatacgactcactatagggctagctatccatgtgcggggagaagtctggtggatagatctcagtcgacctgcaggcatgcaagcttggcgtaatcatggtcatagctgtttcctgtgtttatcag

SEQ ID NO: 303 - DNA, 170 bp, Artificial Sequence
  Description of Artificial Sequence Synthetic polynucleotide
ctgggatcctctagaaatatggattacttggtagaacagaattctaatacgactcactatagggctagctccccggtaatttcgatgttcgccccggctagatctcagtcgacctgcaggcatgcaagcttggcgtaatcatggtcatagctgtttcctgtgtttatcag

SEQ ID NO: 304 - DNA, 170 bp, Artificial Sequence
  Description of Artificial Sequence Synthetic polynucleotide
ctgggatcctctagaaatatggattacttggtagaacagaattctaatacgactcactatagggctagcttgggctgctagtaagtcgggagcccttatagatctcagtcgacctgcaggcatgcaagcttggcgtaatcatggtcatagctgtttcctgtgtttatcag

SEQ ID NO: 305 - DNA, 170 bp, Artificial Sequence
  Description of Artificial Sequence Synthetic polynucleotide
ctgggatcctctagaaatatggattacttggtagaacagaattctaatacgactcactatagggctagctcaggccgctaacctaaactcgacctcgatagatctcagtcgacctgcaggcatgcaagcttggcgtaatcatggtcatagctgtttcctgtgtttatcag

SEQ ID NO: 306 - DNA, 170 bp, Artificial Sequence
  Description of Artificial Sequence Synthetic polynucleotide
ctgggatcctctagaaatatggattacttggtagaacagaattctaatacgactcactatagggctagctcaacgcaagcgctcttggtttaagctgttagatctcagtcgacctgcaggcatgcaagcttggcgtaatcatggtcatagctgtttcctgtgtttatcag

SEQ ID NO: 307 - DNA, 170 bp, Artificial Sequence
  Description of Artificial Sequence Synthetic polynucleotide
ctgggatcctctagaaatatggattacttggtagaacagaattctaatacgactcactatagggctagctggcacacaacgatctccaaatgaaccaatagatctcagtcgacctgcaggcatgcaagcttggcgtaatcatggtcatagctgtttcctgtgtttatcag

SEQ ID NO: 308 - DNA, 170 bp, Artificial Sequence
  Description of Artificial Sequence Synthetic polynucleotide
ctgggatcctctagaaatatggattacttggtagaacagaattctaatacgactcactatagggctagctgtagcgcgacctctttaactgtagaaggtagatctcagtcgacctgcaggcatgcaagcttggcgtaatcatggtcatagctgtttcctgtgtttatcag

SEQ ID NO: 309 - DNA, 170 bp, Artificial Sequence
  Description of Artificial Sequence Synthetic polynucleotide
ctgggatcctctagaaatatggattacttggtagaacagaattctaatacgactcactatagggctagctgcttatcgcattccgggtgaaaacctagtagatctcagtcgacctgcaggcatgcaagcttggcgtaatcatggtcatagctgtttcctgtgtttatcag

SEQ ID NO: 310 - DNA, 170 bp, Artificial Sequence
  Description of Artificial Sequence Synthetic polynucleotide
ctgggatcctctagaaatatggattacttggtagaacagaattctaatacgactcactatagggctagctagggtaatgaagccccgggatcgaacattagatctcagtcgacctgcaggcatgcaagcttggcgtaatcatggtcatagctgtttcctgtgtttatcag

SEQ ID NO: 311 - DNA, 170 bp, Artificial Sequence
  Description of Artificial Sequence Synthetic polynucleotide
ctgggatcctctagaaatatggattacttggtagaacagaattctaatacgactcactatagggctagcttaactagcggcctatacccgctgcacattagatctcagtcgacctgcaggcatgcaagcttggcgtaatcatggtcatagctgtttcctgtgtttatcag

SEQ ID NO: 312 - DNA, 170 bp, Artificial Sequence
  Description of Artificial Sequence Synthetic polynucleotide
ctgggatcctctagaaatatggattacttggtagaacagaattctaatacgactcactatagggctagctcagagataggtctcgacacttacgcggttagatctcagtcgacctgcaggcatgcaagcttggcgtaatcatggtcatagctgtttcctgtgtttatcag

SEQ ID NO: 313 - DNA, 170 bp, Artificial Sequence
  Description of Artificial Sequence Synthetic polynucleotide
ctgggatcctctagaaatatggattacttggtagaacagaattctaatacgactcactatagggctagctgcctaacggctgcagcacgattgcgcaatagatctcagtcgacctgcaggcatgcaagcttggcgtaatcatggtcatagctgtttcctgtgtttatcag

SEQ ID NO: 314 - DNA, 170 bp, Artificial Sequence
  Description of Artificial Sequence Synthetic polynucleotide
ctgggatcctctagaaatatggattacttggtagaacagaattctaatacgactcactatagggctagctgacgccattaaggtgggtcaccttttcatagatctcagtcgacctgcaggcatgcaagcttggcgtaatcatggtcatagctgtttcctgtgtttatcag

SEQ ID NO: 315 - DNA, 170 bp, Artificial Sequence
  Description of Artificial Sequence Synthetic polynucleotide
ctgggatcctctagaaatatggattacttggtagaacagaattctaatacgactcactatagggctagctgcatatggctaaattcgctagtcgagcgtagatctcagtcgacctgcaggcatgcaagcttggcgtaatcatggtcatagctgtttcctgtgtttatcag

SEQ ID NO: 316 - DNA, 1761 bp, Saccharomyces cerevisiae
atgctttctgaaaacacgactattctgatggctaacggtgaaattaaagacatcgcaaacgtcacggctaactcttacgttatgtgcgcagatggctccgctgcccgcgtcataaatgtcacacagggctatcagaaaatctataatatacagcaaaaaaccaaacacagagcttttgaaggtgaacctggtaggttagatcccaggcgtagaacagtttatcagcgtcttgcattacaatgtactgcaggtcataaattgtcagtcagggtccctaccaaaccactgttggaaaaaagtggtagaaatgccaccaaatataaagtgagatggagaaatctgcagcaatgtcagacgcttgatggtaggataataataattccaaaaaaccatcataagacattcccaatgacagttgaaggtgagtttgccgcaaaacgcttcatagaagaaatggagcgctctaaaggagaatatttcaactttgacattgaagttagagatttggattatcttgatgctcaattgagaatttctagctgcataagatttggtccagtactcgcaggaaatggtgttttatctaaatttctcactggacgtagtgaccttgtaactcctgctgtaaaaagtatggcttggatgcttggtctgtggttaggtgacagtacaacaaaagagccagaaatctcagtagatagcttggatcctaagctaatggagagtttaagagaaaatgcgaaaatctggggtctctaccttacggtttgtgacgatcacgttccgctacgtgccaaacatgtaaggcttcattatggagatggtccagatgaaaacaggaagacaaggaatttgaggaaaaataatccattctggaaagctgtcacaattttaaagtttaaaagggatcttgatggagagaagcaaatccctgaatttatgtacggcgagcatatagaagttcgtgaagcattcttagccggcttgatcgactcagatgggtacgttgtgaaaaagggcgaaggccctgaatcttataaaatagcaattcaaactgtttattcatccattatggacggaattgtccatatttcaagatctcttggtatgtcagctactgtgacgaccaggtcagctagggaggaaatcattgaaggaagaaaagtccaatgtcaatttacatacgactgtaatgttgctgggggaacaacttcacagaatgttttgtcatattgtcgaagtggtcacaaaacaagagaagttccgccaattataaaaagggaacccgtatatttcagcttcacggatgatttccagggtgagagtactgtatatgggcttacgatagaaggccataaaaatttcttgcttggcaacaaaatagaagtgaaatcatgtcgaggctgctgtgtgggagaacagcttaaaatatcacaaaaaaagaatctaaaacactgtgttgcttgtcccagaaagggaatcaagtatttttataaagattggagtggtaaaaatcgagtatgtgctagatgctatggaagatacaaattcagcggtcatcactgtataaattgcaagtatgtaccagaagcacgtgaagtgaaaaaggcaaaagacaaaggcgaaaaattgggcattacgcccgaaggtttgccagttaaaggaccagagtgtataaaatgtggcggaatcttacagtttgatgctgtccgcgggcctcataagagttgtggtaacaacgcaggtgcgcgcatctgctaa

SEQ ID NO: 317 - DNA, 1455 bp, Bacillus subtilis
ctgattatttcgatttgtatttatatggcgggaatgctgctaatcggctactttgcttacaagcgtacgtcgaatctgacggattatatgctgggaggacgctctttgggtccggcagtaaccgctctcagtgccggcgctgccgatatgagcggctggctgctaatggggcttccgggcgccatgttttctactggtctgagcggcgcttggatcgtcatcggactttgccttggagcgtgggcgaactggctttacgtcgcaccacggctgagaacctatactgaaaaagcggggaattctattacaattcccggattcctggaaaatcgcttcggagatcaaacaaagcttctcagactgttttcgggaattgtcattttagtctttttcacgttttatgtatcgtccggaatggtgtccggcggcgtcttattcaacagcatcttggggatggattatcacaccggcctgtggattgtgacgggtgttgttgtggcatataccctgtttggcggctttttggcagtcagctggacagattttgttcaggggattattatgtttgccgcactcattcttgtgccgatcgttacgtttttccacacgggaggagcaggtgatacagttgctgaaatccgctctgtagatcctgatatgtttaatattttcaaaggaacgagcgtccttggcattatttctttgtttgcctgggggctgggatactttggccagccgcatattattgtgcgttttatggcgataacgtctgtcaaagagatcaaaagagcgcgcagaattgggatgggctggatgattttgtctgcagtaggcgcagtgctgaccggtttgggcggaatcgcttattaccatcagcgcggcatgacactgaaggacccagagacgatttttattcaattagggaatatcttgttccatccgattatcacgggttttctgatttcagcgattttggccgcgattatgagtacaatttcttcccagctgcttgtgacatcaagctctctggtagaagatttgtataagtcgatgttcagacgctcagcttcagataaagaactggtgtttttaggccgactggctgttctggctgtttcaattgtcgcgttggttctggcttgggagaaaaataacaccatccttggattggtcagctacgcgtgggcgggcttcggtgcatcattcgggccagttgtactgctcagtctgttttggaaacgaatgaccaaatggggggcactcgccggcatgattgtgggagcagcgactgtaatcatttgggcgaatgccggtctttcggactttctgtatgaaatgatacctggttttgctgcgagtctattatctgtcttttttgtcagcatattgacgcaggctccgtcacaagctgtcacagaccagttcaacgactaccaagatacaatgtcgcaataa

SEQ ID NO: 318 - DNA, 2142 bp, Bacillus thuringiensis
atgttgaaagtaaaacgtccatttgatgcctatttagatgaaatgaataaaattacaattttgctcccacatgcgtatggaacaagtcggacatttcgtttgcaagaaggaagtaatgtgaaagatttaccgattgctcatactattgcgcttccagatgcaacgaagtatgagtgctttatagaagagccgctagatgtggggaagtattatacagtacgagatgaacggaatgaagaaactgatttacaaataggtgctgttattcgaacaaaaattttcgatgaaaagtattattacgaaggtacagatttaggtgctgtgtatcaaaaacaagaaacgacatttaaagtatgggccccaactgcaaggcttgcgaaagtaagaatttataagagtgataaagaatataccgatcatgaaatgtacaggggagaaaacggagtttggattcaaacattacaaggtgatttagacggagcacgctatacatttcttgtttgtattaatttaatatggaacgaagcggtggatccttacgcaaagtcagtgactgtaaatggaaagtatggcattgttatcgatttagaaaagacgactgtaacgaaacgagagcaattaccaccattacaagcaatgacagatgccatattgtatgaattgcatattcgtgatgctactattcatccgaacagtggagtgagtaaaaaagggacgtataaagggctaatggaagaaggaacaacaggacgaaatgggacattgacaggtttgtctcatataaaagatttaggcgttacgcatgttgaattattacctttatattgtttcggtggtgtagatgaggcgaatccttcgtccgcctataattggggatataatccattatattacaatgcaccaacaggattttatgctacaaacccctctgatccgtataacaggatagttgagtgcaaacaactaattgaaacatttcatgaacaaggtattagggtgattatagatgttgtatataatcatgtttatgaaagagagctatcatcatttgagaagcttgttccaggatattattttcgtcatggtgaagatggtatgccttctaatgggacgggagtcggaaatgatgtagcgtctgaacggaaaatgatgaggaaattcattgtagagtctattttatattggcttactgaatacaatgtcgatggattccgattcgacttaatgggaattttagatgttgatacaatgaatatgatagagaaagaagtgcgaaacataaaacgagatgctttgttattaggagaaggatgggatttacaaacaccgcttcctcttgaggaaaaagcaacgttaaataatgcgaagaaaatgccacatatcgcgcagtttaatgatcaatttcgtgatgggataaaagggagtacatttcatataaataagcgtggttttgcatttggtgggcatgtagactgtaatcatttgcagtatattgcagcaggaagtcttttaagtatgaaagaaacagggctgtttttagagccggcgcaaagcattaactatgtagaatgtcatgataatatgacgatgtgggataaactagtgcgcagtaatgaagaatcagaaacgattttgaaaaaacgtcacctattagcgagcgcaatggttattctttcacaaggcattcctttcttacacgcagggcaagagttttatcgtacgaagaaaggaaatgaaaatagttataatgcaaatgatgagatcaatcaattagattgggatcaaaaagagaaagagatagagaccgttaactatataaaaggtttaattgcgattcgtaaagagcatggggcattccgattacaaaatgcagaccttataaaaaaacatatgacttttttacaaacatctacagaagtacttgcatatcacctagagcatgcagaatcgtttgggccgtggaaagagattatcgtcttatttaatagcggtttagaagctaaaactgtgcaacttccgaaagaagcaacgtggcatgtgttagtaaacgaaaacgaagcgaaaatagagccaatttcttcatttagaggaaaggaacttaggctcgctccaattagcacgtatatattgacgataatgtga

SEQ ID NO: 319 - DNA, 1059 bp, Bacillus subtilis
atgtggaaaggacttatccatcaatataaagaatttttacctgtaacagatcaaacaccggcgctaactttacatgaaggaaacacacctcttattcacctgccgaagctgtctgagcagctcggaattgagcttcatgtcaaaacggaaggcgtcaatcctacgggatcatttaaagatcgcggaatggttatggctgtggcaaaggcaaaagaagaaggcaatgacacgattatgtgcgcgtcaacaggtaacacttccgctgctgcggcagcatatgcagcccgtgctaacatgaaatgcattgtcatcatcccgaacggaaaaattgcatttggaaaactcgctcaagctgtcatgtacggagccgagattatcgcaattgacggaaactttgacgatgcgcttaaaattgtccgttccatctgtgagaaatcaccgattgcccttgtcaactcagtcaacccttaccgcattgaaggccaaaaaactgctgccttcgaagtgtgcgaacagctgggggaagcgcctgatgttttagcgatcccggtcggaaatgctggaaacatcactgcgtactggaagggcttcaaggaatatcatgagaaaaacggcacaggccttccgaaaatgcgcggctttgaagctgagggtgcggcggcaatcgtgcgcaatgaagtgattgaaaatccggaaacaatagcgacagccattcgtatcggaaacccggcaagctgggacaaagctgtaaaggcagccgaggaatccaatgggaaaattgacgaagtcactgatgatgaaatccttcacgcatatcagctgatcgcccgtgtagaaggcgtgtttgcagaaccaggttcttgcgcgtctatcgcaggagtgctgaaacaggtgaaatccggagaaattccgaaaggcagcaaggtcgtagctgtgttaacaggaaacggactgaaagatccgaacacagcggtcgacatttcagaaatcaagcctgtcacattgccgactgatgaagacagcatccttgaatatgtaaaaggagcggcccgtgtatga

SEQ ID NO: 320 - DNA, 906 bp, Bacillus subtilis
ttgcctattaatataccaacacacctgccggcaaaacaggtgcttgaaagtgaacatatatttgtaatggatgaaagcagagcatttcatcaggatatccggccgcaaaaaattattatattaaatttgatgccgaaaaaaatccaaacagagactcagcttctcagattgcttggaaattcgcctttacaggttcatttcacatttttaatcccaagcacacacacgccgaaaaatactgcgagagaacaccttgacgaattttacacgactttttccaatatccgtcacaagaggtttgacggcatgattatcaccggtgcgccgatcgagcatttggcgtttgaagaggtttcatattgggaagagctcaaggaaatcatggagtggagcaaaacaaatgtcacttcaaccttgcatatttgctggggtgctcaagcaggtttgtattaccattacggtgtggaaaaaattcaaatgccgaaaaaaatcttcggtgtgttcgaacataccgttctttcaaaacacgaaagattggtaagaggttttgacgagctatactacgtgccgcattcccggcatacagatataaatatggaacagcttcaagcagtgcccgagttaaacattcttaccgcatcaaaggaagccggggtttgcttaattgtgtctaaagacgaaaaacaagtctttttgacagggcaccctgaatatgatacaaatacgcttcttcaagagtatgaaagagatttggaacgaaacctttctactgtcgaggcccctaaacattattttgcaaaaggcagcaacgagccagtgaatcgctggaaagcgcatgccacattattgtttatgaattggctgaattattacgtttaccaagaaactccttatgaatgggactaa

SEQ ID NO: 321 - DNA, 750 bp, Bacillus subtilis
atgcttgaaaaaatcatcaaacaaaagaaagaagaagtgaaaacactggttctgccggtagagcagcctttcgagaaacgttcatttaaggaggcgctggcaagcccgaatcggtttatcgggttgattgccgaagtgaagaaagcatcgccgtcaaaagggcttattaaagaggattttgtacctgtgcagattgcaaaagactatgaggctgcgaaggcagatgcgatttccgttttaacagacaccccgttttttcaaggggaaaacagctatttatcagacgtaaagcgtgctgtttcgattcctgtacttagaaaagattttattgattctcttcaagtagaggaatcaagaagaatcggagcggatgccatattgttaatcggcgaggtgcttgatcccttacaccttcatgaattatatcttgaagcaggtgaaaaggggatggacgtgttagtggaggttcatgatgcatcaacgctagaacaaatattgaaagtgttcacacccgacattctcggcgtaaataatcgaaacctaaaaacgtttgaaacatctgtaaagcagacagaacaaatcgcatctctcgttccgaaagaatccttgcttgtcagcgaaagcggaatcggttctttagaacatttaacatttgtcaatgaacatggggcgcgagctgtacttatcggtgaatcattgatgagacaaacttctcagcgtaaagcaatccatgctttgtttagggagtga

SEQ ID NO: 322 - DNA, 858 bp, Bacillus subtilis
atgaaagtcggttatttaggtccagcagctacatttacacatctagcagtcagttcttgttttcaaaacggcgccgaacatgttgcttaccgcaccattccggagtgtatagatgcagccgttgcaggcgaagttgattttgcttttgttcctttggaaaatgctttagaaggatctgttaatctaacaatagactatttaatacatgaacagcctttgccaatcgtgggtgaaatgacgttgccgattcaccagcacttgctcgtccatccctcaagagagaatgcatggaaagagctcgacaaaatttactcacattcacacgcgattgcgcaatgccataaatttcttcatcgacattttccttccgttccatatgaatacgccaattctaccggggcggcagcaaagtttgtcagtgaccatcccgagctgaatatcggggtcattgccaatgatatggcagcttctacatacgaattaaaaatcgtgaaacgggatatacaggattatagggacaatcatacaagatttgttatcctgtctcccgatgaaaacatatcttttgaagtgaattcaaaattgagctctaggcccaaaacgaccttaatggtcatgctgccgcaggatgatcagtccggggcgctgcatagagtgctgtctgcattttcttggagaaatttaaacctgtcaaaaattgagtcacgtccgactaaaaccggattaggccattatttctttattattgatattgagaaagcgtttgatgatgtattgattccaggggccatgcaggagcttgaagcactcggctgcaaagtgaggcttctgggtgcataccagtcttaccaattataa

SEQ ID NO: 323 - DNA, 1056 bp, Bacillus thuringiensis
atgtataaaggactattaaaacaatatgcgtcttatttaccggtgaatgaaaatacacctgatgtcagcttaatggaaggaaacacaccacttattccattattaaatatatcaaaacaattagggattcagctatatggtaagtacgaaggagcgaatccgacaggttcttttaaagatcgtggtatggtcatggcagttgctaaggcgaaagaagaaggttcagaagcaatcatttgtgcatcaacaggtaatacatcagcgtcagctgcagcatatgcggcacgtcttgggatgaaatgcattatcgtaataccagaaggaaagattgcacatggaaaattagcgcaagcggtcgcttatggagctgaaattatttcaatagaagggaatttcgatgatgcgcttaaggctgtaagaaatattgctgcagaagagccgattacgttagtaaactcagtaaatccttatcgaattgaagggcaaaaaacagcagcgtttgaaatttgtgaccaattgcaaaatgcaccagatgttctagctatccctgttgggaatgcagggaatattacagcgtattggaaaggcttctgtgaatatgagaaagaaaaaggttataaaaagccaagaattcatggctttgaagcggaaggagcagctgcaattgtaaaagggcatgtaattgaagagcctgaaacaattgcaacagcgattcgcatcggtaacccagcaagttggtcgtatgcagtagaggctgccgagcagtctcgtggtgaaatagatatggtatcagatgaagaaattttacatgcgtataggttattagcaaaaactgaaggagttttcgctgaaccaggatcaaatgcttcattagccggcgtaattaaacatgttcaatctgggaaaatcaaaaagggagaaacagttgttgcggtattaactggaaatggtttgaaggatcctgatatcgctatttcttcaaatcaactagacatcgcaagtgtgtcaaatgatatagaacaaattaaagatcatattaaaggggtgattatgtcg

SEQ ID NO: 324 - DNA, 903 bp, Bacillus thuringiensis
atgccgatcataattgataaagatttaccggctcgcaaagtgttacaaaaagaaaatatttttgtaatgacgaaggagcgagcggaaacacaagatatacgcgctttgaaaattgctatattaaatttaatgccaacgaagcaagacacagaggcacaattacttcgcttaattggaaacacaccgttacaattagatgttcatttgcttcatatggaatcacatctatctcgtaatgtaacgcaagaacatttaacaagcttttataaaacgtttcgtgatattgaaaatgaaaagtttgatgggctcattattacaggagcgccagtggagacccttgcttttgaagaggtagattattgggaagagcttaaacatattatggagtattcaaaaacgaatgtaacatctacgctccatatttgctggggagcacaggctggcttgtactatcattatggtgttccaaagtatccccttaaggaaaaaatgttcggtgtatttgaacatgaagtttgcgagcaacatgtgaaattgttacaaggatttgatgaattgttttttgctccgcattctcgtcatacagaagtacgagagaatgatattagaggagtgaaagacttaacattgttagcaaactctgaagaagcaggtgttcaccttgttattgggccagaaggaaagcaagtgtttgcccttggacatagcgaatatagttgtgagacgttaaaacaagaatacgaacgagatcgtgacaagggattaaatattgatgtgccaaaaaactattttaaacataataatccagatgaaaagccgcttgttaggtggaggagtcatggtaatttattgttttctaattggttgaattattacgtgtatcaagaaaccccttatatattg

SEQ ID NO: 325 - DNA, 759 bp, Bacillus thuringiensis
gtggggacgattttagataaaattgtagaacagaagaaaaaggaagttgcggagttatatgaaatatatacaccagtaaaaacaaaaagaaagacacattcacttgtagaagcgttacagcagtttactgtcattgcagaagtaaagcgagcatcaccatcaaaaggagatatcaatttacacgttgacgtacgaaaacaagtgggaacatatgagaaatgtggtgcaggagcagtatctgttttaacagatggtcaattttttaaaggatcgtttcacgatttacaaacagcaagagaagaaagtaacattccgcttttatgtaaagatttcataatcgataagattcaaatcgatagagcgtatgaagcgggagcagatattattttactaatcgtagcagcgttaacgaaagagaagttaaaagagttgtatagctacgtgttagaaaaaggattggaagcgattgttgaagttcataatgaacaggaattagaaactgcgattgtattaaatccgcacgttattggtattaataaccgtaatttaaaaacgttcgaagtagatttaagccaaactgaaaagcttggaaaaagattgaatgaggagaaattactttggattagtgaaagcgggattcattcaaaagaggatattattcgtgttaaaaaagcaggagcaaaaggtgtattaattggagaggcacttatgacatcatcttctattagtaacttttttgaagactgtaaggtgaatata

SEQ ID NO: 326 - DNA, 849 bp, Bacillus thuringiensis
atgattcgagtaggatatttaggaccagaagcaacatttacaaatatggcggtgagtcgtttttttccggaagcagagcatgtaccatatcgaacgattccagattgtatagatgcagctgcaaatgaaaatgtagacttcgcagttgtaccgttagaaaatgcaatagaaggttcagtgaatataacggttgattaccttgtacatgagcagccgctttccattgtaggagaaattacagtaccaattcagcagcatttacttgtacatccgcagtatgcagaagtgtgggatgaagtatatgcagtgcattctcatccgcatgccattgcacagtgccataaatttttaaatgaagaattaaaaggagtaactgttcgagatatgacatctacaagtgctgctgcgcaatatgtgaaagaacatcctgaagagaaaattgccgctattgcaaatgaagcggctgcagaaaaatacggattaacaattgtgagacgtagtattcatacgcataaaaataatcacacacgtttcttggtactccataagaaaaagaaagcagtactcccgaataacggagagaatcgcggagagaaaacgacgcttatgataacgttacctgcagattatgcaggtgccctatatcaagtgttatcagctttcgcatggagaaaattaaacttatcaaaaatcgaatcgcgtccgatgaaaacaggtcttggaaattactttttcttaattgatgttgataaagcatacgatgacgtattattgccaggcgtaacgatggaacttgaagcactcggtttttctgttactgtgctggggagttattcttcttattggttg

SEQ ID NO: 327 - DNA, 663 bp, Saccharomyces cerevisiae
atgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtag

SEQ ID NO: 328 - DNA, 1095 bp, Saccharomyces cerevisiae
atgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgttccacttccagatgaggcgctggaagcctccaagaaggctgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaa

SEQ ID NO: 329 - DNA, 4179 bp, Saccharomyces cerevisiae
atgactaacgaaaaggtctggatagagaagttggataatccaactctttcagtgttaccacatgactttttacgcccacaacaagaaccttatacgaaacaagctacatattcgttacagctacctcagctcgatgtgcctcatgatagtttttctaacaaatacgctgtcgctttgagtgtatgggctgcattgatatatagagtaaccggtgacgatgatattgttctttatattgcgaataacaaaatcttaagattcaatattcaaccaacgtggtcatttaatgagctgtattctacaattaacaatgagttgaacaagctcaattctattgaggccaatttttcctttgacgagctagctgaaaaaattcaaagttgccaagatctggaaaggacccctcagttgttccgtttggcctttttggaaaaccaagatttcaaattagacgagttcaagcatcatttagtggactttgctttgaatttggataccagtaataatgcgcatgttttgaacttaatttataacagcttactgtattcgaatgaaagagtaaccattgttgcggaccaatttactcaatatttgactgctgcgctaagcgatccatccaattgcataactaaaatctctctgatcaccgcatcatccaaggatagtttacctgatccaactaagaacttgggctggtgcgatttcgtggggtgtattcacgacattttccaggacaatgctgaagccttcccagagagaacctgtgttgtggagactccaacactaaattccgacaagtcccgttctttcacttatcgcgacatcaaccgcacttctaacatagttgcccattatttgattaaaacaggtatcaaaagaggtgatgtagtgatgatctattcttctaggggtgtggatttgatggtatgtgtgatgggtgtcttgaaagccggcgcaaccttttcagttatcgaccctgcatatcccccagccagacaaaccatttacttaggtgttgctaaaccacgtgggttgattgttattagagctgctggacaattggatcaactagtagaagattacatcaatgatgaattggagattgtttcaagaatcaattccatcgctattcaagaaaatggtaccattgaaggtggcaaattggacaatggcgaggatgttttggctccatatgatcactacaaagacaccagaacaggtgttgtagttggaccagattccaacccaaccctatctttcacatctggttccgaaggtattcctaagggtgttcttggtagacatttttccttggcttattatttcaattggatgtccaaaaggttcaacttaacagaaaatgataaattcacaatgctgagcggtattgcacatgatccaattcaaagagatatgtttacaccattatttttaggtgcccaattgtatgtccctactcaagatgatattggtacaccgggccgtttagcggaatggatgagtaagtatggttgcacagttacccatttaacacctgccatgggtcaattacttactgcccaagctactacaccattccctaagttacatcatgcgttctttgtgggtgacattttaacaaaacgtgattgtctgaggttacaaaccttggcagaaaattgccgtattgttaatatgtacggtaccactgaaacacagcgtgcagtttcttatttcgaagttaaatcaaaaaatgacgatccaaactttttgaaaaaattgaaagatgtcatgcctgctggtaaaggtatgttgaacgttcagctactagttgttaacaggaacgatcgtactcaaatatgtggtattggcgaaataggtgagatttatgttcgtgcaggtggtttggccgaaggttatagaggattaccagaattgaataaagaaaaatttgtgaacaactggtttgttgaaaaagatcactggaattatttggataaggataatggtgaaccttggagacaattctggttaggtccaagagatagattgtacagaacgggtgatttaggtcgttatctaccaaacggtgactgtgaatgttgcggtagggctgatgatcaagttaaaattcgtgggttcagaatcgaattaggagaaatagatacgcacatttcccaacatccattggtaagagaaaacattactttagttcgcaaaaatgccgacaatgagccaacattgatcacatttatggtcccaagatttgacaagccagatgacttgtctaagttccaaagtgatgttccaaaggaggttgaaactgaccctatagttaagggcttaatcggttaccatcttttatccaaggacatcaggactttcttaaagaaaagattggctagctatgctatgccttccttgattgtggttatggataaactaccattgaatccaaatggtaaagttgataagcctaaacttcaattcccaactcccaagcaattaaatttggtagctgaaaatacagtttctgaaactgacgactctcagtttaccaatgttgagcgcgaggttagagacttatggttaagtatattacctaccaagccagcatctgtatcaccagatgattcgtttttcgatttaggtggtcattctatcttggctaccaaaatgatttttaccttaaagaaaaagctgcaagttgatttaccattgggcacaattttcaagtatccaacgataaaggcctttgccgcggaaattgacagaattaaatcatcgggtggatcatctcaaggtgaggtcgtcgaaaatgtcactgcaaattatgcggaagacgccaagaaattggttgagacgctaccaagttcgtacccctctcgagaatattttgttgaacctaatagtgccgaaggaaaaacaacaattaatgtgtttgttaccggtgtcacaggatttctgggctcctacatccttgcagatttgttaggacgttctccaaagaactacagtttcaaagtgtttgcccacgtcagggccaaggatgaagaagctgcatttgcaagattacaaaaggcaggtatcacctatggtacttggaacgaaaaatttgcctcaaatattaaagttgtattaggcgatttatctaaaagccaatttggtctttcagatgagaagtggatggatttggcaaacacagttgatataattatccataatggtgcgttagttcactgggtttatccatatgccaaattgagggatccaaatgttatttcaactatcaatgttatgagcttagccgccgtcggcaagccaaagttctttgactttgtttcctccacttctactcttgacactgaatactactttaatttgtcagataaacttgttagcgaagggaagccaggcattttagaatcagacgatttaatgaactctgcaagcgggctcactggtggatatggtcagtccaaatgggctgctgagtacatcattagacgtgcaggtgaaaggggcctacgtgggtgtattgtcagaccaggttacgtaacaggtgcctctgccaatggttcttcaaacacagatgatttcttattgagatttttgaaaggttcagtccaattaggtaagattccagatatcgaaaattccgtgaatatggttccagtagatcatgttgctcgtgttgttgttgctacgtctttgaatcctcccaaagaaaatgaattggccgttgctcaagtaacgggtcacccaagaatattattcaaagactacttgtatactttacacgattatggttacgatgtcgaaatcgaaagctattctaaatggaagaaatcattggaggcgtctgttattgacaggaatgaagaaaatgcgttgtatcctttgctacacatggtcttagacaacttacctgaaagtaccaaagctccggaactagacgataggaacgccgtggcatctttaaagaaagacaccgcatggacaggtgttgattggtctaatggaataggtgttactccagaagaggttggtatatatattgcatttttaaacaaggttggatttttacctccaccaactcataatgacaaacttccactgccaagtatagaactaactcaagcgcaaataagtctagttgcttcaggtgctggtgctcgtggaagctccgcagcagcttaa

SEQ ID NO: 330 - DNA, 1335 bp, Saccharomyces cerevisiae
atgccatctcatttcgatactgttcaactacacgccggccaagagaaccctggtgacaatgctcacagatccagagctgtaccaatttacgccaccacttcttatgttttcgaaaactctaagcatggttcgcaattgtttggtctagaagttccaggttacgtctattcccgtttccaaaacccaaccagtaatgttttggaagaaagaattgctgctttagaaggtggtgctgctgctttggctgtttcctccggtcaagccgctcaaacccttgccatccaaggtttggcacacactggtgacaacatcgtttccacttcttacttatacggtggtacttataaccagttcaaaatctcgttcaaaagatttggtatcgaggctagatttgttgaaggtgacaatccagaagaattcgaaaaggtctttgatgaaagaaccaaggctgtttatttggaaaccattggtaatccaaagtacaatgttccggattttgaaaaaattgttgcaattgctcacaaacacggtattccagttgtcgttgacaacacatttggtgccggtggttacttctgtcagccaattaaatacggtgctgatattgtaacacattctgctaccaaatggattggtggtcatggtactactatcggtggtattattgttgactctggtaagttcccatggaaggactacccagaaaagttccctcaattctctcaacctgccgaaggatatcacggtactatctacaatgaagcctacggtaacttggcatacatcgttcatgttagaactgaactattaagagatttgggtccattgatgaacccatttgcctctttcttgctactacaaggtgttgaaacattatctttgagagctgaaagacacggtgaaaatgcattgaagttagccaaatggttagaacaatccccatacgtatcttgggtttcataccctggtttagcatctcattctcatcatgaaaatgctaagaagtatctatctaacggtttcggtggtgtcttatctttcggtgtaaaagacttaccaaatgccgacaaggaaactgacccattcaaactttctggtgctcaagttgttgacaatttaaagcttgcctctaacttggccaatgttggtgatgccaagaccttagtcattgctccatacttcactacccacaaacaattaaatgacaaagaaaagttggcatctggtgttaccaaggacttaattcgtgtctctgttggtatcgaatttattgatgacattattgcagacttccagcaatcttttgaaactgttttcgctggccaaaaaccatga

SEQ ID NO: 331 - DNA, 804 bp, Saccharomyces cerevisiae
atgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaa

SEQ ID NO: 332 - DNA, 429 bp, Bacillus subtilis
atggcggtcgtgagagcaacgagtgcggatgtcgatttgatggcaaggctgctcagagcggaagcggaaggcgaaggcaagcaggggatgctgcttgtcggcaacgttggaattaatcggctgcgggcgaattgctcagattttaaaggcctccgcaccatcaggcagatgatttatcagccacacgcgtttgaggctgtgactcatggatatttttatcaaagggcgcgagatagcgagcgtgcccttgcacgccggtcgattaatggtgaaaggcgctggcctgcaaaatttagtttatggtacttcaggccgcagggggactgtccagcccagtggtataaccagccgtttgtggccagatttaagtcacactgcttttatcagccgacggcggagacgtgtgaaaatgtatataacacattttag

SEQ ID NO: 333 - DNA, 918 bp, Bacillus subtilis
atgaagtccaaaggatcgattatggcatgtctcatccttttttcctttacaataacgacgtttattaatactgaaacgatctctgccttttcgaatcaggtcattcaaagaggggcaacaggggatgatgtggtcgagcttcaggcgcgtcttcaatacaacggatattataacggaaaaattgacggggtttatggatgggggacgtactgggcagttcgaaattttcaggatcaattcgggttaaaagaggttgacggccttgtaggagctaaaacaaagcaaaccttaatatgtaaatcaaaatactatcgtgaatatgtcatggaacagctcaataaagggaatacattcacgcattacggaaaaattccgctaaagtatcagacgaaaccatcaaaagcagcaacacaaaaggcaagacaacaagcagaagcacggcagaaacagcctgcggaaaaaacaacgcagaagcctaaagcgaatgcgaataaacagcaaaacaatacaccagcaaaagcaagaaaacaggatgcggtagcagcgaacatgcctggtggattttccaacaacgatatcaggctgcttgctcaagcggtttatggcgaagcccggggcgagccgtacgaggggcaggttgctattgcagcagtcattttaaaccgtttgaacagcccgttatttccaaattcagtagcgggggttatttttgagccgcttgccttcacagcagtagccgacggacaaatttacatgcagccgaatgaaacggcacgagaagcagtgctggatgccatcaatggctgggacccatcagaggaagcactttactactttaatccggatacggctacaagtccgtggatttgggggcgtccgcagattaaaagaatcggtaaacacattttctgtgagtag

SEQ ID NO: 334 - DNA, 1098 bp, Bacillus subtilis
atgagccaaaaacagactccactgaaacttaatacctttcaaggtatttctattgttgcaaatacaatgcttggggccggacttttaacattgccgcgagcgctgaccactaaggctaatacgcctgacggctggatcacgctgatattagaaggtttcatttttattttcttcatttatttaaacacgctcattcagaaaaaacatcaatacccttcactctttgaatatttgaaggaagggcttgggaaatggatcggcagcatcatcggccttttgatctgcggctatttcctcggcgtagccagcttcgagacacgggcaatggctgaaatggtgaagtttttcctgctggagagaaccccaattcaagtcattattttaacgtttatttgctgcggcatttatttaatggttgggggcttaagcgatgtgtcgcggctgtttcccttttatttaacggtaaccattattattttgctgattgtgttcgggatcagttttaaaatttttgatatcaataatttgcgtcctgttttaggcgaaggccttggacccattgcaaactcccttaccgtcgtttccatctcttttttaggaatggaagtgatgctgtttcttcctgaacatatgaagaaaaagaaatacacgttcagatatgcgtctctaggatttctgattccgattatcttatatatccttacgtatattatcgttgtcggagctttgaccgctcccgaggtgaaaacgctgatttggccgactatttctctctttcagtcctttgagcttaaaggcatatttattgaacggtttgaatcctttttactggtggtctggattatccagtttttcaccacatttgtcatttacggatactttgccgctaacgggctaaagaagacatttggattatcgactaaaacaagcatggtgattatcggcataacggtcttttatttttctctctggccggatgacgccaatcaagtcatgatgtatagcgattatcttggctatatttttgtctccctgtttctgcttccgttcattctctttttcattgtagctctcaagaggaggattacaacaaaatga

SEQ ID NO: 335 - DNA, 1107 bp, Bacillus subtilis
atgaggaaatcagagcataaactgacatttatgcagacgctcattatgatcagcagcacattgattggtgccggggtgctgacccttccccgctcagccgccgaaaccggcagtccgagcggatggctaatgatactgctccagggcgttatttttattatcatcgttctgctttttttgccttttcttcaaaaaaacagcggaaaaactctttttaagctcaacagcattgtagctggaaaattcatcggctttctattgaatttatatatctgtctatatttcattgggattgtttgctttcaagctcggattttgggagaggttgtcggattctttttgttgaaaaatacgccaatggcagttgtggtgtttatatttcttgcagttgccatctatcatgtaggcggaggcgtttattcaattgcaaaagtatacgcttatatttttcctataacccttattatttttatgatgcttctgatgttcagctttcgcttgttccagcttgattttatccggccggtatttgaaggaggctatcaaagctttttctctttattcccaaaaacattattatatttctccggatttgaaatcattttttacctggtcccctttatgagagatccaaagcaagtgaaaaaggctgttgctctgggcatcgcgacttccacattgttctacagcattactttgctcattgtgattggctgtatgactgtggctgaggcaaaaacggtgacatggccgaccatttcccttattcacgcattagaggttccgggtatttttattgaacgatttgatttgttcttacagctgacctggacagcccagcaatttgcctgtatgctcggatcctttaaaggcgcccatatcgggttaacggaaatctttcatctaaaaaataaaaacaatgcatggctgctgactgcaatgctggctgcgacgtttttcataacgatgtatcccaaggacttgaatgacgtgttttattacgggacgcttcttggatatgctttcttaattgtgattacaattccattcttcgtctggtttttaagctggatacagaaaaaaatagggagggggcagctgcaatga

SEQ ID NO: 336 - DNA, 1122 bp, Bacillus subtilis
atggaaaaagccagaataagcataaggcagttgtttgtcatgattatcatttttgaactgggcagctccttattgattacaccgggatcaatggcgggcagggatgcttggatagcagttttattaggctgtgcgatcggactgtttcttttctatttgtatcaaggcatttatcaatgttatccgaattcttctccgaaagaatatatggatgatatgctgggaaccaagctgagctggctgttttcatttctctatatcttgtactttgcctatattgccgcaagggtgctgcgtgactttggagaaatgctgctgacgtttgcttatcatgatacacctatcattattgtaaatgctttattaatggtagtaagcatatatgccgtaagaaaaggaattgaagtactcgcacgcgcggccgaacttcttttcggggccatgtatttactgggcgcgatcggccttgtgctgattattgtctcagggactatagacccgcacaatttaaagccggttttggcaaacggcatttctcctgtccttcactctgttttcacacagaccatgtacgttccatttggtgaagttgttttatttgtgatgattttccctaacctgaatgacagaaaagatgtgaaaaaaatggggatgatcgccatggctattagcggattgattgtggcgctcaccgtggcgattaatattagcgtgcttgatgttgatcttacactcagatcccagtttccgcttttaagcacgatccagacaatcaaggtcgaagaatttttagaccggctcgatgtcttttttatgctggcgctgatcatcggcggcttctttaaggtcagcctctacttatatgccactgtggttgggacatctacgctctttaaggaaaagaacccctctcaattggcttatccgatgggattgggcatcttaatcctttctataacgatcgcgactaacttttcggagcacttgaatgaaggcctgaacgtggtgccgctgtatattcatttgccttttcaattattgtttccgctctttctgtttattgtggccgtttggaaaaagaaacgaagagagaagtcaaaaggggaggaagcaaaaaaatga

SEQ ID NO: 337 - DNA, 795 bp, Bacillus thuringiensis
atgaaattactaaaactaaaacatataatccctttatctgcagctgcaattacattcgtatgtagtcaaagcacagctgaagcttctaccattcatacagtaaaaaagaacgatacactttggggcattagtaaacaatacggagtttcaatacaatccattaaacaggctaataataaaggaaacgatcaaacctttattggcgaacagttacatattccagggtccgtgaaatcaaatgaaatcactgttcctcaaaacgccaagcctgcgaacatttctggacaaattatttatcaagtacaaccgggagattcattagaaacgatagcaaggcgttacaatgtcactgtccaatctataaaacaaattaacaatacaatcggaaacaagctttatacaggacaacatttaaaaatcaactcaagtatttcacaaaaagaaaaagacttaatggcacgcttagttaccgctgaagcaggtggcgaatcctataaaggaaaagtagcagtagcgaaagttatactaaatcgtgtaaatgcaaaaggttttccaaatacaataacaggcgttatttatgaacctattaaatacggatatgcatttactcctgttacagatggaagaattaatcagcctgcaagcgcagaagcaaaaatggctgtagaagaggctatctccacaaatggagtacattctgactggctttatttctacaatccaaaaacatcaacagacaagtggattacgacacgtcagacagtagcagtaattggtaatcatgtgtttgctaaa

SEQ ID NO: 338 - DNA, 777 bp, Bacillus thuringiensis
atgcgccaaaaagctatttttaaaatagcagttttacttgcgttcataggactgtctttaatggtcagtagtatacaattaaagaatgtagaggctttttctaatcaagttattcaaagaggagcatctggcgaagatgtcattgaactgcaatctcgtttgaaatataacgggttttatacgggaaaagtggatggtgttttcggatggggtacatattgggcacttcggaattttcaagagaaattcggattacccgttgatggtttagctggagctaaaacgaagcaaatgctcgtgaaggcaacgaagtatgacaagtcaactgctaataaagggacgacaacgaataaaggaaatagcggtggcactgcacaagagaataaaccacttcaaaataaagggacaaatgttccgaacggttattctcaaaatgatattcagcttatggcaaacgcagtatacggagaatcgcgtggtgaaccgtatttaggacaagttgcagtagctgccgttattttgaatcgtgttacaagtgcgtcattcccaaatactgtttcaggtgtaatctttgaacctagagcatttacagcggtagccgatggacaaatatatttaacgccaaatgaaacagcaaaaaaagcagttttagatgcgattaacggatgggatccaacaggaaatgctttatattatttcaatccagatactgcgactagtaaatggatttggactcgtccacaaattaaaaaaatcggtaaacatattttctgtaaa

SEQ ID NO: 339 - DNA, 1098 bp, Bacillus thuringiensis
atgagtaaagttcaagaaaaatatcaaatatctcctgtctttgttttcttcttaattcacggtgcacagtttggtgctggcgttctcggcttcgctcgaatcatcgcaaaggccgctggatacgacggatggatgggcgttctaatcacgggactctgtatccatctccttatatggatgatgtattttttattaaaagaaacaaatggaaatttaattgatttacaaaggcaaacgttcggaaagtggatagggaatatctttaatatcatctttgcagcttattttttaaacgtcagcatttcggttattcgaacatatgttgaaattattcaagtatggatgtttcctaccgcttccacctttatgttaacactctttttatgcttagttagctattacattatctcttctggatttcgtgtcattactggcatttgtgtcatctctgttggtggtacattaggatatttatttctaagcttattcgttttgaaatattcacattgggataatttactacctatattcacacattctttttcagacattttaaaatccgctcaactgtcaatctacagtatgaccggttttgagatttacctcatggtatatccatttgtgaaaaagcctatgcaatctcataaatacgctcagcttggagcactattttcaaatcttctatatttatttagcacccttttagcctttgcattttttagtgaaaaacaattattaaaaacgatatgggcacaactttctatgacccaagtcattcaattaccatttatagaaagactcgaatatattgcaatttcaggctacgcactcgttattatttcaagttttatcctccctttatgggcgtcaatgagagcaactcatgaaatattccgtgtaaaacaaagaggggttttaatcgcttttatccttatcaccctcattgtttcacaacttttaacaaacagacacgatattaataactttattagtaatgtatcaaaggttagtttttggctcatttatgtatacatcccaatcctatttattattgtgtgggtgaaaagaaaatggaaaaaatcaaaagcacaagcaaat

SEQ ID NO: 340 - DNA, 1104 bp, Bacillus thuringiensis
gtgacaaacacgcaaagtaaaatttcacttgttcaatttacctttttcgttattcaatgccaaattggggtcggtatactatctctacctaaccggcttcatccgatagcaaaaggtggtggttggatttctgcgctcatcgcggggcttgccattcaacttattattttacttatgtggcttttcttaaaacgatttcctgatgcagatatgtataaaagtgtttgtatgctatttggtaataagttaggaaaactattgggcttttcttatgttttttattttacccttatcggtatgacggttatgttgaatgcttgtaacgtcattaaagtatgggttttacacgcaactccgtggtctgcaattctcatattatgtacagtagcttgttgttatgtagcttataacacatttaaagtcatcgtacgattttatgtcatggcctctattctcattattccaatggcccttttaattgccctagggctttcacgagctgatttctcttatatttttcctattacagaagccggatggtggaatataattcaagcatccaaggaaacaattacagccatgtatggttttgaaattattcttatcgctttcccaaaagtaaatgggagttctattgcaaaactgaaggccatttctatcgccaatggatttgtaactcttttttatacttttaccgtttggatttgctttatcgtatttagtccaaagcaaattgagcttattccagagccagttgcttacttattacgctccttgcatattggaattatagatcgaacggacctaatcttcatcccaatttggatgataacagttgtcgcttctattgctagttattactgcgctgcttcaatcggaattggacatatattcaaccttgctaatcataaaaaggctgttccaatcgttggtattattgcttttagtgtagccttatttatagatacacctgaagaattaaaagtaatttcaactttcactgataagttcacctatattttcatcgttgtcctccctcttttatttctactttattcaataataagaaagaagaaaggagaacagtatgttcccaaaaagagt

SEQ ID NO: 341 - DNA, 28 bp, Artificial Sequence
  Description of Artificial Sequence Synthetic oligonucleotide
tcgtaaggacgatacccgaatgttaaac

SEQ ID NO: 342 - DNA, 28 bp, Artificial Sequence
  Description of Artificial Sequence Synthetic oligonucleotide
tctggctgacacggactagtaatattgc

SEQ ID NO: 343 - DNA, 28 bp, Artificial Sequence
  Description of Artificial Sequence Synthetic oligonucleotide
  modified_base (1)..(3): a, c, t, g, unknown or other
nnnggggcaacggacgtgaggcgtcatc

SEQ ID NO: 344 - DNA, 28 bp, Artificial Sequence
  Description of Artificial Sequence Synthetic oligonucleotide
tagggggcaacggacgtgaggcgtcatc

SEQ ID NO: 345 - DNA, 26 bp, Artificial Sequence
  Description of Artificial Sequence Synthetic oligonucleotide
aagtcgacctgcaggcatgcaagctt

SEQ ID NO: 346 - DNA, 24 bp, Artificial Sequence
  Description of Artificial Sequence Synthetic oligonucleotide
aagtcgacctgcaggcatgcaatt

SEQ ID NO: 347 - DNA, 25 bp, Artificial Sequence
  Description of Artificial Sequence Synthetic oligonucleotide
aagtcgacctgcagcatgcaagctt

SEQ ID NO: 348 - DNA, 28 bp, Artificial Sequence
  Description of Artificial Sequence Synthetic oligonucleotide
gctttgtctacgaaatgccaacttttgt

SEQ ID NO: 349 - DNA, 28 bp, Artificial Sequence
  Description of Artificial Sequence Synthetic oligonucleotide
gctctatttacccgcgagcctaccagtg

SEQ ID NO: 350 - DNA, 28 bp, Artificial Sequence
  Description of Artificial Sequence Synthetic oligonucleotide
aaggtcagcgggactgacgtgaaggtcc

SEQ ID NO: 351 - DNA, 28 bp, Artificial Sequence
  Description of Artificial Sequence Synthetic oligonucleotide
  modified_base (6)..(8): a, c, t, g, unknown or other
  modified_base (10)..(10): a, c, t, g, unknown or other
  modified_base (13)..(19): a, c, t, g, unknown or other
  modified_base (21)..(21): a, c, t, g, unknown or other
  modified_base (25)..(25): a, c, t, g, unknown or other
  modified_base (27)..(28): a, c, t, g, unknown or other
atctgnnncnacnnnnnnnangttntnn

SEQ ID NO: 352 - DNA, 160 bp, Bacillus thuringiensis
gtccgagaaagtcaaacatgcgaagcgacttagtgatgagcggaatttacttcaagatccaaactttagagggatcaatagacaactagaccgtggctggagaggaagtacggatattaccatccaaggaggcgatgacgtattcaaagagaattacgtt

SEQ ID NO: 353 - DNA, 123 bp, Bacillus thuringiensis
caaacatgcgaagcgacttagtgatgagcggaatttacttcaagatccaaactctagagggatcaatagacaactagaccgtggctggagaggaagtacggatattaccatccaaggaggcgg

SEQ ID NO: 354 - DNA, 121 bp, Bacillus thuringiensis
caaacatgcgaagcgacttagtgatgagcggaatttacttcaagatccaaactttagagggatcaatagacaactagaccgtggctggagaggaagtacggatattaccatccaaggaggc

SEQ ID NO: 355 - DNA, 123 bp, Bacillus thuringiensis
caaacatgcgaagcgacttagtgatgagcggaatttacttcaagatccaaactttagagggatcaatagacaactagaccgtggctggagaggaagtacggatattaccatccaaggaggcga

SEQ ID NO: 356 - DNA, 118 bp, Bacillus thuringiensis
catgcgaagcgacttagtgatgagcggaatttacttcaagatccaaactctagagggatcaatagacaactagaccgtggctggagaggaagtacggatattaccatccaaggaggcg

SEQ ID NO: 357 - DNA, 124 bp, Bacillus thuringiensis
tcaaacatgcgaagcgacttagtgatgagcggaatttacttcaagatccaaacttcagagggatcaatagacaactagaccgtggctggagaggaagtacggatattaccatccaaggaggcga

SEQ ID NO: 358 - DNA, 120 bp, Bacillus thuringiensis
acatgcgaagcgacttagtgatgagcggaatttacttcaagatccaaacttcagaggggtcaatagacaactagaccgtggctggagaggaagtacggatattaccatccaaggaggcga